(19) United States
(12) Patent Application Publication
(10) Pub. No.: US 2024/0181028 A1
(43) Pub. Date: Jun. 6, 2024

(54) Immunogenic compositions comprising conjugated capsular saccharide antigens and uses thereof

(71) Applicant: Pfizer Inc., New York, NY (US)

(72) Inventors: Catherine Alex, Westwood, NJ (US); Annaliesa Sybil Anderson, Upper Saddle River, NJ (US); Bishwa Raj Bhetuwal, Nanuet, NY (US); Zecheng Chen, New City, NY (US); Kaushik Dutta, River Edge, NJ (US); Caitlyn Gallagher, Ringwood, NJ (US); Jianxin Gu, Paramus, NJ (US); Isis Kanevsky, New York, NY (US); Jin-Hwan Kim, Suffern, NY (US); Justin Keith Moran, West Nyack, NY (US); Suddham Singh, Monroe, NY (US); Naveen Surendran, Dumont, NJ (US); Abhishek Ravindra Vartak, Westwood, NJ (US); YuYing Yang, Stamford, CT (US)

(21) Appl. No.: 18/513,738

(22) Filed: Nov. 20, 2023

Related U.S. Application Data
(60) Provisional application No. 63/593,565, filed on Oct. 27, 2023.
(60) Provisional application No. 63/384,626, filed on Nov. 22, 2022.

(51) Int. Cl. A61K 39/09; A61K 47/64

(52) U.S. Cl. CPC A61K 39/092; A61K 47/646

(57) ABSTRACT

The present invention relates to new immunogenic compositions comprising conjugated Streptococcus pneumoniae capsular saccharide antigens (glycoconjugates) and uses thereof. Immunogenic compositions of the present invention will typically comprise at least one glycoconjugate from a S. pneumoniae serotype not found in Prevnar, Synflorix and/or Prevnar 13. The invention also relates to vaccination of human subjects, in particular infants and elderly, against pneumoccocal infections using said novel immunogenic compositions.

### REFERENCE TO SEQUENCE LISTING

The instant application contains a sequence listing which has been submitted electronically in .xml format and is hereby incorporated by reference in its entirety. The .xml file, named “PC072837A Sequence Listing.xml”, was created on Nov. 13, 2023, and is 5 KB in size.

### FIELD OF THE INVENTION

The present invention relates to new immunogenic compositions comprising conjugated capsular saccharide antigens (glycoconjugates) and uses thereof. Immunogenic compositions of the present invention will typically comprise glycoconjugates, wherein the saccharides are derived from serotypes of Streptococcuspneumoniae. The invention also relates to vaccination of human subjects, in particular infants and elderly, against pneumoccocal infections using said novel immunogenic compositions.

### BACKGROUND OF THE INVENTION

Infections caused by pneumococci are a major cause of morbidity and mortality all over the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

In Europe and the United States, pneumococcal pneumonia is the most common community-acquired bacterial pneumonia, estimated to affect approximately 100 per 100,000 adults each year. The corresponding figures for febrile bacteraemia and meningitis are 15-19 per 100 000 and 1-2 per 100,000, respectively. The risk for one or more of these manifestations is much higher in infants and elderly people, as well as immune compromised persons of any age. Even in economically developed regions, invasive pneumococcal disease carries high mortality; for adults with pneumococcal pneumonia the mortality rate averages 10%-20%, whilst it may exceed 50% in the high-risk groups. Pneumonia is by far the most common cause of pneumococcal death worldwide.

The etiological agent of pneumococcal diseases, Streptococcus pneumoniae (pneumococcus), is a Gram-positive encapsulated coccus, surrounded by a polysaccharide capsule. Differences in the composition of this capsule permit serological differentiation between about 91 capsular types, some of which are frequently associated with pneumococcal disease, others rarely. Invasive pneumococcal infections include pneumonia, meningitis and febrile bacteremia; among the common non-invasive manifestations are otitis media, sinusitis and bronchitis.

Pneumococcal conjugate vaccines (PCVs) are pneumococcal vaccines used to protect against disease caused by S. pneumoniae (pneumococcus). There are currently five PCV vaccines available on the global market: Prevnar® (called Prevenar in some countries) (heptavalent vaccine), SYNFLORIX® (a decavalent vaccine), Prevnar 13® (tridecavalent vaccine), Vaxneuvance™ (a 15-valent vaccine) and Prevnar 20™ (a 20-valent vaccine).

The recent development of widespread microbial resistance to essential antibiotics and the increasing number of immunocompromised persons underline the need for pneumococcal vaccines with even broader protection.

In particular, there is a need to address remaining unmet medical need for coverage of pneumococcal disease due to serotypes not found in Prevnar 13® and potential for serotype replacement over time. The specific serotypes causing disease beyond the 13 in Prevnar 13® vary by region, population, and may change over time due to acquisition of antibiotic resistance, pneumococcal vaccine introduction and secular trends of unknown origin. There is a need for immunogenic compositions that can be used to induce an immune response against additional Streptococcus pneumoniae serotypes in humans and in particular in children less than 2 years old.

An object of the new immunogenic compositions of the present invention is to provide for appropriate protection against additional S. pneumoniae serotypes not found in Prevnar 13®. In one aspect, an object of the immunogenic compositions of the present invention is to provide for appropriate protection against additional S. pneumoniae serotypes not found in PREVNAR® (heptavalent vaccine), SYNFLORIX® and/or PREVNAR 13® while maintaining an immune response against serotypes currently covered by said vaccines.

### FIGURES

FIG. 1 shows a repeating polysaccharide structure of S. pneumoniae serotype 23A (Pn-23A) capsular polysaccharide.

FIG. 2 shows 1D 1H proton spectra of Pn-23A polysaccharide sized using sonication (mechanical) and acid hydrolysis done for 2 hours (2 hrs), 4 hours (4 Hrs) and 6 hours (6 Hrs). Left panel: anomeric region; Middle panel: increased threshold of the anomeric region; Right panel: methyl region of 1H spectrum of Pn-23A polysaccharide. The anomeric and methyl signals are annotated.

FIG. 3 shows the change in the normalized intensity of selected resonances chosen from each sugar in the repeating unit of Pn-23A polysaccharide.

FIG. 4 Right panel: 1D ³¹P spectra of Pn-23A polysaccharide sized using sonication (mechanical) and acid hydrolysis done for 2 hours (2 hrs), 4 hours (4 hrs) and 6 hours (6 hrs). Left panel: 2D ¹H-³¹P HMBC spectrum of hydrolyzed (6 hrs) Pn-23A polysaccharide. The phosphorus-carbon correlations in different populations are annotated using dotted lines.

FIG. 5 Structural changes observed upon hydrolysis of Pn-23A polysaccharide.

FIG. 6 1D ¹H proton spectra of Pn-23A polysaccharide sized using sonication (Sized Sonication), homogenization (Sized Homogenized) and hydrolysis done for 2 hrs (Hydrolized (2 hrs)). Left panel: anomeric region; Middle panel: increased threshold of the anomeric region; Right panel: methyl region of ¹H spectrum of Pn-23A polysaccharide. The anomeric and methyl signals are annotated.

FIG. 7 shows a repeating polysaccharide structure of S. pneumoniae serotype 24F (Pn-24F) capsular polysaccharide.

FIG. 8 shows 1D ¹H proton spectrum of Pn-24F polysaccharide following hydrolysis. The anomeric and methyl signals are annotated. The table shows the excellent agreement between the normalized and expected peak area for the anomeric and methyl protons except for the residue E1 suggesting a loss of Ribf sugar.

FIG. 9 shows 1D ¹H proton spectrum of Pn-24F polysaccharide following hydrolysis. The anomeric and methyl signals are annotated. The table shows the excellent agreement between the normalized and expected peak area fo the anomeric and methyl protons (but for the Ribfsugar (E1)).

FIG. 10 shows 10 ¹H proton spectra of Pn-24F polysaccharide either mechanically sized (lower spectra) or hydrolyzed using two different conditions (upper and middle spectra). The anomeric and methyl signals are annotated in the reference spectra. The tables show the 1D ¹H NMR signal of the anomeric protons.

FIG. 11 Structural changes seen in Pn-24F polysaccharide after hydrolysis.

### 1 Glycoconjugates of the Invention

The present invention is directed in part to conjugated capsular saccharide antigens (also named glycoconjugates). For the purpose of the invention the term ‘glycoconjugate’ indicates a capsular saccharide conjugated to a carrier protein via covalent or non-covalent bonds. In an embodiment, the capsular saccharide is conjugated to a carrier protein via non-covalent bonds (such as the rhizavidin/biotin system, see e.g. WO2012155007, WO2020056202). Preferably, the capsular saccharide is conjugated via covalent bonds. In one embodiment the capsular saccharide is conjugated directly to a carrier protein. In a second embodiment the capsular saccharide is conjugated to a carrier protein through a spacer/linker.

The term “saccharide” throughout this specification may indicate polysaccharide or oligosaccharide and includes both. In frequent embodiments, the saccharide is a polysaccharide, in particular a S. pneumoniae capsular polysaccharide.

S. pneumoniae capsular saccharides can be prepared by techniques known to those of ordinary skill in the art (see for example methods disclosed in US2006/0228380, US2006/0228381, US2008/0102498, WO2008/118752 and WO2020170190). Typically, capsular polysaccharides are produced by growing each S. pneumoniae serotype in a medium (e.g., in a soy-based medium), the polysaccharides are then prepared from the bacteria culture. Bacterial strains of S. pneumoniae used to make the respective polysaccharides that are used in the glycoconjugates of the invention may be obtained from established culture collections (such as for example from the Streptococcal Reference Laboratory (Centers for Disease Control and Prevention, Atlanta, GA USA)) or clinical specimens.

The population of the organism (each S. pneumoniae serotype) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see for example WO 2006/110381, WO 2008/118752, and U.S. Patent App. Pub. Nos. 2006/0228380, 2006/0228381, 2008/0102498 and 2008/0286838).

The individual polysaccharides are typically purified through centrifugation, precipitation, ultra-filtration, and/or column chromatography (see for example WO 2006/110352, WO 2008/118752 and WO2020170190).

Purified polysaccharides may be activated (e.g., chemically activated) to make them capable of reacting (e.g., either directly to the carrier protein of via a linker such as an eTEC spacer) and then incorporated into glycoconjugates of the invention, as further described herein.

S. pneumoniae capsular polysaccharides comprise repeating oligosaccharide units which may contain up to 8 sugar residues.

In an embodiment, capsular saccharide of the invention may be one oligosaccharide unit, or a shorter than native length saccharide chain of repeating oligosaccharide units. In an embodiment, capsular saccharide of the invention is one repeating oligosaccharide unit of the relevant serotype.

In an embodiment, capsular saccharide of the invention may be oligosaccharides. Oligosaccharides have a low number of repeat units (typically 5-15 repeat units) and are typically derived synthetically or by hydrolysis of polysaccharides.

In an embodiment, the capsular saccharides of the present invention are polysaccharides. High molecular weight capsular polysaccharides are able to induce certain antibody immune responses due to the epitopes present on the antigenic surface. The isolation and purification of high molecular weight capsular polysaccharides is preferably contemplated for use in the conjugates, compositions and methods of the present invention.

### 1.1 S. pneumoniae Serotype 15A Glycoconjugate of the Invention

In an aspect the invention relates to S. pneumoniae serotype 15A glycoconjugates.

The structure of Streptococcus pneumoniae serotype 15A polysaccharide is known in the art (see e.g. Geno K et al. (2015) Clin Microbiol Rev Vol 28:3, p 871-899).

In an embodiment, the capsular S. pneumoniae serotype 15A saccharide used in the present invention is a synthetic carbohydrate.

In a preferred embodiment though, the source of bacterial polysaccharide according to this invention can be Streptococcus pneumoniae serotype 15A bacterial cells. Bacterial strains which can be used as source of Streptococcus pneumoniae serotype 15A polysaccharides may be obtained from established culture collections (such as for example from the Streptococcal Reference Laboratory (Centers for Disease Control and Prevention, Atlanta, GA USA)) or clinical specimens.

Serotype 15A saccharides can be obtained directly from bacteria using isolation procedures known to one of ordinary skill in the art. They can also be purchased (such as for example from the American Type Culture Collection (ATCC, Manassas, VA USA) (e.g., reference No. ATCC (537-X)).

In case the serotype 15A saccharide is obtained directly from bacteria, the bacterial cells can be grown in a medium, preferably in a soy based medium. Following fermentation of bacterial cells that produce S. pneumoniae serotype 15A capsular polysaccharides, the bacterial cells can be lysed to produce a cell lysate. The serotype 15A polysaccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020170190). The purified serotype 15A capsular polysaccharide can then be used for the preparation of glycoconjugates.

The isolated serotype 15A capsular saccharide obtained by purification of serotype 15A polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In a preferred embodiment, the isolated serotype 15A capsular polysaccharide (i.e. purified before further treatment) has a weight average molecular weight between 100 kDa and 2500 kDa. In an embodiment, the isolated serotype 15A capsular polysaccharide has a weight average molecular weight between 250 kDa and 1500 kDa. In an embodiment, the isolated serotype 15A capsular polysaccharide has a weight average molecular weight between 500 kDa and 1000 kDa.

Any whole number integer within any of the above ranges is contemplated as an embodiment of the disclosure.

In order to generate serotype 15A conjugates with advantageous filterability characteristics, immunogenicity and/or yields, sizing of the polysaccharide to a target molecular weight range can be performed prior to the conjugation to a carrier protein. Advantageously, the size of the purified serotype 15A polysaccharide is reduced while preserving critical features of the structure of the polysaccharide. Mechanical or chemical sizing maybe employed.

In an embodiment, the size of the purified serotype 15A polysaccharide is reduced by chemical hydrolysis. Chemical hydrolysis maybe conducted using a mild acid (e.g acetic acid, formic acid, propanoic acid). Chemical hydrolysis may also be conducted using a diluted strong acid (such as diluted hydrochloric acid, diluted sulfuric acid, diluted phosphoric acid, diluted nitric acid or diluted perchloric acid).

Preferably though, the size of the purified serotype 15A polysaccharide is reduced by mechanical homogenization. In an embodiment, the size of the purified serotype 15A polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

In an embodiment, the isolated serotype 15A capsular polysaccharide is sized to a weight average molecular weight of between 50 kDa and 500 kDa. In a preferred embodiment, the isolated serotype 15A capsular polysaccharide is sized to a weight average molecular weight of between 75 kDa and 250 kDa. Preferably, the isolated serotype 15A capsular polysaccharide is sized to a weight average molecular weight below 175 kDa. In an even preferred embodiment, the isolated serotype 15A capsular polysaccharide is sized to a weight average molecular weight of between 75 kDa and 175 kDa. In a most preferred embodiment, the isolated serotype 15A capsular polysaccharide is sized to a weight average molecular weight of between 100 kDa and 175 kDa. Preferably, the isolated serotype 15A polysaccharide is sized by mechanical homogenization, preferably by high pressure homogenization.

In an embodiment, the isolated serotype 15A capsular polysaccharide is not sized.

In an embodiment, the isolated serotype 15A capsular polysaccharide before conjugation has a weight average molecular weight between 50 kDa and 500 kDa. In an embodiment, the isolated serotype 15A capsular polysaccharide before conjugation has a weight average molecular weight between 75 kDa and 250 kDa. In a preferred embodiment, the isolated serotype 15A capsular polysaccharide before conjugation has a weight average molecular weight between 75 kDa and 175 kDa. In an even preferred embodiment, the isolated serotype 15A capsular polysaccharide before conjugation has a weight average molecular weight between 90 kDa and 150 kDa. In a most preferred embodiment, the isolated serotype 15A capsular polysaccharide before conjugation has a weight average molecular weight between 100 kDa and 175 kDa.

The weight average molecular weight (Mw) of the saccharide before conjugation refers to the Mw before activation of the polysaccharide (i.e. after an eventual sizing step but before reacting the polysaccharide with an activating agent). In the context of the present invention the Mw of the 15A polysaccharide is not substantially modified by the activation step and the Mw of the 15A polysaccharide incorporated in the conjugate is similar to the Mw of the polysaccharide as measured before activation.

In an embodiment, the serotype 15A glycoconjugate of the present invention comprises a serotype 15A capsular polysaccharide wherein the weight average molecular weight (Mw) of said polysaccharide before conjugation is between 50 kDa and 500 kDa. In an embodiment, the weight average molecular weight (Mw) is between 75 kDa and 250 kDa. In a preferred embodiment, the weight average molecular weight (Mw) is between 75 kDa and 175 kDa. In a most preferred embodiment, the weight average molecular weight (Mw) is between 90 kDa and 150 kDa.

In some embodiments, the serotype 15A glycoconjugate of the invention has a weight average molecular weight (Mw) of between 500 kDa and 10,000 kDa. In other embodiments, the serotype 15A glycoconjugate has a weight average molecular weight (Mw) of between 1,000 kDa and 10,000 kDa. Preferably, the serotype 15A glycoconjugate has a weight average molecular weight (Mw) of between 1,000 kDa and 6,000 kDa.

The serotype 15A glycoconjugates of the invention may also be characterized by the ratio (weight/weight) of saccharide to carrier protein. In some embodiments, the ratio of serotype 15A polysaccharide to carrier protein in the glycoconjugate (w/w) is between 0.5 and 3.0. Preferably, the saccharide to carrier protein ratio (w/w) is between 0.5 and 1.5. Even more preferably, the saccharide to carrier protein ratio (w/w) is between 0.7 and 1.1.

Another way to characterize the serotype 15A glycoconjugates of the invention is by the number of lysine residues in the carrier protein (e.g., CRM197, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 15A glycoconjugate of the invention is between 2 and 20. Preferably, the degree of conjugation of the serotype 15A glycoconjugate of the invention is between 5 and 10.

The serotype 15A glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In an embodiment, the serotype 15A glycoconjugate comprises less than about 40% of free serotype 15A polysaccharide compared to the total amount of serotype 15A polysaccharide. In a preferred embodiment the serotype 15A glycoconjugate comprises less than about 25% of free serotype 15A polysaccharide compared to the total amount of serotype 15A polysaccharide.

The serotype 15A glycoconjugates may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V0), (Kd=0), and the fraction representing the maximum retention (Vi), (Kd=1). The fraction at which a specified sample attribute is reached (Ve), is related to Kd by the expression, Kd=(Ve−V0)/(Vi−V0).

In an embodiment, at least 40% of the serotype 15A glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 90% of the serotype 15A glycoconjugate has a Kd below or equal to 0.3 in a CL-4B column.

In an embodiment, the serotype 15A saccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, the serotype 15A glycoconjugates of the invention are prepared using reductive amination chemistry. According to the present invention, reductive amination involves two steps, (1) oxidation (activation) of a purified saccharide, (2) reduction of the activated saccharide and a carrier protein to form a glycoconjugate (see e.g. WO2006/110381, WO2008/079653, WO2008/143709, WO2008/079732, WO2011/110531, WO2012/119972, WO2015110941, WO2015110940, WO2018/144439, WO2018/156491).

In an embodiment, the serotype 15A glycoconjugates of the invention are prepared by conjugating an isolated serotype 15A capsular polysaccharide to a carrier protein by a process comprising the step of:
(a) reacting said isolated serotype 15A capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

As mentioned above, before oxidation, sizing of the isolated serotype 15A capsular polysaccharide to a target molecular weight (MW) range can be performed. Therefore, in an embodiment, the isolated serotype 15A capsular polysaccharide is sized before oxidation.

In an embodiment, the isolated serotype 15A capsular polysaccharide is not sized before oxidation.

In an embodiment, the oxidation step (a) is carried out at a pH of between 4.0 and 6.0. Preferably, the oxidation step (a) is carried out at a pH of between 4.5 and 5.5.

In an embodiment, the oxidation step (a) is carried out at a pH of about 5.0.

Following the oxidation step (a) the saccharide is said to be activated and is referred to as “activated polysaccharide”.

In an embodiment, the activated serotype 15A polysaccharide of the present invention has a weight average molecular weight (Mw) of between 50 kDa and 500 kDa. In an embodiment, the weight average molecular weight (Mw) is between 75 kDa and 250 kDa. In a preferred embodiment, the weight average molecular weight (Mw) is between 75 kDa and 175 kDa. In a most preferred embodiment, the weight average molecular weight (Mw) is between 90 kDa and 150 kDa.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO⁴⁻) and orthoperiodate (IO₆⁵⁻) and the various salts of periodate (e.g., sodium periodate and potassium periodate).

In a preferred embodiment, the oxidizing agent is sodium periodate. In an embodiment, the periodate used for the oxidation is metaperiodate. In a most preferred embodiment, the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the polysaccharide with 0.1-2 molar equivalents of periodate. Preferably step a) comprises reacting the polysaccharide with 0.5-1.5 molar equivalents of periodate. Most preferably, step a) comprises reacting the polysaccharide with 0.8-1.2 molar equivalents of periodate.

In an embodiment the degree of oxidation (also named “degree of activation” in the present document) of the activated serotype 15A polysaccharide is between 2 and 20. In a preferred embodiment the degree of oxidation of the activated serotype 15A polysaccharide is between 2 and 8. In a most preferred embodiment the degree of oxidation of the activated serotype 15A polysaccharide is 5±2.5.

In one embodiment the activated serotype 15A polysaccharide and the carrier protein are lyophilised before step b). Preferably lyophilisation occurs after step a). In one embodiment the activated polysaccharide is lyophilised after step a) and the carrier protein is also lyophilised. In one embodiment the activated serotype 15A polysaccharide is lyophilised after step a) and the carrier protein is also lyophilised, and the activated polysaccharide and the carrier protein are reconstituted in the same solution.

In an embodiment, the activated serotype 15A polysaccharide and the carrier protein are lyophilised independently (discrete lyophilization). In an embodiment, the activated serotype 15A polysaccharide and the carrier protein are lyophilised together (co-lyophilized).

In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is sucrose.

In an embodiment the initial input ratio (weight by weight) of activated serotype 15A capsular polysaccharide to carrier protein at step b) is between 3:1 and 0.5:1. In an embodiment the initial input ratio (weight by weight) of activated serotype 15A capsular polysaccharide to carrier protein is between 1.5:1 and 0.5:1. Preferably, the initial input ratio (weight by weight) of activated serotype 15A capsular polysaccharide to carrier protein is between 1.1:1 and 0.9:1.

In an embodiment, the reduction reaction (c) is carried out in aqueous solvent. Preferably, the reduction reaction (c) is carried out in aprotic solvent.

In an embodiment, the reduction reaction (c) is carried out in the presence of dimethylsulphoxide (DMSO) or dimethylformamide (DMF). Preferably, the reduction reaction (c) is carried out in the presence of dimethylsulphoxide (DMSO).

In an embodiment, the reduction reaction (c) is carried out in DMSO (dimethylsulfoxide) or in DMF (dimethylformamide)) solvent.

Most preferably, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeiPrN-BH3, benzylamine-BH3 or 5-ethyl-2-methylpyridine borane (PEMB). In an embodiment, the reducing agent is sodium triacetoxyborohydride. In an embodiment, the reducing agent is sodium cyanoborohydride in the present of nickel (see WO2018144439). In a preferred embodiment, the reducing agent is sodium cyanoborohydride.

In one embodiment between 0.2 and 5 molar equivalents of reducing agent is used in step c). Preferably, between 0.5 and 2 molar equivalents of reducing agent is used in step c). Most preferably, between 0.9 and 1.1 molar equivalents of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

Following conjugation to the carrier protein, the serotype 15A glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

Therefore, in one embodiment the process for producing the serotype 15A glycoconjugate of the present invention comprises the step of purifying the glycoconjugate after it is produced.

### 1.2 S. pneumoniae Serotype 23A Glycoconjugate of the Invention

In an aspect the invention relates to S. pneumoniae serotype 23A glycoconjugates.

The structure of Streptococcus pneumoniae serotype 23A polysaccharide is known in the art (see e.g. Ravenscroft N et al. (2017) Carbohydrate Res. Vol. 450, p 19-29 and WO2019050814). The structure of the serotype 23A capsular polysaccharide is: →4)-β-D-Glcp-(1→3)-[[α-L-Rhap-(1→2)]-[Gro-(2→P→3)]-β-D-Galp-(1→4)]-β-L-Rhap-(1→.

In an embodiment, the capsular S. pneumoniae serotype 23A saccharide used in the present invention is a synthetic carbohydrate.

In a preferred embodiment though, the source of bacterial polysaccharide according to this invention can be Streptococcus pneumoniae serotype 23A bacterial cells. Bacterial strains which can be used as source of Streptococcus pneumoniae serotype 23A polysaccharides may be obtained from established culture collections (such as for example from the Streptococcal Reference Laboratory (Centers for Disease Control and Prevention, Atlanta, GA USA)) or clinical specimens.

Serotype 23A saccharides can be obtained directly from bacteria using isolation procedures known to one of ordinary skill in the art. They can also be purchased (such as for example from the American Type Culture Collection (ATCC, Manassas, VA USA) (e.g., reference No. ATCC 545-X, No. ATCC 546-X, No. ATCC 547-X)).

In case the serotype 23A saccharide is obtained directly from bacteria, the bacterial cells can be grown in a medium, preferably in a soy based medium. Following fermentation of bacterial cells that produce S. pneumoniae serotype 23A capsular polysaccharides, the bacterial cells can be lysed to produce a cell lysate. The serotype 23A polysaccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020170190). The purified serotype 23A capsular polysaccharide can then be used for the preparation of glycoconjugates.

The isolated serotype 23A capsular saccharide obtained by purification of serotype 23A polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In a preferred embodiment, the isolated serotype 23A capsular polysaccharide (i.e. purified before further treatment) has a weight average molecular weight between 100 kDa and 2500 kDa. In an embodiment, the isolated serotype 23A capsular polysaccharide has a weight average molecular weight between 250 kDa and 1500 kDa. In an embodiment, the isolated serotype 23A capsular polysaccharide has a weight average molecular weight between 500 kDa and 1000 kDa.

Any whole number integer within any of the above ranges is contemplated as an embodiment of the disclosure.

In order to generate serotype 23A conjugates with advantageous filterability characteristics, immunogenicity and/or yields, sizing of the polysaccharide to a target molecular weight range can be performed prior to the conjugation to a carrier protein. Advantageously, the size of the purified serotype 23A polysaccharide is reduced while preserving critical features of the structure of the polysaccharide. Mechanical or chemical sizing maybe employed.

Most preferably, the size of the purified serotype 23A polysaccharide is reduced by mechanical homogenization.

It has been discovered that the use of acid hydrolysis, as for example recommended in WO2019050814 or in WO2019050818 is not appropriate to reduce the size of serotype 23A polysaccharide.

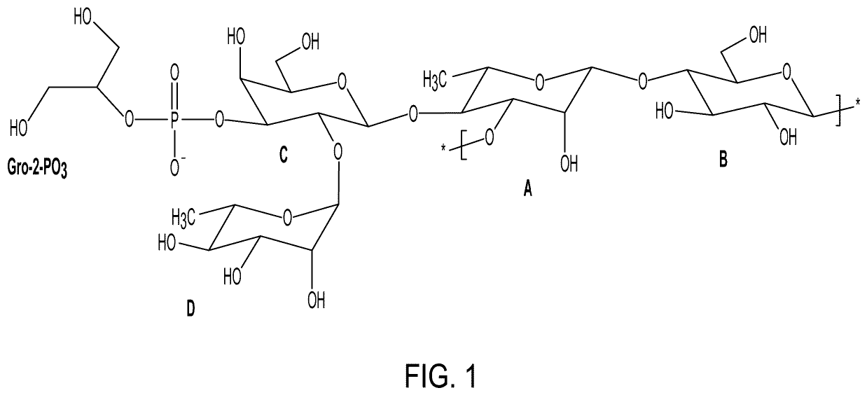

It has been found that acid hydrolysis can affect serotype 23A polysaccharide structure integrity. Even relatively mild hydrolysis (e.g. treatment with acetic acid at 100 mM at 80° C. for 2 hours) has been found to lead to a 25% loss of the branched rhamnose (α-L-Rhap-(1→2)) (see FIG. 1).

Mechanical sizing allows not to loose this residue, which can be important from an immunological point of view. Furthermore, this residue is the main activation site for periodate oxidation. It is therefore important to retain it in case periodate oxidation of the polysaccharide is used (e.g. activation step in the reductive amination chemistry).

Therefore, is a preferred embodiment, the size of the purified serotype 23A polysaccharide is reduced by mechanical homogenization.

In an embodiment, the size of the purified serotype 23A polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

In a most preferred embodiment, the process to prepare serotype 23A glycoconjugate of the invention does not comprise a step of sizing of the serotype 23A polysaccharide by acid hydrolysis.

In an embodiment, the isolated serotype 23A capsular polysaccharide is sized to a weight average molecular weight of between 50 kDa and 500 kDa. In a preferred embodiment, the isolated serotype 23A capsular polysaccharide is sized to a weight average molecular weight of between 75 kDa and 400 kDa. In an even preferred embodiment, the isolated serotype 23A capsular polysaccharide is sized to a weight average molecular weight of between 100 kDa and 350 kDa. In a most preferred embodiment, the isolated serotype 23A capsular polysaccharide is sized to a weight average molecular weight of between 125 kDa and 225 kDa. Preferably, the isolated serotype 23A polysaccharide is sized by mechanical homogenization, most preferably by high pressure homogenization.

In an embodiment, the isolated serotype 23A capsular polysaccharide is not sized.

In an embodiment, the isolated serotype 23A capsular polysaccharide before conjugation has a weight average molecular weight between 50 kDa and 500 kDa. In an embodiment, the isolated serotype 23A capsular polysaccharide before conjugation has a weight average molecular weight between 75 kDa and 400 kDa. In an even preferred embodiment, the isolated serotype 23A capsular polysaccharide before conjugation has a weight average molecular weight between 100 kDa and 350 kDa. In a most preferred embodiment, the isolated serotype 23A capsular polysaccharide before conjugation has a weight average molecular weight of between 125 kDa and 225 kDa.

The weight average molecular weight (Mw) of the saccharide before conjugation refers to the Mw before activation of the polysaccharide (i.e. after an eventual sizing step but before reacting the polysaccharide with an activating agent). In the context of the present invention the Mw of the 23A polysaccharide is not substantially modified by the activation step and the Mw of the 23A polysaccharide incorporated in the conjugate is similar to the Mw of the polysaccharide as measured before activation.

In an embodiment, the serotype 23A glycoconjugate of the present invention comprises a serotype 23A capsular polysaccharide wherein the weight average molecular weight (Mw) of said polysaccharide before conjugation is between 50 kDa and 400 kDa. In an embodiment, the weight average molecular weight (Mw) is between 100 kDa and 300 kDa. In a most preferred embodiment, the weight average molecular weight (Mw) is between 120 kDa and 240 kDa.

In some embodiments, the serotype 23A glycoconjugate of the invention has a weight average molecular weight (Mw) of between 500 kDa and 10,000 kDa. In other embodiments, the serotype 23A glycoconjugate has a weight average molecular weight (Mw) of between 1,000 kDa and 7,500 kDa. Preferably, the serotype 23A glycoconjugate has a weight average molecular weight (Mw) of between 2,000 kDa and 5,000 kDa.

The serotype 23A glycoconjugates of the invention may also be characterized by the ratio (weight/weight) of saccharide to carrier protein. In some embodiments, the ratio of serotype 23A polysaccharide to carrier protein in the glycoconjugate (w/w) is between 0.5 and 3.0. Preferably, the saccharide to carrier protein ratio (w/w) is between 0.7 and 1.5. Even more preferably, the saccharide to carrier protein ratio (w/w) is between 0.8 and 1.4.

Another way to characterize the serotype 23A glycoconjugates of the invention is by the number of lysine residues in the carrier protein (e.g., CRM197, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 23A glycoconjugate of the invention is between 2 and 20. Preferably, the degree of conjugation of the serotype 23A glycoconjugate of the invention is between 5 and 15.

The serotype 23A glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In an embodiment, the serotype 23A glycoconjugate comprises less than about 40% of free serotype 23A polysaccharide compared to the total amount of serotype 23A polysaccharide. In a preferred embodiment the serotype 23A glycoconjugate comprises less than about 25% of free serotype 23A polysaccharide compared to the total amount of serotype 23A polysaccharide.

The serotype 23A glycoconjugates may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V0), (Kd=0), and the fraction representing the maximum retention (Vi), (Kd=1). The fraction at which a specified sample attribute is reached (Ve), is related to Kd by the expression, Kd=(Ve−V0)/(Vi−V0).

In an embodiment, at least 40% of the serotype 23A glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 90% of the serotype 23A glycoconjugate has a Kd below or equal to 0.3 in a CL-4B column.

The serotype 23A glycoconjugates may also be characterized by the amount of branched Rhamnose residue which remains in the 23A polysaccharide. As mentioned above, it has been found that serotype 23A polysaccharide can loose the branched Rhamnose residue (see FIG. 1).

Therefore, in an embodiment, the S. pneumoniae serotype 23A glycoconjugate of the present invention comprises a S. pneumoniae serotype 23A capsular polysaccharide wherein said S. pneumoniae serotype 23A capsular polysaccharide has a branched Rhamnose content greater than 75% when compared to native S. pneumoniae serotype 23A capsular polysaccharide wherein the branched Rhamnose content is considered to be about 100%. In an embodiment, the S. pneumoniae serotype 23A glycoconjugate of the present invention comprises a S. pneumoniae serotype 23A capsular polysaccharide wherein said S. pneumoniae serotype 23A capsular polysaccharide has a branched Rhamnose content greater than 90% when compared to native S. pneumoniae serotype 23A capsular polysaccharide wherein the branched Rhamnose content is considered to be about 100%. Preferably, the S. pneumoniae serotype 23A glycoconjugate of the present invention comprises a S. pneumoniae serotype 23A capsular polysaccharide wherein said S. pneumoniae serotype 23A capsular polysaccharide has a branched Rhamnose content greater than 95% when compared to native S. pneumoniae serotype 23A capsular polysaccharide wherein the branched Rhamnose content is considered to be about 100%.

In a most preferred embodiment, the S. pneumoniae serotype 23A glycoconjugate of the present invention comprises a S. pneumoniae serotype 23A capsular polysaccharide wherein said S. pneumoniae serotype 23A capsular polysaccharide has a branched Rhamnose content of about 100% when compared to native S. pneumoniae serotype 23A capsular polysaccharide wherein the branched Rhamnose content is considered to be about 100%.

In an embodiment, the serotype 23A saccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, the serotype 23A glycoconjugates of the invention are prepared using reductive amination chemistry. According to the present invention, reductive amination involves two steps, (1) oxidation (activation) of a purified saccharide, (2) reduction of the activated saccharide and a carrier protein to form a glycoconjugate (see e.g. WO2006/110381, WO2008/079653, WO2008/143709, WO2008/079732, WO2011/110531, WO2012/119972, WO2015110941, WO2015110940, WO2018/144439, WO2018/156491).

In an embodiment, the serotype 23A glycoconjugates of the invention are prepared by conjugating an isolated serotype 23A capsular polysaccharide to a carrier protein by a process comprising the step of:
(a) reacting said isolated serotype 23A capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

As mentioned above, before oxidation, sizing of the isolated serotype 23A capsular polysaccharide to a target molecular weight (MW) range can be performed. Therefore, in an embodiment, the isolated serotype 23A capsular polysaccharide is sized before oxidation.

In a preferred embodiment, the size of the isolated serotype 23A capsular polysaccharide is reduced by mechanical homogenization. In an embodiment, the size of the isolated serotype 23A polysaccharide is reduced by high pressure homogenization. In a most preferred embodiment, the isolated serotype 23A capsular polysaccharide is not sized by acid hydrolysis.

In an embodiment, the isolated serotype 23A capsular polysaccharide is not sized before oxidation.

In an embodiment, the oxidation step (a) is carried out at a pH of between 4.5 and 6.5. Preferably, the oxidation step (a) is carried out at a pH of between 5.0 and 6.0.

In an embodiment, the oxidation step (a) is carried out at a pH of about 5.0.

Following the oxidation step (a) the saccharide is said to be activated and is referred to as “activated polysaccharide”.

In an embodiment, the activated serotype 23A polysaccharide of the present invention has a weight average molecular weight (Mw) of between 50 kDa and 400 kDa. In an embodiment, the weight average molecular weight (Mw) is between 100 kDa and 250 kDa. In a most preferred embodiment, the weight average molecular weight (Mw) is between 120 kDa and 200 kDa.

In an embodiment, the activated serotype 23A polysaccharide of the present invention retains at least 80% of the branched Rhamnose. In an embodiment, the activated serotype 23A polysaccharide of the present invention retains at least 85% of the branched Rhamnose. In an embodiment, the activated serotype 23A polysaccharide of the present invention retains at least 90% of the branched Rhamnose. In a preferred embodiment, the activated serotype 23A polysaccharide of the present invention retains at least 95% of the branched Rhamnose.

In a preferred embodiment, the activated serotype 23A polysaccharide of the present invention retains at least 96% of the branched Rhamnose.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid, the term also includes both metaperiodate (IO⁴⁻) and orthoperiodate (IO₆⁵⁻) and the various salts of periodate (e.g., sodium periodate and potassium periodate).

In a preferred embodiment, the oxidizing agent is sodium periodate. In an embodiment, the periodate used for the oxidation is metaperiodate. In a most preferred embodiment, the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the polysaccharide with 0.1-2 molar equivalents of periodate. Preferably step a) comprises reacting the polysaccharide with 0.2-1.5 molar equivalents of periodate. Most preferably, step a) comprises reacting the polysaccharide with 0.3-0.5 molar equivalents of periodate.

In an embodiment the degree of oxidation (also named “degree of activation” in the present document) of the activated serotype 23A polysaccharide is between 2 and 20. In a preferred embodiment the degree of oxidation of the activated serotype 23A polysaccharide is between 2 and 8. In a most preferred embodiment the degree of oxidation of the activated serotype 23A polysaccharide is 5±2.5.

In one embodiment the activated serotype 23A polysaccharide and the carrier protein are lyophilised before step b). Preferably lyophilisation occurs after step a). In one embodiment the activated polysaccharide is lyophilised after step a) and the carrier protein is also lyophilised. In one embodiment the activated serotype 23A polysaccharide is lyophilised after step a) and the carrier protein is also lyophilised, and the activated polysaccharide and the carrier protein are reconstituted in the same solution.

In an embodiment, the activated serotype 23A polysaccharide and the carrier protein are lyophilised independently (discrete lyophilization). In an embodiment, the activated serotype 23A polysaccharide and the carrier protein are lyophilised together (co-lyophilized).

In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is sucrose.

In an embodiment the initial input ratio (weight by weight) of activated serotype 23A capsular polysaccharide to carrier protein at step b) is between 2:1 and 0.5:1. In an embodiment the initial input ratio (weight by weight) of activated serotype 23A capsular polysaccharide to carrier protein is between 1.2:1 and 0.6:1. Preferably, the initial input ratio (weight by weight) of activated serotype 23A capsular polysaccharide to carrier protein is between 0.9:1 and 0.7:1.

In an embodiment, the reduction reaction (c) is carried out in aqueous solvent. Preferably, the reduction reaction (c) is carried out in aprotic solvent.

In an embodiment, the reduction reaction (c) is carried out in the presence of dimethylsulphoxide (DMSO) or dimethylformamide (DMF). Preferably, the reduction reaction (c) is carried out in the presence of dimethylsulphoxide (DMSO).

In an embodiment, the reduction reaction (c) is carried out in DMSO (dimethylsulfoxide) or in DMF (dimethylformamide)) solvent.

Most preferably, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeiPrN-BH3, benzylamine-BH3 or 5-ethyl-2-methylpyridine borane (PEMB). In an embodiment, the reducing agent is sodium triacetoxyborohydride. In an embodiment, the reducing agent is sodium cyanoborohydride in the present of nickel (see WO2018144439). In a preferred embodiment, the reducing agent is sodium cyanoborohydride.

In one embodiment between 0.2 and 5 molar equivalents of reducing agent is used in step c). Preferably, between 0.2 and 1.5 molar equivalents of reducing agent is used in step c). Most preferably, between 0.5 and 1.0 molar equivalent of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

Following conjugation to the carrier protein, the serotype 23A glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the serotype 23A glycoconjugate of the present invention comprises the step of purifying the glycoconjugate after it is produced.

### 1.3 S. pneumoniae Serotype 23B Glycoconjugate of the Invention

In an aspect the invention relates to S. pneumoniae serotype 23B glycoconjugates.

The structure of Streptococcus pneumoniae serotype 23B polysaccharide is known in the art (see e.g. Ravenscroft N et al. (2017) Carbohydrate Res. Vol. 450, p 19-29 and WO2019050814). The structure of the serotype 23A capsular polysaccharide is:

→4)-β-D-Glcp-(1→4)-[Gro-(2→P→3)]-β-D-Galp-(1→4)-β-L-Rhap-(1→.

In an embodiment, the capsular S. pneumoniae serotype 23B saccharide used in the present invention is a synthetic carbohydrate.

In a preferred embodiment though, the source of bacterial polysaccharide according to this invention can be Streptococcus pneumoniae serotype 23B bacterial cells. Bacterial strains which can be used as source of Streptococcus pneumoniae serotype 23B polysaccharides may be obtained from established culture collections (such as for example from the Streptococcal Reference Laboratory (Centers for Disease Control and Prevention, Atlanta, GA USA)) or clinical specimens.

Serotype 23B saccharides can be obtained directly from bacteria using isolation procedures known to one of ordinary skill in the art. They can also be purchased (such as for example from the American Type Culture Collection (ATCC, Manassas, VA USA) (e.g., reference No. ATCC 548-X, No. ATCC 549-X, No. ATCC 550-X)).

In case the serotype 23B saccharide is obtained directly from bacteria, the bacterial cells can be grown in a medium, preferably in a soy based medium. Following fermentation of bacterial cells that produce S. pneumoniae serotype 23B capsular polysaccharides, the bacterial cells can be lysed to produce a cell lysate. The serotype 23B polysaccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020170190). The purified serotype 23B capsular polysaccharide can then be used for the preparation of glycoconjugates.

The isolated serotype 23B capsular saccharide obtained by purification of serotype 23B polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In a preferred embodiment, the isolated serotype 23B capsular polysaccharide (i.e. purified before further treatment) has a weight average molecular weight between 100 kDa and 2500 kDa. In an embodiment, the isolated serotype 23B capsular polysaccharide has a weight average molecular weight between 250 kDa and 2000 kDa. In an embodiment, the isolated serotype 23B capsular polysaccharide has a weight average molecular weight between 300 kDa and 1000 kDa.

Any whole number integer within any of the above ranges is contemplated as an embodiment of the disclosure.

In order to generate serotype 23B conjugates with advantageous filterability characteristics, immunogenicity and/or yields, sizing of the polysaccharide to a target molecular weight range can be performed prior to the conjugation to a carrier protein. Advantageously, the size of the purified serotype 23B polysaccharide is reduced while preserving critical features of the structure of the polysaccharide. Mechanical or chemical sizing maybe employed.

In an embodiment, the size of the purified serotype 23B polysaccharide is reduced by chemical hydrolysis. Chemical hydrolysis maybe conducted using a mild acid (e.g acetic acid, formic acid, propanoic acid). Chemical hydrolysis may also be conducted using a diluted strong acid (such as diluted hydrochloric acid, diluted sulfuric acid, diluted phosphoric acid, diluted nitric acid or diluted perchloric acid).

Preferably though, the size of the purified serotype 23B polysaccharide is reduced by mechanical homogenization. In an embodiment, the size of the purified serotype 23B polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

In an embodiment, the isolated serotype 23B capsular polysaccharide is sized to a weight average molecular weight of between 50 kDa and 750 kDa. In a preferred embodiment, the isolated serotype 23B capsular polysaccharide is sized to a weight average molecular weight of between 75 kDa and 400 kDa. In an even preferred embodiment, the isolated serotype 23B capsular polysaccharide is sized to a weight average molecular weight of between 100 kDa and 250 kDa. Preferably, the isolated serotype 23B polysaccharide is sized by mechanical homogenization, most preferably by high pressure homogenization.

In an embodiment, the isolated serotype 23B capsular polysaccharide is not sized.

In an embodiment, the isolated serotype 23B capsular polysaccharide before conjugation has a weight average molecular weight between 50 kDa and 750 kDa. In an embodiment, the isolated serotype 23B capsular polysaccharide before conjugation has a weight average molecular weight between 75 kDa and 400 kDa. In an even preferred embodiment, the isolated serotype 23B capsular polysaccharide before conjugation has a weight average molecular weight between 100 kDa and 250 kDa.

The weight average molecular weight (Mw) of the saccharide before conjugation refers to the Mw before activation of the polysaccharide (i.e. after an eventual sizing step but before reacting the polysaccharide with an activating agent). In the context of the present invention the Mw of the 23B polysaccharide is not substantially modified by the activation step and the Mw of the 23B polysaccharide incorporated in the conjugate is similar to the Mw of the polysaccharide as measured before activation.

In an embodiment, the serotype 23B glycoconjugate of the present invention comprises a serotype 23B capsular polysaccharide wherein the weight average molecular weight (Mw) of said polysaccharide before conjugation is between 40 kDa and 600 kDa. In an embodiment, the weight average molecular weight (Mw) is between 50 kDa and 300 kDa. In a most preferred embodiment, the weight average molecular weight (Mw) is between 100 kDa and 200 kDa.

In some embodiments, the serotype 23B glycoconjugate of the invention has a weight average molecular weight (Mw) of between 250 kDa and 7,500 kDa. In other embodiments, the serotype 23B glycoconjugate has a weight average molecular weight (Mw) of between 500 kDa and 4,000 kDa. Preferably, the serotype 23B glycoconjugate has a weight average molecular weight (Mw) of between 700 kDa and 2,000 kDa.

The serotype 23B glycoconjugates of the invention may also be characterized by the ratio (weight/weight) of saccharide to carrier protein. In some embodiments, the ratio of serotype 23B polysaccharide to carrier protein in the glycoconjugate (w/w) is between 0.4 and 3.0. Preferably, the saccharide to carrier protein ratio (w/w) is between 0.5 and 1.5. Even more preferably, the saccharide to carrier protein ratio (w/w) is between 0.6 and 1.3.

Another way to characterize the serotype 23B glycoconjugates of the invention is by the number of lysine residues in the carrier protein (e.g., CRM197, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 23B glycoconjugate of the invention is between 2 and 15. Preferably, the degree of conjugation of the serotype 23B glycoconjugate of the invention is between 5 and 12.

The serotype 23B glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In an embodiment, the serotype 23B glycoconjugate comprises less than about 40% of free serotype 23B polysaccharide compared to the total amount of serotype 23B polysaccharide. In a preferred embodiment the serotype 23B glycoconjugate comprises less than about 25% of free serotype 23B polysaccharide compared to the total amount of serotype 23B polysaccharide.

The serotype 23B glycoconjugates may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V0), (Kd=0), and the fraction representing the maximum retention (Vi), (Kd=1). The fraction at which a specified sample attribute is reached (Ve), is related to Kd by the expression, Kd=(Ve−V0)/(Vi−V0).

In an embodiment, at least 30% of the serotype 23B glycoconjugate has a Kd below or equal to 0.3 in a CL-4B column. In a preferred embodiment, at least 35% of the glycoconjugate has a Kd below or equal to 0.3 in a CL-4B column. In a preferred embodiment, between 40% and 60% of the serotype 23B glycoconjugate has a Kd below or equal to 0.3 in a CL-4B column.

In an embodiment, the serotype 23B saccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, the serotype 23B glycoconjugates of the invention are prepared using reductive amination chemistry. According to the present invention, reductive amination involves two steps, (1) oxidation (activation) of a purified saccharide, (2) reduction of the activated saccharide and a carrier protein to form a glycoconjugate (see e.g. WO2006/110381, WO2008/079653, WO2008/143709, WO2008/079732, WO2011/110531, WO2012/119972, WO2015110941, WO2015110940, WO2018/144439, WO2018/156491).

In an embodiment, the serotype 23B glycoconjugates of the invention are prepared by conjugating an isolated serotype 23B capsular polysaccharide to a carrier protein by a process comprising the step of:
(a) reacting said isolated serotype 23B capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

As mentioned above, before oxidation, sizing of the isolated serotype 23B capsular polysaccharide to a target molecular weight (MW) range can be performed. Therefore, in an embodiment, the isolated serotype 23B capsular polysaccharide is sized before oxidation.

In a preferred embodiment, the size of the isolated serotype 23B capsular polysaccharide is reduced by mechanical homogenization. In an embodiment, the size of the isolated serotype 23B polysaccharide is reduced by high pressure homogenization.

In an embodiment, the isolated serotype 23B capsular polysaccharide is not sized before oxidation.

In an embodiment, the oxidation step (a) is carried out at a pH of between 4.0 and 6.5. Preferably, the oxidation step (a) is carried out at a pH of between 4.5 and 5.5.

In an embodiment, the oxidation step (a) is carried out at a pH of about 5.0.

Following the oxidation step (a) the saccharide is said to be activated and is referred to as “activated polysaccharide”.

In an embodiment, the activated serotype 23B polysaccharide of the present invention has a weight average molecular weight (Mw) of between 40 kDa and 600 kDa. In an embodiment, the weight average molecular weight (Mw) is between 50 kDa and 300 kDa. In a most preferred embodiment, the weight average molecular weight (Mw) is between 100 kDa and 200 kDa.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid, the term also includes both metaperiodate (IO⁴⁻) and orthoperiodate (IO₆⁵⁻) and the various salts of periodate (e.g., sodium periodate and potassium periodate).

In a preferred embodiment, the oxidizing agent is sodium periodate. In an embodiment, the periodate used for the oxidation is metaperiodate. In a most preferred embodiment, the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the polysaccharide with 0.05-1 molar equivalents of periodate. Preferably step a) comprises reacting the polysaccharide with 0.1-0.3 molar equivalents of periodate. Most preferably, step a) comprises reacting the polysaccharide with about 0.2 molar equivalents of periodate.

In an embodiment the degree of oxidation (also named “degree of activation” in the present document) of the activated serotype 23B polysaccharide is between 2 and 20. In a preferred embodiment the degree of oxidation of the activated serotype 23B polysaccharide is between 4 and 15. In a most preferred embodiment the degree of oxidation of the activated serotype 23B polysaccharide is 9±3.

In one embodiment the activated serotype 23B polysaccharide and the carrier protein are lyophilised before step b). Preferably lyophilisation occurs after step a). In one embodiment the activated polysaccharide is lyophilised after step a) and the carrier protein is also lyophilised.

In one embodiment the activated serotype 23B polysaccharide is lyophilised after step a) and the carrier protein is also lyophilised, and the activated polysaccharide and the carrier protein are reconstituted in the same solution.

In an embodiment, the activated serotype 23B polysaccharide and the carrier protein are lyophilised independently (discrete lyophilization). In an embodiment, the activated serotype 23B polysaccharide and the carrier protein are lyophilised together (co-lyophilized).

In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is sucrose.

In an embodiment the initial input ratio (weight by weight) of activated serotype 23B capsular polysaccharide to carrier protein at step b) is between 2:1 and 0.5:1. In an embodiment the initial input ratio (weight by weight) of activated serotype 23B capsular polysaccharide to carrier protein is between 1.2:1 and 0.6:1. Preferably, the initial input ratio (weight by weight) of activated serotype 23B capsular polysaccharide to carrier protein is between 0.9:1 and 0.7:1.

In an embodiment, the reduction reaction (c) is carried out in aqueous solvent. Preferably, the reduction reaction (c) is carried out in aprotic solvent.

In an embodiment, the reduction reaction (c) is carried out in the presence of dimethylsulphoxide (DMSO) or dimethylformamide (DMF). Preferably, the reduction reaction (c) is carried out in the presence of dimethylsulphoxide (DMSO).

In an embodiment, the reduction reaction (c) is carried out in DMSO (dimethylsulfoxide) or in DMF (dimethylformamide)) solvent.

Most preferably, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeiPrN-BH3, benzylamine-BH3 or 5-ethyl-2-methylpyridine borane (PEMB). In an embodiment, the reducing agent is sodium triacetoxyborohydride. In an embodiment, the reducing agent is sodium cyanoborohydride in the present of nickel (see WO2018144439). In a preferred embodiment, the reducing agent is sodium cyanoborohydride.

In one embodiment between 0.2 and 5 molar equivalents of reducing agent is used in step c). Preferably, between 0.5 and 1.5 molar equivalents of reducing agent is used in step c). Most preferably, between 0.9 and 1.1 molar equivalent of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

Following conjugation to the carrier protein, the serotype 23B glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the serotype 23B glycoconjugate of the present invention comprises the step of purifying the glycoconjugate after it is produced.

### 1.4 S. pneumoniae Serotype 24F Glycoconjugate of the Invention

In an aspect the invention relates to S. pneumoniae serotype 24F glycoconjugates.

The structure of Streptococcus pneumoniae serotype 24F polysaccharide is known in the art (see e.g. WO2019050815).

In an embodiment, the capsular S. pneumoniae serotype 24F saccharide used in the present invention is a synthetic carbohydrate.

In a preferred embodiment though, the source of bacterial polysaccharide according to this invention can be Streptococcus pneumoniae serotype 24F bacterial cells. Bacterial strains which can be used as source of Streptococcus pneumoniae serotype 24F polysaccharides may be obtained from established culture collections (such as for example from the Streptococcal Reference Laboratory (Centers for Disease Control and Prevention, Atlanta, GA USA)) or clinical specimens.

Serotype 24F saccharides can be obtained directly from bacteria using isolation procedures known to one of ordinary skill in the art. They can also be purchased (such as for example from the American Type Culture Collection (ATCC, Manassas, VA USA) (e.g., reference No. ATCC 551-X, No. ATCC 552-X, No. ATCC 553-X)).

In case the serotype 24F saccharide is obtained directly from bacteria, the bacterial cells can be grown in a medium, preferably in a soy based medium. Following fermentation of bacterial cells that produce S. pneumoniae serotype 24F capsular polysaccharides, the bacterial cells can be lysed to produce a cell lysate. The serotype 24F polysaccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020170190). The purified serotype 24F capsular polysaccharide can then be used for the preparation of glycoconjugates.

The isolated serotype 24F capsular saccharide obtained by purification of serotype 24F polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In a preferred embodiment, the isolated serotype 24F capsular polysaccharide (i.e. purified before further treatment) has a weight average molecular weight between 100 kDa and 2500 kDa. In an embodiment, the isolated serotype 24F capsular polysaccharide has a weight average molecular weight between 250 kDa and 2000 kDa. In an embodiment, the isolated serotype 24F capsular polysaccharide has a weight average molecular weight between 500 kDa and 1000 kDa.

Any whole number integer within any of the above ranges is contemplated as an embodiment of the disclosure.

In order to generate serotype 24F conjugates with advantageous filterability characteristics, immunogenicity and/or yields, sizing of the polysaccharide to a target molecular weight range can be performed prior to the conjugation to a carrier protein. Advantageously, the size of the purified serotype 24F polysaccharide is reduced while preserving critical features of the structure of the polysaccharide. Mechanical or chemical sizing maybe employed.

Most preferably, the size of the purified serotype 24F polysaccharide is reduced by mechanical homogenization.

It has been discovered that the use of acid hydrolysis, as for example recommended in WO2019050815 or in WO2019050818 is not appropriate to reduce the size of serotype 24F polysaccharide.

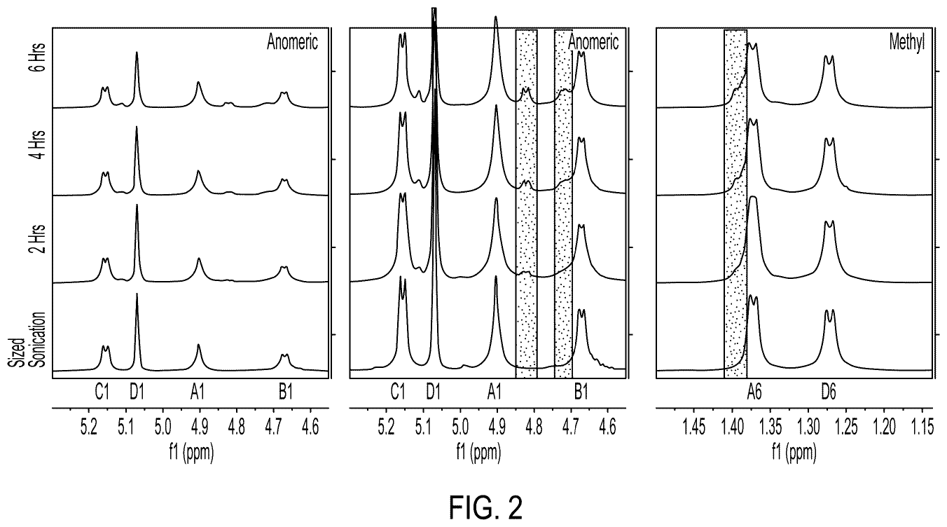

It has been found that acid hydrolysis can affect serotype 24F polysaccharide structure integrity. Even relatively mild hydrolysis (e.g. treatment with acetic acid at 100 mM at 80° C. for about 2 hour) has been found to lead to a 25% loss of the branched ribose residue (see FIG. 2).

Mechanical sizing allows not to loose this residue, which can be important from an immunological point of view.

Therefore, in a preferred embodiment, the size of the purified serotype 24F polysaccharide is reduced by mechanical homogenization.

In an embodiment, the size of the purified serotype 24F polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

In a most preferred embodiment, the process to prepare serotype 24F glycoconjugate of the invention does not comprise a step of sizing of the serotype 24F polysaccharide by acid hydrolysis.

In an embodiment, the isolated serotype 24F capsular polysaccharide is sized to a weight average molecular weight of between 50 kDa and 500 kDa. In a preferred embodiment, the isolated serotype 24F capsular polysaccharide is sized to a weight average molecular weight of between 75 kDa and 400 kDa. In an even preferred embodiment, the isolated serotype 24F capsular polysaccharide is sized to a weight average molecular weight of between 125 kDa and 275 kDa. In a most preferred embodiment, the isolated serotype 24F capsular polysaccharide is sized to a weight average molecular weight of between 125 kDa and 225 kDa. Preferably, the isolated serotype 24F polysaccharide is sized by mechanical homogenization, most preferably by high pressure homogenization.

In an embodiment, the isolated serotype 24F capsular polysaccharide is not sized.

In an embodiment, the isolated serotype 24F capsular polysaccharide before conjugation has a weight average molecular weight between 50 kDa and 500 kDa. In an embodiment, the isolated serotype 24F capsular polysaccharide before conjugation has a weight average molecular weight between 75 kDa and 400 kDa. In an even preferred embodiment, the isolated serotype 24F capsular polysaccharide before conjugation has a weight average molecular weight between 125 kDa and 275 kDa. In a most preferred embodiment, the isolated serotype 24F capsular polysaccharide before conjugation has a weight average molecular weight between 125 kDa and 225 kDa.

The weight average molecular weight (Mw) of the saccharide before conjugation refers to the Mw before activation of the polysaccharide (i.e. after an eventual sizing step but before reacting the polysaccharide with an activating agent). In the context of the present invention the Mw of the 24F polysaccharide is not substantially modified by the activation step and the Mw of the 24F polysaccharide incorporated in the conjugate is similar to the Mw of the polysaccharide as measured before activation.

In an embodiment, the serotype 24F glycoconjugate of the present invention comprises a serotype 24F capsular polysaccharide wherein the weight average molecular weight (Mw) of said polysaccharide before conjugation is between 50 kDa and 400 kDa. In an embodiment, the weight average molecular weight (Mw) is between 120 kDa and 250 kDa. In a most preferred embodiment, the weight average molecular weight (Mw) is between 120 kDa and 200 kDa.

In some embodiments, the serotype 24F glycoconjugate of the invention has a weight average molecular weight (Mw) of between 500 kDa and 10,000 kDa. In other embodiments, the serotype 24F glycoconjugate has a weight average molecular weight (Mw) of between 1,500 kDa and 7,500 kDa. Preferably, the serotype 24F glycoconjugate has a weight average molecular weight (Mw) of between 3,000 kDa and 6,000 kDa.

The serotype 24F glycoconjugates of the invention may also be characterized by the ratio (weight/weight) of saccharide to carrier protein. In some embodiments, the ratio of serotype 24F polysaccharide to carrier protein in the glycoconjugate (w/w) is between 0.5 and 3.0. Preferably, the saccharide to carrier protein ratio (w/w) is between 0.7 and 1.5. Even more preferably, the saccharide to carrier protein ratio (w/w) is between 0.8 and 1.4.

Another way to characterize the serotype 24F glycoconjugates of the invention is by the number of lysine residues in the carrier protein (e.g., CRM197, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 24F glycoconjugate of the invention is between 2 and 15. Preferably, the degree of conjugation of the serotype 24F glycoconjugate of the invention is between 5 and 12.

The serotype 24F glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In an embodiment, the serotype 24F glycoconjugate comprises less than about 40% of free serotype 24F polysaccharide compared to the total amount of serotype 24F polysaccharide. In a preferred embodiment the serotype 24F glycoconjugate comprises less than about 25% of free serotype 24F polysaccharide compared to the total amount of serotype 24F polysaccharide.

The serotype 24F glycoconjugates may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V0), (Kd=0), and the fraction representing the maximum retention (Vi), (Kd=1). The fraction at which a specified sample attribute is reached (Ve), is related to Kd by the expression, Kd=(Ve−V0)/(Vi−V0).

In an embodiment, at least 40% of the serotype 24F glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 90% of the serotype 24F glycoconjugate has a Kd below or equal to 0.3 in a CL-4B column.

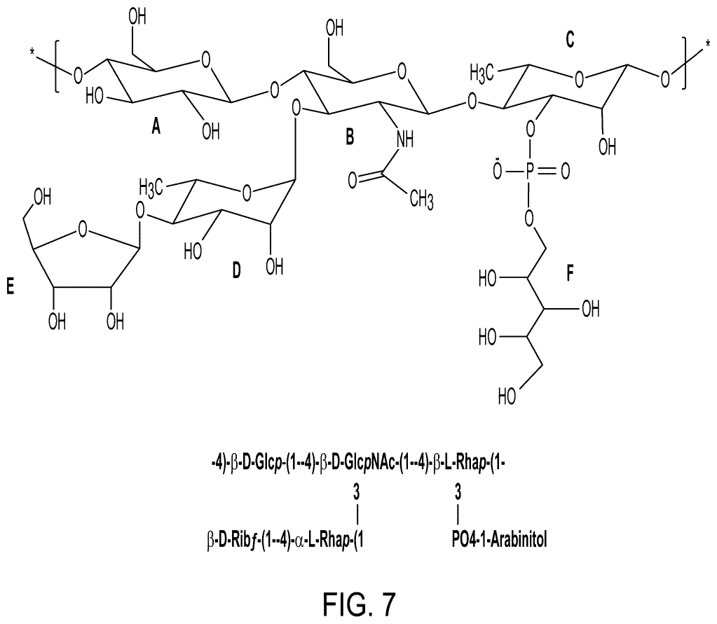

The serotype 24F glycoconjugates may also be characterized by the amount of branched Ribose residue which remains in the 24F polysaccharide. As mentioned above, it has been found that serotype 24F polysaccharide can loose the branched ribose residue (see FIG. 7).

Therefore, in an embodiment, the S. pneumoniae serotype 24F glycoconjugate of the present invention comprises a S. pneumoniae serotype 24F capsular polysaccharide wherein said S. pneumoniae serotype 24F capsular polysaccharide has a Ribose content greater than 75% when compared to native S. pneumoniae serotype 24F capsular polysaccharide wherein the Ribose content is considered to be about 100%. In an embodiment, the S. pneumoniae serotype 24F glycoconjugate of the present invention comprises a S. pneumoniae serotype 24F capsular polysaccharide wherein said S. pneumoniae serotype 24F capsular polysaccharide has a Ribose content greater than 90% when compared to native S. pneumoniae serotype 24F capsular polysaccharide wherein the Ribose content is considered to be about 100%. Preferably, the S. pneumoniae serotype 24F glycoconjugate of the present invention comprises a S. pneumoniae serotype 24F capsular polysaccharide wherein said S. pneumoniae serotype 24F capsular polysaccharide has a Ribose content greater than 95% when compared to native S. pneumoniae serotype 24F capsular polysaccharide wherein the Ribose content is considered to be about 100%.

In a most preferred embodiment, the S. pneumoniae serotype 24F glycoconjugate of the present invention comprises a S. pneumoniae serotype 24F capsular polysaccharide wherein said S. pneumoniae serotype 24F capsular polysaccharide has a Ribose content of about 100% when compared to native S. pneumoniae serotype 24F capsular polysaccharide wherein the Ribose content is considered to be about 100%.

In an embodiment, the serotype 24F saccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, the serotype 24F glycoconjugates of the invention are prepared using reductive amination chemistry. According to the present invention, reductive amination involves two steps, (1) oxidation (activation) of a purified saccharide, (2) reduction of the activated saccharide and a carrier protein to form a glycoconjugate.

In an embodiment, the serotype 24F glycoconjugates of the invention are prepared by conjugating an isolated serotype 24F capsular polysaccharide to a carrier protein by a process comprising the step of: (a) reacting said isolated serotype 24F capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

As mentioned above, before oxidation, sizing of the isolated serotype 24F capsular polysaccharide to a target molecular weight (MW) range can be performed. Therefore, in an embodiment, the isolated serotype 24F capsular polysaccharide is sized before oxidation.

In a preferred embodiment, the size of the isolated serotype 24F capsular polysaccharide is reduced by mechanical homogenization. In an embodiment, the size of the isolated serotype 24F polysaccharide is reduced by high pressure homogenization. In a most preferred embodiment, the isolated serotype 24F capsular polysaccharide is not sized by acid hydrolysis.

In an embodiment, the isolated serotype 24F capsular polysaccharide is not sized before oxidation.

In an embodiment, the oxidation step (a) is carried out at a pH of between 4.5 and 6.5. Preferably, the oxidation step (a) is carried out at a pH of between 5.0 and 6.0.

In an embodiment, the oxidation step (a) is carried out at a pH of about 5.0.

Following the oxidation step (a) the saccharide is said to be activated and is referred to as “activated polysaccharide”.

In an embodiment, the activated serotype 24F polysaccharide of the present invention has a weight average molecular weight (Mw) of between 50 kDa and 400 kDa. In an embodiment, the weight average molecular weight (Mw) is between 120 kDa and 250 kDa. In a most preferred embodiment, the weight average molecular weight (Mw) is between 120 kDa and 200 kDa.

In an embodiment, the activated serotype 24F polysaccharide of the present invention retains at least 75% of the branched Ribose. In an embodiment, the activated serotype 24F polysaccharide of the present invention retains at least 90% of the branched Ribose. In a preferred embodiment, the activated serotype 24F polysaccharide of the present invention retains at least 95% of the branched Ribose.

In a most preferred embodiment, the activated serotype 24F polysaccharide of the present invention retains about 100% of the branched Ribose.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO⁴⁻) and orthoperiodate (IO₆⁵⁻) and the various salts of periodate (e.g., sodium periodate and potassium periodate).

In a preferred embodiment, the oxidizing agent is sodium periodate. In an embodiment, the periodate used for the oxidation is metaperiodate. In a most preferred embodiment, the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the polysaccharide with 0.1-2 molar equivalents of periodate. Preferably step a) comprises reacting the polysaccharide with 0.5-1.5 molar equivalents of periodate. Most preferably, step a) comprises reacting the polysaccharide with 0.9-1.1 molar equivalents of periodate.

In an embodiment the degree of oxidation (also named “degree of activation” in the present document) of the activated serotype 24F polysaccharide is between 2 and 20. In a preferred embodiment the degree of oxidation of the activated serotype 24F polysaccharide is between 4 and 15. In a most preferred embodiment the degree of oxidation of the activated serotype 24F polysaccharide is 9±3.

In one embodiment the activated serotype 24F polysaccharide and the carrier protein are lyophilised before step b). Preferably lyophilisation occurs after step a). In one embodiment the activated polysaccharide is lyophilised after step a) and the carrier protein is also lyophilised. In one embodiment the activated serotype 24F polysaccharide is lyophilised after step a) and the carrier protein is also lyophilised, and the activated polysaccharide and the carrier protein are reconstituted in the same solution.

In an embodiment, the activated serotype 24F polysaccharide and the carrier protein are lyophilised independently (discrete lyophilization). In an embodiment, the activated serotype 24F polysaccharide and the carrier protein are lyophilised together (co-lyophilized). In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is sucrose.

In an embodiment the initial input ratio (weight by weight) of activated serotype 24F capsular polysaccharide to carrier protein at step b) is between 2:1 and 0.5:1. In an embodiment the initial input ratio (weight by weight) of activated serotype 24F capsular polysaccharide to carrier protein is between 1.2:1 and 0.6:1. Preferably, the initial input ratio (weight by weight) of activated serotype 24F capsular polysaccharide to carrier protein is between 0.9:1 and 0.7:1.

In an embodiment, the reduction reaction (c) is carried out in aqueous solvent. Preferably, the reduction reaction (c) is carried out in aprotic solvent.

In an embodiment, the reduction reaction (c) is carried out in the presence of dimethylsulphoxide (DMSO) or dimethylformamide (DMF). Preferably, the reduction reaction (c) is carried out in the presence of dimethylsulphoxide (DMSO).

In an embodiment, the reduction reaction (c) is carried out in DMSO (dimethylsulfoxide) or in DMF (dimethylformamide)) solvent.

Most preferably, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeiPrN-BH3, benzylamine-BH3 or 5-ethyl-2-methylpyridine borane (PEMB). In an embodiment, the reducing agent is sodium triacetoxyborohydride. In an embodiment, the reducing agent is sodium cyanoborohydride in the present of nickel (see WO2018144439). In a preferred embodiment, the reducing agent is sodium cyanoborohydride.

In one embodiment between 0.2 and 5 molar equivalents of reducing agent is used in step c). Preferably, between 0.5 and 2.5 molar equivalents of reducing agent is used in step c). Most preferably, between 1.5 and 2.5 molar equivalent of reducing agent is used in step c).

In one embodiment about 2 molar equivalents of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

In an embodiment capping is achieved by mixing the product of step c) with about 2 molar equivalents of sodium borohydride.

Following conjugation to the carrier protein, the serotype 24F glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the serotype 24F glycoconjugate of the present invention comprises the step of purifying the glycoconjugate after it is produced.

### 1.5 S. pneumoniae Serotype 35B Glycoconjugate of the Invention

In an aspect the invention relates to S. pneumoniae serotype 23B glycoconjugates.

The structure of Streptococcus pneumoniae serotype 35B polysaccharide is known in the art (see e.g. Geno K et al. (2015) Clin Microbiol Rev Vol 28:3, p 871-899).

In an embodiment, the capsular S. pneumoniae serotype 35B saccharide used in the present invention is a synthetic carbohydrate.

In a preferred embodiment though, the source of bacterial polysaccharide according to this invention can be Streptococcus pneumoniae serotype 35B bacterial cells. Bacterial strains which can be used as source of Streptococcus pneumoniae serotype 35B polysaccharides may be obtained from established culture collections (such as for example from the Streptococcal Reference Laboratory (Centers for Disease Control and Prevention, Atlanta, GA USA)) or clinical specimens.

Serotype 35B saccharides can be obtained directly from bacteria using isolation procedures known to one of ordinary skill in the art. They can also be purchased (such as for example from the American Type Culture Collection (ATCC, Manassas, VA USA) (e.g., reference No. ATCC 539-X, No. ATCC 540-X, No. ATCC 541-X)).

In case the serotype 35B saccharide is obtained directly from bacteria, the bacterial cells can be grown in a medium, preferably in a soy based medium. Following fermentation of bacterial cells that produce S. pneumoniae serotype 35B capsular polysaccharides, the bacterial cells can be lysed to produce a cell lysate. The serotype 35B polysaccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020170190). The purified serotype 35B capsular polysaccharide can then be used for the preparation of glycoconjugates.

The isolated serotype 35B capsular saccharide obtained by purification of serotype 35B polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In a preferred embodiment, the isolated serotype 35B capsular polysaccharide (i.e. purified before further treatment) has a weight average molecular weight between 100 kDa and 5000 kDa. In an embodiment, the isolated serotype 35B capsular polysaccharide has a weight average molecular weight between 300 kDa and 2000 kDa. In a preferred embodiment, the isolated serotype 35B capsular polysaccharide has a weight average molecular weight between 500 kDa and 1000 kDa.

Any whole number integer within any of the above ranges is contemplated as an embodiment of the disclosure.

The size of the purified serotype 35B polysaccharide may be reduced while preserving critical features of the structure of the polysaccharide. Mechanical or chemical sizing maybe employed.

However, S. pneumoniae serotype 35B polysaccharide has been found to be cleaved during activation with periodate, which is the classical oxidant used in the commonly used reductive amination process. It appears that periodate oxidation occurs on the backbone of serotype 35B polysaccharide and cleaves the mannitol or ribitol, leading to size reduction. The activation with periodate results in a decrease in polysaccharide Mw. Therefore, in the case where activation with periodate, is used, the serotype 35B capsular polysaccharide is not sized.

Therefore, in an embodiment, the isolated serotype 35B capsular polysaccharide is not sized.

In an embodiment, the isolated serotype 35B capsular polysaccharide before conjugation has a weight average molecular weight between 100 kDa and 5,000 kDa. In an embodiment, the isolated serotype 35B capsular polysaccharide before conjugation has a weight average molecular weight between 300 kDa and 2000 kDa. In an even preferred embodiment, the isolated serotype 35B capsular polysaccharide before conjugation has a weight average molecular weight between 500 kDa and 1000 kDa.

The weight average molecular weight (Mw) of the saccharide before conjugation refers to the Mw before activation of the polysaccharide (i.e. before reacting the polysaccharide with an activating agent).

In an embodiment, the serotype 35B glycoconjugate of the present invention comprises a serotype 35B capsular polysaccharide wherein the weight average molecular weight (Mw) of said polysaccharide is between 15 kDa and 100 kDa. In an embodiment, the weight average molecular weight (Mw) is between 25 kDa and 50 kDa. In a most preferred embodiment, the weight average molecular weight (Mw) is between about 30 kDa and about 40 kDa.

In some embodiments, the serotype 35B glycoconjugate of the invention has a weight average molecular weight (Mw) of between 250 kDa and 7,500 kDa. In other embodiments, the serotype 35B glycoconjugate has a weight average molecular weight (Mw) of between 500 kDa and 5,000 kDa. Preferably, the serotype 35B glycoconjugate has a weight average molecular weight (Mw) of between 1,000 kDa and 4,000 kDa.

The serotype 35B glycoconjugates of the invention may also be characterized by the ratio (weight/weight) of saccharide to carrier protein. In some embodiments, the ratio of serotype 35B polysaccharide to carrier protein in the glycoconjugate (w/w) is between 0.4 and 3.0. Preferably, the saccharide to carrier protein ratio (w/w) is between 0.4 and 2.0. Even more preferably, the saccharide to carrier protein ratio (w/w) is between 0.5 and 1.5.

Another way to characterize the serotype 35B glycoconjugates of the invention is by the number of lysine residues in the carrier protein (e.g., CRM197, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 35B glycoconjugate of the invention is between 2 and 15. Preferably, the degree of conjugation of the serotype 35B glycoconjugate of the invention is between 5 and 10.

The serotype 35B glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In an embodiment, the serotype 35B glycoconjugate comprises less than about 40% of free serotype 35B polysaccharide compared to the total amount of serotype 35B polysaccharide. In an embodiment, the serotype 35B glycoconjugate comprises less than about 20% of free serotype 35B polysaccharide compared to the total amount of serotype 35B polysaccharide. In a preferred embodiment the serotype 35B glycoconjugate comprises less than about 10% of free serotype 35B polysaccharide compared to the total amount of serotype 35B polysaccharide.

The serotype 35B glycoconjugates may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V0), (Kd=0), and the fraction representing the maximum retention (Vi), (Kd=1). The fraction at which a specified sample attribute is reached (Ve), is related to Kd by the expression, Kd=(Ve−V0)/(Vi−V0).

In an embodiment, at least 40% of the serotype 35B glycoconjugate has a Kd below or equal to 0.3 in a CL-4B column. In a preferred embodiment, at least 60% of the glycoconjugate has a Kd below or equal to 0.3 in a CL-4B column. In a preferred embodiment, between 50% and 80% of the serotype 35B glycoconjugate has a Kd below or equal to 0.3 in a CL-4B column.

In an embodiment, the serotype 35B saccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (preferably CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In a preferred embodiment, the serotype 35B glycoconjugates of the invention are prepared using reductive amination chemistry. According to the present invention, reductive amination involves two steps, (1) oxidation (activation) of a purified saccharide, (2) reduction of the activated saccharide and a carrier protein to form a glycoconjugate.

In an embodiment, the serotype 35B glycoconjugates of the invention are prepared by conjugating an isolated serotype 35B capsular polysaccharide to a carrier protein by a process comprising the step of: (a) reacting said isolated serotype 35B capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

Preferably, the isolated serotype 35B capsular polysaccharide is not sized before oxidation.

In a preferred embodiment, step (a) is quenched by addition of a quenching agent to stop oxidation.

Therefore in a preferred embodiment, the serotype 35B glycoconjugates of the invention are prepared by conjugating an isolated serotype 35B capsular polysaccharide to a carrier protein by a process comprising the step of: (a) reacting said isolated serotype 35B capsular polysaccharide with an oxidizing agent; (a′) quenching the oxidation reaction by addition of a quenching agent resulting in an activated serotype 35B capsular polysaccharide; (b) compounding the activated polysaccharide of step (a′) with a carrier protein; and (c) reacting the compounded activated polysaccharide and carrier protein with a reducing agent to form a glycoconjugate.

In an embodiment, the oxidation step (a) is carried out at a pH of between 5.0 and 7.0. Preferably, the oxidation step (a) is carried out at a pH of between 5.5 and 6.5.

In an embodiment, the oxidation step (a) is carried out at a pH of about 6.0.

Following the oxidation step (a)-(a′) the saccharide is said to be activated and is referred to as “activated polysaccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid, the term also includes both metaperiodate (IO⁴⁻) and orthoperiodate (IO₆⁵⁻) and the various salts of periodate (e.g., sodium periodate and potassium periodate).

In a preferred embodiment, the oxidizing agent is sodium periodate. In an embodiment, the periodate used for the oxidation is metaperiodate. In a most preferred embodiment, the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the polysaccharide with 0.05-0.2 molar equivalents of periodate. Preferably step a) comprises reacting the polysaccharide with 0.09-0.11 molar equivalents of periodate. Most preferably, step a) comprises reacting the polysaccharide with about 0.1 molar equivalents of periodate.

In one embodiment, the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

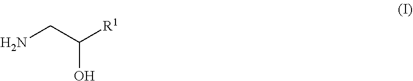

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms.

Preferably, the quenching agent is a compound of formula (II):

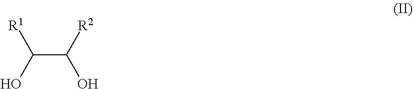

wherein R¹ and R² are each independently selected from H, methyl, ethyl, propyl or isopropyl.

In a preferred embodiment, the quenching agent is glycerol, ethylene glycol, propan-1,2-diol, butan-1,2-diol or butan-2,3-diol, or ascorbic acid. In a most preferred embodiment, the quenching agent is butan-2,3-diol.

In a preferred embodiment, the isolated serotype 35B polysaccharide is activated by a process comprising the step of:
(a) reacting the isolated serotype 35B polysaccharide with periodate;
(b) quenching the oxidation reaction by addition of butan-2,3-diol resulting in an activated serotype 35B polysaccharide.

Following the oxidation step of the polysaccharide, the polysaccharide is said to be activated and is referred to as “activated polysaccharide” here below.

In an embodiment, the activated serotype 35B polysaccharide of the present invention has a weight average molecular weight (Mw) of between 15 kDa and 100 kDa. In an embodiment, the weight average molecular weight (Mw) is between 25 kDa and 50 kDa. In a most preferred embodiment, the weight average molecular weight (Mw) is between 30 kDa and 40 kDa.

In an embodiment the degree of oxidation (also named “degree of activation” in the present document) of the activated serotype 35B polysaccharide is between 2 and 20. In a preferred embodiment the degree of oxidation of the activated serotype 35B polysaccharide is between 4 and 15. In a most preferred embodiment the degree of oxidation of the activated serotype 35B polysaccharide is 9±3.

In one embodiment the activated serotype 35B polysaccharide and the carrier protein are lyophilised before step b). Preferably lyophilisation occurs after step a). In one embodiment the activated polysaccharide is lyophilised after step a) and the carrier protein is also lyophilised. In one embodiment the activated serotype 35B polysaccharide is lyophilised after step a) and the carrier protein is also lyophilised, and the activated polysaccharide and the carrier protein are reconstituted in the same solution.

In an embodiment, the activated serotype 35B polysaccharide and the carrier protein are lyophilised independently (discrete lyophilization). In an embodiment, the activated serotype 35B polysaccharide and the carrier protein are lyophilised together (co-lyophilized).

In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is sucrose.

In an embodiment the initial input ratio (weight by weight) of activated serotype 35B capsular polysaccharide to carrier protein at step b) is between 2:1 and 0.5:1. In an embodiment the initial input ratio (weight by weight) of activated serotype 35B capsular polysaccharide to carrier protein is between 1.2:1 and 0.6:1. Preferably, the initial input ratio (weight by weight) of activated serotype 35B capsular polysaccharide to carrier protein is between 0.9:1 and 0.7:1.

In an embodiment, the reduction reaction (c) is carried out in aqueous solvent. Preferably, the reduction reaction (c) is carried out in aprotic solvent.

In an embodiment, the reduction reaction (c) is carried out in the presence of dimethylsulphoxide (DMSO) or dimethylformamide (DMF). Preferably, the reduction reaction (c) is carried out in the presence of dimethylsulphoxide (DMSO).

In an embodiment, the reduction reaction (c) is carried out in DMSO (dimethylsulfoxide) or in DMF (dimethylformamide)) solvent.

Most preferably, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeiPrN-BH3, benzylamine-BH3 or 5-ethyl-2-methylpyridine borane (PEMB). In an embodiment, the reducing agent is sodium triacetoxyborohydride. In an embodiment, the reducing agent is sodium cyanoborohydride in the present of nickel (see WO2018144439). In a preferred embodiment, the reducing agent is sodium cyanoborohydride.

In one embodiment between 0.2 and 5 molar equivalents of reducing agent is used in step c). Preferably, between 0.5 and 1.5 molar equivalents of reducing agent is used in step c). Most preferably, between 0.9 and 1.1 molar equivalent of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

Following conjugation to the carrier protein, the serotype 35B glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the serotype 35B glycoconjugate of the present invention comprises the step of purifying the glycoconjugate after it is produced.

### 1.6 S. pneumoniae Serotype 3 Glycoconjugates of the Invention

In an aspect the invention relates to compositions comprising a S. pneumoniae serotype 3 glycoconjugate.

The structure of Streptococcus pneumoniae serotype 3 polysaccharide is known in the art. The polysaccharide repeating unit of serotype 3 consists of a linear disaccharide unit with one glucopyranose (Glcp) and one glucuronic acid (GlcpA) (see e.g. Geno K et al. (2015) Clin Microbiol Rev Vol 28:3, p 871-899).

In an embodiment, the capsular S. pneumoniae serotype 3 saccharide used in the present invention is a synthetic carbohydrate. Preparation of a synthetic Streptococcus pneumoniae type 3 capsular saccharide can for example be conducted as disclosed in WO2017178664 or WO2015040140.

In a preferred embodiment though, the source of bacterial polysaccharide according to this invention can be Streptococcus pneumoniae serotype 3 bacterial cells. Bacterial strains which can be used as source of Streptococcus pneumoniae serotype 3 polysaccharides may be obtained from established culture collections (such as for example from the Streptococcal Reference Laboratory (Centers for Disease Control and Prevention, Atlanta, GA USA)) or clinical specimens.

Serotype 3 polysaccharides can be obtained directly from bacteria using isolation procedures known to one of ordinary skill in the art (see for example methods disclosed in US2006/0228380, US2006/0228381, US2007/0184071, US2007/0184072, US2007/0231340, and US2008/0102498 and WO2008/118752). They can also be produced using synthetic protocols known to the man skilled in the art. They can also be purchased (such as for example from the American Type Culture Collection (ATCC, Manassas, VA USA) (e.g., reference No. ATCC 172-X or ATCC 33-X)).

In case the serotype 3 polysaccharide is obtained directly from bacteria, the bacterial cells can be grown in a medium, preferably in a soy based medium. Following fermentation of bacterial cells that produce S. pneumoniae serotype 3 capsular polysaccharides, the bacterial cells can be lysed to produce a cell lysate. The serotype 3 polysaccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381 and WO2008/118752). The purified serotype 3 capsular polysaccharide can then be used for the preparation of immunogenic conjugates.

The isolated serotype 3 capsular polysaccharide obtained by purification of serotype 3 polysaccharide from the S. pneumoniae lysate and optionally sizing of the purified polysaccharide can be characterized by different parameters including, for example the weight average molecular weight (Mw).

The molecular weight of the polysaccharide can be measured by Size Exclusion Chromatography (SEC) combined with Multiangle Laser Light Scattering detector (MALLS).

In an embodiment, the isolated serotype 3 capsular polysaccharide (i.e. purified before further treatment) has a weight average molecular weight between 5 kDa and 5,000 kDa. In an embodiment, the isolated capsualr polysaccharide has a weight average molecular weight between 100 kDa and 4,000 kDa. In a preferred embodiment, the isolated capsular polysaccharide has a weight average molecular weight between 1,000 kDa and 3,500 kDa.

Preferably, in order to generate serotype 3 conjugates with advantageous filterability characteristics, immunogenicity and/or yields, sizing of the polysaccharide to a target molecular weight range is performed prior to the conjugation to a carrier protein. Advantageously, the size of the purified serotype 3 polysaccharide is reduced while preserving critical features of the structure of the polysaccharide. Mechanical or chemical sizing maybe employed.

In an embodiment, the size of the purified serotype 3 polysaccharide is reduced by chemical hydrolysis. Chemical hydrolysis maybe conducted using a mild acid (e.g acetic acid, formic acid, propanoic acid). In an embodiment, chemical hydrolysis is conducted using formic acid. In an embodiment, chemical hydrolysis is conducted using propanoic acid. In a preferred embodiment, chemical hydrolysis is conducted using acetic acid. Chemical hydrolysis may also be conducted using a diluted strong acid (such as diluted hydrochloric acid, diluted sulfuric acid, diluted phosphoric acid, diluted nitric acid or diluted perchloric acid). In an embodiment, chemical hydrolysis is conducted using diluted hydrochloric acid. In an embodiment, chemical hydrolysis is conducted using diluted sulfuric acid. In an embodiment, chemical hydrolysis is conducted using diluted phosphoric acid. In an embodiment, chemical hydrolysis is conducted using diluted nitric acid. In an embodiment, chemical hydrolysis is conducted using diluted perchloric acid.

The size of the purified serotype 3 polysaccharide can also be reduced by mechanical homogenization. In an embodiment, the size of the purified serotype 3 polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer. The high-pressure homogenization process can be appropriate for reducing the size of the purified serotype 3 polysaccharide while preserving the structural features of the polysaccharide.

In an embodiment, the isolated serotype 3 capsular polysaccharide is sized to a weight average molecular weight between 5 kDa and 1000 kDa. In an embodiment, the isolated serotype 3 capsular polysaccharide is sized to a weight average molecular weight between 50 kDa and 300 kDa. In a preferred embodiment, the isolated serotype 3 capsular polysaccharide is sized to a weight average molecular weight between 100 kDa and 300 kDa.

In an embodiment, the isolated serotype 3 capsular polysaccharide is sized to a weight average molecular weight of between about 200 kDa and about 300 kDa.

In an embodiment, the isolated serotype 3 capsular polysaccharide is sized to a weight average molecular weight of between about 100 kDa and about 200 kDa.

In an embodiment, the isolated serotype 3 capsular polysaccharide is not sized.

In an embodiment, the serotype 3 glycoconjugate of the present invention comprises a serotype 3 capsular polysaccharide wherein the weight average molecular weight (Mw) of said polysaccharide before conjugation is between 50 kDa and 1,000 kDa. Preferably, the weight average molecular weight (Mw) is between 100 kDa and 300 kDa.

The weight average molecular weight (Mw) of the serotype 3 saccharide before conjugation refers to the Mw before the activation of the serotype 3 polysaccharide (i.e. after an eventual sizing step but before reacting the polysaccharide with an activating agent). In the context of the present invention the Mw of the serotype 3 polysaccharide is not substantially modified by the activation step and the Mw of the serotype 3 polysaccharide incorporated in the conjugate is similar to the Mw of the polysaccharide as measured before activation.

In an embodiment, the serotype 3 glycoconjugate of the present invention comprises a serotype 3 capsular polysaccharide wherein the weight average molecular weight (Mw) of said polysaccharide before conjugation is between 100 kDa and 200 kDa.

In an embodiment, the serotype 3 glycoconjugate of the present invention comprises a serotype 3 capsular polysaccharide wherein the weight average molecular weight (Mw) of said polysaccharide before conjugation is between 200 kDa and 300 kDa.

In some embodiments, the serotype 3 glycoconjugate of the invention has a weight average molecular weight (Mw) of between 250 kDa and 20,000 kDa. In other embodiments, the serotype 3 glycoconjugate has a weight average molecular weight (Mw) of between 500 kDa and 15,000 kDa. In yet other embodiments, the serotype 3 glycoconjugate has a weight average molecular weight (Mw) of between 500 kDa and 10,000 kDa. Preferably, the serotype 3 glycoconjugate has a weight average molecular weight (Mw) of between 500 kDa and 5,000 kDa.

In an embodiment, the serotype 3 glycoconjugate has a weight average molecular weight (Mw) of between 600 kDa and 3,000 kDa.

The molecular weight of the polysaccharide can be measured by Size Exclusion Chromatography (SEC) combined with Multiangle Laser Light Scattering detector (MALLS).

Another way to characterize the serotype 3 glycoconjugates of the invention is by the number of lysine residues in the carrier protein (e.g., CRM₁₉₇ or SCP) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 3 glycoconjugate of the invention is between 2 and 15.

In a preferred embodiment, the degree of conjugation of the serotype 3 glycoconjugate of the invention is between 4 and 7. In some such embodiments, the carrier protein is CRM₁₉₇. In other such embodiments, the carrier protein is SCP.

The serotype 3 glycoconjugates of the invention may also be characterized by the ratio (weight/weight) of saccharide to carrier protein. In some embodiments, the ratio of serotype 3 polysaccharide to carrier protein in the glycoconjugate (w/w) is between 0.5 and 3.0. In other embodiments, the saccharide to carrier protein ratio (w/w) is between 0.5 and 1.5. In a preferred embodiment, the ratio of serotype 3 capsular polysaccharide to carrier protein in the conjugate is between 0.9 and 1.1.

The serotype 3 glycoconjugates of the invention may also be characterized by the number of covalent linkages between the carrier protein and the saccharide as a function of repeat units of the saccharide. In one embodiment, the serotype 3 glycoconjugate of the invention comprises at least one covalent linkage between the carrier protein and the polysaccharide for every 4 saccharide repeat units of the polysaccharide. In another embodiment, the covalent linkage between the carrier protein and the polysaccharide occurs at least once in every 10 saccharide repeat units of the polysaccharide. In another embodiment, the covalent linkage between the carrier protein and the polysaccharide occurs at least once in every 15 saccharide repeat units of the polysaccharide. In a further embodiment, the covalent linkage between the carrier protein and the polysaccharide occurs at least once in every 25 saccharide repeat units of the polysaccharide. In a further embodiment, the covalent linkage between the carrier protein and the polysaccharide occurs at least once in every 50 saccharide repeat units of the polysaccharide. In yet a further embodiment, the covalent linkage between the carrier protein and the polysaccharide occurs at least once in every 100 saccharide repeat units of the polysaccharide.

In other embodiments, the serotype 3 glycoconjugate of the invention comprises at least one covalent linkage between the carrier protein and the polysaccharide for every 5 to 10 saccharide repeat units of the polysaccharide.

In other embodiments, the serotype 3 glycoconjugate of the invention comprises at least one covalent linkage between the carrier protein and the polysaccharide for every 10 to 20 saccharide repeat units of the polysaccharide.

In some embodiments, the carrier protein is CRM₁₉₇ and the covalent linkage between the CRM₁₉₇ and the polysaccharide occurs at least once in every 4, 10, 15 or 25 saccharide repeat units of the polysaccharide. In frequent embodiments, the carrier protein is SCP and the covalent linkage between the SCP and the polysaccharide occurs at least once in every 4, 10, 15 or 25 saccharide repeat units of the polysaccharide.

The serotype 3 glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In a preferred embodiment, the serotype 3 glycoconjugate comprises less than about 50% of free serotype 3 polysaccharide compared to the total amount of serotype 3 polysaccharide. In a preferred embodiment the serotype 3 glycoconjugate comprises less than about 40% of free serotype 3 polysaccharide compared to the total amount of serotype 3 polysaccharide. In a yet preferred embodiment, the serotype 3 glycoconjugate comprises less than about 25% of free serotype 3 polysaccharide compared to the total amount of serotype 3 polysaccharide. In an even preferred embodiment, the serotype 3 glycoconjugate comprises less than about 20% of free serotype 3 polysaccharide compared to the total amount of serotype 3 polysaccharide. In a yet preferred embodiment, the serotype 3 glycoconjugate comprises less than about 15% of free serotype 3 polysaccharide compared to the total amount of serotype 3 polysaccharide.

The serotype 3 glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vi), (K_{d}=1). The fraction at which a specified sample attribute is reached (Vₑ), is related to K_{d} by the expression, K_{d}=(Ve−V₀)/(Vi−V₀).

In a preferred embodiment, at least 30% of the serotype 3 glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 45%, 50%, 55%, 60%, 65%, 70%, 75%, 80%, or 85% of the serotype 3 glycoconjugate has a K_{d} below or equal to 0.3 in a CL-4B column. In a preferred embodiment, at least 60% of the serotype 3 glycoconjugate has a K_{d} below or equal to 0.3 in a CL-4B column. In a preferred embodiment, between 50% and 80% of the serotype 3 glycoconjugate has a K_{d} below or equal to 0.3 in a CL-4B column. In a preferred embodiment, between 65% and 80% of the serotype 3 glycoconjugate has a K_{d} below or equal to 0.3 in a CL-4B column.

In an embodiment, the serotype 3 glycoconjugates of the present invention are prepared using reductive amination chemistry (see WO2006110381, WO2008143709, PCT/IB2022/054920).

According to the present invention, reductive amination involves two steps, (1) oxidation (activation) of a purified saccharide, (2) reduction of the activated saccharide and a carrier protein (e.g., CRM₁₉₇, TT or SCP) to form a glycoconjugate.

As mentioned above, before oxidation, sizing of the polysaccharide to a target molecular weight (MW) range can be performed.

Therefore, in an embodiment, the isolated polysaccharide is sized before oxidation.

In an embodiment, the isolated polysaccharide is sized to any of the target molecular weight (MW) range defined above.

In an embodiment, the isolated serotype 3 capsular polysaccharide is conjugated to a carrier protein by a process comprising the step of:
(a) reacting said isolated polysaccharide with an oxidizing agent;
(b) compounding the activated polysaccharide of step (a) with a carrier protein; and
(c) reacting the compounded activated polysaccharide and carrier protein with a reducing agent to form a glycoconjugate.

Following the oxidation step (a) the saccharide is said to be activated and is referred to as “activated polysaccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO₄₋) and orthoperiodate (IO₆⁵⁻) and the various salts of periodate (e.g., sodium periodate and potassium periodate).

In an embodiment, the oxidizing agent is periodate in the presence of bivalent cations (see WO2008/143709).

In an embodiment, the oxidizing agent is periodic acid. In an embodiment, the oxidizing agent is periodic acid in the presence of bivalent cations. In an embodiment, the oxidizing agent is periodic acid in the presence of Mg²⁺. In an embodiment, the oxidizing agent is periodic acid in the presence of Ca²⁺. In an embodiment, the oxidizing agent is orthoperiodate.

In an embodiment, the oxidizing agent is sodium periodate. In an embodiment, the periodate used for the oxidation is metaperiodate. In an embodiment the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the polysaccharide with 0.01-2 molar equivalents of periodate. Preferably step a) comprises reacting the polysaccharide with 0.1-2 molar equivalents of periodate.

In one embodiment step a) comprises reacting the polysaccharide with 0.01-2 molar equivalents of periodic acid. Preferably step a) comprises reacting the polysaccharide with 0.2-2 molar equivalents of periodic acid.

In a preferred embodiment the degree of oxidation (also named “degree of activation” in the present document) of the activated serotype 3 polysaccharide is between 2 and 30. In a preferred embodiment the degree of oxidation of the activated serotype 3 polysaccharide is between 2 to 20.

In an embodiment the degree of oxidation of the activated serotype 3 polysaccharide is between 2 to 8.

In an embodiment the degree of oxidation of the activated serotype 3 polysaccharide is between 11 to 19.

In one embodiment the activated polysaccharide and the carrier protein are lyophilised before step b). Preferably lyophilisation occurs after step a). In one embodiment the activated polysaccharide is lyophilised after step a) and the carrier protein is also lyophilised.

In one embodiment the activated polysaccharide is lyophilised after step a) and the carrier protein is also lyophilised, and the activated polysaccharide and the carrier protein are reconstituted in the same solution, this acts as compounding the activated polysaccharide and the carrier protein together.

In an embodiment, the activated polysaccharide and the carrier protein are lyophilised independently (discrete lyophilization). In an embodiment, the activated polysaccharide and the carrier protein are lyophilised together (co-lyophilized).

In one embodiment the lyophilization takes place in the presence of a non-reducing sugar, possible non-reducing sugars include sucrose, trehalose, raffinose, stachyose, melezitose, dextran, mannitol, lactitol and palatinit. In an embodiment the sugar is selected from the group consisting of sucrose, trehalose, and mannitol. In an embodiment the sugar is sucrose, trehalose or mannitol. In an embodiment the sugar is trehalose. In an embodiment the sugar is sucrose.

In an embodiment the initial input ratio (weight by weight) of activated serotype 3 capsular polysaccharide to carrier protein at step b) is between 4:1 and 0.1:1. In an embodiment the initial input ratio (weight by weight) of activated serotype 3 capsular polysaccharide to carrier protein is between 2:1 and 0.4:1.

In an embodiment, the reduction reaction (c) is carried out in aqueous solvent.

In an embodiment, the reduction reaction (c) is carried out in aprotic solvent. In an embodiment, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH3 or 5-ethyl-2-methylpyridine borane (PEMB). In an embodiment, the reducing agent is sodium triacetoxyborohydride. In a preferred embodiment, the reducing agent is sodium cyanoborohydride. In an embodiment, the reducing agent is sodium cyanoborohydride in the present of nickel (see WO2018144439).

In one embodiment between 0.2 and 20 molar equivalents of reducing agent is used in step c). In one embodiment between 0.5 and 3 molar equivalents of reducing agent is used in step c).

At the end of the reduction reaction, there may be unreacted aldehyde groups remaining in the conjugates, these may be capped using a suitable capping agent. In one embodiment this capping agent is sodium borohydride (NaBH₄).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

In an embodiment, the glycoconjugate of the present invention is prepared using CDI and/or CDT chemistry (see PCT/IB2022/054920).

CDI and/or CDT chemistry involves two steps, (1) reacting the isolated saccharide with CDI and/or CDT in an aprotic solvent to produce an activated saccharide (activation), (2) reacting the activated saccharide with a carrier protein (e.g. CRM₁₉₇ or SCP) to form a glycoconjugate.

In an embodiment, the activating agent of step (1) is 1,1′-carbonyldiimidazole (CDI). In an embodiment, the activating agent of step (1) is 1,1′-Carbonyl-di-(1,2,4-triazole) (CDT).

In an embodiment, the isolated serotype 3 capsular polysaccharide is conjugated to a carrier protein by a process comprising the step of:
(a) reacting said isolated polysaccharide with CDI and/or CDT in an aprotic solvent;
(b) reacting the activated polysaccharide of step (a) with a carrier protein in an aprotic solvent to form a glycoconjugate.

In an embodiment, serotype 3 glycoconjugates of the present invention are prepared using click chemistry (see e.g. PCT/IB2022/054914).

According to the present invention, click chemistry comprises three steps, (a) reacting an isolated serotype 3 capsular polysaccharide with a carbonic acid derivative and an azido linker in an aprotic solvent to produce an activated azido polysaccharide (activation of the polysaccharide), (b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein (activation of the carrier protein), (c) reacting the activated azido polysaccharide of step (a) with the activated alkyne-carrier protein of step (b) by Cu⁺¹ mediated azide-alkyne cycloaddition reaction to form a glycoconjugate.

Following step (a) the polysaccharide is said to be activated and is referred to herein as “activated polysaccharide” or “activated azido polysaccharide”.

Following step (b) the carrier is said to be activated and is referred to as “activated carrier”.

As mentioned above, before the activation (a), sizing of the polysaccharide to a target molecular weight (MW) range can be performed.

Therefore, in an embodiment, the isolated polysaccharide is sized before activation with a carbonic acid derivative and an azido linker.

In an embodiment, the isolated polysaccharide is sized to any of the target molecular weight (MVV) range defined above.

In an embodiment, said carbonic acid derivative is 1,1′-carbonyldiimidazole (CDI) or 1,1′-Carbonyl-di-(1,2,4-triazole) (CDT). Preferably, said carbonic acid derivative is 1,1′-carbonyldiimidazole (CDI).

In an embodiment, said azido linker is a compound of formula (I),

H₂N—X—N₃ (I)

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

In an embodiment, said azido linker is a compound of formula (II),

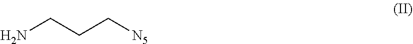

In an embodiment, said azido linker is 3-azido-propylamine.

In an embodiment, said agent bearing an N-Hydroxysuccinimide (NHS) moiety and an alkyne group is an agent bearing an N-Hydroxysuccinimide (NHS) moiety and a terminal alkyne.

In an embodiment, said agent bearing an N-Hydroxysuccinimide (NHS) moiety and an alkyne group is an agent bearing an N-Hydroxysuccinimide (NHS) moiety and a cycloalkyne.

In an embodiment, said agent bearing an N-Hydroxysuccinimide (NHS) moiety and an alkyne group is a compound of formula (III),

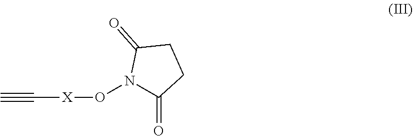

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an embodiment, said agent bearing an N-Hydroxysuccinimide (NHS) moiety and an alkyne group is a compound of formula (IV):

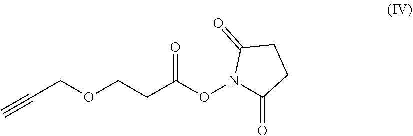 [SMILES: C#CCOCCC(=O)ON1C(=O)CCC1=O]

In an embodiment, step a) comprises reacting the polysaccharide with a carbonic acid derivative followed by reacting the carbonic acid derivative-activated polysaccharide with an azido linker in an aprotic solvent to produce an activated azido polysaccharide.

In an embodiment, at step a) the isolated polysaccharide is reacted with a carbonic acid derivative in an aprotic solvent.

In a preferred embodiment the isolated polysaccharide is reacted with a carbonic acid derivative in a solution consisting essentially of dimethylsulphoxide (DMSO).

In a preferred embodiment the isolated polysaccharide is reacted with CDI in dimethylsulphoxide (DMSO). In an embodiment the isolated polysaccharide is reacted with CDI in anhydrous DMSO.

Once the polysaccharide has been reacted with carbonic acid derivative, and following an eventual quenching of carbonic acid derivative with water, the carbonic acid derivative-activated polysaccharide is reacted with an azido linker.

In one embodiment step a) further comprises reacting the carbonic acid derivative-activated polysaccharide with an amount of azido linker that is between 0.01-10 molar equivalent to the amount of polysaccharide Repeat Unit of the activated polysaccharide (molar equivalent of RU).

In an embodiment, the conjugation reaction c) is carried out in aqueous buffer. In an embodiment, the conjugation reaction c) is carried out in aqueous buffer in the presence of copper (I) as catalyst. In an embodiment, the conjugation reaction c) is carried out in aqueous buffer in the presence an oxidant and of copper (I) as catalyst. In a preferred embodiment, the conjugation reaction c) is carried out in aqueous buffer in the presence of copper (I) as catalyst and ascorbate as oxidant. In an embodiment, THPTA (tris(3-hydroxypropyltriazolylmethyl)amine) and aminoguanidine may be further added to protect the protein from side reactions. Therefore, in a preferred embodiment, the conjugation reaction c) is carried out in aqueous buffer in the presence of copper (I) as catalyst and ascorbate as oxidant, wherein the reaction mixture further comprises THPTA (tris(3-hydroxypropyltriazolylmethyl)amine) and aminoguanidine.

Following the click conjugation reaction, there may remain unreacted azido groups in the conjugates, these may be capped using a suitable azido group capping agent. Therefore, in an embodiment, following step c), unreacted azido groups in the conjugates, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne.

In an embodiment, said azido group capping agent is a compound of formula (V),

≡-X—OH (V)

wherein X is (CH₂)ₙ wherein n is selected from 1 to 15.

In one embodiment this azido group capping agent is propargyl alcohol.

Therefore, in an embodiment, following step (c) the process further comprises a step of capping the unreacted azido groups remained in the conjugates with an azido group capping agent.

Following the click conjugation reaction, unreacted alkyne groups may remain present in the conjugates, these may be capped using a suitable alkyne group capping agent. In one embodiment this alkyne group capping agent is an agent bearing an azido group.

In an embodiment, said alkyne group capping agent is a compound of formula (VI),

N₃—X—OH (VI)

wherein X is (CH₂)ₙ wherein n is selected from 1 to 15.

In one embodiment this alkyne group capping agent is 3-azido-1-propanol.

Therefore, in an embodiment, following step (c) the process further comprises a step of capping the unreacted alkyne groups remained in the conjugates with an alkyne group capping agent.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

In an aspect, the serotype 3 glycoconjugate of the invention us produced according to click chemistry as disclosed above and in application PCT/IB2022/054914, which is incorporated by reference in its entirety. Therefore, in an embodiment, the serotype 3 glycoconjugate of the invention comprises a serotype 3 saccharide covalently conjugated to a carrier protein (CP) through a spacer and have the general formula (VII):

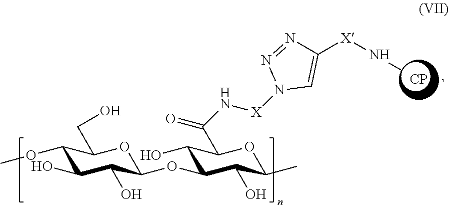

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4, and wherein X is selected from the group consisting of CH₂O(CH₂)ₙ₋CH₂C═O, CH₂O(CH₂CH₂O)_{m′}(CH₂)_{n″}CH₂C═O, where n″ is selected from 0 to 10 and m′ is selected from 0 to 4.

Formula (VII) is a schematic representation of serotype 3 glycoconjugates of the invention. It should not be understood that a linkage is present at every repeating unit of the saccharide. Rather, a majority of the S. pneumoniae serotype 3 saccharide repeating unit remains unmodified and covalent linkages between the carrier protein and the saccharide is for a minority of the saccharide repeat units. Additionally, an individual carrier protein (CP) molecule may be linked to more than one S. pneumoniae serotype 3 saccharide molecule and an individual S. pneumoniae serotype 3 saccharide molecule can be linked to more than one individual carrier protein (CP) molecule.

In a preferred embodiment, the serotype 3 glycoconjugate of the invention comprises a serotype 3 saccharide covalently conjugated to a carrier protein (CP) through a spacer and have the general formula (VII), wherein X is CH₂(CH₂)_{n′}, where n′ is 2 and wherein X is CH₂O(CH₂)_{n″}CH₂C═O where n″ is 1.

In an embodiment, the serotype 3 glycoconjugate of the invention comprises a serotype 3 saccharide covalently conjugated to a carrier protein (CP) through a spacer and have the general formula (VII), wherein X is CH₂(CH₂)_{n′}, where n′ is selected from 1 to 10 and wherein X is CH₂O(CH₂)_{n″}CH₂C═O where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0.

In an embodiment, the serotype 3 glycoconjugate of the invention comprises a serotype 3 saccharide covalently conjugated to a carrier protein (CP) through a spacer and have the general formula (VII), wherein X is CH₂(CH₂)_{n′}, where n′ is selected from 1 to 10 and wherein CH₂O(CH₂CH₂O)_{m′}(CH₂)_{n″}—CH₂C═O, where n″ is selected from 0 to 10 and m′ is selected from 0 to 4.

In an embodiment, the serotype 3 glycoconjugate of the invention comprises a serotype 3 saccharide covalently conjugated to a carrier protein (CP) through a spacer and have the general formula (VII), wherein X is (CH₂CH₂O)ₘCH₂CH₂, where m is selected from 1 to 4 and wherein X is CH₂O(CH₂)_{n″}CH₂C═O, where n″ is selected from 0 to 10. In an embodiment, m is selected from 1 to 3 and n″ is selected from 0 to 10.

In an embodiment, the serotype 3 glycoconjugate of the invention comprises a serotype 3 saccharide covalently conjugated to a carrier protein (CP) through a spacer and have the general formula (VII), wherein X is (CH₂CH₂O)ₘCH₂CH₂, where m is selected from 1 to 4 and wherein X is CH₂O(CH₂CH₂O)_{m′}(CH₂)_{n″}CH₂C═O, where n″ is selected from 0 to 10 and m′ is selected from 0 to 4.

In an embodiment, the serotype 3 glycoconjugate of the invention comprises a serotype 3 saccharide covalently conjugated to a carrier protein (CP) through a spacer and have the general formula (VII), wherein X is NHCO(CH₂)_{n′}, where n′ is selected from 1 to 10 and wherein X is CH₂O(CH₂)_{n″}CH₂C═O, where n″ is selected from 0 to 10.

In an embodiment, the serotype 3 glycoconjugate of the invention comprises a serotype 3 saccharide covalently conjugated to a carrier protein (CP) through a spacer and have the general formula (VII), wherein X is NHCO(CH₂)_{n′}, where n′ is selected from 1 to 10 and wherein X is CH₂O(CH₂CH₂O)_{m′}(CH₂)_{n″}—CH₂C═O, where n″ is selected from 0 to 10 and m′ is selected from 0 to 4.

In an embodiment, the serotype 3 glycoconjugate of the invention comprises a serotype 3 saccharide covalently conjugated to a carrier protein (CP) through a spacer and have the general formula (VII), wherein X is NHCO(CH₂CH₂O)ₘCH₂CH₂, where m is selected from 1 to 4 and wherein X′ is CH₂O(CH₂)_{n″}CH₂C═O, where n″ is selected from 0 to 10. In an embodiment, m is selected from 1 to 3 and n″ is selected from 0 to 10.

In an embodiment, the serotype 3 glycoconjugate of the invention comprises a serotype 3 saccharide covalently conjugated to a carrier protein (CP) through a spacer and have the general formula (VII), wherein X is NHCO(CH₂CH₂O)ₘCH₂CH₂, where m is selected from 1 to 4 and wherein X′ is CH₂O(CH₂CH₂O)_{m′}(CH₂)_{n″}CH₂C═O, where n″ is selected from 0 to 10 and m′ is selected from 0 to 4.

In an embodiment, the serotype 3 glycoconjugate of the invention comprises a serotype 3 saccharide covalently conjugated to a carrier protein (CP) through a spacer and have the general formula (VII), wherein X is OCH₂(CH₂)_{n′}, where n′ is selected from 1 to 10 and wherein X is CH₂O(CH₂)_{n″}CH₂C═O, where n″ is selected from 0 to 10.

In an embodiment, the serotype 3 glycoconjugate of the invention comprises a serotype 3 saccharide covalently conjugated to a carrier protein (CP) through a spacer and have the general formula (VII), wherein X is OCH₂(CH₂)_{n′}, where n′ is selected from 1 to 10 and wherein X is CH₂O(CH₂CH₂O)_{m′}(CH₂)_{n″}—CH₂C═O, where n″ is selected from 0 to 10 and m′ is selected from 0 to 4.

In an embodiment, the serotype 3 glycoconjugate of the invention comprises a serotype 3 saccharide covalently conjugated to a carrier protein (CP) through a spacer and have the general formula (VII), wherein X is O(CH₂CH₂O)ₘCH₂CH₂, where m is selected from 1 to 4 and wherein X′ is CH₂O(CH₂)_{n″}CH₂C═O, where n″ is selected from 0 to 10.

In an embodiment, the serotype 3 glycoconjugate of the invention comprises a serotype 3 saccharide covalently conjugated to a carrier protein (CP) through a spacer and have the general formula (VII), wherein X is O(CH₂CH₂O)ₘCH₂CH₂, where m is selected from 1 to 4 and wherein X′ is CH₂O(CH₂CH₂O)_{m′}(CH₂)_{n″}—CH₂C═O, where n″ is selected from 0 to 10 and m′ is selected from 0 to 4.

Following conjugation to the carrier protein, the serotype 3 glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

### 1.7 S. pneumoniae serotypes 1, 4, 5, 6A, 6B, 7F, 8, 9V, 10A, 11A, 12F, 15B, 14, 18C, 19A, 19F, 22F, 23F and 33F glycoconjugates of the invention

In an aspect the invention relates to compositions comprising S. pneumoniae serotype 1, 4, 5, 6A, 6B, 7F, 8, 9V, 10A, 11A, 12F, 14, 15B, 18C, 19A, 19F, 22F, 23F and/or 33F glycoconjugate(s).

The structures of Streptococcus pneumoniae serotypes 1, 4, 5, 6A, 6B, 7F, 8, 9V, 10A, 11A, 12F, 14, 15B, 18C, 19A, 19F, 22F, 23F and 33F polysaccharides are known in the art (see e.g. Geno K et al. (2015) Clin Microbiol Rev Vol 28:3, p 871-899).

In an embodiment, the S. pneumoniae serotypes 1, 4, 5, 6A, 6B, 7F, 8, 9V, 10A, 11A, 12F, 14, 15B, 18C, 19A, 19F, 22F, 23F and/or 33F capsular saccharide used in the present invention is a synthetic carbohydrate. Preparation of a synthetic S. pneumoniae type 1 capsular saccharide can for example be conducted as disclosed in WO2015004041. Preparation of a synthetic S. pneumoniae type 4 capsular saccharide can for example be conducted as disclosed in WO2016091399. Preparation of a synthetic S. pneumoniae type 5 capsular saccharide can for example be conducted as disclosed in WO2016198170. Preparation of a synthetic S. pneumoniae type 8 capsular saccharide can for example be conducted as disclosed in WO2017220753.

In a preferred embodiment though, the source of bacterial polysaccharides according to this invention can be S. pneumoniae bacterial cells. Bacterial strains which can be used as source of S. pneumoniae capsular polysaccharides may be obtained from established culture collections (such as for example from the Streptococcal Reference Laboratory (Centers for Disease Control and Prevention, Atlanta, GA USA)) or clinical specimens.

S. pneumoniae serotypes 1, 4, 5, 6A, 6B, 7F, 8, 9V, 10A, 11A, 12F, 14, 15B, 18C, 19A, 19F, 22F, 23F and/or 33F capsular polysaccharides can be obtained directly from bacteria using isolation procedures known to one of ordinary skill in the art. They can also be purchased (such as for example from the American Type Culture Collection (ATCC, Manassas, VA USA) (e.g., reference No. ATCC 13-X, ATCC 36-X, ATCC 41-X, ATCC 280-X, ATCC 107-X, ATCC 284-X, ATCC 505-X, ATCC 331-X, ATCC 511-X, ATCC 514-X, ATCC 517-X, ATCC 520-X, ATCC 81-X, ATCC 289-X, ATCC 304-X, ATCC 101-X, ATCC 527-X, ATCC 104-X, ATCC 535-X)).

In case the capsular polysaccharide is obtained directly from bacteria, the bacterial cells can be grown in a medium, preferably in a soy based medium. Following fermentation of bacterial cells that produce S. pneumoniae capsular polysaccharides, the bacterial cells can be lysed to produce a cell lysate. The caspular polysaccharides may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381, WO2008/118752 and WO2020170190). The purified capsular polysaccharide can then be used for the preparation of glycoconjugates.

The isolated capsular saccharides can be characterized by different parameters including, for example the weight average molecular weight (Mw).

The molecular weight of the capsular saccharides can be measured by Size Exclusion Chromatography (SEC) combined with Multiangle Laser Light Scattering detector (MALLS).

In a preferred embodiment, the isolated capsular polysaccharides (i.e. purified before further treatment) have a weight average molecular weight between 5 kDa and 5,000 kDa. In an embodiment, the isolated capsualr polysaccharide has a weight average molecular weight between 10 kDa and 3,000 kDa. In an embodiment, the isolated capsular polysaccharide has a weight average molecular weight between 50 kDa and 1,000 kDa.

Any whole number integer within any of the above ranges is contemplated as an embodiment of the disclosure.

In order to generate conjugates with advantageous filterability characteristics, immunogenicity and/or yields, sizing of the capsular polysaccharide to a target molecular weight range can be performed prior to the conjugation to a carrier protein. Advantageously, the size of the purified serotypes 1, 4, 5, 6A, 6B, 7F, 8, 9V, 10A, 11A, 12F, 14, 15B, 18C, 19A, 19F, 22F, 23F and/or 33F capsular polysaccharide is reduced while preserving critical features of the structure of the polysaccharide. Mechanical or chemical sizing maybe employed (see e.g. WO2006/110381, WO2015110941).

In an embodiment, the size of the purified capsular polysaccharide is reduced by chemical hydrolysis. Chemical hydrolysis maybe conducted using a mild acid (e.g acetic acid, formic acid, propanoic acid). Chemical hydrolysis may also be conducted using a diluted strong acid (such as diluted hydrochloric acid, diluted sulfuric acid, diluted phosphoric acid, diluted nitric acid or diluted perchloric acid).

The size of the purified polysaccharide can also be reduced by mechanical homogenization. In an embodiment, the size of the purified polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

In an embodiment, the isolated serotypes 1, 4, 5, 6A, 6B, 7F, 8, 9V, 10A, 11A, 12F, 14, 15B, 18C, 19A, 19F, 22F, 23F and/or 33F capsular polysaccharide is not sized.

The isolated serotype 1 capsular polysaccharide may be de-O-acetylated (see e.g. WO 2008/079653). Therefore, in an embodiment, the isolated serotype 1 capsular polysaccharide is partially de-O-acetyalted. In an embodiment, de-O-acetylation is conducted using a mild base. The partial de-O-acetylation can be performed using sodium bicarbonate/carbonate buffer.

In an embodiment, the isolated serotype 1 capsular polysaccharide before conjugation has a weight average molecular weight between 100 kDa and 1000 kDa. In an embodiment, the isolated serotype 1 capsular polysaccharide before conjugation has a weight average molecular weight between 150 kDa and 900 kDa. In a preferred embodiment, the isolated serotype 1 capsular polysaccharide before conjugation has a weight average molecular weight between 150 kDa and 700 kDa. The weight average molecular weight (Mw) of the isolated saccharide before conjugation refers to the Mw before activation of the polysaccharide (i.e. after an eventual sizing step but before reacting the polysaccharide with an activating agent).

In an embodiment, the isolated serotype 4 capsular polysaccharide before conjugation has a weight average molecular weight between 100 kDa and 1000 kDa. In an embodiment, the isolated serotype 4 capsular polysaccharide before conjugation has a weight average molecular weight between 300 kDa and 900 kDa.

In an embodiment, the isolated serotype 5 capsular polysaccharide before conjugation has a weight average molecular weight between 100 kDa and 1,000 kDa. In an embodiment, the isolated serotype 5 capsular polysaccharide before conjugation has a weight average molecular weight between 200 kDa and 600 kDa.

In an embodiment, the isolated serotype 6A capsular polysaccharide before conjugation has a weight average molecular weight between 100 kDa and 1000 kDa. In an embodiment, the isolated serotype 6A capsular polysaccharide before conjugation has a weight average molecular weight between 300 kDa and 900 kDa.

In an embodiment, the isolated serotype 6B capsular polysaccharide before conjugation has a weight average molecular weight between 100 kDa and 1000 kDa. In an embodiment, the isolated serotype 6B capsular polysaccharide before conjugation has a weight average molecular weight between 200 kDa and 900 kDa.

In an embodiment, the isolated serotype 7F capsular polysaccharide before conjugation has a weight average molecular weight between 100 kDa and 1000 kDa. In an embodiment, the isolated serotype 7F capsular polysaccharide before conjugation has a weight average molecular weight between 200 kDa and 900 kDa.

In an embodiment, the isolated serotype 8 capsular polysaccharide before conjugation has a weight average molecular weight between 100 kDa and 1000 kDa. In an embodiment, the isolated serotype 8 capsular polysaccharide before conjugation has a weight average molecular weight between 200 kDa and 400 kDa.

In an embodiment, the isolated serotype 9V capsular polysaccharide before conjugation has a weight average molecular weight between 100 kDa and 1000 kDa. In an embodiment, the isolated serotype 9V capsular polysaccharide before conjugation has a weight average molecular weight between 200 kDa and 900 kDa.

In an embodiment, the isolated serotype 10A capsular polysaccharide before conjugation has a weight average molecular weight between 100 kDa and 1000 kDa. In an embodiment, the isolated serotype 10A capsular polysaccharide before conjugation has a weight average molecular weight between 200 kDa and 900 kDa.

In an embodiment, the isolated serotype 11A capsular polysaccharide before conjugation has a weight average molecular weight between 100 kDa and 1000 kDa. In an embodiment, the isolated serotype 11A capsular polysaccharide before conjugation has a weight average molecular weight between 100 kDa and 400 kDa.

In an embodiment, the isolated serotype 12F capsular polysaccharide before conjugation has a weight average molecular weight between 100 kDa and 1000 kDa. In an embodiment, the isolated serotype 12F capsular polysaccharide before conjugation has a weight average molecular weight between 150 kDa and 400 kDa.

In an embodiment, the isolated serotype 14 capsular polysaccharide before conjugation has a weight average molecular weight between 100 kDa and 1000 kDa. In an embodiment, the isolated serotype 14 capsular polysaccharide before conjugation has a weight average molecular weight between 200 kDa and 900 kDa.

In an embodiment, the isolated serotype 15B capsular polysaccharide before conjugation has a weight average molecular weight between 100 kDa and 1000 kDa. In an embodiment, the isolated serotype 15B capsular polysaccharide before conjugation has a weight average molecular weight between 150 kDa and 300 kDa.

In an embodiment, the isolated serotype 18C capsular polysaccharide before conjugation has a weight average molecular weight between 20 kDa and 1000 kDa. In an embodiment, the isolated serotype 18C capsular polysaccharide before conjugation has a weight average molecular weight between 20 kDa and 500 kDa.

In an embodiment, the isolated serotype 19A capsular polysaccharide before conjugation has a weight average molecular weight between 100 kDa and 1000 kDa. In an embodiment, the isolated serotype 19A capsular polysaccharide before conjugation has a weight average molecular weight between 250 kDa and 700 kDa.

In an embodiment, the isolated serotype 19F capsular polysaccharide before conjugation has a weight average molecular weight between 100 kDa and 1000 kDa. In an embodiment, the isolated serotype 19F capsular polysaccharide before conjugation has a weight average molecular weight between 250 kDa and 800 kDa.

In an embodiment, the isolated serotype 22F capsular polysaccharide before conjugation has a weight average molecular weight between 100 kDa and 1000 kDa. In an embodiment, the isolated serotype 22F capsular polysaccharide before conjugation has a weight average molecular weight between 400 kDa and 700 kDa.

In an embodiment, the isolated serotype 23F capsular polysaccharide before conjugation has a weight average molecular weight between 100 kDa and 1000 kDa. In an embodiment, the isolated serotype 23F capsular polysaccharide before conjugation has a weight average molecular weight between 200 kDa and 800 kDa.

In an embodiment, the isolated serotype 33F capsular polysaccharide before conjugation has a weight average molecular weight between 300 kDa and 2000 kDa. In an embodiment, the isolated serotype 33F capsular polysaccharide before conjugation has a weight average molecular weight between 500 kDa and 2000 kDa.

In an embodiment, the serotype 1 glycoconjugate of the present invention comprises a serotype 1 capsular polysaccharide wherein the weight average molecular weight (Mw) of said polysaccharide before conjugation is between 50 kDa and 1,000 kDa. In an embodiment, the weight average molecular weight (Mw) is between 200 kDa and 750 kDa. In a preferred embodiment, the weight average molecular weight (Mw) is between 250 kDa and 600 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 4 glycoconjugate of the present invention comprises a serotype 4 capsular polysaccharide wherein the weight average molecular weight (Mw) of said polysaccharide before conjugation is between 50 kDa and 1,000 kDa. In an embodiment, the weight average molecular weight (Mw) is between 200 kDa and 1,000 kDa. In a preferred embodiment, the weight average molecular weight (Mw) is between 400 kDa and 900 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 5 glycoconjugate of the present invention comprises a serotype 5 capsular polysaccharide wherein the weight average molecular weight (Mw) of said polysaccharide before conjugation is between 100 kDa and 1,000 kDa. In an embodiment, the weight average molecular weight (Mw) is between 150 kDa and 800 kDa. In a preferred embodiment, the weight average molecular weight (Mw) is between 200 kDa and 500 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 6A glycoconjugate of the present invention comprises a serotype 6A capsular polysaccharide wherein the weight average molecular weight (Mw) of said polysaccharide before conjugation is between 50 kDa and 1,000 kDa. In an embodiment, the weight average molecular weight (Mw) is between 200 kDa and 1,000 kDa. In a preferred embodiment, the weight average molecular weight (Mw) is between 300 kDa and 800 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 6B glycoconjugate of the present invention comprises a serotype 6B capsular polysaccharide wherein the weight average molecular weight (Mw) of said polysaccharide before conjugation is between 50 kDa and 1,000 kDa. In an embodiment, the weight average molecular weight (Mw) is between 200 kDa and 1,000 kDa. In a preferred embodiment, the weight average molecular weight (Mw) is between 300 kDa and 800 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 7F glycoconjugate of the present invention comprises a serotype 7F capsular polysaccharide wherein the weight average molecular weight (Mw) of said polysaccharide before conjugation is between 50 kDa and 1,000 kDa. In an embodiment, the weight average molecular weight (Mw) is between 200 kDa and 1,000 kDa. In a preferred embodiment, the weight average molecular weight (Mw) is between 300 kDa and 800 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 8 glycoconjugate of the present invention comprises a serotype 8 capsular polysaccharide wherein the weight average molecular weight (Mw) of said polysaccharide before conjugation is between 50 kDa and 1,000 kDa. In an embodiment, the weight average molecular weight (Mw) is between 200 kDa and 800 kDa. In a preferred embodiment, the weight average molecular weight (Mw) is between 300 kDa and 600 kDa. In a most preferred embodiment, the weight average molecular weight (Mw) is between 200 kDa and 400 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 9V glycoconjugate of the present invention comprises a serotype 9V capsular polysaccharide wherein the weight average molecular weight (Mw) of said polysaccharide before conjugation is between 50 kDa and 1,000 kDa. In an embodiment, the weight average molecular weight (Mw) is between 200 kDa and 900 kDa. In a preferred embodiment, the weight average molecular weight (Mw) is between 300 kDa and 600 kDa. In a most preferred embodiment, the weight average molecular weight (Mw) is between 100 kDa and 400 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 10A glycoconjugate of the present invention comprises a serotype 10A capsular polysaccharide wherein the weight average molecular weight (Mw) of said polysaccharide before conjugation is between 50 kDa and 1,000 kDa. In an embodiment, the weight average molecular weight (Mw) is between 200 kDa and 800 kDa. In a preferred embodiment, the weight average molecular weight (Mw) is between 300 kDa and 600 kDa. In a most preferred embodiment, the weight average molecular weight (Mw) is between 100 kDa and 400 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 11A glycoconjugate of the present invention comprises a serotype 11A capsular polysaccharide wherein the weight average molecular weight (Mw) of said polysaccharide before conjugation is between 50 kDa and 1,000 kDa. In an embodiment, the weight average molecular weight (Mw) is between 75 kDa and 600 kDa. In a preferred embodiment, the weight average molecular weight (Mw) is between 100 kDa and 400 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 12F glycoconjugate of the present invention comprises a serotype 12F capsular polysaccharide wherein the weight average molecular weight (Mw) of said polysaccharide before conjugation is between 100 kDa and 1,000 kDa. In an embodiment, the weight average molecular weight (Mw) is between 150 kDa and 600 kDa. In a preferred embodiment, the weight average molecular weight (Mw) is between 150 kDa and 400 kDa. In a most preferred embodiment, the weight average molecular weight (Mw) is between 250 kDa and 350 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 14 glycoconjugate of the present invention comprises a serotype 14 capsular polysaccharide wherein the weight average molecular weight (Mw) of said polysaccharide before conjugation is between 50 kDa and 1,000 kDa. In an embodiment, the weight average molecular weight (Mw) is between 100 kDa and 800 kDa. In a preferred embodiment, the weight average molecular weight (Mw) is between 200 kDa and 600 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 15B glycoconjugate of the present invention comprises a serotype 15B capsular polysaccharide wherein the weight average molecular weight (Mw) of said polysaccharide before conjugation is between 50 kDa and 1,000 kDa. In an embodiment, the weight average molecular weight (Mw) is between 100 kDa and 600 kDa. In a preferred embodiment, the weight average molecular weight (Mw) is between 150 kDa and 300 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 18C glycoconjugate of the present invention comprises a serotype 18C capsular polysaccharide wherein the weight average molecular weight (Mw) of said polysaccharide before conjugation is between 20 kDa and 800 kDa. In an embodiment, the weight average molecular weight (Mw) is between 20 kDa and 400 kDa. In a preferred embodiment, the weight average molecular weight (Mw) is between 20 kDa and 200 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 19A glycoconjugate of the present invention comprises a serotype 19A capsular polysaccharide wherein the weight average molecular weight (Mw) of said polysaccharide before conjugation is between 50 kDa and 1,000 kDa. In an embodiment, the weight average molecular weight (Mw) is between 100 kDa and 700 kDa. In a preferred embodiment, the weight average molecular weight (Mw) is between 250 kDa and 500 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 19F glycoconjugate of the present invention comprises a serotype 19F capsular polysaccharide wherein the weight average molecular weight (Mw) of said polysaccharide before conjugation is between 50 kDa and 1,000 kDa. In an embodiment, the weight average molecular weight (Mw) is between 100 kDa and 900 kDa. In a preferred embodiment, the weight average molecular weight (Mw) is between 250 kDa and 600 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 22F glycoconjugate of the present invention comprises a serotype 22F capsular polysaccharide wherein the weight average molecular weight (Mw) of said polysaccharide before conjugation is between 50 kDa and 1,000 kDa. In an embodiment, the weight average molecular weight (Mw) is between 100 kDa and 900 kDa. In a preferred embodiment, the weight average molecular weight (Mw) is between 400 kDa and 700 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 23F glycoconjugate of the present invention comprises a serotype 23F capsular polysaccharide wherein the weight average molecular weight (Mw) of said polysaccharide before conjugation is between 50 kDa and 1,000 kDa. In an embodiment, the weight average molecular weight (Mw) is between 100 kDa and 900 kDa. In a preferred embodiment, the weight average molecular weight (Mw) is between 200 kDa and 600 kDa. Where the weight average molecular weight (Mw) before conjugation refers to the Mw after activation of the polysaccharide (i.e. after an eventual sizing step and after reacting the polysaccharide with an activating agent).

In an embodiment, the serotype 33F glycoconjugate of the present invention comprises a serotype 33F capsular polysaccharide wherein the weight average molecular weight (Mw) of said polysaccharide before conjugation is between 50 kDa and 2,500 kDa. In an embodiment, the weight average molecular weight (Mw) is between 100 kDa and 2,000 kDa. In a preferred embodiment, the weight average molecular weight (Mw) is between 600 kDa and 2,000 kDa. Where the weight average molecular weight (Mw) before conjugation refers to the Mw after activation of the polysaccharide (i.e. after an eventual sizing step and after reacting the polysaccharide with an activating agent).

In some embodiments, the serotype 1 glycoconjugate of the invention has a weight average molecular weight (Mw) of between 500 kDa and 15,000 kDa. In other embodiments, the serotype 1 glycoconjugate has a weight average molecular weight (Mw) of between 1,000 kDa and 10,000 kDa. In preferred embodiments, the serotype 1 glycoconjugate has a weight average molecular weight (Mw) of between 2,000 kDa and 5,000 kDa.

In some embodiments, the serotype 4 glycoconjugate of the invention has a weight average molecular weight (Mw) of between 500 kDa and 15,000 kDa. In other embodiments, the serotype 4 glycoconjugate has a weight average molecular weight (Mw) of between 1,000 kDa and 10,000 kDa. In preferred embodiments, the serotype 4 glycoconjugate has a weight average molecular weight (Mw) of between 2,000 kDa and 5,000 kDa.

In some embodiments, the serotype 5 glycoconjugate of the invention has a weight average molecular weight (Mw) of between 500 kDa and 15,000 kDa. In other embodiments, the serotype 5 glycoconjugate has a weight average molecular weight (Mw) of between 1,000 kDa and 10,000 kDa. In preferred embodiments, the serotype 5 glycoconjugate has a weight average molecular weight (Mw) of between 2,000 kDa and 5,000 kDa.

In some embodiments, the serotype 6A glycoconjugate of the invention has a weight average molecular weight (Mw) of between 500 kDa and 15,000 kDa. In other embodiments, the serotype 6A glycoconjugate has a weight average molecular weight (Mw) of between 1,000 kDa and 10,000 kDa. In preferred embodiments, the serotype 6A glycoconjugate has a weight average molecular weight (Mw) of between 2,000 kDa and 5,000 kDa.

In some embodiments, the serotype 6B glycoconjugate of the invention has a weight average molecular weight (Mw) of between 500 kDa and 15,000 kDa. In other embodiments, the serotype 6B glycoconjugate has a weight average molecular weight (Mw) of between 1,000 kDa and 10,000 kDa. In preferred embodiments, the serotype 6B glycoconjugate has a weight average molecular weight (Mw) of between 2,000 kDa and 5,500 kDa.

In some embodiments, the serotype 7F glycoconjugate of the invention has a weight average molecular weight (Mw) of between 500 kDa and 15,000 kDa. In other embodiments, the serotype 7F glycoconjugate has a weight average molecular weight (Mw) of between 1,000 kDa and 10,000 kDa. In preferred embodiments, the serotype 7F glycoconjugate has a weight average molecular weight (Mw) of between 2,000 kDa and 5,500 kDa.

In some embodiments, the serotype 8 glycoconjugate of the invention has a weight average molecular weight (Mw) of between 500 kDa and 15,000 kDa. In other embodiments, the serotype 8 glycoconjugate has a weight average molecular weight (Mw) of between 1,000 kDa and 10,000 kDa. In preferred embodiments, the serotype 8 glycoconjugate has a weight average molecular weight (Mw) of between 2,500 kDa and 8,000 kDa.

In some embodiments, the serotype 9V glycoconjugate of the invention has a weight average molecular weight (Mw) of between 500 kDa and 15,000 kDa. In other embodiments, the serotype 9V glycoconjugate has a weight average molecular weight (Mw) of between 1,000 kDa and 10,000 kDa. In preferred embodiments, the serotype 9V glycoconjugate has a weight average molecular weight (Mw) of between 2,000 kDa and 5,500 kDa.

In some embodiments, the serotype 10A glycoconjugate of the invention has a weight average molecular weight (Mw) of between 500 kDa and 15,000 kDa. In other embodiments, the serotype 10A glycoconjugate has a weight average molecular weight (Mw) of between 1,000 kDa and 10,000 kDa. In preferred embodiments, the serotype 10A glycoconjugate has a weight average molecular weight (Mw) of between 2,000 kDa and 5,500 kDa.

In some embodiments, the serotype 11A glycoconjugate of the invention has a weight average molecular weight (Mw) of between 500 kDa and 15,000 kDa. In other embodiments, the serotype 11A glycoconjugate has a weight average molecular weight (Mw) of between 1,000 kDa and 10,000 kDa. In preferred embodiments, the serotype 11A glycoconjugate has a weight average molecular weight (Mw) of between 700 kDa and 4,500 kDa.

In some embodiments, the serotype 12F glycoconjugate of the invention has a weight average molecular weight (Mw) of between 500 kDa and 15,000 kDa. In other embodiments, the serotype 12F glycoconjugate has a weight average molecular weight (Mw) of between 1,000 kDa and 10,000 kDa. In preferred embodiments, the serotype 12F glycoconjugate has a weight average molecular weight (Mw) of between 1,500 kDa and 4,000 kDa.

In some embodiments, the serotype 14 glycoconjugate of the invention has a weight average molecular weight (Mw) of between 500 kDa and 15,000 kDa. In other embodiments, the serotype 14 glycoconjugate has a weight average molecular weight (Mw) of between 1,000 kDa and 10,000 kDa. In preferred embodiments, the serotype 14 glycoconjugate has a weight average molecular weight (Mw) of between 2,000 kDa and 5,500 kDa.

In some embodiments, the serotype 15B glycoconjugate of the invention has a weight average molecular weight (Mw) of between 1,000 kDa and 25,000 kDa. In other embodiments, the serotype 15B glycoconjugate has a weight average molecular weight (Mw) of between 2,000 kDa and 20,000 kDa. In preferred embodiments, the serotype 15B glycoconjugate has a weight average molecular weight (Mw) of between 5,000 kDa and 15,000 kDa.

In some embodiments, the serotype 18C glycoconjugate of the invention has a weight average molecular weight (Mw) of between 150 kDa and 15,000 kDa. In other embodiments, the serotype 18C glycoconjugate has a weight average molecular weight (Mw) of between 250 kDa and 7,000 kDa. In preferred embodiments, the serotype 18C glycoconjugate has a weight average molecular weight (Mw) of between 300 kDa and 4,000 kDa.

In some embodiments, the serotype 19A glycoconjugate of the invention has a weight average molecular weight (Mw) of between 500 kDa and 15,000 kDa. In other embodiments, the serotype 19A glycoconjugate has a weight average molecular weight (Mw) of between 1,000 kDa and 10,000 kDa. In preferred embodiments, the serotype 19A glycoconjugate has a weight average molecular weight (Mw) of between 2,000 kDa and 7,500 kDa.

In some embodiments, the serotype 19F glycoconjugate of the invention has a weight average molecular weight (Mw) of between 500 kDa and 15,000 kDa. In other embodiments, the serotype 19F glycoconjugate has a weight average molecular weight (Mw) of between 750 kDa and 10,000 kDa. In preferred embodiments, the serotype 19F glycoconjugate has a weight average molecular weight (Mw) of between 1,000 kDa and 7,500 kDa.

In some embodiments, the serotype 22F glycoconjugate of the invention has a weight average molecular weight (Mw) of between 500 kDa and 10,000 kDa. In other embodiments, the serotype 22F glycoconjugate has a weight average molecular weight (Mw) of between 1,000 kDa and 7,500 kDa. In preferred embodiments, the serotype 22F glycoconjugate has a weight average molecular weight (Mw) of between 2,500 kDa and 5,500 kDa.

In some embodiments, the serotype 23F glycoconjugate of the invention has a weight average molecular weight (Mw) of between 500 kDa and 15,000 kDa. In other embodiments, the serotype 23F glycoconjugate has a weight average molecular weight (Mw) of between 750 kDa and 10,000 kDa. In preferred embodiments, the serotype 23F glycoconjugate has a weight average molecular weight (Mw) of between 1,000 kDa and 7,500 kDa.

In some embodiments, the serotype 33F glycoconjugate of the invention has a weight average molecular weight (Mw) of between 500 kDa and 15,000 kDa. In other embodiments, the serotype 33F glycoconjugate has a weight average molecular weight (Mw) of between 750 kDa and 10,000 kDa. In preferred embodiments, the serotype 33F glycoconjugate has a weight average molecular weight (Mw) of between 2,000 kDa and 6,000 kDa.

The glycoconjugates of the invention may also be characterized by the ratio (weight/weight) of saccharide to carrier protein.

In some embodiments, the ratio of serotype 1 polysaccharide to carrier protein in the glycoconjugate (w/w) is between 0.4 and 3.0. Preferably, the saccharide to carrier protein ratio (w/w) is between 0.6 and 2.0. In some such embodiments, the carrier protein is TT. Preferably, the carrier protein is CRM₁₉₇.

In some embodiments, the ratio of serotype 4 polysaccharide to carrier protein in the glycoconjugate (w/w) is between 0.4 and 3.0. Preferably, the saccharide to carrier protein ratio (w/w) is between 0.9 and 2.1. Even more preferably, the saccharide to carrier protein ratio (w/w) is between 1.0 and 1.9. In some such embodiments, the carrier protein is TT. Preferably, the carrier protein is CRM₁₉₇.

In some embodiments, the ratio of serotype 5 polysaccharide to carrier protein in the glycoconjugate (w/w) is between 0.4 and 3.0. Preferably, the saccharide to carrier protein ratio (w/w) is between 1.3 and 2.5. In some such embodiments, the carrier protein is TT. Preferably, the carrier protein is CRM₁₉₇.

In some embodiments, the ratio of serotype 6A polysaccharide to carrier protein in the glycoconjugate (w/w) is between 0.4 and 3.0. Preferably, the saccharide to carrier protein ratio (w/w) is between 0.7 and 1.6. In some such embodiments, the carrier protein is TT. Preferably, the carrier protein is CRM₁₉₇.

In some embodiments, the ratio of serotype 6B polysaccharide to carrier protein in the glycoconjugate (w/w) is between 0.4 and 3.0. Preferably, the saccharide to carrier protein ratio (w/w) is between 0.4 and 0.8. In some such embodiments, the carrier protein is TT. Preferably, the carrier protein is CRM₁₉₇.

In some embodiments, the ratio of serotype 7F polysaccharide to carrier protein in the glycoconjugate (w/w) is between 0.4 and 3.0. Preferably, the saccharide to carrier protein ratio (w/w) is between 0.7 and 1.5. In some such embodiments, the carrier protein is TT. Preferably, the carrier protein is CRM₁₉₇.

In some embodiments, the ratio of serotype 8 polysaccharide to carrier protein in the glycoconjugate (w/w) is between 0.4 and 3.0. Preferably, the saccharide to carrier protein ratio (w/w) is between 0.6 and 1.6. In some such embodiments, the carrier protein is TT. Preferably, the carrier protein is CRM₁₉₇.

In some embodiments, the ratio of serotype 9V polysaccharide to carrier protein in the glycoconjugate (w/w) is between 0.4 and 3.0. Preferably, the saccharide to carrier protein ratio (w/w) is between 1.2 and 2.3. In some such embodiments, the carrier protein is TT. Preferably, the carrier protein is CRM₁₉₇.

In some embodiments, the ratio of serotype 10A polysaccharide to carrier protein in the glycoconjugate (w/w) is between 0.4 and 3.0. Preferably, the saccharide to carrier protein ratio (w/w) is between 0.7 and 1.6. In some such embodiments, the carrier protein is TT. Preferably, the carrier protein is CRM₁₉₇.

In some embodiments, the ratio of serotype 11A polysaccharide to carrier protein in the glycoconjugate (w/w) is between 0.4 and 3.0. Preferably, the saccharide to carrier protein ratio (w/w) is between 0.9 and 1.5. In some such embodiments, the carrier protein is TT. Preferably, the carrier protein is CRM₁₉₇.

In some embodiments, the ratio of serotype 12F polysaccharide to carrier protein in the glycoconjugate (w/w) is between 0.4 and 3.0. Preferably, the saccharide to carrier protein ratio (w/w) is between 0.7 and 1.5. In some such embodiments, the carrier protein is TT. Preferably, the carrier protein is CRM₁₉₇.

In some embodiments, the ratio of serotype 14 polysaccharide to carrier protein in the glycoconjugate (w/w) is between 0.4 and 3.0. Preferably, the saccharide to carrier protein ratio (w/w) is between 1.4 and 2.6. In some such embodiments, the carrier protein is TT. Preferably, the carrier protein is CRM₁₉₇.

In some embodiments, the ratio of serotype 15B polysaccharide to carrier protein in the glycoconjugate (w/w) is between 0.4 and 3.0. Preferably, the saccharide to carrier protein ratio (w/w) is between 0.6 and 1.6. In some such embodiments, the carrier protein is TT. Preferably, the carrier protein is CRM₁₉₇.

In some embodiments, the ratio of serotype 18C polysaccharide to carrier protein in the glycoconjugate (w/w) is between 0.4 and 3.0. Preferably, the saccharide to carrier protein ratio (w/w) is between 0.7 and 1.5. In some such embodiments, the carrier protein is TT. Preferably, the carrier protein is CRM₁₉₇.

In some embodiments, the ratio of serotype 19A polysaccharide to carrier protein in the glycoconjugate (w/w) is between 0.4 and 3.0. Preferably, the saccharide to carrier protein ratio (w/w) is between 0.4 and 0.9. In some such embodiments, the carrier protein is TT. Preferably, the carrier protein is CRM₁₉₇.

In some embodiments, the ratio of serotype 19F polysaccharide to carrier protein in the glycoconjugate (w/w) is between 0.4 and 3.0. Preferably, the saccharide to carrier protein ratio (w/w) is between 0.5 and 1.0. In some such embodiments, the carrier protein is TT. Preferably, the carrier protein is CRM₁₉₇.

In some embodiments, the ratio of serotype 22F polysaccharide to carrier protein in the glycoconjugate (w/w) is between 0.4 and 3.0. Preferably, the saccharide to carrier protein ratio (w/w) is between 0.8 and 1.2. In some such embodiments, the carrier protein is TT. Preferably, the carrier protein is CRM₁₉₇.

In some embodiments, the ratio of serotype 23F polysaccharide to carrier protein in the glycoconjugate (w/w) is between 0.4 and 3.0. Preferably, the saccharide to carrier protein ratio (w/w) is between 0.4 and 1.0. In some such embodiments, the carrier protein is TT. Preferably, the carrier protein is CRM₁₉₇.

In some embodiments, the ratio of serotype 33F polysaccharide to carrier protein in the glycoconjugate (w/w) is between 0.4 and 3.0. Preferably, the saccharide to carrier protein ratio (w/w) is between 1.0 and 2.2. In some such embodiments, the carrier protein is TT. Preferably, the carrier protein is CRM₁₉₇.

Another way to characterize the glycoconjugates of the invention is by the number of lysine residues in the carrier protein (e.g., CRM197, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials.

In a preferred embodiment, the degree of conjugation of the serotype 1 glycoconjugate of the invention is between 2 and 15.

In a preferred embodiment, the degree of conjugation of the serotype 4 glycoconjugate of the invention is between 2 and 15.

In a preferred embodiment, the degree of conjugation of the serotype 5 glycoconjugate of the invention is between 2 and 15.

In a preferred embodiment, the degree of conjugation of the serotype 6A glycoconjugate of the invention is between 2 and 15.

In a preferred embodiment, the degree of conjugation of the serotype 6B glycoconjugate of the invention is between 2 and 15.

In a preferred embodiment, the degree of conjugation of the serotype 7F glycoconjugate of the invention is between 2 and 15.

In a preferred embodiment, the degree of conjugation of the serotype 8 glycoconjugate of the invention is between 2 and 15.

In a preferred embodiment, the degree of conjugation of the serotype 9V glycoconjugate of the invention is between 2 and 15.

In a preferred embodiment, the degree of conjugation of the serotype 10A glycoconjugate of the invention is between 2 and 15.

In a preferred embodiment, the degree of conjugation of the serotype 11A glycoconjugate of the invention is between 2 and 15.

In a preferred embodiment, the degree of conjugation of the serotype 12F glycoconjugate of the invention is between 2 and 15.

In a preferred embodiment, the degree of conjugation of the serotype 14 glycoconjugate of the invention is between 2 and 15.

In a preferred embodiment, the degree of conjugation of the serotype 15B glycoconjugate of the invention is between 2 and 15.

In a preferred embodiment, the degree of conjugation of the serotype 18C glycoconjugate of the invention is between 2 and 15.

In a preferred embodiment, the degree of conjugation of the serotype 19A glycoconjugate of the invention is between 2 and 15.

In a preferred embodiment, the degree of conjugation of the serotype 19F glycoconjugate of the invention is between 2 and 15.

In a preferred embodiment, the degree of conjugation of the serotype 22F glycoconjugate of the invention is between 2 and 15.

In a preferred embodiment, the degree of conjugation of the serotype 23F glycoconjugate of the invention is between 2 and 15.

In a preferred embodiment, the degree of conjugation of the serotype 33F glycoconjugate of the invention is between 2 and 15.

The serotype 1, 4, 5, 6A, 6B, 7F, 8, 9V, 10A, 11A, 12F, 14, 15B, 18C, 19A, 19F, 22F, 23F or 33F glycoconjugate of the invention and immunogenic compositions comprising said glycoconjugate(s) may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In an embodiment, the serotype 1 glycoconjugate of the invention comprises less than about 40% of free serotype 1 polysaccharide compared to the total amount of serotype 1 polysaccharide. In a preferred embodiment the serotype 1 glycoconjugate comprises less than about 20% of free serotype 1 polysaccharide compared to the total amount of serotype 1 polysaccharide.

In an embodiment, the serotype 1 glycoconjugate of the invention comprises less than about 40% of free serotype 1 polysaccharide compared to the total amount of serotype 1 polysaccharide. In a preferred embodiment the serotype 1 glycoconjugate comprises less than about 20% of free serotype 1 polysaccharide compared to the total amount of serotype 1 polysaccharide.

In an embodiment, the serotype 4 glycoconjugate of the invention comprises less than about 40% of free serotype 4 polysaccharide compared to the total amount of serotype 4 polysaccharide. In a preferred embodiment the serotype 4 glycoconjugate comprises less than about 30% of free serotype 4 polysaccharide compared to the total amount of serotype 4 polysaccharide.

In an embodiment, the serotype 5 glycoconjugate of the invention comprises less than about 45% of free serotype 5 polysaccharide compared to the total amount of serotype 5 polysaccharide. In a preferred embodiment the serotype 5 glycoconjugate comprises less than about 40% of free serotype 5 polysaccharide compared to the total amount of serotype 5 polysaccharide.

In an embodiment, the serotype 6A glycoconjugate of the invention comprises less than about 40% of free serotype 6A polysaccharide compared to the total amount of serotype 6A polysaccharide. In a preferred embodiment the serotype 6A glycoconjugate comprises less than about 30% of free serotype 6A polysaccharide compared to the total amount of serotype 6A polysaccharide.

In an embodiment, the serotype 6B glycoconjugate of the invention comprises less than about 30% of free serotype 6B polysaccharide compared to the total amount of serotype 6B polysaccharide. In a preferred embodiment the serotype 6B glycoconjugate comprises less than about 20% of free serotype 6B polysaccharide compared to the total amount of serotype 6B polysaccharide.

In an embodiment, the serotype 7F glycoconjugate of the invention comprises less than about 30% of free serotype 7F polysaccharide compared to the total amount of serotype 7F polysaccharide. In a preferred embodiment the serotype 7F glycoconjugate comprises less than about 20% of free serotype 7F polysaccharide compared to the total amount of serotype 7F polysaccharide.

In an embodiment, the serotype 8 glycoconjugate of the invention comprises less than about 30% of free serotype 8 polysaccharide compared to the total amount of serotype 8 polysaccharide. In a preferred embodiment the serotype 8 glycoconjugate comprises less than about 20% of free serotype 8 polysaccharide compared to the total amount of serotype 8 polysaccharide.

In an embodiment, the serotype 9V glycoconjugate of the invention comprises less than about 40% of free serotype 9V polysaccharide compared to the total amount of serotype 9V polysaccharide. In a preferred embodiment the serotype 9V glycoconjugate comprises less than about 35% of free serotype 9V polysaccharide compared to the total amount of serotype 9V polysaccharide.

In an embodiment, the serotype 10A glycoconjugate of the invention comprises less than about 40% of free serotype 10A polysaccharide compared to the total amount of serotype 10A polysaccharide. In a preferred embodiment the serotype 10A glycoconjugate comprises less than about 20% of free serotype 10A polysaccharide compared to the total amount of serotype 10A polysaccharide.

In an embodiment, the serotype 11A glycoconjugate of the invention comprises less than about 40% of free serotype 11A polysaccharide compared to the total amount of serotype 11A polysaccharide. In a preferred embodiment the serotype 11A glycoconjugate comprises less than about 30% of free serotype 11A polysaccharide compared to the total amount of serotype 11A polysaccharide.

In an embodiment, the serotype 12F glycoconjugate of the invention comprises less than about 40% of free serotype 12F polysaccharide compared to the total amount of serotype 12F polysaccharide. In a preferred embodiment the serotype 12F glycoconjugate comprises less than about 30% of free serotype 12F polysaccharide compared to the total amount of serotype 12F polysaccharide.

In an embodiment, the serotype 14 glycoconjugate of the invention comprises less than about 40% of free serotype 14 polysaccharide compared to the total amount of serotype 14 polysaccharide. In a preferred embodiment the serotype 14 glycoconjugate comprises less than about 35% of free serotype 14 polysaccharide compared to the total amount of serotype 14 polysaccharide.

In an embodiment, the serotype 15B glycoconjugate of the invention comprises less than about 40% of free serotype 15B polysaccharide compared to the total amount of serotype 15B polysaccharide. In a preferred embodiment the serotype 15B glycoconjugate comprises less than about 35% of free serotype 15B polysaccharide compared to the total amount of serotype 15B polysaccharide.

In an embodiment, the serotype 18C glycoconjugate of the invention comprises less than about 30% of free serotype 18C polysaccharide compared to the total amount of serotype 18C polysaccharide. In a preferred embodiment the serotype 18C glycoconjugate comprises less than about 20% of free serotype 18C polysaccharide compared to the total amount of serotype 18C polysaccharide.

In an embodiment, the serotype 19A glycoconjugate of the invention comprises less than about 40% of free serotype 19A polysaccharide compared to the total amount of serotype 19A polysaccharide. In a preferred embodiment the serotype 19A glycoconjugate comprises less than about 30% of free serotype 19A polysaccharide compared to the total amount of serotype 19A polysaccharide.

In an embodiment, the serotype 19F glycoconjugate of the invention comprises less than about 30% of free serotype 19F polysaccharide compared to the total amount of serotype 19F polysaccharide. In a preferred embodiment the serotype 19F glycoconjugate comprises less than about 20% of free serotype 19F polysaccharide compared to the total amount of serotype 19F polysaccharide.

In an embodiment, the serotype 22F glycoconjugate of the invention comprises less than about 40% of free serotype 22F polysaccharide compared to the total amount of serotype 22F polysaccharide. In a preferred embodiment the serotype 22F glycoconjugate comprises less than about 20% of free serotype 22F polysaccharide compared to the total amount of serotype 22F polysaccharide.

In an embodiment, the serotype 23F glycoconjugate of the invention comprises less than about 30% of free serotype 23F polysaccharide compared to the total amount of serotype 23F polysaccharide. In a preferred embodiment the serotype 23F glycoconjugate comprises less than about 20% of free serotype 23F polysaccharide compared to the total amount of serotype 23F polysaccharide.

In an embodiment, the serotype 33F glycoconjugate of the invention comprises less than about 30% of free serotype 33F polysaccharide compared to the total amount of serotype 33F polysaccharide. In a preferred embodiment the serotype 33F glycoconjugate comprises less than about 20% of free serotype 33F polysaccharide compared to the total amount of serotype 33F polysaccharide.

The serotype 1, 4, 5, 6A, 6B, 7F, 8, 9V, 10A, 11A, 12F, 14, 15B, 18C, 19A, 19F, 22F, 23F or 33F glycoconjugate of the invention may also be characterized by their molecular size distribution (Kd). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of Kd, columns are calibrated to establish the fraction at which molecules are fully excluded (V₀), (Kd=0), and the fraction representing the maximum retention (Vi), (Kd=1). The fraction at which a specified sample attribute is reached (Ve), is related to Kd by the expression, Kd=(Ve−V₀)/(Vi−V₀).

In an embodiment, at least 40% of the serotype 1 glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 80% of the serotype 1 glycoconjugate has a Kd below or equal to 0.3 in a CL-4B column.

In an embodiment, at least 30% of the serotype 4 glycoconjugate has a Kd below or equal to 0.3 in a CL-4B column. In a preferred embodiment, at least 40% of the glycoconjugate has a Kd below or equal to 0.3 in a CL-4B column. In a preferred embodiment, between 40% and 80% of the serotype 4 glycoconjugate has a Kd below or equal to 0.3 in a CL-4B column.

In an embodiment, at least 40% of the serotype 5 glycoconjugate has a Kd below or equal to 0.3 in a CL-4B column. In a preferred embodiment, at least 55% of the glycoconjugate has a Kd below or equal to 0.3 in a CL-4B column. In a preferred embodiment, between 55% and 80% of the serotype 5 glycoconjugate has a Kd below or equal to 0.3 in a CL-4B column.

In an embodiment, at least 50% of the serotype 6A glycoconjugate has a Kd below or equal to 0.3 in a CL-4B column. In a preferred embodiment, at least 60% of the glycoconjugate has a Kd below or equal to 0.3 in a CL-4B column. In a preferred embodiment, between 60% and 90% of the serotype 6A glycoconjugate has a Kd below or equal to 0.3 in a CL-4B column.

In an embodiment, at least 30% of the serotype 6B glycoconjugate has a Kd below or equal to 0.3 in a CL-4B column. In a preferred embodiment, at least 35% of the glycoconjugate has a Kd below or equal to 0.3 in a CL-4B column. In a preferred embodiment, between 35% and 80% of the serotype 6B glycoconjugate has a Kd below or equal to 0.3 in a CL-4B column.

In an embodiment, at least 50% of the serotype 7F glycoconjugate has a Kd below or equal to 0.3 in a CL-4B column. In a preferred embodiment, at least 65% of the glycoconjugate has a Kd below or equal to 0.3 in a CL-4B column. In a preferred embodiment, between 65% and 90% of the serotype 7F glycoconjugate has a Kd below or equal to 0.3 in a CL-4B column.

In an embodiment, at least 60% of the serotype 8 glycoconjugate has a Kd below or equal to 0.3 in a CL-4B column. In a preferred embodiment, at least 70% of the glycoconjugate has a Kd below or equal to 0.3 in a CL-4B column. In a preferred embodiment, between 70% and 90% of the serotype 8 glycoconjugate has a Kd below or equal to 0.3 in a CL-4B column.

In an embodiment, at least 30% of the serotype 9V glycoconjugate has a Kd below or equal to 0.3 in a CL-4B column. In a preferred embodiment, at least 40% of the glycoconjugate has a Kd below or equal to 0.3 in a CL-4B column. In a preferred embodiment, between 40% and 80% of the serotype 9V glycoconjugate has a Kd below or equal to 0.3 in a CL-4B column.

In an embodiment, at least 40% of the serotype 10A glycoconjugate has a Kd below or equal to 0.3 in a CL-4B column. In a preferred embodiment, at least 55% of the glycoconjugate has a Kd below or equal to 0.3 in a CL-4B column. In a preferred embodiment, between 55% and 85% of the serotype 10A glycoconjugate has a Kd below or equal to 0.3 in a CL-4B column.

In an embodiment, at least 50% of the serotype 11A glycoconjugate has a Kd below or equal to 0.3 in a CL-4B column. In a preferred embodiment, at least 60% of the glycoconjugate has a Kd below or equal to 0.3 in a CL-4B column. In a preferred embodiment, between 60% and 85% of the serotype 11A glycoconjugate has a Kd below or equal to 0.3 in a CL-4B column.

In an embodiment, at least 40% of the serotype 12F glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 80% of the serotype 12F glycoconjugate has a Kd below or equal to 0.3 in a CL-4B column.

In an embodiment, at least 40% of the serotype 14 glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 80% of the serotype 14 glycoconjugate has a Kd below or equal to 0.3 in a CL-4B column.

In an embodiment, at least 30% of the serotype 15B glycoconjugate has a Kd below or equal to 0.3 in a CL-4B column. In a preferred embodiment, at least 40% of the glycoconjugate has a Kd below or equal to 0.3 in a CL-4B column. In a preferred embodiment, between 40% and 80% of the serotype 15B glycoconjugate has a Kd below or equal to 0.3 in a CL-4B column.

In an embodiment, at least 30% of the serotype 18C glycoconjugate has a Kd below or equal to 0.3 in a CL-4B column. In a preferred embodiment, at least 40% of the glycoconjugate has a Kd below or equal to 0.3 in a CL-4B column. In a preferred embodiment, between 40% and 80% of the serotype 18C glycoconjugate has a Kd below or equal to 0.3 in a CL-4B column.

In an embodiment, at least 40% of the serotype 19A glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 80% of the serotype 19A glycoconjugate has a Kd below or equal to 0.3 in a CL-4B column.

In an embodiment, at least 40% of the serotype 19F glycoconjugate has a Kd below or equal to 0.3 in a CL-4B column. In a preferred embodiment, at least 50% of the glycoconjugate has a Kd below or equal to 0.3 in a CL-4B column. In a preferred embodiment, between 50% and 80% of the serotype 19F glycoconjugate has a Kd below or equal to 0.3 in a CL-4B column.

In an embodiment, at least 30% of the serotype 22F glycoconjugate has a Kd below or equal to 0.3 in a CL-4B column. In a preferred embodiment, at least 40% of the glycoconjugate has a Kd below or equal to 0.3 in a CL-4B column. In a preferred embodiment, between 40% and 80% of the serotype 22F glycoconjugate has a Kd below or equal to 0.3 in a CL-4B column.

In an embodiment, at least 30% of the serotype 23F glycoconjugate has a Kd below or equal to 0.3 in a CL-4B column. In a preferred embodiment, at least 35% of the glycoconjugate has a Kd below or equal to 0.3 in a CL-4B column. In a preferred embodiment, between 35% and 80% of the serotype 23F glycoconjugate has a Kd below or equal to 0.3 in a CL-4B column.

In an embodiment, at least 50% of the serotype 33F glycoconjugate has a Kd below or equal to 0.3 in a CL-4B column. In a preferred embodiment, at least 60% of the glycoconjugate has a Kd below or equal to 0.3 in a CL-4B column. In a preferred embodiment, between 60% and 95% of the serotype 33F glycoconjugate has a Kd below or equal to 0.3 in a CL-4B column.

In an embodiment, the serotype 1, 4, 5, 6A, 6B, 7F, 8, 9V, 10A, 11A, 12F, 14, 15B, 18C, 19A, 19F, 22F, 23F and/or 33F saccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with 1,1′-carbonyldiimidazole (CDI) (see Bethell et al. (1979) J. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In an embodiment, the serotype 1, 4, 5, 6A, 6B, 7F, 8, 9V, 10A, 11A, 12F, 14, 15B, 18C, 19A, 19F, 22F, 23F and/or 33F glycoconjugates of the invention are prepared using reductive amination chemistry. According to the present invention, reductive amination involves two steps, (1) oxidation (activation) of a purified saccharide, (2) reduction of the activated saccharide and a carrier protein to form a glycoconjugate (see e.g. WO2006/110381, WO2008/079653, WO2008/143709, WO2008/079732, WO2011/110531, WO2012/119972, WO2015110941, WO2015110940, WO2018/144439, WO2018/156491).

In an embodiment, the serotypes 1, 4, 5, 6A, 6B, 7F, 8, 9V, 10A, 11A, 12F, 14, 15B, 18C, 19A, 19F, 22F, 23F and 33F glycoconjugates of the invention are prepared using reductive amination chemistry.

In another embodiment, the serotypes 1, 4, 5, 6A, 6B, 7F, 8, 9V, 10A, 11A, 12F, 14, 15B, 18C, 19A, 19F, 22F and 23F glycoconjugates of the invention are prepared using reductive amination chemistry.

As mentioned above, before oxidation, sizing of the isolated serotype 1, 4, 5, 6A, 6B, 7F, 8, 9V, 10A, 11A, 12F, 14, 15B, 18C, 19A, 19F, 22F, 23F and/or 33F capsular polysaccharide to a target molecular weight (MW) range can be performed. Therefore, in an embodiment, the isolated serotype 1, 4, 5, 6A, 6B, 7F, 8, 9V, 10A, 11A, 12F, 14, 15B, 18C, 19A, 19F, 22F, 23F and/or 33F capsular polysaccharide is sized before oxidation.

Therefore, in an embodiment, the isolated serotype 1, 4, 5, 6A, 6B, 7F, 8, 9V, 10A, 11A, 12F, 14, 15B, 18C, 19A, 19F, 22F, 23F and 33F capsular polysaccharide are sized before oxidation.

In an embodiment, the isolated serotype 1, 4, 5, 6A, 6B, 7F, 8, 9V, 10A, 11A, 12F, 14, 15B, 18C, 19A, 19F, 22F, 23F and/or 33F capsular polysaccharide is conjugated to a carrier protein by a process comprising the step of:
(a) reacting said isolated serotype 1, 4, 5, 6A, 6B, 7F, 8, 9V, 10A, 11A, 12F, 14, 15B, 18C, 19A, 19F, 22F, 23F and/or 33F capsular polysaccharide with an oxidizing agent to produce an activated polysaccharide; (b) compounding the activated polysaccharide of step (a) with a carrier protein; and (c) reacting the compounded activated polysaccharide and carrier protein with a reducing agent to form a glycoconjugate.

Following the oxidation step (a) the saccharide is said to be activated and is referred to as “activated polysaccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO⁴⁻) and orthoperiodate (IO₆⁵⁻) and the various salts of periodate (e.g., sodium periodate and potassium periodate).

In an embodiment, the isolated serotypes 1, 4, 5, 6A, 6B, 7F, 8, 9V, 10A, 11A, 12F, 14, 15B, 18C, 19A, 19F, 22F, 23F and 33F capsular polysaccharides are reacted with periodate.

In an embodiment, the isolated serotypes 1, 4, 5, 6A, 6B, 7F, 8, 9V, 10A, 11A, 14, 15B, 18C, 19A, 19F, 22F and 23F capsular polysaccharides are reacted with periodate.

In a preferred embodiment, the oxidizing agent is sodium periodate. In an embodiment, the periodate used for the oxidation is metaperiodate. In a most preferred embodiment the periodate used for the oxidation is sodium metaperiodate.

When a polysaccharide reacts with periodate, periodate oxidises vicinal hydroxyl groups to form carbonyl or aldehyde groups and causes cleavage of a C-C bond. For this reason, the term “reacting a polysaccharide with periodate” includes oxidation of vicinal hydroxyl groups by periodate.

In one embodiment step a) comprises reacting the serotype 1, 4, 5, 6A, 6B, 7F, 8, 9V, 10A, 11A, 12F, 14, 15B, 18C, 19A, 19F, 22F, 23F and/or 33F polysaccharide with periodate.

In one embodiment step a) comprises reacting the serotype 1, 4, 5, 6A, 6B, 7F, 8, 9V, 10A, 11A, 12F, 14, 15B, 18C, 19A, 19F, 22F, 23F and/or 33F polysaccharide with 0.05-2 molar equivalents of periodate.

In one embodiment step a) comprises reacting the serotype 1, 4, 5, 6A, 6B, 7F, 8, 9V, 10A, 11A, 12F, 14, 15B, 18C, 19A, 19F, 22F, 23F and/or 33F polysaccharide with 0.05-0.2 molar equivalents of periodate.

In one embodiment step a) comprises reacting the serotype 1, 4, 5, 6A, 6B, 7F, 8, 9V, 10A, 11A, 12F, 14, 15B, 18C, 19A, 19F, 22F, 23F and/or 33F polysaccharide with 0.2-0.5 molar equivalents of periodate

In one embodiment step a) comprises reacting the serotype 1, 4, 5, 6A, 6B, 7F, 8, 9V, 10A, 11A, 12F, 14, 15B, 18C, 19A, 19F, 22F, 23F and/or 33F polysaccharide with 0.5-1.5 molar equivalents of periodate

In one embodiment step a) comprises reacting the serotype 1, 4, 5, 6A, 6B, 7F, 8, 9V, 10A, 11A, 12F, 14, 15B, 18C, 19A, 19F, 22F, 23F and/or 33F polysaccharide with 1.5-2.0 molar equivalents of periodate.

In some embodiment, the reaction of step (a) is quenched. Therefore, following step (a) a quenching step (a′) can be performed (see WO2015110940). Therefore, in an embodiment, the isolated serotype 1, 4, 5, 6A, 6B, 7F, 8, 9V, 10A, 11A, 12F, 14, 15B, 18C, 19A, 19F, 22F, 23F and/or 33F capsular polysaccharide is conjugated to a carrier protein by a process comprising the step of:
(a) reacting said isolated serotype 1, 4, 5, 6A, 6B, 7F, 8, 9V, 10A, 11A, 12F, 14, 15B, 18C, 19A, 19F, 22F, 23F and/or 33F capsular polysaccharide with an oxidizing agent to produce an activated polysaccharide; (a′) quenching the oxidation reaction by addition of a quenching agent resulting in an activated polysaccharide; (b) compounding the activated polysaccharide of step (a′) with a carrier protein; and (c) reacting the compounded activated polysaccharide and carrier protein with a reducing agent to form a glycoconjugate.

In an embodiment, the oxidizing agent is 2,2,6,6-tetramethyl-1-piperidinyloxy (TEMPO) free radical and N-Chlorosuccinimide (NCS) as the cooxidant. This oxidizing agent is particularly suitable for oxidizing serotype 12F capsular polysaccharides. In such embodiment, the glycoconjugates from S. pneumoniae serotype 12F are prepared using 2,2,6,6-tetramethyl-1-piperidinyloxy (TEMPO) free radical to oxidize primary alcohols of the saccharide to aldehydes using N-Chlorosuccinimide (NCS) as the cooxidant (hereinafter “TEMPO/NCS oxidation”), such as described at Example 7 and in WO 2014/097099. Therefore, in one aspect, the glycoconjugates from S. pneumoniae serotype 12F of the invention are obtainable by a method comprising the steps of: a) reacting an isolated serotype 12F capsular polysaccharide with 2,2,6,6-tetramethyl-1-piperidinyloxy (TEMPO) and N-chlorosuccinimide (NCS) to produce an activated polysaccharide; (b) compounding the activated polysaccharide of step (a) with a carrier protein and (c) reacting the compounded activated polysaccharide and carrier protein with a reducing agent to form a glycoconjugate (hereinafter “TEMPO/NCS-reductive amination”). In one aspect, the glycoconjugates from S. pneumoniae serotype 12F are obtained by said method.

In an embodiment, the isolated serotypes 1, 4, 5, 6A, 6B, 7F, 8, 9V, 10A, 11A, 14, 15B, 18C, 19A, 19F, 22F, 23F and 33F capsular polysaccharides are reacted with periodate and the isolated serotype 12F capsular polysaccharide is reacted with TEMPO/NCS.

In an embodiment, the isolated serotypes 1, 4, 5, 6A, 6B, 7F, 8, 9V, 10A, 11A, 14, 15B, 18C, 19A, 19F, 22F and 23F capsular polysaccharides are reacted with periodate and the isolated serotype 12F capsular polysaccharide is reacted with TEMPO/NCS.

In a preferred embodiment the degree of oxidation (also named “degree of activation” in the present document) of the activated serotype 1, 4, 5, 6A, 6B, 7F, 8, 9V, 10A, 11A, 12F, 14, 15B, 18C, 19A, 19F, 22F, 23F and/or 33F polysaccharide is between 2 and 30.

In an embodiment the degree of oxidation of the activated serotype 1 polysaccharide is between 1 and 15 when the carrier protein is TT (see e.g. Table 2 of WO2019/152921).

In a preferred embodiment the degree of oxidation of the activated serotype 1 polysaccharide is between 4 and 10 when the carrier protein is CRM₁₉₇. See Table 1 of WO2019/152921.

In an embodiment the degree of oxidation of the activated serotype 4 polysaccharide is between 1 and 15. In a preferred embodiment the degree of oxidation of the activated serotype 4 polysaccharide is between 1 and 5 when the carrier protein is CRM₁₉₇. See Table 1 of WO2019/152921.

In an embodiment the degree of oxidation of the activated serotype 5 polysaccharide is between 1 and 15. In a preferred embodiment the degree of oxidation of the activated serotype 5 polysaccharide is between 2 and 6 when the carrier protein is CRM₁₉₇. See Table 1 of WO2019/152921.

In an embodiment the degree of oxidation of the activated serotype 5 polysaccharide is between 1 and 15 when the carrier protein is TT. See Table 2 of WO2019/152921.

In an embodiment the degree of oxidation of the activated serotype 6A polysaccharide is between 1 and 15. In a preferred embodiment the degree of oxidation of the activated serotype 6A polysaccharide is between 5 and 15 when the carrier protein is CRM₁₉₇. See Table 1 of WO2019/152921.

In an embodiment the degree of oxidation of the activated serotype 6B polysaccharide is between 1 and 15. In a preferred embodiment the degree of oxidation of the activated serotype 6B polysaccharide is between 7 and 13 when the carrier protein is CRM₁₉₇. See Table 1 of WO2019/152921.

In an embodiment the degree of oxidation of the activated serotype 7F polysaccharide is between 1 and 15. In a preferred embodiment the degree of oxidation of the activated serotype 7F polysaccharide is between 2 and 8 when the carrier protein is CRM₁₉₇. See Table 1 of WO2019/152921.

In an embodiment the degree of oxidation of the activated serotype 8 polysaccharide is between 1 and 20. In a preferred embodiment the degree of oxidation of the activated serotype 8 polysaccharide is between 1 and 17 when the carrier protein is CRM₁₉₇. See Table 1 of WO2019/152921.

In an embodiment the degree of oxidation of the activated serotype 9V polysaccharide is between 1 and 15. In a preferred embodiment the degree of oxidation of the activated serotype 9V polysaccharide is between 4 and 9 when the carrier protein is CRM₁₉₇. See Table 1 of WO2019/152921.

In an embodiment the degree of oxidation of the activated serotype 10A polysaccharide is between 1 and 15. In a preferred embodiment the degree of oxidation of the activated serotype 10A polysaccharide is between 1 and 12 when the carrier protein is CRM₁₉₇. See Table 1 of WO2019/152921.

In an embodiment the degree of oxidation of the activated serotype 11A polysaccharide is between 1 and 20. In a preferred embodiment the degree of oxidation of the activated serotype 11A polysaccharide is between 1 and 15 when the carrier protein is CRM₁₉₇. See Table 1 of WO2019/152921.

In an embodiment the degree of oxidation of the activated serotype 12F polysaccharide is between 1 and 15. In a preferred embodiment the degree of oxidation of the activated serotype 12F polysaccharide is between 1 and 9 when the carrier protein is CRM₁₉₇. See Table 1 of WO2019/152921.

In an embodiment the degree of oxidation of the activated serotype 14 polysaccharide is between 1 and 20. In a preferred embodiment the degree of oxidation of the activated serotype 14 polysaccharide is between 6 and 13 when the carrier protein is CRM₁₉₇. See Table 1 of WO2019/152921.

In an embodiment the degree of oxidation of the activated serotype 15B polysaccharide is between 1 and 20. In a preferred embodiment the degree of oxidation of the activated serotype 15B polysaccharide is between 1 and 17 when the carrier protein is CRM₁₉₇. See Table 1 of WO2019/152921.

In an embodiment the degree of oxidation of the activated serotype 15B polysaccharide is between 1 and 15 when the carrier protein is TT. See Table 2 of WO2019/152925.

In an embodiment the degree of oxidation of the activated serotype 18C polysaccharide is between 1 and 20. In a preferred embodiment the degree of oxidation of the activated serotype 18C polysaccharide is between 6 and 14 when the carrier protein is CRM₁₉₇. See Table 1 of WO2019/152921.

In an embodiment the degree of oxidation of the activated serotype 19A polysaccharide is between 1 and 20. In a preferred embodiment the degree of oxidation of the activated serotype 19A polysaccharide is between 7 and 13 when the carrier protein is CRM₁₉₇. See Table 1 of WO2019/152921.

In an embodiment the degree of oxidation of the activated serotype 19F polysaccharide is between 1 and 20. In a preferred embodiment the degree of oxidation of the activated serotype 19F polysaccharide is between 6 and 12 when the carrier protein is CRM₁₉₇. See Table 1 of WO2019/152921.

In an embodiment the degree of oxidation of the activated serotype 22F polysaccharide is between 1 and 20. In a preferred embodiment the degree of oxidation of the activated serotype 22F polysaccharide is between 1 and 16 when the carrier protein is CRM₁₉₇. See Table 1 of WO2019/152921.

In an embodiment the degree of oxidation of the activated serotype 22F polysaccharide is between 1 and 20 when the carrier protein is TT. See Table 2 of WO2019/152925.

In an embodiment the degree of oxidation of the activated serotype 23F polysaccharide is between 1 and 20. In a preferred embodiment the degree of oxidation of the activated serotype 23F polysaccharide is between 6 and 14 when the carrier protein is CRM₁₉₇. See Table 1 of WO2019/152921.

In an embodiment the degree of oxidation of the activated serotype 33F polysaccharide is between 1 and 20. In a preferred embodiment the degree of oxidation of the activated serotype 33F polysaccharide is between 1 and 15 when the carrier protein is CRM₁₉₇. See Table 1 of WO2019/152921.

The activated polysaccharide and the carrier protein may be lyophilised (freeze-dried), either independently (discrete lyophilization) or together (co-lyophilized). In one embodiment the activated polysaccharide and the carrier protein are co-lyophilized. In another embodiment the activated polysaccharide and the carrier protein are lyophilized independently.

In one embodiment the lyophilization takes place in the presence of a non-reducing sugar, possible non-reducing sugars include sucrose, trehalose, raffinose, stachyose, melezitose, dextran, mannitol, lactitol and palatinit.

In an embodiment the initial input ratio (weight by weight) of activated serotype 1, 4, 5, 6A, 6B, 7F, 8, 9V, 10A, 11A, 12F, 14, 15B, 18C, 19A, 19F, 22F, 23F and/or 33F capsular polysaccharide to carrier protein at step b) is between 4:1 and 0.1:1. In an embodiment the initial input ratio (weight by weight) of activated serotype 1, 4, 5, 6A, 6B, 7F, 8, 9V, 10A, 11A, 12F, 14, 15B, 18C, 19A, 19F, 22F, 23F and/or 33F capsular polysaccharide to carrier protein is between 1.5:1 and 0.5:1.

In an embodiment, the reduction reaction (c) is carried out in aprotic solvent. In one embodiment the reduction reaction (c) is carried out in a solution consisting essentially of dimethylsulphoxide (DMSO). In an embodiment, the reduction reaction (c) is carried out in DMSO (dimethylsulfoxide) solvent.

In an embodiment, the reduction reaction (c) is carried out in aqueous solvent.

In an embodiment, the reduction reaction (c) is carried out in DMSO (dimethylsulfoxide) solvent for serotypes 6A, 6B, 7F, 8, 10A, 15B, 19A, 19F, 22F and 23F and the reduction reaction (c) is carried out in aqueous solvent for serotypes 1, 4, 5, 9V, 11A, 12F, 14 and 18C.

In an embodiment, the reduction reaction (c) is carried out in DMSO (dimethylsulfoxide) solvent for serotypes 6A, 6B, 7F, 8, 10A, 15B, 19A, 19F, 22F and 23F and the reduction reaction (c) is carried out in aqueous solvent for serotypes 1, 4, 5, 9V, 11A, 12F, 14, 18C and 33F.

In an embodiment, the reduction reaction (c) is carried out in DMSO (dimethylsulfoxide) solvent for serotypes 6A, 6B, 7F, 18C, 19A, 19F and 23F and the reduction reaction (c) is carried out in aqueous solvent for serotypes 1, 4, 5, 9V, 14, 22F and 33F.

In an embodiment, the reduction reaction (c) is carried out in DMSO (dimethylsulfoxide) solvent for serotypes 6A, 6B, 7F, 11A, 12F, 19A, 19F and 23F and the reduction reaction (c) is carried out in aqueous solvent for serotypes 1, 4, 5, 8, 9V, 10A, 14, 15B, 18C, 22F and 33F.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeiPrN-BH3, benzylamine-BH3 or 5-ethyl-2-methylpyridine borane (PEMB). In an embodiment, the reducing agent is sodium triacetoxyborohydride. In a preferred embodiment, the reducing agent is sodium cyanoborohydride. In an embodiment, the reducing agent is sodium cyanoborohydride in the present of nickel (see WO2018144439).

In one embodiment between 0.2 and 10 molar equivalents of reducing agent is used in step c). Preferably, between 0.5 and 5 molar equivalents of reducing agent is used in step c).

At the end of the reduction reaction, there may be unreacted aldehyde groups remaining in the conjugates, these may be capped using a suitable capping agent. In one embodiment this capping agent is sodium borohydride (NaBH4).

In an embodiment capping is achieved by mixing the product of step c) with 1 to 20 molar equivalents of sodium borohydride. In an embodiment capping is achieved by mixing the product of step c) with 1 to 3 molar equivalents of sodium borohydride.

Following conjugation to the carrier protein, the serotype 1, 4, 5, 6A, 6B, 7F, 8, 9V, 10A, 11A, 12F, 14, 15B, 18C, 19A, 19F, 22F, 23F and/or 33F glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the serotype 1 glycoconjugate of the present invention comprises the step of purifying the glycoconjugate after it is produced.

In certain embodiments, the serotype 33F glycoconjugates of the invention are prepared using reductive amination.

In other embodiments, the serotype 33F glycoconjugates of the invention are prepared using eTEC conjugation (herinafter “serotype 33F eTEC linked glycoconjugates”), such as described in WO 2014/027302 or WO2015110941 (see Examples 1, 2 and 3). Said 33F glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC linked glycoconjugates of the invention may be represented by the general formula (III):

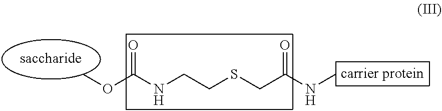

wherein the atoms that comprise the eTEC spacer are contained in the central box.

The eTEC spacer includes seven linear atoms (i.e., —C(O)NH(CH₂)₂SCH₂C(O)—) and provides stable thioether and amide bonds between the saccharide and carrier protein. Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond.

In the serotype 33F eTEC linked glycoconjugates of the invention, the saccharide may be a polysaccharide or an oligosaccharide. The carrier protein may be selected from any suitable carrier as described herein or known to those of skill in the art. Preferably, the saccharide is a polysaccharide. In some such embodiments, the carrier protein is CRM₁₉₇. In some such embodiments, the eTEC linked glycoconjugate comprises a S. pneumoniae serotype 33F capsular polysaccharide.

In particularly preferred embodiments, the eTEC linked glycoconjugate comprises a Pn-33F capsular polysaccharide, which is covalently conjugated to CRM₁₉₇ through an eTEC spacer (serotype 33F eTEC linked glycoconjugates).

In an embodiment, the serotypes 1, 4, 5, 6A, 6B, 7F, 8, 9V, 10A, 11A, 12F, 14, 15B, 18C, 19A, 19F, 22F and 23F glycoconjugates of the invention are prepared using reductive amination chemistry and the serotype 33F glycoconjugate of the invention is prepared using eTEC conjugation.

### 1.8 Carrier Protein of the Invention

A component of the glycoconjugate of the invention is a carrier protein to which the saccharide is conjugated. The terms “protein carrier” or “carrier protein” or “carrier” may be used interchangeably herein. Carrier proteins should be amenable to standard conjugation procedures. A capsular saccharide can be conjugated to a carrier protein via covalent or non-covalent bonds. In an embodiment, the capsular saccharide is conjugated to a carrier protein via non-covalent bonds (such as the rhizavidin/biotin system, see e.g. WO2012155007, WO2020056202). Preferably, the capsular saccharide is conjugated via covalent bonds.

In an embodiment, the carrier protein of the glycoconjugate of the invention is: DT (Diphtheria Toxoid), TT (Tetanus Toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRMs, CRM₁₀₂, CRM₁₀₃ or CRM₁07; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA) or recombinant Pseudomonas aeruginosa exotoxin A (in particular non-toxic mutants thereof (such as exotoxin A bearing a substitution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In an embodiment, the carrier protein of the glycoconjugates of the invention is the fusion protein CP1. The CP1 fusion protein comprises a biotin-binding protein such as, e.g., a truncated rhizavidin protein (e.g, amino acids 45-179 of a wild-type rhizavidin protein), a first linker (e.g, a GGGGSSS linker), a SP1500 polypeptide (e.g., amino acids 27-278 of a full-length S. pneumoniae SP1500 polypeptide), a second linker (e.g, the amino acid sequence AAA), and a SP0785 polypeptide (e.g, amino acids 33-399 of a full length S. pneumoniae SP0785 polypeptide) see e.g. WO2020056202.

In another embodiment, the carrier protein of the glycoconjugate of the invention is PD (H. influenzae protein D; see, e.g., EP0594610 B).

Preferably, the carrier protein of the glycoconjugates of the invention is DT, TT, CRM₁₉₇ or a C5a peptidase from Streptococcus (SCP).

In an embodiment, the carrier protein of the glycoconjugate of the invention is DT (Diphtheria toxoid).

In another embodiment, the carrier protein of the glycoconjugate of the invention is TT (tetanus toxoid).

In a preferred embodiment, the carrier protein of the glycoconjugate of the invention is CRM₁₉₇ or a C5a peptidase from Streptococcus (SCP).

In a preferred embodiment, the carrier protein of the glycoconjugate of the invention is CRM₁₉₇. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the carrier protein of the glycoconjugate of the invention is the A chain of CRM₁₉₇ (see CN103495161). In an embodiment, the carrier protein of the glycoconjugate of the invention is the A chain of CRM₁₉₇ obtained via expression by genetically recombinant E. coli (see CN103495161).

In other preferred embodiments, the carrier protein of the glycoconjugate of the invention is SCP (Streptococcal C5a Peptidase). Two important species of β-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of β-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type III (Fn) domains (Fn1, commonly residues 583/584-712/713; Fn2, commonly residues 712/713-928/929/930; commonly Fn3, residues 929/930-1029/1030/1031) and a cell wall anchor domain (commonly redisues 1029/1030/1031 to the C-terminus).

In an embodiment, the carrier protein of the glycoconjugate of the invention is an SCP from GBS (SCPB). An example of SCPB is provided at SEQ ID. NO: 3 of WO97/26008. See also SEQ ID NO: 3 of WO00/34487.

In another preferred embodiments, the carrier protein of the glycoconjugate of the invention is an SCP from GAS (SCPA). Examples of SCPA can be found at SEQ ID. No. 1 and SEQ ID. No. 2 of WO97/26008. See also SEQ ID NO: 1, 2 and 23 of WO00/34487.

In a preferred embodiment, the carrier protein of the glycoconjugate of the invention is an enzymatically inactive SCP.

In other preferred embodiments, the carrier protein of the glycoconjugate of the invention is an enzymatically inactive SCP from GBS (SCPB).

In another preferred embodiments, the carrier protein of the glycoconjugate of the invention is an enzymatically inactive SCP from GAS (SCPA).

In an embodiment, the carrier protein of the glycoconjugate of the invention is a fragment of an SCP. In an embodiment, the carrier protein of the glycoconjugate of the invention is a fragment of an SCPA. Preferably, the carrier protein of the glycoconjugate of the invention is a fragment of an SCPB.

In an embodiment, the carrier protein of the glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the glycoconjugate of the invention is an enzymatically inactive fragment of an SCP which comprises the protease domain, the protease-associated domain (PA domain) and two of the three fibronectin type III (Fn) domains but does not comprise the export signal presequence, the pro-sequence and the cell wall anchor domain.

In an embodiment, the carrier protein of the glycoconjugate of the invention is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence and the cell wall anchor domain.

In an embodiment, the carrier protein of the glycoconjugate of the invention is an enzymatically inactive fragment of an SCPA. In an embodiment, said enzymatically inactive fragment of an SCPA comprises the protease domain, the protease-associated domain (PA domain) and the three fibronectin type III (Fn) domains but does not comprise the export signal presequence, the pro-sequence and the cell wall anchor domain.

In a preferred embodiment, the carrier protein of the glycoconjugate of the invention is an enzymatically inactive fragment of SCPB. Preferably, said enzymatically inactive fragment of SCPB comprises the protease domain, the protease-associated domain (PA domain) and the three fibronectin type III (Fn) domains but does not comprise the export signal presequence, the pro-sequence and the cell wall anchor domain.

In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 of WO00/34487.

Therefore, in an embodiment, the carrier protein of the glycoconjugate of the invention is an enzymatically inactive SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive SCPA where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive SCPB where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of an SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the glycoconjugate of the invention is an enzymatically inactive SCP where said inactivation is accomplished by replacing at least two amino acids of the wild type sequence. Preferably, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive SCPA where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive SCPB where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of an SCP where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least one amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the glycoconjugate of the invention is an enzymatically inactive SCP where said inactivation is accomplished by replacing at least three amino acids of the wild type sequence. Preferably, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive SCPA where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive SCPB where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of an SCP where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the enzymatic activity of SCP is inactivated by replacing at least four amino acids of the wild type sequence. In an embodiment, said at least four amino acids replacements are D130A, H193A, N295A and S512A.

Therefore, in an embodiment, the carrier protein of the glycoconjugate of the invention is an enzymatically inactive SCP where said inactivation is accomplished by replacing at least four amino acids of the wild type sequence. Preferably, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive SCPA where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive SCPB where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive fragment of an SCP where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least one amino acids is in part 2 of the protease domain. In an embodiment, said at least four amino acids replacements are D130A, H193A, N295A and S512A

In an embodiment, the carrier protein of the glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acids is in part 2 of the protease domain. In an embodiment, said at least four amino acids replacements are D130A, H193A, N295A and S512A

In a particular embodiment, the carrier protein of the glycoconjugate of the invention is an enzymatically inactive fragment of SCP which consists of SEQ ID NO: 1.

In a particular embodiment, the carrier protein of the glycoconjugate of the invention is an enzymatically inactive fragment of SCP which consists of SEQ ID NO: 2.

SEQ ID NO: 1:
MAKTADTPATSKATIRDLNDPSQVKTLQEKAGKGAGTVVAVIAAG
FDKNHEAWRLTDKAKARYQSKEDLEKAKKEHGITYGEWVNDKVAY
YHDYSKDGKTAVDQEHGTHVSGILSGNAPSETKEPYRLEGAMPEA
QLLLMRVEIVNGLADYARNYAQAIRDAINLGAKVINMSFGNAALA
YANLPDETKKAFDYAKSKGVSIVTSAGNDSSFGGKTRLPLADHPD
YGVVGTPAAADSTLTVASYSPDKQLTETVTVKTADQQDKEMPVLS
TNRFEPNKAYDYAYANRGTKEDDFKDVKGKIALIERGDIDFKDKI
AKAKKAGAVGVLIYDNQDKGFPIELPNVDQMPAAFISRKDGLLLK
DNPQKTITFNATPKVLPTASGTKLSRFSSWGLTADGNIKPDIAAP
GQDILSSVANNKYAKLSGTAMSAPLVAGIMGLLQEQYETQYPDMT
PSERLDLAKKVLMSSATALYDEDEKAYFSPRQQGAGAVDAKKASA
ATMYVTDKDNTSSKVHLNNVSDKFEVTVTVHNKSDKPQELYYQAT
VQTDKVDGKHFALAPKALYETSWQKITIPANSSKQVTVPIDASRF
SKDLLAQMKNGYFLEGFVRFKQDPKKEELMSIPYIGFRGDFGNLS
ALEKPIYDSKDGSSYYHEANSDAKDQLDGDGLQFYALKNNFTALT
TESNPWTIIKAVKEGVENIEDIESSEITETIFAGTFAKQDDDSHY
YIHRHANGKPYAAISPNGDGNRDYVQFQGTFLRNAKNLVAEVLDK
EGNVVWTSEVTEQWVKNYNNDLASTLGSTRFEKTRWDGKDKDGKV
VANGTYTYRVRYTPISSGAKEQHTDFDVIVDNTTPEVATSATFST
EDRRLTLASKPKTSQPVYRERIAYTYMDEDLPTTEYISPNEDGTF
TLPEEAETMEGATVPLKMSDFTYVVEDMAGNITYTPVTKLLEGHS
NKPEQ

SEQ ID NO: 1 is 950 amino acids long.

SEQ ID NO: 2:
AKTADTPATSKATIRDLNDPSQVKTLQEKAGKGAGTVVAVIAAGF
DKNHEAWRLTDKAKARYQSKEDLEKAKKEHGITYGEWVNDKVAYY
HDYSKDGKTAVDQEHGTHVSGILSGNAPSETKEPYRLEGAMPEAQ
LLLMRVEIVNGLADYARNYAQAIRDAINLGAKVINMSFGNAALAY
ANLPDETKKAFDYAKSKGVSIVTSAGNDSSFGGKTRLPLADHPDY
GVVGTPAAADSTLTVASYSPDKQLTETVTVKTADQQDKEMPVLST
NRFEPNKAYDYAYANRGTKEDDFKDVKGKIALIERGDIDFKDKIA
KAKKAGAVGVLIYDNQDKGFPIELPNVDQMPAAFISRKDGLLLKD
NPQKTITFNATPKVLPTASGTKLSRFSSWGLTADGNIKPDIAAPG
QDILSSVANNKYAKLSGTAMSAPLVAGIMGLLQEQYETQYPDMTP
SERLDLAKKVLMSSATALYDEDEKAYFSPRQQGAGAVDAKKASAA
TMYVTDKDNTSSKVHLNNVSDKFEVTVTVHNKSDKPQELYYQATV
QTDKVDGKHFALAPKALYETSWQKITIPANSSKQVTVPIDASRFS
KDLLAQMKNGYFLEGFVRFKQDPKKEELMSIPYIGFRGDFGNLSA
LEKPIYDSKDGSSYYHEANSDAKDQLDGDGLQFYALKNNFTALTT
ESNPWTIIKAVKEGVENIEDIESSEITETIFAGTFAKQDDDSHYY
IHRHANGKPYAAISPNGDGNRDYVQFQGTFLRNAKNLVAEVLDKE
GNVVWTSEVTEQWVKNYNNDLASTLGSTRFEKTRWDGKDKDGKVV
ANGTYTYRVRYTPISSGAKEQHTDFDVIVDNTTPEVATSATFSTE
DRRLTLASKPKTSQPVYRERIAYTYMDEDLPTTEYISPNEDGTFT
LPEEAETMEGATVPLKMSDFTYVVEDMAGNITYTPVTKLLEGHSN
KPEQ

SEQ ID NO: 2 is 949 amino acids long.

In a particular embodiment, the carrier protein of the glycoconjugate of the invention is an enzymatically inactive fragment of SCP consisting of a polypeptide having at least 90% identity with SEQ ID NO: 1.

In a particular embodiment, the carrier protein of the glycoconjugate of the invention is an enzymatically inactive fragment of SCP consisting of a polypeptide having at least 95% identity with SEQ ID NO: 1.

In a particular embodiment, the carrier protein of the glycoconjugate of the invention is an enzymatically inactive fragment of SCP consisting of a polypeptide having at least 99% identity with SEQ ID NO: 1.

In a particular embodiment, the carrier protein of the glycoconjugate of the invention is an enzymatically inactive fragment of SCP consisting of a polypeptide having at least 99.5% identity with SEQ ID NO: 1.

In a particular embodiment, the carrier protein of the glycoconjugate of the invention is an enzymatically inactive fragment of SCP consisting of a polypeptide having at least 99.8% identity with SEQ ID NO: 1.

In a particular embodiment, the carrier protein of the glycoconjugate of the invention is an enzymatically inactive fragment of SCP consisting of a polypeptide having at least 99.85% identity with SEQ ID NO: 1.

In a particular embodiment, the carrier protein of the glycoconjugate of the invention is an enzymatically inactive fragment of SCP consisting of a polypeptide having at least 90% identity with SEQ ID NO: 2.

In a particular embodiment, the carrier protein of the glycoconjugate of the invention is an enzymatically inactive fragment of SCP consisting of a polypeptide having at least 95% identity with SEQ ID NO: 2.

In a particular embodiment, the carrier protein of the glycoconjugate of the invention is an enzymatically inactive fragment of SCP consisting of a polypeptide having at least 99% identity with SEQ ID NO: 2.

In a particular embodiment, the carrier protein of the glycoconjugate of the invention is an enzymatically inactive fragment of SCP consisting of a polypeptide having at least 99.5% identity with SEQ ID NO: 2.

In a particular embodiment, the carrier protein of the glycoconjugate of the invention is an enzymatically inactive fragment of SCP consisting of a polypeptide having at least 99.8% identity with SEQ ID NO: 2.

In a particular embodiment, the carrier protein of the glycoconjugate of the invention is an enzymatically inactive fragment of SCP consisting of a polypeptide having at least 99.85% identity with SEQ ID NO: 2.

### 2 Immunogenic Compositions of the Invention

### 2.1 Combinations of Glycoconjugates of the Invention

In an embodiment the invention relates to an immunogenic composition comprising a glycoconjugate of the invention (such as the ones disclosed at section 1 above).

Preferably, the number of different S. pneumoniae capsular saccharides can range from 1 different serotypes (or “v”, valences) to 25 different serotypes (25v).

If the protein carrier is the same for 2 or more saccharides in the composition, the saccharides can be conjugated to the same molecule of the protein carrier (carrier molecules having 2 or more different saccharides conjugated to it) [see for instance WO2020121159].

In a preferred embodiment though, the saccharides are each individually conjugated to different molecules of the protein carrier (each molecule of protein carrier only having one type of saccharide conjugated to it). In said embodiment, the capsular saccharides are said to be individually conjugated to the carrier protein.

In an embodiment the invention relates to an immunogenic composition comprising 1 to 25 different glycoconjugates of the invention (as disclosed at section 1 above).

In an embodiment the invention relates to an immunogenic composition comprising 1 to 25 glycoconjugates from different serotypes of S. pneumoniae of the invention (1 to 25 pneumococcal conjugates).

In an embodiment the invention relates to an immunogenic composition comprising one pneumococcal glycoconjugate of the invention.

In an embodiment the invention relates to an immunogenic composition comprising two pneumococcal glycoconjugates of the invention.

In an embodiment the invention relates to an immunogenic composition comprising three pneumococcal glycoconjugates of the invention.

In an embodiment the invention relates to an immunogenic composition comprising four pneumococcal glycoconjugates of the invention.

In an embodiment the invention relates to an immunogenic composition comprising five pneumococcal glycoconjugates of the invention.

In an embodiment the invention relates to an immunogenic composition comprising ten pneumococcal glycoconjugates of the invention.

In an embodiment the invention relates to an immunogenic composition comprising eleven pneumococcal glycoconjugates of the invention.

In an embodiment the invention relates to an immunogenic composition comprising thirteen pneumococcal glycoconjugates of the invention.

In an embodiment the invention relates to an immunogenic composition comprising fifteen pneumococcal glycoconjugates of the invention.

In an embodiment the invention relates to an immunogenic composition comprising nineteen pneumococcal glycoconjugates of the invention.

In an embodiment the invention relates to an immunogenic composition comprising twenty pneumococcal glycoconjugates of the invention.

In an embodiment the invention relates to an immunogenic composition comprising twenty-one pneumococcal glycoconjugates of the invention.

In an embodiment the invention relates to an immunogenic composition comprising twenty-two pneumococcal glycoconjugates of the invention.

In an embodiment the invention relates to an immunogenic composition comprising twenty-three pneumococcal glycoconjugates of the invention.

In an embodiment the invention relates to an immunogenic composition comprising twenty-four pneumococcal glycoconjugates of the invention.

In a preferred embodiment the invention relates to an immunogenic composition comprising twenty-five pneumococcal glycoconjugates of the invention.

In an embodiment the invention relates to an immunogenic composition comprising a glycoconjugate from S. pneumoniae serotype 15A (such as the one of section 1 above).

In an embodiment the invention relates to an immunogenic composition comprising a glycoconjugate from S. pneumoniae serotype 23A (such as the one of section 1 above).

In an embodiment the invention relates to an immunogenic composition comprising a glycoconjugate from S. pneumoniae serotype 23B (such as the one of section 1 above).

In an embodiment the invention relates to an immunogenic composition comprising a glycoconjugate from S. pneumoniae serotype 24F (such as the one of section 1 above).

In an embodiment the invention relates to an immunogenic composition comprising a glycoconjugate from S. pneumoniae serotype 35B (such as the one of section 1 above).

In an embodiment the invention relates to an immunogenic composition comprising glycoconjugates from S. pneumoniae serotype 15A and 23A (such as the ones of section 1 above).

In an embodiment the invention relates to an immunogenic composition comprising glycoconjugates from S. pneumoniae serotype 15A and 23B (such as the ones of section 1 above).

In an embodiment the invention relates to an immunogenic composition comprising glycoconjugates from S. pneumoniae serotype 15A and 24F (such as the ones of section 1 above).

In an embodiment the invention relates to an immunogenic composition comprising glycoconjugates from S. pneumoniae serotype 15A and 35B (such as the ones of section 1 above).

In an embodiment the invention relates to an immunogenic composition comprising glycoconjugates from S. pneumoniae serotype 23A and 23B (such as the ones of section 1 above).

In an embodiment the invention relates to an immunogenic composition comprising glycoconjugates from S. pneumoniae serotype 23A and 24F (such as the ones of section 1 above).

In an embodiment the invention relates to an immunogenic composition comprising glycoconjugates from S. pneumoniae serotype 23A and 35B (such as the ones of section 1 above).

In an embodiment the invention relates to an immunogenic composition comprising glycoconjugates from S. pneumoniae serotype 23B and 24F (such as the ones of section 1 above).

In an embodiment the invention relates to an immunogenic composition comprising glycoconjugates from S. pneumoniae serotype 23B and 35B (such as the ones of section 1 above).

In an embodiment the invention relates to an immunogenic composition comprising glycoconjugates from S. pneumoniae serotype 24F and 35B (such as the ones of section 1 above).

In an embodiment the invention relates to an immunogenic composition comprising glycoconjugates from S. pneumoniae serotype 15A, 23A and 23B (such as the ones of section 1 above).

In an embodiment the invention relates to an immunogenic composition comprising glycoconjugates from S. pneumoniae serotype 15A, 23A and 24F (such as the ones of section 1 above).

In an embodiment the invention relates to an immunogenic composition comprising glycoconjugates from S. pneumoniae serotype 15A, 23A and 35B (such as the ones of section 1 above).

In an embodiment the invention relates to an immunogenic composition comprising glycoconjugates from S. pneumoniae serotype 15A, 23B and 24F (such as the ones of section 1 above).

In an embodiment the invention relates to an immunogenic composition comprising glycoconjugates from S. pneumoniae serotype 15A, 23B and 35B (such as the ones of section 1 above).

In an embodiment the invention relates to an immunogenic composition comprising glycoconjugates from S. pneumoniae serotype 15A, 24F and 35B (such as the ones of section 1 above).

In an embodiment the invention relates to an immunogenic composition comprising glycoconjugates from S. pneumoniae serotype 23A, 23B and 24F (such as the ones of section 1 above).

In an embodiment the invention relates to an immunogenic composition comprising glycoconjugates from S. pneumoniae serotype 23A, 23B and 35B (such as the ones of section 1 above).

In an embodiment the invention relates to an immunogenic composition comprising glycoconjugates from S. pneumoniae serotype 23A, 24F and 35B (such as the ones of section 1 above).

In an embodiment the invention relates to an immunogenic composition comprising glycoconjugates from S. pneumoniae serotype 23B, 24F and 35B (such as the ones of section 1 above).

In an embodiment the invention relates to an immunogenic composition comprising glycoconjugates from S. pneumoniae serotype 15A, 23A, 23B and 24F (such as the ones of section 1 above).

In an embodiment the invention relates to an immunogenic composition comprising glycoconjugates from S. pneumoniae serotype 15A, 23A, 23B and 35B (such as the ones of section 1 above).

In an embodiment the invention relates to an immunogenic composition comprising glycoconjugates from S. pneumoniae serotype 15A, 23A, 24F and 35B (such as the ones of section 1 above).

In an embodiment the invention relates to an immunogenic composition comprising glycoconjugates from S. pneumoniae serotype 15A, 23B, 24F and 35B (such as the ones of section 1 above).

In an embodiment the invention relates to an immunogenic composition comprising glycoconjugates from S. pneumoniae serotype 23A, 23B, 24F and 35B (such as the ones of section 1 above).

Preferably, the invention relates to an immunogenic composition comprising glycoconjugates from S. pneumoniae serotype 15A, 23A, 23B, 24F and 35B (such as the ones of section 1 above).

In an embodiment the invention relates to an immunogenic composition comprising 5, 6, 7, 8, 9, 10, 11, 12, 13, 14, 15, 16, 17, 18, 19, 20, 21, 22, 23, 24 or 25 pneumococcal glycoconjugates from different serotypes of S. pneumoniae, wherein said serotypes are selected from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

In an embodiment the invention relates to an immunogenic composition comprising from 14 to 25 pneumococcal glycoconjugates from different serotypes of S. pneumoniae, wherein said serotypes are selected from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

In an embodiment the invention relates to an immunogenic composition comprising from 21 to 25 pneumococcal glycoconjugates from different serotypes of S. pneumoniae, wherein said serotypes are selected from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises from 1 to 5 glycoconjugates from S. pneumoniae serotypes 15A, 23A, 23B, 24F and/or 35B. In an embodiment the immunogenic composition is a 21, 22, 23, 24 or 25-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises one glycoconjugate from S. pneumoniae serotypes 15A, 23A, 23B, 24F or 35B. In an embodiment the immunogenic composition is a 21-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises 2 glycoconjugates selected from S. pneumoniae serotypes 15A, 23A, 23B, 24F and 35B. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises 3 glycoconjugates selected from S. pneumoniae serotypes 15A, 23A, 23B, 24F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises 4 glycoconjugates selected from S. pneumoniae serotypes 15A, 23A, 23B, 24F and 35B. In an embodiment the immunogenic composition is a 24-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F. In an embodiment the immunogenic composition is a 21-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F. In an embodiment the immunogenic composition is a 21-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F. In an embodiment the immunogenic composition is a 21-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F. In an embodiment the immunogenic composition is a 21-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B. In an embodiment the immunogenic composition is a 21-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F and 33F. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F and 33F. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F and 33F. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 33F and 35B. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F and 33F. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F and 33F. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 33F and 35B. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F and 33F. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 33F and 35B. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F, 33F and 35B. In an embodiment the immunogenic composition is a 22-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F and 33F. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 24F and 33F. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 33F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F and 33F. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 33F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F, 33F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F and 33F. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 33F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F, 33F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F, 33F and 35B. In an embodiment the immunogenic composition is a 23-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F and 33F. In an embodiment the immunogenic composition is a 24-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 33F and 35B. In an embodiment the immunogenic composition is a 24-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 33F, 24F and 35B. In an embodiment the immunogenic composition is a 24-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In an embodiment the immunogenic composition is a 24-valent pneumococcal conjugate compositions.

In an embodiment the invention relates to an immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F, 33F and 35B. In an embodiment the immunogenic composition is a 24-valent pneumococcal conjugate compositions.

Preferably, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In a preferred embodiment the immunogenic composition is a 25-valent pneumococcal conjugate compositions.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugates of the invention are conjugated to CRM₁₉₇.

In an embodiment of any of the above immunogenic compositions, the glycoconjugate from S. pneumoniae serotype 3 is conjugated to CRM₁₉₇.

In an embodiment of any of the above immunogenic compositions, the glycoconjugate from S. pneumoniae serotype 3 is conjugated to SCP.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM197. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM197. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇.

In an embodiment of any of the above immunogenic compositions, the glycoconjugates of any of the above immunogenic compositions are all individually conjugated to CRM₁₉₇.

In an embodiment of any of the above immunogenic compositions, the glycoconjugate from S. pneumoniae serotype 3 is conjugated to SCP and the other glycoconjugates are all individually conjugated to CRM₁₉₇.

In an embodiment of any of the above immunogenic compositions, the glycoconjugate from S. pneumoniae serotype 3 is conjugated to SCP, at least one other glycoconjugate is conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, one other glycoconjugate is conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least two other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, two other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least three other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, three other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least four other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, four other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least five other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, five other glycoconjugates are conjugated to TT and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least one other glycoconjugate is conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, one other glycoconjugate is conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least two other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, two other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least three other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, three other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least four other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, four other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, at least five other glycoconjugates are conjugated to SCP and the other glycoconjugate(s) is/are all individually conjugated to CRM₁₉₇.

In an embodiment of any of the above immunogenic compositions, the glycoconjugate from S. pneumoniae serotype 3 is conjugated to SCP, five other glycoconjugates are conjugated to SCP and the other glycoconjugate(s) is/are all individually conjugated to CRM₁₉₇.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the other glycoconjugates are all individually conjugated to CRM₁₉₇. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugates are all individually conjugated to CRM₁₉₇. In a preferred embodiment the immunogenic composition is a 25-valent pneumococcal conjugate compositions.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

### 2.2 Dosage of the Immunogenic Compositions of the Invention

The amount of glycoconjugate(s) in each dose is selected as an amount which induces an immunoprotective response without significant, adverse side effects in typical vaccines. Such amount will vary depending upon which specific immunogen is employed and how it is presented.

The amount of a particular glycoconjugate in an immunogenic composition can be calculated based on total saccharide for that conjugate (conjugated and non-conjugated). For example, a glycoconjugate with 20% free saccharide will have about 80 μg of conjugated saccharide and about 20 μg of nonconjugated saccharide in a 100 μg saccharide dose. The amount of glycoconjugate can vary depending upon the bacteria and bacteria serotype. The saccharide concentration can be determined by the uronic acid assay.

The “immunogenic amount” of the different saccharide components in the immunogenic composition, may diverge and each may comprise about 0.5 μg, about 0.75 μg, about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular saccharide antigen.

Generally, each dose will comprise 0.1 μg to 100 μg of saccharide for a given serotype. In a preferred embodiment each dose will comprise 0.5 μg to 20 μg of saccharide for a given serotype. In an even preferred embodiment, each dose will comprise 2.0 μg to 10.0 μg of saccharide for a given serotype. Any whole number integer within any of the above ranges is contemplated as an embodiment of the disclosure.

In an embodiment, each dose will comprise about 0.5 μg of saccharide for each particular glycoconjugate. In an embodiment, each dose will comprise about 1.0 μg of saccharide for each particular glycoconjugate. In an embodiment, each dose will comprise about 1.1 μg of saccharide for each particular glycoconjugate. In an embodiment, each dose will comprise about 1.5 μg of saccharide for each particular glycoconjugate. In an embodiment, each dose will comprise about 2.0 μg of saccharide for each particular glycoconjugate. In an embodiment, each dose will comprise about 2.2 μg of saccharide for each particular glycoconjugate. In an embodiment, each dose will comprise about 2.5 μg of saccharide for each particular glycoconjugate. In an embodiment, each dose will comprise about 3.0 μg of saccharide for each particular glycoconjugate. In an embodiment, each dose will comprise about 3.5 μg of saccharide for each particular glycoconjugate. In an embodiment, each dose will comprise about 4.0 μg of saccharide for each particular glycoconjugate. In an embodiment, each dose will comprise about 4.4 μg of saccharide for each particular glycoconjugate. In an embodiment, each dose will comprise about 5.0 μg of saccharide for each particular glycoconjugate. In an embodiment, each dose will comprise about 5.5 μg of saccharide for each particular glycoconjugate. In an embodiment, each dose will comprise about 6.0 μg of saccharide for each particular glycoconjugate. In an embodiment, each dose will comprise about 6.5 μg of saccharide for each particular glycoconjugate. In an embodiment, each dose will comprise about 7.0 μg of saccharide for each particular glycoconjugate. In an embodiment, each dose will comprise about 7.5 μg of saccharide for each particular glycoconjugate. In an embodiment, each dose will comprise about 8.0 μg of saccharide for each particular glycoconjugate. In an embodiment, each dose will comprise about 8.5 μg of saccharide for each particular glycoconjugate. In an embodiment, each dose will comprise about 9.0 μg of saccharide for each particular glycoconjugate.

In an embodiment, each dose will comprise about 0.5 μg, about 1.0 μg, about 1.5 μg, about 2.0 μg, about 2.2 μg, about 2.5 μg, about 3.0 μg, about 3.5 μg, about 4.0 μg, about 4.5 μg, or about 5.0 μg of polysaccharide for glycoconjugates from S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B.

In an embodiment, each dose will comprise about 2.0 μg or about 2.2 μg of polysaccharide for glycoconjugates from S. pneumoniae serotype 1, 3, 4, 5, 6A, 7F, 8, 9V, 10A, 11A, 12F, 14, 15A, 15B, 18C, 19A, 19F, 22F, 23A, 23B, 23F, 24F, 33F and/or 35B.

In an embodiment, each dose will comprise about 2.0 μg or about 2.2 μg of polysaccharide for glycoconjugates from S. pneumoniae serotype 1, 4, 5, 6A, 7F, 8, 9V, 10A, 11A, 12F, 14, 15A, 15B, 18C, 19A, 19F, 22F, 23A, 23B, 23F, 24F, 33F and/or 35B.

In an embodiment, each dose will comprise about 3.0 μg, about 3.5 μg, about 4.0 μg, about 4.4 μg or about 5.0 μg of polysaccharide for glycoconjugate from S. pneumoniae serotype 6B.

In an embodiment, each dose will comprise about 3.0 μg, about 3.5 μg, about 4.0 μg, about 4.4 μg or about 5.0 μg of polysaccharide for glycoconjugate from S. pneumoniae serotype 3.

In an embodiment, each dose will comprise about 2.0 μg of polysaccharide for glycoconjugate from S. pneumoniae serotype 3.

In an embodiment, each dose will comprise about 2.2 μg of polysaccharide for glycoconjugate from S. pneumoniae serotype 3.

In an embodiment, each dose will comprise about 4.0 μg of polysaccharide for glycoconjugate from S. pneumoniae serotype 3.

In an embodiment, each dose will comprise about 4.4 μg of polysaccharide for glycoconjugate from S. pneumoniae serotype 3.

In an embodiment, each dose will comprise about 8.0 μg of polysaccharide for glycoconjugate from S. pneumoniae serotype 3.

In an embodiment, each dose will comprise about 8.8 μg of polysaccharide for glycoconjugate from S. pneumoniae serotype 3.

In an embodiment, each dose will comprise about 2.0 μg to about 2.5 μg of polysaccharide for each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg to about 4.8 μg of polysaccharide for glycoconjugate from S. pneumoniae serotype 6B.

In an embodiment, each dose will comprise about 2.0 μg to about 2.5 μg of polysaccharide for each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg to about 4.8 μg of polysaccharide for glycoconjugate from S. pneumoniae serotypes 3 and 6B.

In an embodiment, each dose will comprise about 2.0 μg to about 2.5 μg of polysaccharide for each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg to about 4.8 μg of polysaccharide for glycoconjugate from S. pneumoniae serotype 6B and about 6.0 μg to about 7.5 μg of polysaccharide for glycoconjugate from S. pneumoniae serotype 3.

In an embodiment, each dose will comprise about 2.0 μg to about 2.5 μg of polysaccharide for each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg to about 4.8 μg of polysaccharide for glycoconjugate from S. pneumoniae serotype 6B and about 8.0 μg to about 10.0 μg of polysaccharide for glycoconjugate from S. pneumoniae serotype 3.

In an embodiment, each dose will comprise about 2.0 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg of polysaccharide for glycoconjugate from S. pneumoniae serotype 6B.

In an embodiment, each dose will comprise about 2.2 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotype 6B.

In an embodiment, each dose will comprise about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg of polysaccharide for glycoconjugate from S. pneumoniae serotypes 3 and 6B.

In an embodiment, each dose will comprise about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotypes 3 and 6B.

In an embodiment, each dose will comprise about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg of polysaccharide for glycoconjugate from S. pneumoniae serotype 6B and about 6.0 μg of polysaccharide for glycoconjugate from S. pneumoniae serotype 3.

In an embodiment, each dose will comprise about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.4 μg of polysaccharide for glycoconjugate from S. pneumoniae serotype 6B and about 6.6 μg of polysaccharide for glycoconjugate from S. pneumoniae serotype 3.

In an embodiment, each dose will comprise about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg of polysaccharide for glycoconjugate from S. pneumoniae serotype 6B and about 8.0 μg of polysaccharide for glycoconjugate from S. pneumoniae serotype 3.

In an embodiment, each dose will comprise about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.4 μg of polysaccharide for glycoconjugate from S. pneumoniae serotype 6B and about 8.8 μg of polysaccharide for glycoconjugate from S. pneumoniae serotype 3.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.0 μg to about 3.0 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.0 μg to about 3.0 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.0 μg to about 3.0 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.0 μg to about 3.0 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.0 μg to about 3.0 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.0 μg to about 3.0 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.5 μg to about 2.5 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.5 μg to about 2.5 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.5 μg to about 2.5 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.5 μg to about 2.5 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.5 μg to about 2.5 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 1.5 μg to about 2.5 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 2.0 μg to about 2.2 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 2.0 μg to about 2.2 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 2.0 μg to about 2.2 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 2.0 μg to about 2.2 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is also about two times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 2.0 μg to about 2.2 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about three times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises an amount (a) of polysaccharide for glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B which is about the same for said polysaccharides, wherein said amount (a) is about 2.0 μg to about 2.2 μg of polysaccharide, wherein the amount of polysaccharide for glycoconjugate from S. pneumoniae serotype 6B is about two times said amount (a), and wherein the amount of polysaccharide for glycoconjugate from S. pneumoniae serotype 3 is about four times said amount (a). In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.2 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotype 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.0 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg of polysaccharide for glycoconjugate from S. pneumoniae serotype 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotypes 3 and 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg of polysaccharide for glycoconjugate from S. pneumoniae serotypes 3 and 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg of polysaccharide for glycoconjugate from S. pneumoniae serotype 6B and about 6.0 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.4 μg of polysaccharide for glycoconjugate from S. pneumoniae serotype 6B and about 6.6 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg of polysaccharide for glycoconjugate from S. pneumoniae serotype 6B and about 8.0 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.4 μg of polysaccharide for glycoconjugate from S. pneumoniae serotype 6B and about 8.8 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.2 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotype 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.0 μg of polysaccharide from each glycoconjugate from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg of polysaccharide for glycoconjugate from S. pneumoniae serotype 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.4 μg of polysaccharide for glycoconjugate from S. pneumoniae serotypes 3 and 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, and about 4.0 μg of polysaccharide for glycoconjugate from S. pneumoniae serotypes 3 and 6B. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.4 μg of polysaccharide for glycoconjugate from S. pneumoniae serotype 6B and about 6.6 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg of polysaccharide for glycoconjugate from S. pneumoniae serotype 6B and about 6.0 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.4 μg of polysaccharide for glycoconjugate from S. pneumoniae serotype 6B and about 8.8 μg of polysaccharide for glycoconjugate from S. pneumoniae serotype 3. In a preferred embodiment the immunogenic composition is a 25-valent pneumococcal conjugate compositions.

In an embodiment, the invention relates to an immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B, about 4.0 μg of polysaccharide for glycoconjugate from S. pneumoniae serotype 6B and about 8.0 μg of polysaccharide for glycoconjugate from S. pneumoniae serotype 3. In a preferred embodiment the immunogenic composition is a 25-valent pneumococcal conjugate compositions.

In a preferred embodiment, the invention relates to a 25-valent pneumococcal conjugate immunogenic wherein said 25 glycoconjugates are glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.0 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B and about 4.0 μg of polysaccharide for glycoconjugates from S. pneumoniae serotypes 3 and 6B. In a preferred embodiment said glycoconjugate from S. pneumoniae serotype 3 is prepared using click chemistry, said glycoconjugates from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F and 35B are prepared using reductive amination and said serotype 33F glycoconjugate is prepared using eTEC conjugation.

In a very preferred embodiment, the invention relates to a 25-valent pneumococcal conjugate immunogenic wherein said 25 glycoconjugates are glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the glycoconjugates from S. pneumoniae serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F and 35B are conjugated to CRM₁₉₇, wherein each dose comprises about 2.2 μg of polysaccharide from each glycoconjugate from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F, 33F and 35B and about 4.4 μg of polysaccharide for glycoconjugates from S. pneumoniae serotypes 3 and 6B. In a preferred embodiment said glycoconjugate from S. pneumoniae serotype 3 is prepared using click chemistry, said glycoconjugates from S. pneumoniae serotypes 1, 4, 5, 6A, 7F, 8, 9V, 10A, 11A, 12F, 14, 15A, 15B, 18C, 19A, 19F, 22F, 23A, 23B, 23F, 24F and 35B are prepared using reductive amination and said serotype 33F glycoconjugate is prepared using eTEC conjugation.

### 2.3 Carrier Amount

Generally, each dose will comprise 10 μg to 150 μg of carrier protein, particularly 15 μg to 100 μg of carrier protein, more particularly 25 μg to 75 μg of carrier protein, and even more particularly 40 μg to 60 μg of carrier protein.

In an embodiment, each dose will comprise 50 μg to 70 μg of carrier protein.

In an embodiment, each dose will comprise about 30 μg, about 35 μg, about 40 μg, about 45 μg, about 50 μg, or about 60 μg of carrier protein.

In an embodiment, each dose will comprise about 65 μg of carrier protein.

Preferably, each dose will comprise from about 45 μg to about 55 μg of carrier protein.

Very preferably, each dose will comprise from about 60 μg to about 70 μg of carrier protein.

In an embodiment, each dose will comprise about 47 μg of carrier protein. In an embodiment, each dose will comprise about 48 μg of carrier protein. In an embodiment, each dose will comprise about 49 μg of carrier protein. In an embodiment, each dose will comprise about 50 μg of carrier protein. In an embodiment, each dose will comprise about 51 μg of carrier protein. In an embodiment, each dose will comprise about 52 μg of carrier protein. In an embodiment, each dose will comprise about 53 μg of carrier protein.

In an embodiment, each dose will comprise about 60 μg of carrier protein. In an embodiment, each dose will comprise about 61 μg of carrier protein. In an embodiment, each dose will comprise about 62 μg of carrier protein. In an embodiment, each dose will comprise about 63 μg of carrier protein. In an embodiment, each dose will comprise about 64 μg of carrier protein. In an embodiment, each dose will comprise about 65 μg of carrier protein. In an embodiment, each dose will comprise about 66 μg of carrier protein. In an embodiment, each dose will comprise about 67 μg of carrier protein. In an embodiment, each dose will comprise about 68 μg of carrier protein. In an embodiment, each dose will comprise about 69 μg of carrier protein. In an embodiment, each dose will comprise about 70 μg of carrier protein.

In a preferred embodiment, each dose will comprise about 65 μg of carrier protein.

In an embodiment, each dose will comprise from about 50 μg to about 70 μg of CRM₁₉₇ and from about 2 μg to about 10 μg of SCP. In an embodiment, each dose will comprise from about 55 μg to about 65 μg of CRM₁₉₇ and from about 3 μg to about 7 μg of SCP.

In a preferred embodiment, each dose will comprise about 60 μg of CRM₁₉₇ and about 5 μg of SCP.

### 2.4 Further Antigens

Immunogenic compositions of the invention comprise conjugated S. pneumoniae saccharide antigen(s) (glycoconjugate(s)). They may also further include antigen(s) from other pathogen(s), particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg—IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is preferred to use one, two or (preferably) three of the following antigens: (1) detoxified pertussis toxin (pertussis toxoid, or PT); (2) filamentous hemagglutinin (FHA); (3) pertactin (also known as the 69 kilodalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred.

Inactivated poliovirus vaccine: Poliovirus causes poliomyelitis. Rather than use oral poliovirus vaccine, preferred embodiments of the invention use IPV. Prior to administration to patients, polioviruses must be inactivated, and this can be achieved by treatment with formaldehyde. Poliomyelitis can be caused by one of three types of poliovirus. The three types are similar and cause identical symptoms, but they are antigenically different and infection by one type does not protect against infection by others. It is therefore preferred to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Preferred diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines. Preferred tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. A preferred HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of −24 kDa. All existing hepatitis B vaccines contain HBsAg, and when this antigen is administered to a normal vaccine it stimulates the production of anti-HBsAg antibodies which protect against HBV infection. For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most preferred form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM₁₉₇, H. influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

In an embodiment the immunogenic compositions of the invention further include a conjugated N. meningitidis serogroup Y capsular saccharide (MenY), and/or a conjugated N. meningitidis serogroup C capsular saccharide (MenC).

In an embodiment the immunogenic compositions of the invention further include a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic compositions of the invention further include a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

### 2.5 Adjuvant(s)

In some embodiments, the immunogenic compositions disclosed herein may further comprise at least one, two or three adjuvants. In some embodiments, the immunogenic compositions disclosed herein may further comprise at least one adjuvant.

In some embodiments, the immunogenic compositions disclosed herein may further comprise one adjuvant.

In some embodiments, the immunogenic compositions disclosed herein may further comprise two adjuvants.

The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide).

In a preferred embodiment, the immunogenic compositions disclosed herein comprise aluminum phosphate or aluminum hydroxide as adjuvant. In an even preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

### 3 Formulation

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer.

In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

### 4 Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, non-human primate, cat, sheep, pig, horse, bovine or dog. Preferably, the subject is a human.

The immunogenic compositions described herein may be used in therapeutic or prophylactic methods for preventing, treating or ameliorating a bacterial infection, disease or condition in a subject. In particular, immunogenic compositions described herein may be used to prevent, treat or ameliorate a S. pneumoniae infection, disease or condition in a subject. Preferably, the immunogenic compositions described herein may be used to prevent, treat or ameliorate S. pneumoniae infection, disease or condition by the serotypes contained in the composition in a subject.

Thus, in one aspect, the disclosure provides a method of preventing, treating or ameliorating an infection, disease or condition associated with S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B in a subject, comprising administering to the subject an immunologically effective amount of an immunogenic composition of the disclosure.

In an aspect, the disclosure provides a method of preventing, treating or ameliorating an infection, disease or condition associated with S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B in a subject, comprising administering to the subject an immunologically effective amount of an immunogenic composition of the disclosure.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the disclosure provides a method of inducing an immune response to S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B in a subject comprising administering to the subject an immunologically effective amount of an immunogenic composition of the invention. In one aspect, the subject is a mammal, such as a human, cat, sheep, pig, horse, bovine or dog. Preferably the subject is a human.

In an embodiment, the disclosure provides a method of inducing an immune response to S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B in a subject comprising administering to the subject an immunologically effective amount of an immunogenic composition of the invention. In one aspect, the subject is a mammal, such as a human, cat, sheep, pig, horse, bovine or dog. Preferably the subject is a human.

In an embodiment, the immunogenic compositions disclosed herein are for use as a vaccine. In such embodiments the immunogenic compositions described herein may be used to prevent S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B infection in a subject. Thus, in one aspect, the invention provides a method of preventing an infection by S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B in a subject comprising administering to the subject an immunologically effective amount of an immunogenic composition of the disclosure. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject is a mammal, such as a human, cat, sheep, pig, horse, bovine or dog. Preferably the subject is a human.

In one aspect, the invention provides a method of preventing an infection by S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B in a subject comprising administering to the subject an immunologically effective amount of an immunogenic composition of the disclosure. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject is a mammal, such as a human, cat, sheep, pig, horse, bovine or dog. Preferably the subject is a human.

The immunogenic composition of the present disclosure can be used to protect or treat a human susceptible to a S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and/or 35B infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

The immunogenic composition of the present disclosure can be used to protect or treat a human susceptible to a S. pneumoniae serotype 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

### 5 Subject to be Treated with the Immunogenic Compositions of the Invention

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age). In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age. For example, the subject to be vaccinated can be about 12 to about 15 months of age. In some cases, as little as one dose of the immunogenic composition according to the invention is needed, but under some circumstances, a second, third or fourth dose may be given (see section 8 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy. Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male or a human female.

### 6 Regimen

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

### 7 Particular Embodiments of the Invention are Set Forth in the Following Numbered Paragraphs 1 to 715

1. A S. pneumoniae serotype 15A glycoconjugate comprising a serotype 15A capsular polysaccharide wherein the weight average molecular weight (Mw) of said polysaccharide is between 50 kDa and 500 kDa.

2. The S. pneumoniae serotype 15A glycoconjugate of paragraph 1 wherein the weight average molecular weight (Mw) of said polysaccharide is between 75 kDa and 250 kDa.

3. The S. pneumoniae serotype 15A glycoconjugate of paragraph 1 wherein the weight average molecular weight (Mw) of said polysaccharide is between 75 kDa and 175 kDa.

4. The S. pneumoniae serotype 15A glycoconjugate of paragraph 1 wherein the weight average molecular weight (Mw) of said polysaccharide is between 90 kDa and 150 kDa.

5. The S. pneumoniae serotype 15A glycoconjugate of any one of paragraphs 1-4 having a weight average molecular weight (Mw) of between 500 kDa and 10,000 kDa.

6. The S. pneumoniae serotype 15A glycoconjugate of any one of paragraphs 1-4 having a weight average molecular weight (Mw) of between 1,000 kDa and 10,000 kDa.

7. The S. pneumoniae serotype 15A glycoconjugate of any one of paragraphs 1-4 having a weight average molecular weight (Mw) of between 1,000 kDa and 6,000 kDa.

8. The S. pneumoniae serotype 15A glycoconjugate of any one of paragraphs 1-7 wherein the serotype 15A polysaccharide to carrier protein ratio (w/w) in the glycoconjugate is between 0.5 and 3.0.

9. The S. pneumoniae serotype 15A glycoconjugate of any one of paragraphs 1-7 wherein the serotype 15A polysaccharide to carrier protein ratio (w/w) in the glycoconjugate is between 0.5 and 1.5.

10. The S. pneumoniae serotype 15A glycoconjugate of any one of paragraphs 1-7 wherein the serotype 15A polysaccharide to carrier protein ratio (w/w) in the glycoconjugate is between 0.7 and 1.1.

11. The S. pneumoniae serotype 15A glycoconjugate of any one of paragraphs 1-10 wherein the degree of conjugation is between 2 and 20.

12. The S. pneumoniae serotype 15A glycoconjugate of any one of paragraphs 1-10 wherein the degree of conjugation is between 5 and 10.

13. The S. pneumoniae serotype 15A glycoconjugate of any one of paragraphs 1-12 comprising less than about 40% of free serotype 15A polysaccharide compared to the total amount of serotype 15A polysaccharide.

14. The S. pneumoniae serotype 15A glycoconjugate of any one of paragraphs 1-12 comprising less than about 25% of free serotype 15A polysaccharide compared to the total amount of serotype 15A polysaccharide.

15. The S. pneumoniae serotype 15A glycoconjugate of any one of paragraphs 1-14 wherein at least 40% of the serotype 15A glycoconjugate has a Kd below or equal to 0.3 in a CL-4B column.

16. The S. pneumoniae serotype 15A glycoconjugate of any one of paragraphs 1-14 wherein at least 50% of the glycoconjugate has a Kd below or equal to 0.3 in a CL-4B column.

17. The S. pneumoniae serotype 15A glycoconjugate of any one of paragraphs 1-14 wherein between 50% and 90% of the serotype 15A glycoconjugate has a Kd below or equal to 0.3 in a CL-4B column.

18. The S. pneumoniae serotype 15A glycoconjugate of any one of paragraphs 1-14 wherein the carrier protein of the glycoconjugates is the fusion protein CP1.

19. The S. pneumoniae serotype 15A glycoconjugate of any one of paragraphs 1-14 wherein the carrier protein of the glycoconjugate is H. influenzae protein D.

20. The S. pneumoniae serotype 15A glycoconjugate of any one of paragraphs 1-14 wherein the carrier protein of the glycoconjugate is TT, DT, CRM₁₉₇ or a C5a peptidase from Streptococcus (SCP).

21. The S. pneumoniae serotype 15A glycoconjugate of any one of paragraphs 1-14 wherein the carrier protein of the glycoconjugate is DT.

22. The S. pneumoniae serotype 15A glycoconjugate of any one of paragraphs 1-14 wherein the carrier protein of the glycoconjugate is TT.

23. The S. pneumoniae serotype 15A glycoconjugate of any one of paragraphs 1-14 wherein the carrier protein of the glycoconjugate is CRM₁₉₇ or a C5a peptidase from Streptococcus (SCP).

24. The S. pneumoniae serotype 15A glycoconjugate of any one of paragraphs 1-14 wherein the carrier protein of the glycoconjugate is a C5a peptidase from Streptococcus (SCP).

25. The S. pneumoniae serotype 15A glycoconjugate of any one of paragraphs 1-14 wherein the carrier protein of the glycoconjugate is CRM₁₉₇.

26. The S. pneumoniae serotype 15A glycoconjugate of any one of paragraphs 1-25 prepared using reductive amination chemistry.

27. A process for preparing a S. pneumoniae serotype 15A glycoconjugate by conjugating an isolated serotype 15A capsular polysaccharide to a carrier protein comprising the step of:
(a) reacting an isolated serotype 15A capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

28. The process of paragraph 27, wherein before the oxidation step (a) the isolated serotype 15A capsular polysaccharide is sized.

29. The process of any one of paragraphs 27-28, wherein the oxidation step (a) is carried out at a pH of between 4.0 and 6.0.

30. The process of any one of paragraphs 27-28, wherein the oxidation step (a) is carried out at a pH of between 4.5 and 5.5.

31. The process of any one of paragraphs 27-28, wherein the oxidation step (a) is carried out at a pH about 5.0.

32. The process of any one of paragraphs 27-31, wherein the activated serotype 15A polysaccharide has a weight average molecular weight (Mw) of between 50 kDa and 500 kDa.

33. The process of any one of paragraphs 27-31, wherein the activated serotype 15A polysaccharide has a weight average molecular weight (Mw) of between 75 kDa and 250 kDa.

34. The process of any one of paragraphs 27-31, wherein the activated serotype 15A polysaccharide has a weight average molecular weight (Mw) of between 75 kDa and 175 kDa.

35. The process of any one of paragraphs 27-31, wherein the activated serotype 15A polysaccharide has a weight average molecular weight (Mw) of between 90 kDa and 150 kDa.

36. The process of any one of paragraphs 27-35, wherein the oxidizing agent is periodate.

37. The process of any one of paragraphs 27-35, wherein the oxidizing agent is sodium periodate.

38. The process of any one of paragraphs 27-35, wherein the oxidizing agent is sodium metaperiodate.

39. The process of any one of paragraphs 27-38, wherein the oxidation step (a) comprises reacting the polysaccharide with 0.1-2 molar equivalents of periodate.

40. The process of any one of paragraphs 27-38, wherein the oxidation step (a) comprises reacting the polysaccharide with 0.5-1.5 molar equivalents of periodate.

41. The process of any one of paragraphs 27-38, wherein the oxidation step (a) comprises reacting the polysaccharide with 0.8-1.2 molar equivalents of periodate.

42. The process of any one of paragraphs 27-41, wherein the degree of oxidation of the activated serotype 15A polysaccharide is between 2 and 20.

43. The process of any one of paragraphs 27-41, wherein the degree of oxidation of the activated serotype 15A polysaccharide is between 2 and 8.

44. The process of any one of paragraphs 27-41, wherein the degree of oxidation of the activated serotype 15A polysaccharide is 5±2.5.

45. The process of any one of paragraphs 27-44, wherein the activated serotype 15A polysaccharide and the carrier protein are lyophilised before step b).

46. The process of any one of paragraphs 27-45, wherein lyophilisation occurs after step a).

47. The process of any one of paragraphs 27-45, wherein the activated polysaccharide is lyophilised after step a) and the carrier protein is also lyophilised.

48. The process of any one of paragraphs 27-45, wherein the activated serotype 15A polysaccharide is lyophilised after step a) and the carrier protein is also lyophilised, and the activated polysaccharide and the carrier protein are reconstituted in the same solution.

49. The process of any one of paragraphs 45-48, wherein the activated serotype 15A polysaccharide and the carrier protein are lyophilised independently.

50. The process of any one of paragraphs 45-48, wherein the activated serotype 15A polysaccharide and the carrier protein are lyophilised together (co-lyophilized).

51. The process of any one of paragraphs 27-50, wherein the initial input ratio (weight by weight) of activated serotype 15A capsular polysaccharide to carrier protein at step b) is between 3:1 and 0.5:1.

52. The process of any one of paragraphs 27-50, wherein the initial input ratio (weight by weight) of activated serotype 15A capsular polysaccharide to carrier protein at step b) is between 1.5:1 and 0.5:1.

53. The process of any one of paragraphs 27-50, wherein the initial input ratio (weight by weight) of activated serotype 15A capsular polysaccharide to carrier protein at step b) is between 1.1:1 and 0.9:1.

54. The process of any one of paragraphs 27-53, wherein the reduction reaction (c) is carried out in aqueous solvent.

55. The process of any one of paragraphs 27-53, wherein the reduction reaction (c) is carried out in aprotic solvent.

56. The process of any one of paragraphs 27-53, wherein the reduction reaction (c) is carried out in the presence of dimethylsulphoxide (DMSO) or dimethylformamide (DMF).

57. The process of any one of paragraphs 27-53, wherein the reduction reaction (c) is carried out in the presence of dimethylsulphoxide (DMSO).

58. The process of any one of paragraphs 27-53, wherein the reduction reaction (c) is carried out in DMSO (dimethylsulfoxide) or in DMF (dimethylformamide)) solvent.

59. The process of any one of paragraphs 27-53, wherein the reduction reaction (c) is carried out in DMSO (dimethylsulfoxide) solvent.

60. The process of any one of paragraphs 27-59, wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN-BH3, benzylamine-BH3 or 5-ethyl-2-methylpyridine borane (PEMB).

61. The process of any one of paragraphs 27-59, wherein the reducing agent is sodium triacetoxyborohydride.

62. The process of any one of paragraphs 27-59, wherein the reducing agent is sodium cyanoborohydride in the present of nickel.

63. The process of any one of paragraphs 27-59, wherein the reducing agent is sodium cyanoborohydride.

64. The process of any one of paragraphs 27-63, wherein between 0.2 and 5 molar equivalents of reducing agent is used in step c).

65. The process of any one of paragraphs 27-63, wherein between 0.5 and 2 molar equivalents of reducing agent is used in step c).

66. The process of any one of paragraphs 27-63, wherein between 0.9 and 1.1 molar equivalent of reducing agent is used in step c).

67. The process of any one of paragraphs 27-66, wherein following the reduction reaction (c), unreacted aldehyde groups remaining in the conjugates are capped using a suitable capping agent.

68. The process of paragraph 67, wherein said capping agent is sodium borohydride (NaBH₄).

69. The process of any one of paragraphs 67-68, wherein capping is achieved by mixing the product of step c) with 1 to 3 molar equivalents of sodium borohydride.

70. The process of any one of paragraphs 27-69, comprising the step of purifying the glycoconjugate after it is produced.

71. The process of any one of paragraphs 27-70, wherein the carrier protein is the fusion protein Cp1.

72. The process of any one of paragraphs 27-70, wherein the carrier protein is H. influenzae protein D.

73. The process of any one of paragraphs 27-70, wherein the carrier protein is TT, DT, CRM₁₉₇ or a C5a peptidase from Streptococcus (SCP).

74. The process of any one of paragraphs 27-70, wherein the carrier protein is DT.

75. The process of any one of paragraphs 27-70, wherein the carrier protein is TT.

76. The process of any one of paragraphs 27-70, wherein the carrier protein is CRM₁₉₇ or a C5a peptidase from Streptococcus (SCP).

77. The process of any one of paragraphs 27-70, wherein the carrier protein is a C5a peptidase from Streptococcus (SCP).

78. The process of any one of paragraphs 27-70, wherein the carrier protein is CRM₁₉₇.

79. A S. pneumoniae serotype 15A glycoconjugate obtained according to the process of any one of paragraphs 27-78.

80. A S. pneumoniae serotype 23A glycoconjugate comprising a serotype 23A capsular polysaccharide wherein the weight average molecular weight (Mw) of said polysaccharide is between 50 kDa and 400 kDa.

81. The S. pneumoniae serotype 23A glycoconjugate of paragraph 80 wherein the weight average molecular weight (Mw) of said polysaccharide is between 100 kDa and 300 kDa.

82. The S. pneumoniae serotype 23A glycoconjugate of paragraph 80 wherein the weight average molecular weight (Mw) of said polysaccharide is between 110 kDa and 250 kDa.

83. The S. pneumoniae serotype 23A glycoconjugate of paragraph 80 wherein the weight average molecular weight (Mw) of said polysaccharide is between 120 kDa and 240 kDa.

84. The S. pneumoniae serotype 23A glycoconjugate of any one of paragraphs 80-83 having a weight average molecular weight (Mw) of between 500 kDa and 10,000 kDa.

85. The S. pneumoniae serotype 23A glycoconjugate of any one of paragraphs 80-83 having a weight average molecular weight (Mw) of between 1,000 kDa and 7,500 kDa.

86. The S. pneumoniae serotype 23A glycoconjugate of any one of paragraphs 80-83 having a weight average molecular weight (Mw) of between 2,000 kDa and 5,000 kDa.

87. The S. pneumoniae serotype 23A glycoconjugate of any one of paragraphs 80-86 wherein the serotype 23A polysaccharide to carrier protein ratio (w/w) in the glycoconjugate is between 0.5 and 3.0.

88. The S. pneumoniae serotype 23A glycoconjugate of any one of paragraphs 80-86 wherein the serotype 23A polysaccharide to carrier protein ratio (w/w) in the glycoconjugate is between 0.5 and 1.7.

89. The S. pneumoniae serotype 23A glycoconjugate of any one of paragraphs 80-86 wherein the serotype 23A polysaccharide to carrier protein ratio (w/w) in the glycoconjugate is between 0.8 and 1.4.

90. The S. pneumoniae serotype 23A glycoconjugate of any one of paragraphs 80-89 wherein the degree of conjugation is between 2 and 20.

91. The S. pneumoniae serotype 23A glycoconjugate of any one of paragraphs 80-89 wherein the degree of conjugation is between 5 and 15.

92. The S. pneumoniae serotype 23A glycoconjugate of any one of paragraphs 80-91 comprising less than about 40% of free serotype 23A polysaccharide compared to the total amount of serotype 23A polysaccharide.

93. The S. pneumoniae serotype 23A glycoconjugate of any one of paragraphs 80-91 comprising less than about 25% of free serotype 23A polysaccharide compared to the total amount of serotype 23A polysaccharide.

94. The S. pneumoniae serotype 23A glycoconjugate of any one of paragraphs 80-93 wherein at least 40% of the serotype 23A glycoconjugate has a Kd below or equal to 0.3 in a CL-4B column.

95. The S. pneumoniae serotype 23A glycoconjugate of any one of paragraphs 80-93 wherein at least 50% of the glycoconjugate has a Kd below or equal to 0.3 in a CL-4B column.

96. The S. pneumoniae serotype 23A glycoconjugate of any one of paragraphs 80-93 wherein between 50% and 90% of the serotype 23A glycoconjugate has a Kd below or equal to 0.3 in a CL-4B column.

97. The S. pneumoniae serotype 23A glycoconjugate of any one of paragraphs 80-96 comprising a S. pneumoniae serotype 23A capsular polysaccharide wherein said S. pneumoniae serotype 23A capsular polysaccharide has a branched Rhamnose content greater than 75% when compared to native S. pneumoniae serotype 23A capsular polysaccharide wherein the branched Rhamnose content is considered to be about 100%.

98. The S. pneumoniae serotype 23A glycoconjugate of any one of paragraphs 80-96 comprising a S. pneumoniae serotype 23A capsular polysaccharide wherein said S. pneumoniae serotype 23A capsular polysaccharide has a branched Rhamnose content greater than 90% when compared to native S. pneumoniae serotype 23A capsular polysaccharide wherein the branched Rhamnose content is considered to be about 100%.

99. The S. pneumoniae serotype 23A glycoconjugate of any one of paragraphs 80-96 comprising a S. pneumoniae serotype 23A capsular polysaccharide wherein said S. pneumoniae serotype 23A capsular polysaccharide has a branched Rhamnose content greater than 95% when compared to native S. pneumoniae serotype 23A capsular polysaccharide wherein the branched Rhamnose content is considered to be about 100%.

100. The S. pneumoniae serotype 23A glycoconjugate of any one of paragraphs 80-96 comprising a S. pneumoniae serotype 23A capsular polysaccharide wherein said S. pneumoniae serotype 23A capsular polysaccharide has a branched Rhamnose content of about 100% when compared to native S. pneumoniae serotype 23A capsular polysaccharide wherein the branched Rhamnose content is considered to be about 100%.

101. The S. pneumoniae serotype 23A glycoconjugate of any one of paragraphs 80-100 wherein the carrier protein of the glycoconjugates is the fusion protein CP1.

102. The S. pneumoniae serotype 23A glycoconjugate of any one of paragraphs 80-100 wherein the carrier protein of the glycoconjugate is H. influenzae protein D.

103. The S. pneumoniae serotype 23A glycoconjugate of any one of paragraphs 80-100 wherein the carrier protein of the glycoconjugate is TT, DT, CRM₁₉₇ or a C5a peptidase from Streptococcus (SCP).

104. The S. pneumoniae serotype 23A glycoconjugate of any one of paragraphs 80-100 wherein the carrier protein of the glycoconjugate is DT.

105. The S. pneumoniae serotype 23A glycoconjugate of any one of paragraphs 80-100 wherein the carrier protein of the glycoconjugate is TT.

106. The S. pneumoniae serotype 23A glycoconjugate of any one of paragraphs 80-100 wherein the carrier protein of the glycoconjugate is CRM₁₉₇ or a C5a peptidase from Streptococcus (SCP).

107. The S. pneumoniae serotype 23A glycoconjugate of any one of paragraphs 80-100 wherein the carrier protein of the glycoconjugate is a C5a peptidase from Streptococcus (SCP).

108. The S. pneumoniae serotype 23A glycoconjugate of any one of paragraphs 80-100 wherein the carrier protein of the glycoconjugate is CRM₁₉₇.

109. The S. pneumoniae serotype 23A glycoconjugate of any one of paragraphs 80-108 prepared using reductive amination chemistry.

110. A process for preparing a S. pneumoniae serotype 23A glycoconjugate by conjugating an isolated serotype 23A capsular polysaccharide to a carrier protein comprising the step of: (a) reacting an isolated serotype 23A capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

111. The process of paragraph 110, wherein before the oxidation step (a) the isolated serotype 23A capsular polysaccharide is sized.

112. The process of paragraph 110, wherein the size of the isolated serotype 23A capsular polysaccharide is reduced by mechanical homogenization.

113. The process of paragraph 110, wherein the size of the isolated serotype 23A polysaccharide is reduced by high pressure homogenization.

114. The process of any one of paragraphs 110-113, wherein the isolated serotype 23A capsular polysaccharide is not sized by acid hydrolysis.

115. The process of paragraph 110, wherein the isolated serotype 23A capsular polysaccharide is not sized before oxidation.

116. The process of any one of paragraphs 110-115, wherein the oxidation step (a) is carried out at a pH of between 4.5 and 6.5.

117. The process of any one of paragraphs 110-115, wherein the oxidation step (a) is carried out at a pH of between 5.0 and 6.0.

118. The process of any one of paragraphs 110-115, wherein the oxidation step (a) is carried out at a pH about 5.0.

119. The process of any one of paragraphs 110-118, wherein the activated serotype 23A polysaccharide has a weight average molecular weight (Mw) of between 50 kDa and 400 kDa.

120. The process of any one of paragraphs 110-118, wherein the activated serotype 23A polysaccharide has a weight average molecular weight (Mw) of between 100 kDa and 250 kDa.

121. The process of any one of paragraphs 110-118, wherein the activated serotype 23A polysaccharide has a weight average molecular weight (Mw) of between 120 kDa and 200 kDa.

122. The process of any one of paragraphs 110-121, wherein the activated serotype 23A polysaccharide retains at least 80% of the branched Rhamnose.

123. The process of any one of paragraphs 110-121, wherein the activated serotype 23A polysaccharide retains at least 85% of the branched Rhamnose.

124. The process of any one of paragraphs 110-121, wherein the activated serotype 23A polysaccharide retains at least 90% of the branched Rhamnose.

125. The process of any one of paragraphs 110-121, wherein the activated serotype 23A polysaccharide retains at least 95% of the branched Rhamnose.

126. The process of any one of paragraphs 110-121, wherein the activated serotype 23A polysaccharide retains at least 96% of the branched Rhamnose.

127. The process of any one of paragraphs 110-126, wherein the oxidizing agent is periodate.

128. The process of any one of paragraphs 110-126, wherein the oxidizing agent is sodium periodate.

129. The process of any one of paragraphs 110-126, wherein the oxidizing agent is sodium metaperiodate.

130. The process of any one of paragraphs 110-129, wherein the oxidation step (a) comprises reacting the polysaccharide with 0.1-2 molar equivalents of periodate.

131. The process of any one of paragraphs 110-129, wherein the oxidation step (a) comprises reacting the polysaccharide with 0.2-1.5 molar equivalents of periodate.

132. The process of any one of paragraphs 110-129, wherein the oxidation step (a) comprises reacting the polysaccharide with 0.3-0.5 molar equivalents of periodate.

133. The process of any one of paragraphs 110-132, wherein the degree of oxidation of the activated serotype 23A polysaccharide is between 2 and 20.

134. The process of any one of paragraphs 110-132, wherein the degree of oxidation of the activated serotype 23A polysaccharide is between 2 and 8.

135. The process of any one of paragraphs 110-132, wherein the degree of oxidation of the activated serotype 23A polysaccharide is 5±2.5.

136. The process of any one of paragraphs 110-135, wherein the activated serotype 23A polysaccharide and the carrier protein are lyophilised before step b).

137. The process of any one of paragraphs 110-136, wherein lyophilisation occurs after step a).

138. The process of any one of paragraphs 110-136, wherein the activated polysaccharide is lyophilised after step a) and the carrier protein is also lyophilised.

139. The process of any one of paragraphs 110-136, wherein the activated serotype 23A polysaccharide is lyophilised after step a) and the carrier protein is also lyophilised, and the activated polysaccharide and the carrier protein are reconstituted in the same solution.

140. The process of any one of paragraphs 136-139, wherein the activated serotype 23A polysaccharide and the carrier protein are lyophilised independently.

141. The process of any one of paragraphs 136-139, wherein the activated serotype 23A polysaccharide and the carrier protein are lyophilised together (co-lyophilized).

142. The process of any one of paragraphs 110-141, wherein the initial input ratio (weight by weight) of activated serotype 23A capsular polysaccharide to carrier protein at step b) is between 2:1 and 0.5:1.

143. The process of any one of paragraphs 110-141, wherein the initial input ratio (weight by weight) of activated serotype 23A capsular polysaccharide to carrier protein at step b) is between 1.2:1 and 0.6:1.

144. The process of any one of paragraphs 110-141, wherein the initial input ratio (weight by weight) of activated serotype 23A capsular polysaccharide to carrier protein at step b) is between 0.9:1 and 0.7:1.

145. The process of any one of paragraphs 110-144, wherein the reduction reaction (c) is carried out in aqueous solvent.

146. The process of any one of paragraphs 110-144, wherein the reduction reaction (c) is carried out in aprotic solvent.

147. The process of any one of paragraphs 110-144, wherein the reduction reaction (c) is carried out in the presence of dimethylsulphoxide (DMSO) or dimethylformamide (DMF).

148. The process of any one of paragraphs 110-144, wherein the reduction reaction (c) is carried out in the presence of dimethylsulphoxide (DMSO).

149. The process of any one of paragraphs 110-144, wherein the reduction reaction (c) is carried out in DMSO (dimethylsulfoxide) or in DMF (dimethylformamide)) solvent.

150. The process of any one of paragraphs 110-144, wherein the reduction reaction (c) is carried out in DMSO (dimethylsulfoxide) solvent.

151. The process of any one of paragraphs 110-150, wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN-BH3, benzylamine-BH3 or 5-ethyl-2-methylpyridine borane (PEMB).

152. The process of any one of paragraphs 110-150, wherein the reducing agent is sodium triacetoxyborohydride.

153. The process of any one of paragraphs 110-150, wherein the reducing agent is sodium cyanoborohydride in the present of nickel.

154. The process of any one of paragraphs 110-150, wherein the reducing agent is sodium cyanoborohydride.

155. The process of any one of paragraphs 110-154, wherein between 0.2 and 5 molar equivalents of reducing agent is used in step c).

156. The process of any one of paragraphs 110-154, wherein between 0.2 and 1.5 molar equivalents of reducing agent is used in step c).

157. The process of any one of paragraphs 110-154, wherein between 0.5 and 1.0 molar equivalent of reducing agent is used in step c).

158. The process of any one of paragraphs 110-157, wherein following the reduction reaction (c), unreacted aldehyde groups remaining in the conjugates are capped using a suitable capping agent.

159. The process of paragraph 158, wherein said capping agent is sodium borohydride (NaBH₄).

160. The process of any one of paragraphs 158-159, wherein capping is achieved by mixing the product of step c) with 1 to 20 molar equivalents of sodium borohydride.

161. The process of any one of paragraphs 158-159, wherein capping is achieved by mixing the product of step c) with 1 to 3 molar equivalents of sodium borohydride.

162. The process of any one of paragraphs 110-161, comprising the step of purifying the glycoconjugate after it is produced.

163. The process of any one of paragraphs 110-162, wherein the carrier protein is the fusion protein CP1.

164. The process of any one of paragraphs 110-162, wherein the carrier protein is H. influenzae protein D.

165. The process of any one of paragraphs 110-162, wherein the carrier protein is TT, DT, CRM₁₉₇ or a C5a peptidase from Streptococcus (SCP).

166. The process of any one of paragraphs 110-162, wherein the carrier protein is DT.

167. The process of any one of paragraphs 110-162, wherein the carrier protein is TT.

168. The process of any one of paragraphs 110-162, wherein the carrier protein is CRM₁₉₇ or a C5a peptidase from Streptococcus (SCP).

169. The process of any one of paragraphs 110-162, wherein the carrier protein is a C5a peptidase from Streptococcus (SCP).

170. The process of any one of paragraphs 110-162, wherein the carrier protein is CRM₁₉₇.

171. A S. pneumoniae serotype 23A glycoconjugate obtained according to the process of any one of paragraphs 110-170.

172. A S. pneumoniae serotype 23B glycoconjugate comprising a serotype 23B capsular polysaccharide wherein the weight average molecular weight (Mw) of said polysaccharide is between 40 kDa and 600 kDa.

173. The S. pneumoniae serotype 23B glycoconjugate of paragraph 172 wherein the weight average molecular weight (Mw) of said polysaccharide is between 50 kDa and 300 kDa.

174. The S. pneumoniae serotype 23B glycoconjugate of paragraph 172 wherein the weight average molecular weight (Mw) of said polysaccharide is between 75 kDa and 250 kDa.

175. The S. pneumoniae serotype 23B glycoconjugate of paragraph 172 wherein the weight average molecular weight (Mw) of said polysaccharide is between 100 kDa and 200 kDa.

176. The S. pneumoniae serotype 23B glycoconjugate of any one of paragraphs 172-175 having a weight average molecular weight (Mw) of between 250 kDa and 7,500 kDa.

177. The S. pneumoniae serotype 23B glycoconjugate of any one of paragraphs 172-175 having a weight average molecular weight (Mw) of between 500 kDa and 4,000 kDa.

178. The S. pneumoniae serotype 23B glycoconjugate of any one of paragraphs 172-175 having a weight average molecular weight (Mw) of between 700 kDa and 2,000 kDa.

179. The S. pneumoniae serotype 23B glycoconjugate of any one of paragraphs 172-178 wherein the serotype 23B polysaccharide to carrier protein ratio (w/w) in the glycoconjugate is between 0.4 and 3.0.

180. The S. pneumoniae serotype 23B glycoconjugate of any one of paragraphs 172-178 wherein the serotype 23B polysaccharide to carrier protein ratio (w/w) in the glycoconjugate is between 0.5 and 1.5.

181. The S. pneumoniae serotype 23B glycoconjugate of any one of paragraphs 172-178 wherein the serotype 23B polysaccharide to carrier protein ratio (w/w) in the glycoconjugate is between 0.6 and 1.3.

182. The S. pneumoniae serotype 23B glycoconjugate of any one of paragraphs 172-181 wherein the degree of conjugation is between 2 and 15.

183. The S. pneumoniae serotype 23B glycoconjugate of any one of paragraphs 172-181 wherein the degree of conjugation is between 5 and 12.

184. The S. pneumoniae serotype 23B glycoconjugate of any one of paragraphs 172-183 comprising less than about 40% of free serotype 23B polysaccharide compared to the total amount of serotype 23B polysaccharide.

185. The S. pneumoniae serotype 23B glycoconjugate of any one of paragraphs 172-183 comprising less than about 25% of free serotype 23B polysaccharide compared to the total amount of serotype 23B polysaccharide.

186. The S. pneumoniae serotype 23B glycoconjugate of any one of paragraphs 172-185 wherein at least 30% of the serotype 23B glycoconjugate has a Kd below or equal to 0.3 in a CL-4B column.

187. The S. pneumoniae serotype 23B glycoconjugate of any one of paragraphs 172-185 wherein at least 35% of the glycoconjugate has a Kd below or equal to 0.3 in a CL-4B column.

188. The S. pneumoniae serotype 23B glycoconjugate of any one of paragraphs 172-185 wherein between 40% and 60% of the serotype 23B glycoconjugate has a Kd below or equal to 0.3 in a CL-4B column.

189. The S. pneumoniae serotype 23B glycoconjugate of any one of paragraphs 172-188 wherein the carrier protein of the glycoconjugates is the fusion protein CP1.

190. The S. pneumoniae serotype 23B glycoconjugate of any one of paragraphs 172-188 wherein the carrier protein of the glycoconjugate is H. influenzae protein D.

191. The S. pneumoniae serotype 23B glycoconjugate of any one of paragraphs 172-188 wherein the carrier protein of the glycoconjugate is TT, DT, CRM₁₉₇ or a C5a peptidase from Streptococcus (SCP).

192. The S. pneumoniae serotype 23B glycoconjugate of any one of paragraphs 172-188 wherein the carrier protein of the glycoconjugate is DT.

193. The S. pneumoniae serotype 23B glycoconjugate of any one of paragraphs 172-188 wherein the carrier protein of the glycoconjugate is TT.

194. The S. pneumoniae serotype 23B glycoconjugate of any one of paragraphs 172-188 wherein the carrier protein of the glycoconjugate is CRM₁₉₇ or a C5a peptidase from Streptococcus (SCP).

195. The S. pneumoniae serotype 23B glycoconjugate of any one of paragraphs 172-188 wherein the carrier protein of the glycoconjugate is a C5a peptidase from Streptococcus (SCP).

196. The S. pneumoniae serotype 23B glycoconjugate of any one of paragraphs 172-188 wherein the carrier protein of the glycoconjugate is CRM₁₉₇.

197. The S. pneumoniae serotype 23B glycoconjugate of any one of paragraphs 172-196 prepared using reductive amination chemistry.

198. A process for preparing a S. pneumoniae serotype 23B glycoconjugate by conjugating an isolated serotype 23B capsular polysaccharide to a carrier protein comprising the step of: (a) reacting an isolated serotype 23B capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

199. The process of paragraph 198, wherein before the oxidation step (a) the isolated serotype 23B capsular polysaccharide is sized.

200. The process of paragraph 198, wherein before the oxidation step (a) the isolated serotype 23B capsular polysaccharide is sized by mechanical homogenization.

201. The process of paragraph 198, wherein before the oxidation step (a) the isolated serotype 23B capsular polysaccharide is by high pressure homogenization.

202. The process of paragraph 198, wherein the isolated serotype 23B capsular polysaccharide is not sized before oxidation.

203. The process of any one of paragraphs 198-202, wherein the oxidation step (a) is carried out at a pH of between 4.0 and 6.5.

204. The process of any one of paragraphs 198-202, wherein the oxidation step (a) is carried out at a pH of between 4.5 and 5.5.

205. The process of any one of paragraphs 198-202, wherein the oxidation step (a) is carried out at a pH about 5.0.

206. The process of any one of paragraphs 198-206, wherein the activated serotype 23B polysaccharide has a weight average molecular weight (Mw) of between 40 kDa and 600 kDa.

207. The process of any one of paragraphs 198-206, wherein the activated serotype 23B polysaccharide has a weight average molecular weight (Mw) of between 50 kDa and 300 kDa.

208. The process of any one of paragraphs 198-206, wherein the activated serotype 23B polysaccharide has a weight average molecular weight (Mw) of between 75 kDa and 250 kDa.

209. The process of any one of paragraphs 198-206, wherein the activated serotype 23B polysaccharide has a weight average molecular weight (Mw) of between 100 kDa and 200 kDa.

210. The process of any one of paragraphs 198-209, wherein the oxidizing agent is periodate.

211. The process of any one of paragraphs 198-209, wherein the oxidizing agent is sodium periodate.

212. The process of any one of paragraphs 198-209, wherein the oxidizing agent is sodium metaperiodate.

213. The process of any one of paragraphs 198-212, wherein the oxidation step (a) comprises reacting the polysaccharide with 0.05-1 molar equivalents of periodate.

214. The process of any one of paragraphs 198-212, wherein the oxidation step (a) comprises reacting the polysaccharide with 0.1-0.3 molar equivalents of periodate.

215. The process of any one of paragraphs 198-212, wherein the oxidation step (a) comprises reacting the polysaccharide with about 0.2 molar equivalents of periodate.

216. The process of any one of paragraphs 198-215, wherein the degree of oxidation of the activated serotype 23B polysaccharide is between 2 and 20.

217. The process of any one of paragraphs 198-215, wherein the degree of oxidation of the activated serotype 23B polysaccharide is between 4 and 15.

218. The process of any one of paragraphs 198-215, wherein the degree of oxidation of the activated serotype 23B polysaccharide is 9±3.

219. The process of any one of paragraphs 198-218, wherein the activated serotype 23B polysaccharide and the carrier protein are lyophilised before step b).

220. The process of any one of paragraphs 198-219, wherein lyophilisation occurs after step a).

221. The process of any one of paragraphs 198-220, wherein the activated polysaccharide is lyophilised after step a) and the carrier protein is also lyophilised.

222. The process of any one of paragraphs 198-220, wherein the activated serotype 23B polysaccharide is lyophilised after step a) and the carrier protein is also lyophilised, and the activated polysaccharide and the carrier protein are reconstituted in the same solution.

223. The process of any one of paragraphs 219-222, wherein the activated serotype 23B polysaccharide and the carrier protein are lyophilised independently.

224. The process of any one of paragraphs 219-222, wherein the activated serotype 23B polysaccharide and the carrier protein are lyophilised together (co-lyophilized).

225. The process of any one of paragraphs 198-224, wherein the initial input ratio (weight by weight) of activated serotype 23B capsular polysaccharide to carrier protein at step b) is between 2:1 and 0.5:1.

226. The process of any one of paragraphs 198-224, wherein the initial input ratio (weight by weight) of activated serotype 23B capsular polysaccharide to carrier protein at step b) is between 1.2:1 and 0.6:1.

227. The process of any one of paragraphs 198-224, wherein the initial input ratio (weight by weight) of activated serotype 23B capsular polysaccharide to carrier protein at step b) is between 0.9:1 and 0.7:1.

228. The process of any one of paragraphs 198-228, wherein the reduction reaction (c) is carried out in aqueous solvent.

229. The process of any one of paragraphs 198-228, wherein the reduction reaction (c) is carried out in aprotic solvent.

230. The process of any one of paragraphs 198-228, wherein the reduction reaction (c) is carried out in the presence of dimethylsulphoxide (DMSO) or dimethylformamide (DMF).

231. The process of any one of paragraphs 198-228, wherein the reduction reaction (c) is carried out in the presence of dimethylsulphoxide (DMSO).

232. The process of any one of paragraphs 198-228, wherein the reduction reaction (c) is carried out in DMSO (dimethylsulfoxide) or in DMF (dimethylformamide)) solvent.

233. The process of any one of paragraphs 198-228, wherein the reduction reaction (c) is carried out in DMSO (dimethylsulfoxide) solvent.

234. The process of any one of paragraphs 198-233, wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN-BH3, benzylamine-BH3 or 5-ethyl-2-methylpyridine borane (PEMB).

235. The process of any one of paragraphs 198-233, wherein the reducing agent is sodium triacetoxyborohydride.

236. The process of any one of paragraphs 198-233, wherein the reducing agent is sodium cyanoborohydride in the present of nickel.

237. The process of any one of paragraphs 198-233, wherein the reducing agent is sodium cyanoborohydride.

238. The process of any one of paragraphs 198-237, wherein between 0.2 and 5 molar equivalents of reducing agent is used in step c).

239. The process of any one of paragraphs 198-237, wherein between 0.5 and 1.5 molar equivalents of reducing agent is used in step c).

240. The process of any one of paragraphs 198-237, wherein between 0.9 and 1.1 molar equivalent of reducing agent is used in step c).

241. The process of any one of paragraphs 198-240, wherein following the reduction reaction (c), unreacted aldehyde groups remaining in the conjugates are capped using a suitable capping agent.

242. The process of paragraph 241, wherein said capping agent is sodium borohydride (NaBH₄).

243. The process of any one of paragraphs 241-242, wherein capping is achieved by mixing the product of step c) with 1 to 20 molar equivalents of sodium borohydride.

244. The process of any one of paragraphs 241-242, wherein capping is achieved by mixing the product of step c) with 1 to 3 molar equivalents of sodium borohydride.

245. The process of any one of paragraphs 198-244, comprising the step of purifying the glycoconjugate after it is produced.

246. The process of any one of paragraphs 198-245, wherein the carrier protein is the fusion protein CP1.

247. The process of any one of paragraphs 198-245, wherein the carrier protein is H. influenzae protein D.

248. The process of any one of paragraphs 198-245, wherein the carrier protein is TT, DT, CRM₁₉₇ or a C5a peptidase from Streptococcus (SCP).

249. The process of any one of paragraphs 198-245, wherein the carrier protein is DT.

250. The process of any one of paragraphs 198-245, wherein the carrier protein is TT.

251. The process of any one of paragraphs 198-245, wherein the carrier protein is CRM₁₉₇ or a C5a peptidase from Streptococcus (SCP).

252. The process of any one of paragraphs 198-245, wherein the carrier protein is a C5a peptidase from Streptococcus (SCP).

253. The process of any one of paragraphs 198-245, wherein the carrier protein is CRM₁₉₇.

254. A S. pneumoniae serotype 23B glycoconjugate obtained according to the process of any one of paragraphs 198-253.

255. A S. pneumoniae serotype 24F glycoconjugate comprising a serotype 24F capsular polysaccharide wherein the weight average molecular weight (Mw) of said polysaccharide is between 50 kDa and 400 kDa.

256. The S. pneumoniae serotype 24F glycoconjugate of paragraph 255 wherein the weight average molecular weight (Mw) of said polysaccharide is between 120 kDa and 250 kDa.

257. The S. pneumoniae serotype 24F glycoconjugate of paragraph 255 wherein the weight average molecular weight (Mw) of said polysaccharide is between 120 kDa and 200 kDa.

258. The S. pneumoniae serotype 24F glycoconjugate of any one of paragraphs 255-257 having a weight average molecular weight (Mw) of between 500 kDa and 10,000 kDa.

259. The S. pneumoniae serotype 24F glycoconjugate of any one of paragraphs 255-257 having a weight average molecular weight (Mw) of between 1,500 kDa and 7,500 kDa.

260. The S. pneumoniae serotype 24F glycoconjugate of any one of paragraphs 255-257 having a weight average molecular weight (Mw) of between 3,000 kDa and 6,000 kDa.

261. The S. pneumoniae serotype 24F glycoconjugate of any one of paragraphs 255-260 wherein the serotype 24F polysaccharide to carrier protein ratio (w/w) in the glycoconjugate is between 0.5 and 3.0.

262. The S. pneumoniae serotype 24F glycoconjugate of any one of paragraphs 255-260 wherein the serotype 24F polysaccharide to carrier protein ratio (w/w) in the glycoconjugate is between 0.7 and 1.5.

263. The S. pneumoniae serotype 24F glycoconjugate of any one of paragraphs 255-260 wherein the serotype 24F polysaccharide to carrier protein ratio (w/w) in the glycoconjugate is between 0.8 and 1.4.

264. The S. pneumoniae serotype 24F glycoconjugate of any one of paragraphs 255-263 wherein the degree of conjugation is between 2 and 15.

265. The S. pneumoniae serotype 24F glycoconjugate of any one of paragraphs 255-263 wherein the degree of conjugation is between 5 and 12.

266. The S. pneumoniae serotype 24F glycoconjugate of any one of paragraphs 255-265 comprising less than about 40% of free serotype 24F polysaccharide compared to the total amount of serotype 24F polysaccharide.

267. The S. pneumoniae serotype 24F glycoconjugate of any one of paragraphs 255-265 comprising less than about 25% of free serotype 24F polysaccharide compared to the total amount of serotype 24F polysaccharide.

268. The S. pneumoniae serotype 24F glycoconjugate of any one of paragraphs 255-267 wherein at least 40% of the serotype 24F glycoconjugate has a Kd below or equal to 0.3 in a CL-4B column.

269. The S. pneumoniae serotype 24F glycoconjugate of any one of paragraphs 255-267 wherein at least 50% of the glycoconjugate has a Kd below or equal to 0.3 in a CL-4B column.

270. The S. pneumoniae serotype 24F glycoconjugate of any one of paragraphs 255-267 wherein between 50% and 90% of the serotype 24F glycoconjugate has a Kd below or equal to 0.3 in a CL-4B column.

271. The S. pneumoniae serotype 24F glycoconjugate of any one of paragraphs 255-270 comprising a S. pneumoniae serotype 24F capsular polysaccharide wherein said S. pneumoniae serotype 24F capsular polysaccharide has a Ribose content greater than 75% when compared to native S. pneumoniae serotype 24F capsular polysaccharide wherein the Ribose content is considered to be about 100%.

272. The S. pneumoniae serotype 24F glycoconjugate of any one of paragraphs 255-270 comprising a S. pneumoniae serotype 24F capsular polysaccharide wherein said S. pneumoniae serotype 24F capsular polysaccharide has a Ribose content greater than 90% when compared to native S. pneumoniae serotype 24F capsular polysaccharide wherein the Ribose content is considered to be about 100%.

273. The S. pneumoniae serotype 24F glycoconjugate of any one of paragraphs 255-270 comprising a S. pneumoniae serotype 24F capsular polysaccharide wherein said S. pneumoniae serotype 24F capsular polysaccharide has a Ribose content greater than 95% when compared to native S. pneumoniae serotype 24F capsular polysaccharide wherein the Ribose content is considered to be about 100%.

274. The S. pneumoniae serotype 24F glycoconjugate of any one of paragraphs 255-270 comprising a S. pneumoniae serotype 24F capsular polysaccharide wherein said S. pneumoniae serotype 24F capsular polysaccharide has a Ribose content of about 100% when compared to native S. pneumoniae serotype 24F capsular polysaccharide wherein the Ribose content is considered to be about 100%.

275. The S. pneumoniae serotype 24F glycoconjugate of any one of paragraphs 255-274 wherein the carrier protein of the glycoconjugates is the fusion protein CP1.

276. The S. pneumoniae serotype 24F glycoconjugate of any one of paragraphs 255-274 wherein the carrier protein of the glycoconjugate is H. influenzae protein D.

277. The S. pneumoniae serotype 24F glycoconjugate of any one of paragraphs 255-274 wherein the carrier protein of the glycoconjugate is TT, DT, CRM₁₉₇ or a C5a peptidase from Streptococcus (SCP).

278. The S. pneumoniae serotype 24F glycoconjugate of any one of paragraphs 255-274 wherein the carrier protein of the glycoconjugate is DT.

279. The S. pneumoniae serotype 24F glycoconjugate of any one of paragraphs 255-274 wherein the carrier protein of the glycoconjugate is TT.

280. The S. pneumoniae serotype 24F glycoconjugate of any one of paragraphs 255-274 wherein the carrier protein of the glycoconjugate is CRM₁₉₇ or a C5a peptidase from Streptococcus (SCP).

281. The S. pneumoniae serotype 24F glycoconjugate of any one of paragraphs 255-274 wherein the carrier protein of the glycoconjugate is a C5a peptidase from Streptococcus (SCP).

282. The S. pneumoniae serotype 24F glycoconjugate of any one of paragraphs 255-274 wherein the carrier protein of the glycoconjugate is CRM₁₉₇.

283. The S. pneumoniae serotype 24F glycoconjugate of any one of paragraphs 255-282 prepared using reductive amination chemistry.

284. A process for preparing a S. pneumoniae serotype 24F glycoconjugate by conjugating an isolated serotype 24F capsular polysaccharide to a carrier protein comprising the step of: (a) reacting an isolated serotype 24F capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

285. The process of paragraph 284, wherein before the oxidation step (a) the isolated serotype 24F capsular polysaccharide is sized.

286. The process of paragraph 284, wherein the size of the isolated serotype 24F capsular polysaccharide is reduced by mechanical homogenization.

287. The process of paragraph 284, wherein the size of the isolated serotype 24F polysaccharide is reduced by high pressure homogenization.

288. The process of any one of paragraphs 284-287, wherein the isolated serotype 24F capsular polysaccharide is not sized by acid hydrolysis.

289. The process of paragraph 284, wherein the isolated serotype 24F capsular polysaccharide is not sized before oxidation.

290. The process of any one of paragraphs 284-289, wherein the oxidation step (a) is carried out at a pH of between 4.5 and 6.5.

291. The process of any one of paragraphs 284-289, wherein the oxidation step (a) is carried out at a pH of between 5.0 and 6.0.

292. The process of any one of paragraphs 284-289, wherein the oxidation step (a) is carried out at a pH about 5.0.

293. The process of any one of paragraphs 284-292, wherein the activated serotype 24F polysaccharide has a weight average molecular weight (Mw) of between 50 kDa and 400 kDa.

294. The process of any one of paragraphs 284-292, wherein the activated serotype 24F polysaccharide has a weight average molecular weight (Mw) of between 120 kDa and 250 kDa.

295. The process of any one of paragraphs 284-292, wherein the activated serotype 24F polysaccharide has a weight average molecular weight (Mw) of between 120 kDa and 200 kDa.

296. The process of any one of paragraphs 284-295, wherein the activated serotype 24F polysaccharide retains at least 75% of the branched Ribose.

297. The process of any one of paragraphs 284-295, wherein the activated serotype 24F polysaccharide retains at least 90% of the branched Ribose.

298. The process of any one of paragraphs 284-295, wherein the activated serotype 24F polysaccharide retains at least 95% of the branched Ribose.

299. The process of any one of paragraphs 284-295, wherein the activated serotype 24F polysaccharide retains about 100% of the branched Ribose.

300. The process of any one of paragraphs 284-299, wherein the oxidizing agent is periodate.

301. The process of any one of paragraphs 284-299, wherein the oxidizing agent is sodium periodate.

302. The process of any one of paragraphs 284-299, wherein the oxidizing agent is sodium metaperiodate.

303. The process of any one of paragraphs 284-301, wherein the oxidation step (a) comprises reacting the polysaccharide with 0.1-2 molar equivalents of periodate.

304. The process of any one of paragraphs 284-301, wherein the oxidation step (a) comprises reacting the polysaccharide with 0.5-1.5 molar equivalents of periodate.

305. The process of any one of paragraphs 284-301, wherein the oxidation step (a) comprises reacting the polysaccharide with 0.9-1.1 molar equivalents of periodate.

306. The process of any one of paragraphs 284-305, wherein the degree of oxidation of the activated serotype 24F polysaccharide is between 2 and 20.

307. The process of any one of paragraphs 284-305, wherein the degree of oxidation of the activated serotype 24F polysaccharide is between 4 and 15.

308. The process of any one of paragraphs 284-305, wherein the degree of oxidation of the activated serotype 24F polysaccharide is 9±3.

309. The process of any one of paragraphs 284-308, wherein the activated serotype 24F polysaccharide and the carrier protein are lyophilised before step b).

310. The process of any one of paragraphs 284-309, wherein lyophilisation occurs after step a).

311. The process of any one of paragraphs 284-310, wherein the activated polysaccharide is lyophilised after step a) and the carrier protein is also lyophilised.

312. The process of any one of paragraphs 284-310, wherein the activated serotype 24F polysaccharide is lyophilised after step a) and the carrier protein is also lyophilised, and the activated polysaccharide and the carrier protein are reconstituted in the same solution.

313. The process of any one of paragraphs 284-312, wherein the activated serotype 24F polysaccharide and the carrier protein are lyophilised independently.

314. The process of any one of paragraphs 284-312, wherein the activated serotype 24F polysaccharide and the carrier protein are lyophilised together (co-lyophilized).

315. The process of any one of paragraphs 284-314, wherein the initial input ratio (weight by weight) of activated serotype 24F capsular polysaccharide to carrier protein at step b) is between 2:1 and 0.5:1.

316. The process of any one of paragraphs 284-314, wherein the initial input ratio (weight by weight) of activated serotype 24F capsular polysaccharide to carrier protein at step b) is between 1.2:1 and 0.6:1.

317. The process of any one of paragraphs 284-314, wherein the initial input ratio (weight by weight) of activated serotype 24F capsular polysaccharide to carrier protein at step b) is between 0.9:1 and 0.7:1.

318. The process of any one of paragraphs 284-317, wherein the reduction reaction (c) is carried out in aqueous solvent.

319. The process of any one of paragraphs 284-317, wherein the reduction reaction (c) is carried out in aprotic solvent.

320. The process of any one of paragraphs 284-317, wherein the reduction reaction (c) is carried out in the presence of dimethylsulphoxide (DMSO) or dimethylformamide (DMF).

321. The process of any one of paragraphs 284-317, wherein the reduction reaction (c) is carried out in the presence of dimethylsulphoxide (DMSO).

322. The process of any one of paragraphs 284-317, wherein the reduction reaction (c) is carried out in DMSO (dimethylsulfoxide) or in DMF (dimethylformamide)) solvent.

323. The process of any one of paragraphs 284-317, wherein the reduction reaction (c) is carried out in DMSO (dimethylsulfoxide) solvent.

324. The process of any one of paragraphs 284-323, wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN-BH3, benzylamine-BH3 or 5-ethyl-2-methylpyridine borane (PEMB).

325. The process of any one of paragraphs 284-323, wherein the reducing agent is sodium triacetoxyborohydride.

326. The process of any one of paragraphs 284-323, wherein the reducing agent is sodium cyanoborohydride in the present of nickel.

327. The process of any one of paragraphs 284-323, wherein the reducing agent is sodium cyanoborohydride.

328. The process of any one of paragraphs 284-327, wherein between 0.2 and 5 molar equivalents of reducing agent is used in step c).

329. The process of any one of paragraphs 284-327, wherein between 0.5 and 2.5 molar equivalents of reducing agent is used in step c).

330. The process of any one of paragraphs 284-327, wherein between 1.5 and 2.5 molar equivalent of reducing agent is used in step c).

331. The process of any one of paragraphs 284-330, wherein following the reduction reaction (c), unreacted aldehyde groups remaining in the conjugates are capped using a suitable capping agent.

332. The process of paragraph 331, wherein said capping agent is sodium borohydride (NaBH₄).

333. The process of any one of paragraphs 284-332, wherein capping is achieved by mixing the product of step c) with 1 to 20 molar equivalents of sodium borohydride.

334. The process of any one of paragraphs 284-332, wherein capping is achieved by mixing the product of step c) with 1 to 3 molar equivalents of sodium borohydride.

335. The process of any one of paragraphs 284-334, comprising the step of purifying the glycoconjugate after it is produced.

336. The process of any one of paragraphs 284-335, wherein the carrier protein is the fusion protein CP1.

337. The process of any one of paragraphs 284-335, wherein the carrier protein is H. influenzae protein D.

338. The process of any one of paragraphs 284-335, wherein the carrier protein is TT, DT, CRM₁₉₇ or a C5a peptidase from Streptococcus (SCP).

339. The process of any one of paragraphs 284-335, wherein the carrier protein is DT.

340. The process of any one of paragraphs 284-335, wherein the carrier protein is TT.

341. The process of any one of paragraphs 284-335, wherein the carrier protein is CRM₁₉₇ or a C5a peptidase from Streptococcus (SCP).

342. The process of any one of paragraphs 284-335, wherein the carrier protein is a C5a peptidase from Streptococcus (SCP).

343. The process of any one of paragraphs 284-335, wherein the carrier protein is CRM₁₉₇.

344. A S. pneumoniae serotype 24F glycoconjugate obtained according to the process of any one of paragraphs 284-343.

345. A S. pneumoniae serotype 35B glycoconjugate comprising a serotype 35B capsular polysaccharide wherein the weight average molecular weight (Mw) of said polysaccharide is between 15 kDa and 100 kDa.

346. The S. pneumoniae serotype 35B glycoconjugate of paragraph 345 wherein the weight average molecular weight (Mw) of said polysaccharide is between 25 kDa and 50 kDa.

347. The S. pneumoniae serotype 35B glycoconjugate of paragraph 345 wherein the weight average molecular weight (Mw) of said polysaccharide is between 30 kDa and 40 kDa.

348. The S. pneumoniae serotype 35B glycoconjugate of any one of paragraphs 345-347 having a weight average molecular weight (Mw) of between 250 kDa and 7,500 kDa.

349. The S. pneumoniae serotype 35B glycoconjugate of any one of paragraphs 345-347 having a weight average molecular weight (Mw) of between 500 kDa and 5,000 kDa.

350. The S. pneumoniae serotype 35B glycoconjugate of any one of paragraphs 345-347 having a weight average molecular weight (Mw) of between 1,000 kDa and 4,000 kDa.

351. The S. pneumoniae serotype 35B glycoconjugate of any one of paragraphs 345-350 wherein the serotype 35B polysaccharide to carrier protein ratio (w/w) in the glycoconjugate is between 0.4 and 3.0.

352. The S. pneumoniae serotype 35B glycoconjugate of any one of paragraphs 345-350 wherein the serotype 35B polysaccharide to carrier protein ratio (w/w) in the glycoconjugate is between 0.4 and 2.0.

353. The S. pneumoniae serotype 35B glycoconjugate of any one of paragraphs 345-350 wherein the serotype 35B polysaccharide to carrier protein ratio (w/w) in the glycoconjugate is between 0.5 and 1.5.

354. The S. pneumoniae serotype 35B glycoconjugate of any one of paragraphs 345-353 wherein the degree of conjugation is between 2 and 15.

355. The S. pneumoniae serotype 35B glycoconjugate of any one of paragraphs 345-353 wherein the degree of conjugation is between 5 and 10.

356. The S. pneumoniae serotype 35B glycoconjugate of any one of paragraphs 345-355 comprising less than about 40% of free serotype 35B polysaccharide compared to the total amount of serotype 35B polysaccharide.

357. The S. pneumoniae serotype 35B glycoconjugate of any one of paragraphs 345-355 comprising less than about 20% of free serotype 35B polysaccharide compared to the total amount of serotype 35B polysaccharide.

358. The S. pneumoniae serotype 35B glycoconjugate of any one of paragraphs 345-355 comprising less than about 10% of free serotype 35B polysaccharide compared to the total amount of serotype 35B polysaccharide.

359. The S. pneumoniae serotype 35B glycoconjugate of any one of paragraphs 345-358 wherein at least 40% of the serotype 35B glycoconjugate has a Kd below or equal to 0.3 in a CL-4B column.

360. The S. pneumoniae serotype 35B glycoconjugate of any one of paragraphs 345-358 wherein at least 60% of the glycoconjugate has a Kd below or equal to 0.3 in a CL-4B column.

361. The S. pneumoniae serotype 35B glycoconjugate of any one of paragraphs 345-358 wherein between 50% and 80% of the serotype 35B glycoconjugate has a Kd below or equal to 0.3 in a CL-4B column.

362. The S. pneumoniae serotype 35B glycoconjugate of any one of paragraphs 345-361 wherein the carrier protein of the glycoconjugates is the fusion protein CP1.

363. The S. pneumoniae serotype 35B glycoconjugate of any one of 345-361 wherein the carrier protein of the glycoconjugate is H. influenzae protein D.

364. The S. pneumoniae serotype 35B glycoconjugate of any one of paragraphs 345-361 wherein the carrier protein of the glycoconjugate is TT, DT, CRM₁₉₇ or a C5a peptidase from Streptococcus (SCP).

365. The S. pneumoniae serotype 35B glycoconjugate of any one of paragraphs 345-361 wherein the carrier protein of the glycoconjugate is DT.

366. The S. pneumoniae serotype 35B glycoconjugate of any one of paragraphs 345-361 wherein the carrier protein of the glycoconjugate is TT.

367. The S. pneumoniae serotype 35B glycoconjugate of any one of paragraphs 345-361 wherein the carrier protein of the glycoconjugate is CRM₁₉₇ or a C5a peptidase from Streptococcus (SCP).

368. The S. pneumoniae serotype 35B glycoconjugate of any one of paragraphs 345-361 wherein the carrier protein of the glycoconjugate is a C5a peptidase from Streptococcus (SCP).

369. The S. pneumoniae serotype 35B glycoconjugate of any one of paragraphs 345-361 wherein the carrier protein of the glycoconjugate is CRM₁₉₇.

370. The S. pneumoniae serotype 35B glycoconjugate of any one of paragraphs 345-369 prepared using reductive amination chemistry.

371. A process for preparing a S. pneumoniae serotype 35B glycoconjugate by conjugating an isolated serotype 35B capsular polysaccharide to a carrier protein comprising the step of: (a) reacting an isolated serotype 35B capsular polysaccharide with an oxidizing agent; (b) compounding the activated polysaccharide of step (a) with a carrier protein; and (c) reacting the compounded activated polysaccharide and carrier protein with a reducing agent to form a glycoconjugate.

372. The process of paragraph 371, wherein the isolated serotype 35B capsular polysaccharide is not sized before the oxidation step (a).

373. The process of paragraphs 371-372, further comprising a step (a′) between step (a) and (b), said step being: (a′) quenching the oxidation reaction by addition of a quenching agent resulting in an activated serotype 35B capsular polysaccharide.

374. A process for preparing a S. pneumoniae serotype 35B glycoconjugate by conjugating an isolated serotype 35B capsular polysaccharide to a carrier protein comprising the step of: (a) reacting said isolated serotype 35B capsular polysaccharide with an oxidizing agent; (a′) quenching the oxidation reaction by addition of a quenching agent resulting in an activated serotype 35B capsular polysaccharide; (b) compounding the activated polysaccharide of step (a′) with a carrier protein; and (c) reacting the compounded activated polysaccharide and carrier protein with a reducing agent to form a glycoconjugate.

375. The process of any one of paragraphs 371-374, wherein the oxidation step (a) is carried out at a pH of between 5.0 and 7.0.

376. The process of any one of paragraphs 371-374, wherein the oxidation step (a) is carried out at a pH of between 5.5 and 6.5.

377. The process of any one of paragraphs 371-374, wherein the oxidation step (a) is carried out at a pH about 6.0.

378. The process of any one of paragraphs 371-377, wherein the oxidizing agent is periodate.

379. The process of any one of paragraphs 371-377, wherein the oxidizing agent is sodium periodate.

380. The process of any one of paragraphs 371-377, wherein the oxidizing agent is sodium metaperiodate.

381. The process of any one of paragraphs 371-380, wherein the oxidation step (a) comprises reacting the polysaccharide with 0.05-0.2 molar equivalents of periodate.

382. The process of any one of paragraphs 371-380, wherein the oxidation step (a) comprises reacting the polysaccharide with 0.09-0.11 molar equivalents of periodate.

383. The process of any one of paragraphs 371-380, wherein the oxidation step (a) comprises reacting the polysaccharide with about 0.1 molar equivalents of periodate.

384. The process of any one of paragraphs 373-383, wherein the quenching agent is selected from vicinal diols, 1,2-aminoalcohols, amino acids, glutathione, sulfite, bisulfate, dithionite, metabisulfite, thiosulfate, phosphites, hypophosphites or phosphorous acid.

385. The process of any one of paragraphs 373-383, wherein the quenching agent is a 1,2-aminoalcohols of formula (I):

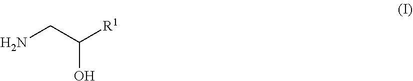

wherein R¹ is selected from H, methyl, ethyl, propyl or isopropyl.

386. The process of any one of paragraphs 373-383, wherein the quenching agent is sodium or potassium salts of sulfite, bisulfate, dithionite, metabisulfite, thiosulfate, phosphites, hypophosphites or phosphorous acid.

387. The process of any one of paragraphs 373-383, wherein the quenching agent is an amino acid.

388. The process of paragraph 387 wherein said amino acid is serine, threonine, cysteine, cystine, methionine, proline, hydroxyproline, tryptophan, tyrosine or histidine.

389. The process of any one of paragraphs 373-383, wherein the quenching agent is a sulfite such as bisulfate, dithionite, metabisulfite, thiosulfate.

390. The process of any one of paragraphs 373-383, wherein the quenching agent is a compound comprising two vicinal hydroxyl groups (vicinal diols).

391. The process of any one of paragraphs 373-383, wherein the quenching agent is a compound comprising two hydroxyl groups covalently linked to two adjacent carbon atoms.

392. The process of any one of paragraphs 373-383, wherein the quenching agent is a compound of formula (II):

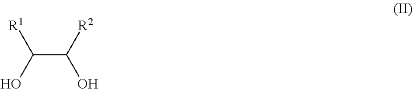

wherein R¹ and R² are each independently selected from H, methyl, ethyl, propyl or isopropyl.

393. The process of any one of paragraphs 373-383, wherein the quenching agent is glycerol, ethylene glycol, propan-1,2-diol, butan-1,2-diol or butan-2,3-diol, or ascorbic acid.

394. The process of any one of paragraphs 373-383, wherein the quenching agent is butan-2,3-diol.

395. An activation process comprising the step of:
(a) reacting an isolated serotype 35B polysaccharide with periodate;
(b) quenching the oxidation reaction by addition of butan-2,3-diol resulting in an activated serotype 35B polysaccharide.

396. The process of any one of paragraphs 373-395, wherein the activated serotype 35B polysaccharide has a weight average molecular weight (Mw) of between 15 kDa and 100 kDa.

397. The process of any one of paragraphs 373-395, wherein the activated serotype 35B polysaccharide has a weight average molecular weight (Mw) of between 25 kDa and 50 kDa.

398. The process of any one of paragraphs 373-395, wherein the activated serotype 35B polysaccharide has a weight average molecular weight (Mw) of between 30 kDa and 40 kDa.

399. The process of any one of paragraphs 373-398, wherein the degree of oxidation of the activated serotype 35B polysaccharide is between 2 and 20.

400. The process of any one of paragraphs 373-398, wherein the degree of oxidation of the activated serotype 35B polysaccharide is between 4 and 15.

401. The process of any one of paragraphs 373-398, wherein the degree of oxidation of the activated serotype 35B polysaccharide is 9±3.

402. The process of any one of paragraphs 373-401, wherein the activated serotype 35B polysaccharide and the carrier protein are lyophilised before step b).

403. The process of any one of paragraphs 373-402, wherein lyophilisation occurs after step a).

404. The process of any one of paragraphs 373-401, wherein the activated polysaccharide is lyophilised after step a) and the carrier protein is also lyophilised.

405. The process of any one of paragraphs 373-401, wherein the activated serotype 35B polysaccharide is lyophilised after step a) and the carrier protein is also lyophilised, and the activated polysaccharide and the carrier protein are reconstituted in the same solution.

406. The process of any one of paragraphs 373-405, wherein the activated serotype 35B polysaccharide and the carrier protein are lyophilised independently.

407. The process of any one of paragraphs 373-405, wherein the activated serotype 35B polysaccharide and the carrier protein are lyophilised together (co-lyophilized).

408. The process of any one of paragraphs 373-407, wherein the initial input ratio (weight by weight) of activated serotype 35B capsular polysaccharide to carrier protein at step b) is between 2:1 and 0.5:1.

409. The process of any one of paragraphs 373-407, wherein the initial input ratio (weight by weight) of activated serotype 35B capsular polysaccharide to carrier protein at step b) is between 1.2:1 and 0.6:1.

410. The process of any one of paragraphs 373-407, wherein the initial input ratio (weight by weight) of activated serotype 35B capsular polysaccharide to carrier protein at step b) is between 0.9:1 and 0.7:1.

411. The process of any one of paragraphs 373-410, wherein the reduction reaction (c) is carried out in aqueous solvent.

412. The process of any one of paragraphs 373-410, wherein the reduction reaction (c) is carried out in aprotic solvent.

413. The process of any one of paragraphs 373-410, wherein the reduction reaction (c) is carried out in the presence of dimethylsulphoxide (DMSO) or dimethylformamide (DMF).

414. The process of any one of paragraphs 373-410, wherein the reduction reaction (c) is carried out in the presence of dimethylsulphoxide (DMSO).

415. The process of any one of paragraphs 373-410, wherein the reduction reaction (c) is carried out in DMSO (dimethylsulfoxide) or in DMF (dimethylformamide)) solvent.

416. The process of any one of paragraphs 373-410, wherein the reduction reaction (c) is carried out in DMSO (dimethylsulfoxide) solvent.

417. The process of any one of paragraphs 373-416, wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN-BH3, benzylamine-BH3 or 5-ethyl-2-methylpyridine borane (PEMB).

418. The process of any one of paragraphs 373-416, wherein the reducing agent is sodium triacetoxyborohydride.

419. The process of any one of paragraphs 373-416, wherein the reducing agent is sodium cyanoborohydride in the present of nickel.

420. The process of any one of paragraphs 373-416, wherein the reducing agent is sodium cyanoborohydride.

421. The process of any one of paragraphs 373-420, wherein between 0.2 and 5 molar equivalents of reducing agent is used in step c).

422. The process of any one of paragraphs 373-420, wherein between 0.5 and 1.5 molar equivalents of reducing agent is used in step c).

423. The process of any one of paragraphs 373-420, wherein between 0.9 and 1.1 molar equivalent of reducing agent is used in step c).

424. The process of any one of paragraphs 373-423, wherein following the reduction reaction (c), unreacted aldehyde groups remaining in the conjugates are capped using a suitable capping agent.

425. The process of paragraph 424, wherein said capping agent is sodium borohydride (NaBH₄).

426. The process of any one of paragraphs 424-425, wherein capping is achieved by mixing the product of step c) with 1 to 20 molar equivalents of sodium borohydride.

427. The process of any one of paragraphs 424-425, wherein capping is achieved by mixing the product of step c) with 1 to 3 molar equivalents of sodium borohydride.

428. The process of any one of paragraphs 373-427, comprising the step of purifying the glycoconjugate after it is produced.

429. The process of any one of paragraphs 373-428, wherein the carrier protein is the fusion protein CP1.

430. The process of any one of paragraphs 373-428, wherein the carrier protein is H. influenzae protein D.

431. The process of any one of paragraphs 373-428, wherein the carrier protein is TT, DT, CRM₁₉₇ or a C5a peptidase from Streptococcus (SCP).

432. The process of any one of paragraphs 373-428, wherein the carrier protein is DT.

433. The process of any one of paragraphs 373-428, wherein the carrier protein is TT.

434. The process of any one of paragraphs 373-428, wherein the carrier protein is CRM₁₉₇ or a C5a peptidase from Streptococcus (SCP).

435. The process of any one of paragraphs 373-428, wherein the carrier protein is a C5a peptidase from Streptococcus (SCP).

436. The process of any one of paragraphs 373-428, wherein the carrier protein is CRM₁₉₇.

437. A S. pneumoniae serotype 35B glycoconjugate obtained according to the process of any one of paragraphs 373-436.

438. An immunogenic composition comprising a glycoconjugate of any one of paragraphs 1-26, 79-109, 171-197, 254-283, 344-370 or 437.

439. The immunogenic composition of paragraph 438 comprising 1 to 25 glycoconjugates from different serotypes of S. pneumoniae.

440. An immunogenic composition comprising a S. pneumoniae serotype 15A glycoconjugate of any one of paragraphs 1-26 or 79.

441. An immunogenic composition comprising a S. pneumoniae serotype 23A glycoconjugate of any one of paragraphs 80-109 or 171.

442. An immunogenic composition comprising a S. pneumoniae serotype 23B glycoconjugate of any one of paragraphs 172-197 or 254.

443. An immunogenic composition comprising a S. pneumoniae serotype 24F glycoconjugate of any one of paragraphs 255-283 or 344.

444. An immunogenic composition comprising a S. pneumoniae serotype 35B glycoconjugate of any one of paragraphs 345-370 or 437.

445. An immunogenic composition comprising glycoconjugates from S. pneumoniae serotype 15A, 23A, 23B, 24F and 35B.

446. The immunogenic composition of paragraph 445 wherein said S. pneumoniae serotype 15A glycoconjugate is any one of paragraphs 1-26 or 79, said S. pneumoniae serotype 23A glycoconjugate is any one of paragraphs 80-109 or 171, said S. pneumoniae serotype 23B glycoconjugate is any one of paragraphs 172-197 or 254, said S. pneumoniae serotype 24F glycoconjugate is any one of paragraphs 255-283 or 344 and said S. pneumoniae serotype 35B glycoconjugate is any one of paragraphs 345-370 or 437.

447. An immunogenic composition comprising from 14 to 25 pneumococcal glycoconjugates from different serotypes of S. pneumoniae, wherein said serotypes are selected from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

448. The immunogenic composition of paragraph 447 wherein said S. pneumoniae serotype 15A glycoconjugate is any one of paragraphs 1-26 or 79, said S. pneumoniae serotype 23A glycoconjugate is any one of paragraphs 80-109 or 171, said S. pneumoniae serotype 23B glycoconjugate is any one of paragraphs 172-197 or 254, said S. pneumoniae serotype 24F glycoconjugate is any one of paragraphs 255-283 or 344 and said S. pneumoniae serotype 35B glycoconjugate is any one of paragraphs 345-370 or 437.

449. An immunogenic composition comprising from 21 to 25 pneumococcal glycoconjugates from different serotypes of S. pneumoniae, wherein said serotypes are selected from serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

450. The immunogenic composition of paragraph 449 wherein said S. pneumoniae serotype 15A glycoconjugate is any one of paragraphs 1-26 or 79, said S. pneumoniae serotype 23A glycoconjugate is any one of paragraphs 80-109 or 171, said S. pneumoniae serotype 23B glycoconjugate is any one of paragraphs 172-197 or 254, said S. pneumoniae serotype 24F glycoconjugate is any one of paragraphs 255-283 or 344 and said S. pneumoniae serotype 35B glycoconjugate is any one of paragraphs 345-370 or 437.

451. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises from 1 to 5 glycoconjugates from S. pneumoniae serotypes 15A, 23A, 23B, 24F and/or 35B.

452. The immunogenic composition of paragraph 451 wherein said S. pneumoniae serotype 15A glycoconjugate is any one of paragraphs 1-26 or 79, said S. pneumoniae serotype 23A glycoconjugate is any one of paragraphs 80-109 or 171, said S. pneumoniae serotype 23B glycoconjugate is any one of paragraphs 172-197 or 254, said S. pneumoniae serotype 24F glycoconjugate is any one of paragraphs 255-283 or 344 and said S. pneumoniae serotype 35B glycoconjugate is any one of paragraphs 345-370 or 437.

453. The immunogenic composition of paragraph 451 or 452 which is a 21, 22, 23, 24 or 25-valent pneumococcal conjugate composition.

454. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprising one glycoconjugate from S. pneumoniae serotypes 15A, 23A, 23B, 24F or 35B.

455. The immunogenic composition of paragraph 454 which is a 21-valent pneumococcal conjugate composition.

456. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises 2 glycoconjugates selected from S. pneumoniae serotypes 15A, 23A, 23B, 24F and 35B.

457. The immunogenic composition of paragraph 456 which is a 22-valent pneumococcal conjugate composition.

458. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises 3 glycoconjugates selected from S. pneumoniae serotypes 15A, 23A, 23B, 24F and 35B.

459. The immunogenic composition of paragraph 458 which is a 23-valent pneumococcal conjugate composition.

460. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises 4 glycoconjugates selected from S. pneumoniae serotypes 15A, 23A, 23B, 24F and 35B.

461. The immunogenic composition of paragraph 460 which is a 24-valent pneumococcal conjugate composition.

462. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.

463. The immunogenic composition of paragraph 462 which is a 21-valent pneumococcal conjugate composition.

464. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F.

465. The immunogenic composition of paragraph 464 which is a 21-valent pneumococcal conjugate composition.

466. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F.

467. The immunogenic composition of paragraph 466 which is a 21-valent pneumococcal conjugate composition.

468. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F.

469. The immunogenic composition of paragraph 468 which is a 21-valent pneumococcal conjugate composition.

470. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B.

471. The immunogenic composition of paragraph 470 which is a 21-valent pneumococcal conjugate composition.

472. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F and 33F.

473. The immunogenic composition of paragraph 472 which is a 22-valent pneumococcal conjugate composition.

474. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F and 33F.

475. The immunogenic composition of paragraph 474 which is a 22-valent pneumococcal conjugate composition.

476. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F and 33F.

477. The immunogenic composition of paragraph 476 which is a 22-valent pneumococcal conjugate composition.

478. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 33F and 35B.

479. The immunogenic composition of paragraph 478 which is a 22-valent pneumococcal conjugate composition.

480. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F and 33F.

481. The immunogenic composition of paragraph 480 which is a 22-valent pneumococcal conjugate composition.

482. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F and 33F.

483. The immunogenic composition of paragraph 482 which is a 22-valent pneumococcal conjugate composition.

484. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 33F and 35B.

485. The immunogenic composition of paragraph 484 which is a 22-valent pneumococcal conjugate composition.

486. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F and 33F.

487. The immunogenic composition of paragraph 486 which is a 22-valent pneumococcal conjugate composition.

488. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 33F and 35B.

489. The immunogenic composition of paragraph 488 which is a 22-valent pneumococcal conjugate composition.

490. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F, 33F and 35B.

491. The immunogenic composition of paragraph 490 which is a 22-valent pneumococcal conjugate composition.

492. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F and 33F.

493. The immunogenic composition of paragraph 492 which is a 23-valent pneumococcal conjugate composition.

494. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 24F and 33F.

495. The immunogenic composition of paragraph 494 which is a 23-valent pneumococcal conjugate composition.

496. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 33F and 35B.

497. The immunogenic composition of paragraph 496 which is a 23-valent pneumococcal conjugate composition.

498. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F and 33F.

499. The immunogenic composition of paragraph 498 which is a 23-valent pneumococcal conjugate composition.

500. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 33F and 35B.

501. The immunogenic composition of paragraph 500 which is a 23-valent pneumococcal conjugate composition.

502. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F, 33F and 35B.

503. The immunogenic composition of paragraph 502 which is a 23-valent pneumococcal conjugate composition.

504. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F and 33F.

505. The immunogenic composition of paragraph 504 which is a 23-valent pneumococcal conjugate composition.

506. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 33F and 35B.

507. The immunogenic composition of paragraph 506 which is a 23-valent pneumococcal conjugate composition.

508. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F, 33F and 35B.

509. The immunogenic composition of paragraph 508 which is a 23-valent pneumococcal conjugate composition.

510. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F, 33F and 35B.

511. The immunogenic composition of paragraph 510 which is a 23-valent pneumococcal conjugate composition.

512. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F and 33F.

513. The immunogenic composition of paragraph 512 which is a 24-valent pneumococcal conjugate composition.

514. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 33F and 35B.

515. The immunogenic composition of paragraph 514 which is a 24-valent pneumococcal conjugate composition.

516. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 33F, 24F and 35B.

517. The immunogenic composition of paragraph 516 which is a 24-valent pneumococcal conjugate composition.

518. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B.

519. The immunogenic composition of paragraph 518 which is a 24-valent pneumococcal conjugate composition.

520. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F, 33F and 35B.

521. The immunogenic composition of paragraph 520 which is a 24-valent pneumococcal conjugate composition.

522. The immunogenic composition of any one of paragraphs 454 to 521 wherein said S. pneumoniae serotype 15A glycoconjugate is any one of paragraphs 1-26 or 79, said S. pneumoniae serotype 23A glycoconjugate is any one of paragraphs 80-109 or 171, said S. pneumoniae serotype 23B glycoconjugate is any one of paragraphs 172-197 or 254, said S. pneumoniae serotype 24F glycoconjugate is any one of paragraphs 255-283 or 344 and/or said S. pneumoniae serotype 35B glycoconjugate is any one of paragraphs 345-370 or 437.

523. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

524. The immunogenic composition of paragraph 523 which is a 25-valent pneumococcal conjugate composition.

525. The immunogenic composition of paragraph 524 or 525 wherein said S. pneumoniae serotype 15A glycoconjugate is any one of paragraphs 1-26 or 79, said S. pneumoniae serotype 23A glycoconjugate is any one of paragraphs 80-109 or 171, said S. pneumoniae serotype 23B glycoconjugate is any one of paragraphs 172-197 or 254, said S. pneumoniae serotype 24F glycoconjugate is any one of paragraphs 255-283 or 344 and/or said S. pneumoniae serotype 35B glycoconjugate is any one of paragraphs 345-370 or 437.

526. The immunogenic composition of any one of paragraphs 454 to 525 wherein all the glycoconjugates are individually conjugated to the carrier protein.

527. The immunogenic composition of any one of paragraphs 454 to 526 wherein the glycoconjugates are conjugated to CRM₁₉₇.

528. The immunogenic composition of any one of paragraphs 454 to 527 wherein the glycoconjugate from S. pneumoniae serotype 3 is conjugated to CRM₁₉₇.

529. The immunogenic composition of any one of paragraphs 454 to 527 wherein the glycoconjugate from S. pneumoniae serotype 3 is conjugated to SCP.

530. The immunogenic composition of any one of paragraphs 454 to 529 wherein the glycoconjugate from S. pneumoniae serotype 22F is conjugated to CRM₁₉₇.

531. The immunogenic composition of any one of paragraphs 454 to 530 wherein the glycoconjugate from S. pneumoniae serotype 33F is conjugated to CRM₁₉₇.

532. The immunogenic composition of any one of paragraphs 454 to 531 wherein the glycoconjugate from S. pneumoniae serotype 15B is conjugated to CRM₁₉₇.

533. The immunogenic composition of any one of paragraphs 454 to 532 wherein the glycoconjugate from S. pneumoniae serotype 12F is conjugated to CRM₁₉₇.

534. The immunogenic composition of any one of paragraphs 454 to 533 wherein the glycoconjugate from S. pneumoniae serotype 10A is conjugated to CRM₁₉₇.

535. The immunogenic composition of any one of paragraphs 454 to 534 wherein the glycoconjugate from S. pneumoniae serotype 11A is conjugated to CRM₁₉₇.

536. The immunogenic composition of any one of paragraphs 454 to 535 wherein the glycoconjugate from S. pneumoniae serotype 8 is conjugated to CRM₁₉₇.

537. The immunogenic composition of any one of paragraphs 454 to 536 wherein the glycoconjugates from S. pneumoniae serotypes 4, 6B, 9V, 14, 18C, 19F and 23F are conjugated to CRM₁₉₇.

538. The immunogenic composition of any one of paragraphs 454 to 537 wherein the glycoconjugates from S. pneumoniae serotypes 1, 5 and 7F are conjugated to CRM₁₉₇.

539. The immunogenic composition of any one of paragraphs 454 to 538 wherein the glycoconjugates from S. pneumoniae serotypes 6A and 19A are conjugated to CRM₁₉₇.

540. The immunogenic composition of any one of paragraphs 454 to 528 wherein the the glycoconjugates are all individually conjugated to CRM₁₉₇.

541. The immunogenic composition of any one of paragraphs 454 to 528 wherein the glycoconjugate from S. pneumoniae serotype 3 is conjugated to SCP and the other glycoconjugates are all individually conjugated to CRM₁₉₇.

542. The immunogenic composition of any one of paragraphs 454 to 528 wherein the glycoconjugate from S. pneumoniae serotype 3 is conjugated to SCP, at least one other glycoconjugate is conjugated to TT and the other glycoconjugate(s) is/are all individually conjugated to CRM₁₉₇.

543. The immunogenic composition of any one of paragraphs 454 to 528 wherein the glycoconjugate from S. pneumoniae serotype 3 is conjugated to SCP, one other glycoconjugate is conjugated to TT and the other glycoconjugate(s) is/are all individually conjugated to CRM₁₉₇.

544. The immunogenic composition of any one of paragraphs 454 to 528 wherein the glycoconjugate from S. pneumoniae serotype 3 is conjugated to SCP, at least two other glycoconjugates are conjugated to TT and the other glycoconjugate(s) is/are all individually conjugated to CRM₁₉₇.

545. The immunogenic composition of any one of paragraphs 454 to 528 wherein the glycoconjugate from S. pneumoniae serotype 3 is conjugated to SCP, two other glycoconjugates are conjugated to TT and the other glycoconjugate(s) is/are all individually conjugated to CRM₁₉₇.

546. The immunogenic composition of any one of paragraphs 454 to 528 wherein the glycoconjugate from S. pneumoniae serotype 3 is conjugated to SCP, at least three other glycoconjugates are conjugated to TT and the other glycoconjugate(s) is/are all individually conjugated to CRM₁₉₇.

547. The immunogenic composition of any one of paragraphs 454 to 528 wherein the glycoconjugate from S. pneumoniae serotype 3 is conjugated to SCP, three other glycoconjugates are conjugated to TT and the other glycoconjugate(s) is/are all individually conjugated to CRM₁₉₇.

548. The immunogenic composition of any one of paragraphs 454 to 528 wherein the glycoconjugate from S. pneumoniae serotype 3 is conjugated to SCP, at least four other glycoconjugates are conjugated to TT and the other glycoconjugate(s) is/are all individually conjugated to CRM₁₉₇.

549. The immunogenic composition of any one of paragraphs 454 to 528 wherein the glycoconjugate from S. pneumoniae serotype 3 is conjugated to SCP, four other glycoconjugates are conjugated to TT and the other glycoconjugate(s) is/are all individually conjugated to CRM₁₉₇.

550. The immunogenic composition of any one of paragraphs 454 to 528 wherein the glycoconjugate from S. pneumoniae serotype 3 is conjugated to SCP, at least five other glycoconjugates are conjugated to TT and the other glycoconjugate(s) is/are all individually conjugated to CRM₁₉₇.

551. The immunogenic composition of any one of paragraphs 454 to 528 wherein the glycoconjugate from S. pneumoniae serotype 3 is conjugated to SCP, five other glycoconjugates are conjugated to TT and the other glycoconjugate(s) is/are all individually conjugated to CRM₁₉₇.

552. The immunogenic composition of any one of paragraphs 454 to 528 wherein the glycoconjugate from S. pneumoniae serotype 3 is conjugated to SCP, at least one other glycoconjugate is conjugated to SCP and the other glycoconjugate(s) is/are all individually conjugated to CRM₁₉₇.

553. The immunogenic composition of any one of paragraphs 454 to 528 wherein the glycoconjugate from S. pneumoniae serotype 3 is conjugated to SCP, one other glycoconjugate is conjugated to SCP and the other glycoconjugate(s) is/are all individually conjugated to CRM₁₉₇.

554. The immunogenic composition of any one of paragraphs 454 to 528 wherein the glycoconjugate from S. pneumoniae serotype 3 is conjugated to SCP, at least two other glycoconjugates are conjugated to SCP and the other glycoconjugate(s) is/are all individually conjugated to CRM₁₉₇.

555. The immunogenic composition of any one of paragraphs 454 to 528 wherein the glycoconjugate from S. pneumoniae serotype 3 is conjugated to SCP, two other glycoconjugates are conjugated to SCP and the other glycoconjugate(s) is/are all individually conjugated to CRM₁₉₇.

556. The immunogenic composition of any one of paragraphs 454 to 528 wherein the glycoconjugate from S. pneumoniae serotype 3 is conjugated to SCP, at least three other glycoconjugates are conjugated to SCP and the other glycoconjugate(s) is/are all individually conjugated to CRM₁₉₇.

557. The immunogenic composition of any one of paragraphs 454 to 528 wherein the glycoconjugate from S. pneumoniae serotype 3 is conjugated to SCP, three other glycoconjugates are conjugated to SCP and the other glycoconjugate(s) is/are all individually conjugated to CRM₁₉₇.

558. The immunogenic composition of any one of paragraphs 454 to 528 wherein the glycoconjugate from S. pneumoniae serotype 3 is conjugated to SCP, at least four other glycoconjugates are conjugated to SCP and the other glycoconjugate(s) is/are all individually conjugated to CRM₁₉₇.

559. The immunogenic composition of any one of paragraphs 454 to 528 wherein the glycoconjugate from S. pneumoniae serotype 3 is conjugated to SCP, four other glycoconjugates are conjugated to SCP and the other glycoconjugate(s) is/are all individually conjugated to CRM₁₉₇.

560. The immunogenic composition of any one of paragraphs 454 to 528 wherein the glycoconjugate from S. pneumoniae serotype 3 is conjugated to SCP, at least five other glycoconjugates are conjugated to SCP and the other glycoconjugate(s) is/are all individually conjugated to CRM₁₉₇.

561. The immunogenic composition of any one of paragraphs 454 to 528 wherein the glycoconjugate from S. pneumoniae serotype 3 is conjugated to SCP, five other glycoconjugates are conjugated to SCP and the other glycoconjugate(s) is/are all individually conjugated to CRM₁₉₇.

562. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the other glycoconjugates are all individually conjugated to CRM₁₉₇.

563. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugate from S. pneumoniae serotype 3 is conjugated to SCP and the other glycoconjugates are all individually conjugated to CRM₁₉₇ which is a 25-valent pneumococcal conjugate compositions.

564. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugates are all individually conjugated to CRM₁₉₇.

565. An immunogenic composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B, wherein the glycoconjugates are all individually conjugated to CRM₁₉₇ which is a 25-valent pneumococcal conjugate compositions.

566. The immunogenic composition of any one of paragraphs 454 to 528 wherein said glycoconjugate from S. pneumoniae serotype 3 comprises a serotype 3 saccharide covalently conjugated to a carrier protein (CP) through a spacer and have the general formula (VII):

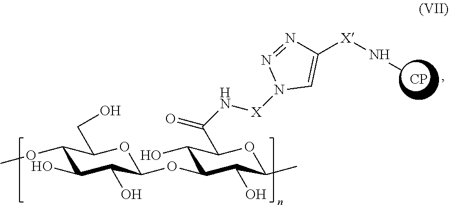

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4, and wherein X is selected from the group consisting of CH₂O(CH₂)_{n″}CH₂C═O, CH₂O(CH₂CH₂O)_{m′}(CH₂)_{n″}CH₂C═O, where n″ is selected from 0 to 10 and m′ is selected from 0 to 4.

567. The immunogenic composition of any one of paragraphs 454 to 528 wherein said glycoconjugate from S. pneumoniae serotype 3 comprises a serotype 3 saccharide covalently conjugated to a carrier protein (CP) through a spacer and have the general formula (VII), wherein X is CH₂(CH₂)_{n′}, where n′ is 2 and wherein X is CH₂O(CH₂)_{n″}CH₂C═O where n″ is 1.

568. The immunogenic composition of any one of paragraphs 454 to 528 wherein said glycoconjugate from S. pneumoniae serotype 3 is produced according to a method, comprising the steps of:
(a) reacting an isolated Streptococcus pneumoniae serotype 3 capsular polysaccharide with a carbonic acid derivative and an azido linker in an aprotic solvent to produce an activated azido polysaccharide,
(b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein,
(c) reacting the activated azido polysaccharide of step (a) with the activated alkyne-carrier protein of step (b) by Cu⁺¹ mediated azide-alkyne cycloaddition reaction to form a glycoconjugate.

569. The immunogenic composition of paragraph 568 wherein the isolated Streptococcus pneumoniae serotype 3 polysaccharide is sized before the activation step (a).

570. The immunogenic composition of paragraph 568 wherein the isolated Streptococcus pneumoniae serotype 3 polysaccharide is sized to a weight average molecular weight between 100 kDa and 200 kDa.

571. The immunogenic composition of any one of paragraphs 568-570 wherein said carbonic acid derivative is selected from the group consisting of 1,1′-carbonyldiimidazole (CDI), 1,1′-carbonyl-di-(1,2,4-triazole) (CDT), disuccinimidyl carbonate (DSC) and N-hydroxysuccinimidyl chloroformate.

572. The immunogenic composition of any one of paragraphs 568-570 wherein said carbonic acid derivative is 1,1′-carbonyldiimidazole (CDI).

573. The immunogenic composition of any one of paragraphs 568-570 wherein said carbonic acid derivative is 1,1′-Carbonyl-di-(1,2,4-triazole) (CDT).

574. The immunogenic composition of any one of paragraphs 568-573 wherein said azido linker is a compound of formula (I),

H₂N—X—N₃ (I)

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

575. The immunogenic composition of any one of paragraphs 568-573 wherein said azido linker is a compound of formula (II),

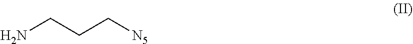

576. The immunogenic composition of any one of paragraphs 568-575 wherein said agent bearing an N-Hydroxysuccinimide (NHS) moiety and an alkyne group is an agent bearing an N-Hydroxysuccinimide (NHS) moiety and a terminal alkyne.

577. The immunogenic composition of any one of paragraphs 568-575 wherein said agent bearing an N-Hydroxysuccinimide (NHS) moiety and an alkyne group is an agent bearing an N-Hydroxysuccinimide (NHS) moiety and a cycloalkyne.

578. The immunogenic composition of any one of paragraphs 568-575 wherein said agent bearing an N-Hydroxysuccinimide (NHS) moiety and an alkyne group is a compound of formula (III),

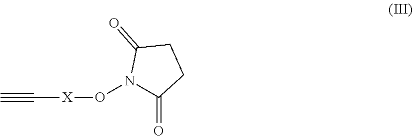

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

579. The immunogenic composition of any one of paragraphs 568-575 wherein said agent bearing an N-Hydroxysuccinimide (NHS) moiety and an alkyne group is a compound of formula (IV):

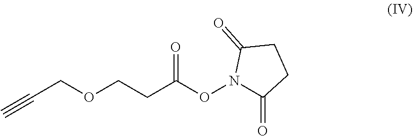 [SMILES: C#CCOCCC(=O)ON1C(=O)CCC1=O]

580. The immunogenic composition of any one of paragraphs 568-579 wherein step a) comprises reacting the isolated Streptococcus pneumoniae serotype 3 capsular polysaccharide with a carbonic acid derivative followed by reacting the carbonic acid derivative-activated polysaccharide with an azido linker in an aprotic solvent to produce an activated azido polysaccharide.

581. The immunogenic composition of any one of paragraphs 568-580 wherein at step a) the isolated Streptococcus pneumoniae serotype 3 capsular polysaccharide is reacted with said carbonic acid derivative in an aprotic solvent.

582. The immunogenic composition of any one of paragraphs 568-581 wherein at step a) the isolated Streptococcus pneumoniae serotype 3 capsular polysaccharide is reacted with a carbonic acid derivative in a solution consisting essentially of dimethylsulphoxide (DMSO).

583. The immunogenic composition of any one of paragraphs 568-582 wherein at step a) the isolated Streptococcus pneumoniae serotype 3 capsular polysaccharide is reacted with CDI in an aprotic solvent comprising 0.1% to 1% (v/v) water.

584. The immunogenic composition of any one of paragraphs 568-582 wherein at step a) the isolated Streptococcus pneumoniae serotype 3 capsular polysaccharide is reacted with CDI in DMSO comprising 0.1% to 1% (v/v) water.

585. The immunogenic composition of any one of paragraphs 568-584 wherein at step a) carbonic acid derivative activation is followed by the addition of water.

586. The immunogenic composition of any paragraph 585 wherein water is added to bring the total water content in the mixture to between about 1% to about 10% (v/v).

587. The immunogenic composition of any one of paragraphs 568-586 wherein step a) further comprises reacting the carbonic acid derivative-activated polysaccharide with an amount of azido linker that is between 0.01-10 molar equivalent to the amount of polysaccharide Repeat Unit of the activated polysaccharide.

588. The immunogenic composition of any one of paragraphs 568-587 wherein the degree of activation of the activated polysaccharide following step a) is between 0.5 to 50%.

589. The immunogenic composition of any one of paragraphs 568-588 wherein step b) comprises reacting the carrier protein with an amount of agent bearing an N-Hydroxysuccinimide (NHS) moiety and an alkyne group that is 0.1-10 molar equivalents to the lysines on the carrier.

590. The immunogenic composition of any one of paragraphs 568-589 wherein the degree of activation of the activated carrier following step b) is between 1 and 50.

591. The immunogenic composition of any one of paragraphs 568-590 wherein the conjugation reaction c) is carried out in aqueous buffer in the presence of copper (I) as catalyst.

592. The immunogenic composition of any one of paragraphs 568-590 wherein the conjugation reaction c) is carried out in aqueous buffer in the presence an oxidant and of copper (I) as catalyst.

593. The immunogenic composition of any one of paragraphs 568-590 wherein the conjugation reaction c) is carried out in aqueous buffer in the presence of copper (I) as catalyst and ascorbate as oxidant, wherein the reaction mixture further comprises THPTA (tris(3-hydroxypropyltriazolylmethyl)amine) and aminoguanidine.

594. The immunogenic composition of any one of paragraphs 568-593 wherein the initial input ratio (weight by weight) of activated azido polysaccharide to activated alkyne-carrier at setp c) is between 0.1 and 3.

595. The immunogenic composition of any one of paragraphs 568-594 wherein following step c), the method further comprises a step of capping the unreacted azido groups remained in the conjugate with an azido group capping agent.

596. The immunogenic composition of paragraph 595 wherein, said azido group capping agent is a compound of formula (V),

≡-X—OH (V)

wherein X is (CH₂)ₙ wherein n is selected from 1 to 15.

597. The immunogenic composition of paragraph 595 wherein said azido group capping agent is propargyl alcohol.

598. The immunogenic composition of any one of paragraphs 595-597 wherein the capping of the unreacted azido groups is performed with an amount of capping agent that is between 0.05 to 20 molar equivalents to the amount of polysaccharide repeat unit of the activated polysaccharide.

599. The immunogenic composition of any one of paragraphs 568-598 wherein following step c), the method further comprises a step of capping the unreacted alkyne groups remained in the conjugate with an alkyne group capping agent.

600. The immunogenic composition of paragraph 599 wherein said alkyne group capping agent is an agent bearing an azido group.

601. The immunogenic composition of paragraph 600 wherein said alkyne group capping agent is a compound of formula (VI),

N₃—X—OH (VI)

wherein X is (CH₂)ₙ wherein n is selected from 1 to 15.

602. The immunogenic composition of paragraphs 600 wherein said alkyne group capping agent is 3-azido-1-propanol.

603. The immunogenic composition of any one of paragraphs 599-602 wherein the capping of the unreacted alkyne groups is performed with an amount of capping agent that is between 0.05 to 20 molar equivalents to the amount of polysaccharide repeat unit of the activated polysaccharide.

604. The immunogenic composition of any one of paragraphs 454 to 528 wherein said glycoconjugate from S. pneumoniae serotype 3 is prepared using reductive amination chemistry.

605. The immunogenic composition of any one of paragraphs 454 to 528 wherein said glycoconjugate from S. pneumoniae serotype 3 is produced according to a method, comprising the steps of:
(a) reacting an isolated Streptococcus pneumoniae serotype 3 capsular polysaccharide with an oxidizing agent;
(b) compounding the activated polysaccharide of step (a) with a carrier protein; and (c) reacting the compounded activated polysaccharide and carrier protein with a reducing agent to form a glycoconjugate.

606. The immunogenic composition of paragraph 605 wherein, the isolated Streptococcus pneumoniae serotype 3 capsular polysaccharide is sized before the activation step (a).

607. The immunogenic composition of paragraph 605 wherein, the isolated Streptococcus pneumoniae serotype 3 capsular polysaccharide is sized before the activation step (a) to a weight average molecular weight between 100 kDa and 200 kDa.

608. The immunogenic composition of any one of paragraphs 454 to 528 wherein said glycoconjugate from S. pneumoniae serotype 3 is produced according to a method, comprising the steps of:
(a) reacting an isolated Streptococcus pneumoniae serotype 3 capsular polysaccharide with an oxidizing agent;
(a′) quenching the oxidation reaction by addition of a quenching agent;
(b) compounding the activated polysaccharide of step (a) or (a′) with a carrier protein; and
(c) reacting the compounded activated polysaccharide and carrier protein with a reducing agent to form a glycoconjugate.

609. The immunogenic composition of paragraph 608 wherein, the isolated Streptococcus pneumoniae serotype 3 capsular polysaccharide is sized before the activation step (a).

610. The immunogenic composition of paragraph 608 wherein, the isolated Streptococcus pneumoniae serotype 3 capsular polysaccharide is sized before the activation step (a) to a weight average molecular weight between 100 kDa and 200 kDa.

611. The immunogenic composition of any one of paragraphs 605 to 610 wherein said oxidizing agent is periodate.

612. The immunogenic composition of any one of paragraphs 605 to 610 wherein said oxidizing agent is periodic acid.

613. The immunogenic composition of any one of paragraphs 605 to 612 wherein step a) comprises reacting the isolated Streptococcus pneumoniae serotype 3 capsular polysaccharide with 0.01-2 molar equivalents of periodate.

614. The immunogenic composition of any one of paragraphs 605 to 613 wherein the degree of oxidation of the activated serotype 3 polysaccharide is between 2 and 8.

615. The immunogenic composition of any one of paragraphs 605 to 613 wherein the degree of oxidation of the activated serotype 3 polysaccharide is between 11 to 19.

616. The immunogenic composition of any one of paragraphs 605 to 613 wherein the degree of oxidation of the activated serotype 3 polysaccharide is about 15.

617. The immunogenic composition of any one of paragraphs 605 to 616 wherein the initial input ratio (weight by weight) of activated serotype 3 capsular polysaccharide to carrier protein at step b) is between 4:1 and 0.1:1.

618. The immunogenic composition of any one of paragraphs 605 to 616 wherein the initial input ratio (weight by weight) of activated serotype 3 capsular polysaccharide to carrier protein is between 1.5:1 and 0.5:1.

619. The immunogenic composition of any one of paragraphs 605 to 618 wherein the reduction reaction (c) is carried out in aqueous solvent.

620. The immunogenic composition of any one of paragraphs 605 to 618 wherein the reduction reaction (c) is carried out in aprotic solvent.

621. The immunogenic composition of any one of paragraphs 605 to 618 wherein the reduction reaction (c) is carried out in a solution consisting essentially of dimethylsulphoxide (DMSO).

622. The immunogenic composition of any one of paragraphs 605 to 621 wherein the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB).

623. The immunogenic composition of any one of paragraphs 605 to 621 wherein the reducing agent is sodium cyanoborohydride.

624. The immunogenic composition of any one of paragraphs 605 to 622 wherein between 0.2 and 20 molar equivalents of reducing agent is used in step c).

625. The immunogenic composition of any one of paragraphs 605 to 622 wherein between 0.5 and 3 molar equivalents of reducing agent is used in step c).

626. The immunogenic composition of any one of paragraphs 605 to 625 wherein, the product of step c) is reacted with 1 to 20 molar equivalents of sodium borohydride for 15 mins-15 hrs.

627. The immunogenic composition of any one of paragraphs 454 to 626 wherein the glycoconjugate from S. pneumoniae serotype 3 comprises a serotype 3 capsular polysaccharide wherein the weight average molecular weight (Mw) of said polysaccharide is between 50 kDa and 1,000 kDa.

628. The immunogenic composition of any one of paragraphs 454 to 626 wherein the glycoconjugate from S. pneumoniae serotype 3 comprises a serotype 3 capsular polysaccharide wherein the weight average molecular weight (Mw) of said polysaccharide is between 100 kDa and 300 kDa.

629. The immunogenic composition of any one of paragraphs 454 to 626 wherein the glycoconjugate from S. pneumoniae serotype 3 comprises a serotype 3 capsular polysaccharide wherein the weight average molecular weight (Mw) of said polysaccharide is between 100 kDa and 200 kDa.

630. The immunogenic composition of any one of paragraphs 454 to 626 wherein the glycoconjugate from S. pneumoniae serotype 3 comprises a serotype 3 capsular polysaccharide wherein the weight average molecular weight (Mw) of said polysaccharide is between 200 kDa and 300 kDa.

631. The immunogenic composition of any one of paragraphs 454 to 626 wherein the glycoconjugate from S. pneumoniae serotype 3 comprises a serotype 3 capsular polysaccharide wherein the weight average molecular weight (Mw) of said polysaccharide is between 100 kDa and 300 kDa.

632. The immunogenic composition of any one of paragraphs 454 to 631 wherein the glycoconjugate from S. pneumoniae serotype 3 has a weight average molecular weight (Mw) of between 250 kDa and 20,000 kDa.

633. The immunogenic composition of any one of paragraphs 454 to 631 wherein the glycoconjugate from S. pneumoniae serotype 3 has a weight average molecular weight (Mw) of between 500 kDa and 15,000 kDa.

634. The immunogenic composition of any one of paragraphs 454 to 631 wherein the glycoconjugate from S. pneumoniae serotype 3 has a weight average molecular weight (Mw) of between 500 kDa and 10,000 kDa.

635. The immunogenic composition of any one of paragraphs 454 to 631 wherein the glycoconjugate from S. pneumoniae serotype 3 has a weight average molecular weight (Mw) of between 500 kDa and 5,000 kDa.

636. The immunogenic composition of any one of paragraphs 454 to 631 wherein the glycoconjugate from S. pneumoniae serotype 3 has a weight average molecular weight (Mw) of between 600 kDa and 3,000 kDa.

637. The immunogenic composition of any one of paragraphs 454 to 636 wherein the degree of conjugation of the serotype 3 glycoconjugate is between 2 and 15.

638. The immunogenic composition of any one of paragraphs 454 to 636 wherein the degree of conjugation of the serotype 3 glycoconjugate is between 4 and 7.

639. The immunogenic composition of any one of paragraphs 454 to 638 wherein the ratio of serotype 3 polysaccharide to carrier protein in the serotype 3 glycoconjugate is between 0.5 and 3.0 (w/w).

640. The immunogenic composition of any one of paragraphs 454 to 638 wherein the ratio of serotype 3 polysaccharide to carrier protein in the serotype 3 glycoconjugate is between 0.5 and 1.5 (w/w).

641. The immunogenic composition of any one of paragraphs 454 to 638 wherein the ratio of serotype 3 polysaccharide to carrier protein in the serotype 3 glycoconjugate is between 0.9 and 1.1 (w/w).

642. The immunogenic composition of any one of paragraphs 454 to 641 wherein said serotype 3 glycoconjugate comprises at least one covalent linkage between the carrier protein and the polysaccharide for every 4 saccharide repeat units of the polysaccharide.

643. The immunogenic composition of any one of paragraphs 454 to 641 wherein said serotype 3 glycoconjugate comprises at least one covalent linkage between the carrier protein and the polysaccharide for every 10 saccharide repeat units of the polysaccharide.

644. The immunogenic composition of any one of paragraphs 454 to 641 wherein said serotype 3 glycoconjugate comprises at least one covalent linkage between the carrier protein and the polysaccharide for every 15 saccharide repeat units of the polysaccharide.

645. The immunogenic composition of any one of paragraphs 454 to 641 wherein said serotype 3 glycoconjugate comprises at least one covalent linkage between the carrier protein and the polysaccharide for every 25 saccharide repeat units of the polysaccharide.

646. The immunogenic composition of any one of paragraphs 454 to 641 wherein said serotype 3 glycoconjugate comprises at least one covalent linkage between the carrier protein and the polysaccharide for every 50 saccharide repeat units of the polysaccharide.

647. The immunogenic composition of any one of paragraphs 454 to 641 wherein said serotype 3 glycoconjugate comprises at least one covalent linkage between the carrier protein and the polysaccharide for every 100 saccharide repeat units of the polysaccharide.

648. The immunogenic composition of any one of paragraphs 454 to 641 wherein said serotype 3 glycoconjugate comprises at least one covalent linkage between the carrier protein and the polysaccharide for every 5 to 10 saccharide repeat units of the polysaccharide.

649. The immunogenic composition of any one of paragraphs 454 to 641 wherein said serotype 3 glycoconjugate comprises at least one covalent linkage between the carrier protein and the polysaccharide for every 10 to 20 saccharide repeat units of the polysaccharide.

650. The immunogenic composition of any one of paragraphs 454 to 649 comprising less than about 50% of free serotype 3 polysaccharide compared to the total amount of serotype 3 polysaccharide.

651. The immunogenic composition of any one of paragraphs 454 to 649 comprising less than about 40% of free serotype 3 polysaccharide compared to the total amount of serotype 3 polysaccharide.

652. The immunogenic composition of any one of paragraphs 454 to 649 comprising less than about 25% of free serotype 3 polysaccharide compared to the total amount of serotype 3 polysaccharide.

653. The immunogenic composition of any one of paragraphs 454 to 649 comprising less than about 20% of free serotype 3 polysaccharide compared to the total amount of serotype 3 polysaccharide.

654. The immunogenic composition of any one of paragraphs 454 to 649 comprising less than about 15% of free serotype 3 polysaccharide compared to the total amount of serotype 3 polysaccharide.

655. The immunogenic composition of any one of paragraphs 454 to 654 wherein at least 30% of the serotype 3 glycoconjugate has a K_{d} below or equal to 0.3 in a CL-4B column.

656. The immunogenic composition of any one of paragraphs 454 to 654 wherein at least 40% of the serotype 3 glycoconjugate has a K_{d} below or equal to 0.3 in a CL-4B column.

657. The immunogenic composition of any one of paragraphs 454 to 654 wherein at least 45%, 50%, 55%, 60%, 65%, 70%, 75%, 80%, or 85% of the serotype 3 glycoconjugate has a K_{d} below or equal to 0.3 in a CL-4B column.

658. The immunogenic composition of any one of paragraphs 454 to 654 wherein at least 60% of the serotype 3 glycoconjugate has a K_{d} below or equal to 0.3 in a CL-4B column.

659. The immunogenic composition of any one of paragraphs 454 to 654 wherein between 50% and 80% of the serotype 3 glycoconjugate has a K_{d} below or equal to 0.3 in a CL-4B column.

660. The immunogenic composition of any one of paragraphs 454 to 654 wherein between 65% and 80% of the serotype 3 glycoconjugate has a K_{d} below or equal to 0.3 in a CL-4B column.

661. The glycoconjugate of paragraph 24, 107, 195, 281 or 368 wherein the carrier protein is an enzymatically inactive SCP.

662. The process of paragraph 77, 169, 252, 342 or 435 wherein the carrier protein is an enzymatically inactive SCP.

663. The immunogenic composition of paragraph 529 or any one of paragraphs 541-561 wherein said SPC is an enzymatically inactive SCP.

664. The immunogenic composition of paragraph 562 wherein said is an enzymatically inactive SCP.

665. The immunogenic composition of paragraph 563 wherein said SPC is an enzymatically inactive SCP.

666. The glycoconjugate of paragraph 24, 107, 195, 281 or 368 wherein the carrier protein is an enzymatically inactive SCP from GBS (SCPB).

667. The process of paragraph 77, 169, 252, 342 or 435 wherein the carrier protein is an enzymatically inactive SCP from GBS (SCPB).

668. The immunogenic composition of paragraph 529 or any one of paragraphs 541-561 wherein said SPC is an enzymatically inactive SCP from GBS (SCPB).

669. The immunogenic composition of paragraph 562 wherein said SPC is an SCP from GBS (SCPB).

670. The immunogenic composition of paragraph 563 wherein said SPC is an enzymatically inactive SCP from GBS (SCPB).

671. The glycoconjugate of paragraph 24, 107, 195, 281 or 368 wherein the carrier protein is a fragment of an SCPB.

672. The process of paragraph 77, 169, 252, 342 or 435 wherein the carrier protein is a fragment of an SCPB.

673. The immunogenic composition of paragraph 529 or any one of paragraphs 541-561 wherein said SPC is a fragment of an SCPB.

674. The immunogenic composition of paragraph 562 wherein said SPC is a fragment of an SCPB.

675. The immunogenic composition of paragraph 563 wherein said SPC is a fragment of an SCPB.

676. The glycoconjugate of paragraph 24, 107, 195, 281 or 368 wherein the carrier protein is a fragment of an SCP which comprises the protease domain, the protease-associated domain (PA domain) and the three fibronectin type III (Fn) domains but does not comprise the export signal presequence, the pro-sequence and the cell wall anchor domain.

677. The process of paragraph 77, 169, 252, 342 or 435 wherein the carrier is a fragment of an SCP which comprises the protease domain, the protease-associated domain (PA domain) and the three fibronectin type III (Fn) domains but does not comprise the export signal presequence, the pro-sequence and the cell wall anchor domain.

678. The immunogenic composition of paragraph 529 or any one of paragraphs 541-561 wherein said SPC is a fragment of an SCP which comprises the protease domain, the protease-associated domain (PA domain) and the three fibronectin type III (Fn) domains but does not comprise the export signal presequence, the pro-sequence and the cell wall anchor domain.

679. The immunogenic composition of paragraph 562 wherein said SPC is a fragment of an SCP which comprises the protease domain, the protease-associated domain (PA domain) and the three fibronectin type III (Fn) domains but does not comprise the export signal presequence, the pro-sequence and the cell wall anchor domain.

680. The immunogenic composition of paragraph 563 wherein said SPC is a fragment of an SCP which comprises the protease domain, the protease-associated domain (PA domain) and the three fibronectin type III (Fn) domains but does not comprise the export signal presequence, the pro-sequence and the cell wall anchor domain.

681. The glycoconjugate of paragraph 24, 107, 195, 281 or 368 wherein the carrier protein is an enzymatically inactive fragment of SCP comprising the protease domain, the protease-associated domain (PA domain) and the three fibronectin type III (Fn) domains but does not comprise the export signal presequence, the pro-sequence and the cell wall anchor domain.

682. The process of paragraph 77, 169, 252, 342 or 435 wherein the carrier protein is an enzymatically inactive fragment of SCP comprising the protease domain, the protease-associated domain (PA domain) and the three fibronectin type III (Fn) domains but does not comprise the export signal presequence, the pro-sequence and the cell wall anchor domain.

683. The immunogenic composition of paragraph 529 or any one of paragraphs 541-561 wherein said SPC is an enzymatically inactive fragment of SCP comprising the protease domain, the protease-associated domain (PA domain) and the three fibronectin type III (Fn) domains but does not comprise the export signal presequence, the pro-sequence and the cell wall anchor domain.

684. The immunogenic composition of paragraph 562 wherein said SPCis an enzymatically inactive fragment of SCP comprising the protease domain, the protease-associated domain (PA domain) and the three fibronectin type III (Fn) domains but does not comprise the export signal presequence, the pro-sequence and the cell wall anchor domain.

685. The immunogenic composition of paragraph 563 wherein said SPC is an enzymatically inactive fragment of SCP comprising the protease domain, the protease-associated domain (PA domain) and the three fibronectin type III (Fn) domains but does not comprise the export signal presequence, the pro-sequence and the cell wall anchor domain.

686. The glycoconjugate of paragraph 24, 107, 195, 281 or 368 wherein the carrier protein is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.

687. The process of paragraph 77, 169, 252, 342 or 435 wherein the carrier protein is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.

688. The immunogenic composition of paragraph 529 or any one of paragraphs 541-561 wherein said SPC is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.

689. The immunogenic composition of paragraph 562 wherein said is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.

690. The immunogenic composition of paragraph 563 wherein said SPC is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.

691. The glycoconjugate of paragraph 24, 107, 195, 281 or 368 wherein the carrier protein is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.

692. The process of paragraph 77, 169, 252, 342 or 435 wherein the carrier is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.

693. The immunogenic composition of paragraph 529 or any one of paragraphs 541-561 wherein said SPC is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.

694. The immunogenic composition of paragraph 562 wherein said SPC is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.

695. The immunogenic composition of paragraph 563 wherein said is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.

696. The glycoconjugate of paragraph 24, 107, 195, 281 or 368 wherein the carrier protein is an enzymatically inactive fragment of SCP consisting of a polypeptide having at least 95% identity with SEQ ID NO: 1.

697. The process of paragraph 77, 169, 252, 342 or 435 wherein the carrier protein is a fragment of an SCPB.

698. The immunogenic composition of paragraph 529 or any one of paragraphs 541-561 wherein said SPC is an enzymatically inactive fragment of SCP consisting of a polypeptide having at least 95% identity with SEQ ID NO: 1.

699. The immunogenic composition of paragraph 562 wherein said SPC is an enzymatically inactive fragment of SCP consisting of a polypeptide having at least 95% identity with SEQ ID NO: 1.

700. The immunogenic composition of paragraph 563 wherein said SPC is an enzymatically inactive fragment of SCP consisting of a polypeptide having at least 95% identity with SEQ ID NO: 1.

701. The glycoconjugate of paragraph 24, 107, 195, 281 or 368 wherein the carrier protein is an enzymatically inactive fragment of SCP consisting of a polypeptide having at least 95% identity with SEQ ID NO: 2.

702. The process of paragraph 77, 169, 252, 342 or 435 wherein the carrier protein is an enzymatically inactive fragment of SCP consisting of a polypeptide having at least 95% identity with SEQ ID NO: 2.

703. The immunogenic composition of paragraph 529 or any one of paragraphs 541-561 wherein said SPC is an enzymatically inactive fragment of SCP consisting of a polypeptide having at least 95% identity with SEQ ID NO: 2.

704. The immunogenic composition of paragraph 562 wherein said SPC is an enzymatically inactive fragment of SCP consisting of a polypeptide having at least 95% identity with SEQ ID NO: 2.

705. The immunogenic composition of paragraph 563 wherein said SPC is an enzymatically inactive fragment of SCP consisting of a polypeptide having at least 95% identity with SEQ ID NO: 2.

706. The glycoconjugate of paragraph 24, 107, 195, 281 or 368 wherein the carrier protein is an enzymatically inactive fragment of SCP which consists of SEQ ID NO: 1.

707. The process of paragraph 77, 169, 252, 342 or 435 wherein the carrier protein is an enzymatically inactive fragment of SCP which consists of SEQ ID NO: 1.

708. The immunogenic composition of paragraph 529 or any one of paragraphs 541-561 wherein said SPC is an enzymatically inactive fragment of SCP which consists of SEQ ID NO: 1.

709. The immunogenic composition of paragraph 562 wherein said SPC is an enzymatically inactive fragment of SCP which consists of SEQ ID NO: 1.

710. The immunogenic composition of paragraph 563 wherein said SPC is an enzymatically inactive fragment of SCP which consists of SEQ ID NO: 1.

711. The glycoconjugate of paragraph 24, 107, 195, 281 or 368 wherein the carrier protein is an enzymatically inactive fragment of SCP which consists of SEQ ID NO: 2.

712. The process of paragraph 77, 169, 252, 342 or 435 wherein the carrier protein is an enzymatically inactive fragment of SCP which consists of SEQ ID NO: 2.

713. The immunogenic composition of paragraph 529 or any one of paragraphs 541-561 wherein said SPC is an enzymatically inactive fragment of SCP which consists of SEQ ID NO: 2.

714. The immunogenic composition of paragraph 562 wherein said SPC is an enzymatically inactive fragment of SCP which consists of SEQ ID NO: 2.

715. The immunogenic composition of paragraph 563 wherein said SPC is an enzymatically inactive fragment of SCP which consists of SEQ ID NO: 2.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance. All publications and patent applications mentioned in the specification are indicative of the level of those skilled in the art to which this invention pertains. All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

### Example 1: Preparation of Serotype 15A Polysaccharide—CRM₁₉₇ Conjugate

1.1. Preparation of Streptococcus pneumoniae serotype 15A polysaccharide Serotype 15A capsular polysaccharides can be obtained directly from bacteria using isolation procedures known to one of ordinary skill in the art (see for example methods disclosed in U.S. Patent App. Pub. No. 2008057688 or WO2008118752). The serotype 15A Streptococcus pneumonia was grown in a bioreactor and the fermentation broth was inactivated. The polysaccharide was then isolated and purified by ultrafiltration and diafiltration.

The purified Streptococcus pneumoniae serotype 15A polysaccharide was sized by high pressure microfluidizer to produce the size-reduced 15A polysaccharide.

### 1.2. Oxidation of Streptococcus pneumoniae Serotype 15A Capsular Polysaccharide

A calculated volume of 1.0 M potassium acetate buffer (pH 5.5) and water-for-injection (WFI) was added to the polysaccharide solution to achieve a final polysaccharide concentration of 2.0 g/L and a final concentration of 50 mM potassium acetate buffer, if required pH was adjusted to 5.0, approximately. The diluted polysaccharide was then heated to 35° C. Oxidation was initiated by the addition of 1.0 molar equivalents (MEq) of sodium periodate. The oxidation reaction was kept for 24 hrs at 35° C.

After reaching the target reaction time, the activated polysaccharide was cooled to room temperature and concentrated using 10K MWCO Millipore ultrafiltration cassettes. The diafiltration was then performed against 20-fold diavolume of WFI. The purified activated saccharide was characterized by (i) Molecular Weight by SEC-MALLS and (ii) Degree of Oxidation (DO).

### 1.3 Conjugation of Activated Streptococcus pneumoniae Serotype 15A Polysaccharide with CRM₁₉₇

The conjugation process consists of the following steps:
a. Compounding and lyophilization of activated polysaccharide with sucrose excipient
b. Reconstitution of the lyophilized polysaccharide and CRM₁₉₇.
c. Conjugation of activated polysaccharide to CRM197 and capping
d. Purification of the conjugate

### a. Compounding with Sucrose

The activated polysaccharide is compounded with sucrose to a ratio of 10 grams of sucrose per gram of activated polysaccharide. The compounded mixture was then shell frozen and lyophilized.

### b. Reconstitution of Lyophilized Activated Polysaccharide and CRM₁₉₇ Protein in DMSO

Lyophilized activated polysaccharide was reconstituted in anhydrous dimethyl sulfoxide (DMSO). the same amount of anhydrous DMSO was added to the calculated CRM₁₉₇ for reconstitution.

### c. Conjugation of Activated Polysaccharide to CRM₁₉₇ and Capping

Reconstituted activated polysaccharide was added to the reconstituted CRM₁₉₇ (in DMSO) in the conjugation reactor. The final polysaccharide concentration is 4 g/L. Conjugation was performed by adding 1.0 MEq of sodium cyanoborohydride to the reaction mixture. The reaction was incubated at 23±2° C. for 24 hrs. Termination of conjugation reaction was done by adding 2 MEq of sodium borohydride. The capping reaction was proceeded for 3 hrs.

### d. Purification of Conjugate

The conjugate solution was then diluted into 5-10× (by volume) 5 mM succinate/saline (pH 6.0) and a 50× diafiltration was performed against 5 mM succinate/saline (pH 6.0) using 100K MWCO Millipore ultrafiltration cassettes. The purified conjugate was filtered through a 0.45 μm/0.22 μm filter, and can be stored at 2-8° C.

Table 1 below comprises characterizing data for serotype 15A polysaccharide-CRM₁₉₇ conjugates obtained by the method of this invention.

TABLE 1
Serotype 15A Conjugates

| Conjugate# | 1 | 2 | 3 | 4 | 5 | 6 |
|---|---|---|---|---|---|---|
| DO | 4.7 | 4.8 | 5.3 | 5.3 | 10.1 | 2.7 |
| Act Poly Mw (kDa) | 107 | 133 | 171 | 152 | 164 | 107 |
| SPR (Input Ratio) | 1.0 | 1.0 | 1.0 | 1.0 | 0.8 | 1.0 |
| Conjugation yield | 65 | 53 | 60 | 56 | 46 | 60 |
| (%) | | | | | | |
| SPR (Output Ratio) | 1.0 | 0.8 | 1.0 | 1.0 | 0.8 | 0.9 |
| Free Saccharide | 9 | <5 | 6 | 15 | 22 | 10 |
| (%) | | | | | | |
| Conjugate Mw | 2386 | 2247 | 3113 | 1832 | 4850 | 1390 |
| (kDa) | | | | | | |
| Modified Lys (AAA) | N/A | N/A | N/A | 15.5 | 4.8 | 10 |

SPR = Saccharide Protein Ratio

### Example 2: Preparation of Serotype 23A Polysaccharide—CRM₁₉₇ Conjugate

### 2.1. Preparation of Streptococcus pneumoniae Serotype 23A Polysaccharide

Purified and size-reduced serotype 23A polysaccharides was obtained in a similar fashion as disclosed in example 1.1.

### 2.2. Oxidation of Streptococcus pneumoniae Serotype 23A Capsular Polysaccharide

A calculated volume of 1.0 M potassium acetate buffer and water-for-injection (WFI) was added to the polysaccharide solution to achieve a final polysaccharide concentration of 2.0 g/L and a final concentration of 50 mM potassium acetate buffer, if required pH was adjusted to 5.5, approximately. Oxidation was initiated by the addition of 0.45 molar equivalents (MEq) of sodium periodate. The oxidation reaction was proceeded for 18 hrs.

After reaching the target reaction time, the activated polysaccharide was concentrated using 10K MWCO Millipore ultrafiltration cassettes. The diafiltration was then performed against 20-fold diavolume of WFI. The purified activated saccharide was characterized by (i) Molecular Weight by SEC-MALLS and (ii) Degree of Oxidation (DO).

### 2.3 Conjugation of Activated Streptococcus pneumoniae Serotype 23A Polysaccharide with CRM₁₉₇

The conjugation process consists of the following steps:
a. Compounding and lyophilization of activated polysaccharide with sucrose excipient
b. Reconstitution of the lyophilized polysaccharide and CRM₁₉₇.
c. Conjugation of activated polysaccharide to CRM197 and capping
d. Purification of the conjugate

### a. Compounding with Sucrose

The activated polysaccharide is compounded with sucrose to a ratio of 25 grams of sucrose per gram of activated polysaccharide. The compounded mixture was then shell frozen and lyophilized.

### b. Reconstitution of Lyophilized Activated Polysaccharide and CRM₁₉₇ Protein in DMSO

Lyophilized activated polysaccharide was reconstituted in anhydrous dimethyl sulfoxide (DMSO). the same amount of anhydrous DMSO was added to the calculated CRM₁₉₇ for reconstitution.

### c. Conjugation of Activated Polysaccharide to CRM₁₉₇ and Capping

Reconstituted activated polysaccharide was added to the reconstituted CRM₁₉₇ (in DMSO) in the conjugation reactor. The final polysaccharide concentration is 1 g/L. Conjugation was performed by adding 1.0 MEq of sodium cyanoborohydride to the reaction mixture. The reaction was incubated at 23±2° C. for 24 hrs. Termination of conjugation reaction was done by adding 2 MEq of sodium borohydride. The capping reaction was proceeded for 3 hrs.

### d. Purification of Conjugate

The conjugate solution was then diluted into 5-10× (by volume) 5 mM succinate/saline (pH 6.0) and a 50× diafiltration was performed against 5 mM succinate/saline (pH 6.0) using 100K MWCO Millipore ultrafiltration cassettes. The purified conjugate was filtered through a 0.45 μm/0.22 μm filter, and can be stored at 2-8° C.

Table 2 below comprises characterizing data for serotype 23A polysaccharide-CRM₁₉₇ conjugates obtained by the method of this invention.

TABLE 2
Serotype 23A Conjugates

| Conjugate# | 1 | 2 | 3 |
|---|---|---|---|
| DO | 5.3 | 5.4 | 7.0 |
| Act Poly Mw (kDa) | 192 | 178 | 184 |
| SPR (Input Ratio) | 0.8 | 0.8 | 0.7 |
| Conjugation yield | 78 | 51 | 76 |
| (%) | | | |
| SPR (Output Ratio) | 0.8 | 0.7 | 0.9 |
| Free Saccharide (%) | 13 | 10 | 5 |
| Conjugate Mw (kDa) | 6150 | 5075 | 5423 |
| Modified Lys (AAA) | N/A | 15 | 14 |

### Example 3: Preparation of Serotype 23B Polysaccharide—CRM₁₉₇ Conjugate

### 3.1. Preparation of Streptococcus pneumoniae Serotype 23B Polysaccharide

Purified and size-reduced serotype 23B polysaccharides was obtained in a similar fashion as disclosed in example 1.1.

### 3.2. Oxidation of Streptococcus pneumoniae Serotype 23B Capsular Polysaccharide

A calculated volume of 1.0 M potassium acetate buffer and water-for-injection (WFI) was added to the polysaccharide solution to achieve a final polysaccharide concentration of 2.0 g/L and a final concentration of 50 mM potassium acetate buffer, if required pH was adjusted to 5.0, approximately. The solution was then cooled to 5° C. Oxidation was initiated by the addition of 0.2 molar equivalents (MEq) of sodium periodate. The oxidation reaction was proceeded for 18 hrs.

After reaching the target reaction time, the activated polysaccharide was concentrated using 30K MWCO Millipore ultrafiltration cassettes. The diafiltration was then performed against 20-fold diavolume of WFI. The purified activated saccharide was characterized by (i) Molecular Weight by SEC-MALLS and (ii) Degree of Oxidation (DO).

### 3.3 Conjugation an Activated Streptococcus pneumoniae Serotype 23B Polysaccharide with CRM₁₉₇

The conjugation process consists of the following steps:
a. Compounding and lyophilization of activated polysaccharide with sucrose excipient
b. Reconstitution of the lyophilized polysaccharide and CRM₁₉₇.
c. Conjugation of activated polysaccharide to CRM197 and capping
d. Purification of the conjugate

### a. Compounding with Sucrose

The activated polysaccharide is compounded with sucrose to a ratio of 10 grams of sucrose per gram of activated polysaccharide. The compounded mixture was then shell frozen and lyophilized.

### b. Reconstitution of Lyophilized Activated Polysaccharide and CRM₁₉₇ Protein in DMSO

Lyophilized activated polysaccharide was reconstituted in anhydrous dimethyl sulfoxide (DMSO). the same amount of anhydrous DMSO was added to the calculated CRM₁₉₇ for reconstitution.

### c. Conjugation of Activated Polysaccharide to CRM₁₉₇ and Capping

Reconstituted activated polysaccharide was added to the reconstituted CRM₁₉₇ (in DMSO) in the conjugation reactor. The final polysaccharide concentration is 3 g/L. Conjugation was performed by adding 1.0 MEq of sodium cyanoborohydride to the reaction mixture. The reaction was incubated at 23±2° C. for 24 hrs. Termination of conjugation reaction was done by adding 2 MEq of sodium borohydride. The capping reaction was proceeded for 3 hrs.

### d. Purification of Conjugate

The conjugate solution was then diluted into 5-10× (by volume) 5 mM succinate/saline (pH 6.0) and a 50× diafiltration was performed against 5 mM succinate/saline (pH 6.0) using 100K MWCO Millipore ultrafiltration cassettes. The purified conjugate was filtered through a 0.45 μm/0.22 μm filter, and can be stored at 2-8° C.

Table 3 below comprises characterizing data for serotype 23B polysaccharide-CRM₁₉₇ conjugates obtained by the method of this invention.

TABLE 3
Serotype 23B Conjugates

| Conjugate# | 1 | 2 | 3 | 4 |
|---|---|---|---|---|
| DO | 10.3 | 9.7 | 5.7 | 12 |
| Act Poly Mw (kDa) | 162 | 173 | 183 | 163 |
| SPR (Input Ratio) | 0.9 | 0.9 | 0.8 | 0.8 |
| Conjugation yield | 58 | 49 | 52 | 58 |
| (%) | | | | |
| SPR (Output Ratio) | 0.75 | 0.8 | 0.6 | 0.75 |
| Free Saccharide (%) | 13 | 9 | 5 | 8 |
| Conjugate Mw (kDa) | 1025 | 1107 | 767 | 1493 |
| Modified Lys (AAA) | 15.9 | N/A | 4.2 | 6.5 |

### Example 4: Preparation of Serotype 24F Polysaccharide—CRM₁₉₇ Conjugate

### 4.1. Preparation of Streptococcus pneumoniae Serotype 24F Polysaccharide

Purified and size-reduced serotype 24F polysaccharides was obtained in a similar fashion as disclosed in example 1.1.

### 4.2. Oxidation of Streptococcus pneumoniae Serotype 24F Capsular Polysaccharide

A calculated volume of 1.0 M potassium acetate buffer and water-for-injection (WFI) was added to the polysaccharide solution to achieve a final polysaccharide concentration of 2.0 g/L and a final concentration of 50 mM potassium acetate buffer, if required pH was adjusted to 5.0, approximately. The solution was then cooled to 5° C. Oxidation was initiated by the addition of 0.3 molar equivalents (MEq) of sodium periodate. The oxidation reaction was proceeded for 18 hrs.

After reaching the target reaction time, the activated polysaccharide was concentrated using 10K MWCO Millipore ultrafiltration cassettes. The diafiltration was then performed against 20-fold diavolume of WFI. The purified activated saccharide was characterized by (i) Molecular Weight by SEC-MALLS and (ii) Degree of Oxidation (DO).

### 4.3 Conjugation an Activated Streptococcus pneumoniae Serotype 24F Polysaccharide with CRM₁₉₇

The conjugation process consists of the following steps:
a. Compounding and lyophilization of activated polysaccharide with sucrose excipient
b. Reconstitution of the lyophilized polysaccharide and CRM₁₉₇.
c. Conjugation of activated polysaccharide to CRM197 and capping
d. Purification of the conjugate

### a. Compounding with Sucrose

The activated polysaccharide is compounded with sucrose to a ratio of 25 grams of sucrose per gram of activated polysaccharide. The compounded mixture was then shell frozen and lyophilized.

### b. Reconstitution of Lyophilized Activated Polysaccharide and CRM₁₉₇ Protein in DMSO

Lyophilized activated polysaccharide was reconstituted in anhydrous dimethyl sulfoxide (DMSO). the same amount of anhydrous DMSO was added to the calculated CRM₁₉₇ for reconstitution.

### c. Conjugation of Activated Polysaccharide to CRM₁₉₇ and Capping

Reconstituted activated polysaccharide was added to the reconstituted CRM₁₉₇ (in DMSO) in the conjugation reactor. The final polysaccharide concentration is 0.7 g/L. Conjugation was performed by adding 1.0 MEq of sodium cyanoborohydride to the reaction mixture. The reaction was incubated at 23±2° C. for 5 hrs. Termination of conjugation reaction was done by adding 2 MEq of sodium borohydride. The capping reaction was proceeded for 2 hrs.

### d. Purification of Conjugate

The conjugate solution was then diluted into 5-10× (by volume) 5 mM succinate/saline (pH 6.0) and a 50× diafiltration was performed against 5 mM succinate/saline (pH 6.0) using 100K MWCO Millipore ultrafiltration cassettes. The purified conjugate was filtered through a 0.45 μm/0.22 μm filter, and can be stored at 2-8° C.

Table 4 below comprises characterizing data for serotype 24F polysaccharide-CRM₁₉₇ conjugates obtained by the method of this invention.

TABLE 4
Serotype 24F Conjugates

| Conjugate# | 1 | 2 | 3 | 4 |
|---|---|---|---|---|
| DO | 9 | 9.5 | 9.7 | 9.4 |
| Act Poly Mw (kDa) | 182 | 178 | 234 | 147 |
| SPR (Input Ratio) | 0.8 | 0.8 | 0.8 | 0.8 |
| Conjugation yield (%) | 82 | 61 | 49 | 78 |
| SPR (Output Ratio) | 0.95 | 0.73 | 0.94 | 1.1 |
| Free Saccharide (%) | 10 | 14 | 9 | 24 |
| Conjugate Mw (kDa) | 5757 | 5084 | 3189 | 2354 |
| Modified Lys (AAA) | 7.8 | N/A | N/A | N/A |

### Example 5: Preparation of Serotype 35B Polysaccharide—CRM₁₉₇ Conjugate

### 5.1. Preparation of Streptococcus pneumoniae Serotype 35B Polysaccharide

Purified serotype 35B polysaccharides was obtained in a similar fashion as disclosed in example 1.1, except no mechanical sizing was used to reduce the size of polysaccharide.

### 5.2. Oxidation of Streptococcus pneumoniae Serotype 35B Capsular Polysaccharide

A calculated volume of 1.0 M potassium phosphate buffer and water-for-injection (WFI) was added to the polysaccharide solution to achieve a final polysaccharide concentration of 2.0 g/L and a final concentration of 50 mM potassium phosphate buffer, if required pH was adjusted to 6.0, approximately. Oxidation was initiated by the addition of 0.1 molar equivalents (MEq) of sodium periodate. The oxidation reaction was proceeded for 2 hrs.

After reaching the target reaction time, the activated polysaccharide was concentrated using 5K MWCO Millipore ultrafiltration cassettes. The diafiltration was then performed against 20-fold diavolume of WFI. The purified activated saccharide was characterized by (i) Molecular Weight by SEC-MALLS and (ii) Degree of Oxidation (DO).

### 5.3 Conjugation an Activated Streptococcus pneumoniae Serotype 35B Polysaccharide with CRM₁₉₇

The conjugation process consists of the following steps:
a. Compounding and lyophilization of activated polysaccharide with sucrose excipient
b. Reconstitution of the lyophilized polysaccharide and CRM₁₉₇.
c. Conjugation of activated polysaccharide to CRM197 and capping
d. Purification of the conjugate

### a. Compounding with Sucrose

The activated polysaccharide is compounded with sucrose to a ratio of 10 grams of sucrose per gram of activated polysaccharide. The compounded mixture was then shell frozen and lyophilized.

### b. Reconstitution of Lyophilized Activated Polysaccharide and CRM₁₉₇ Protein in DMSO

Lyophilized activated polysaccharide was reconstituted in anhydrous dimethyl sulfoxide (DMSO). the same amount of anhydrous DMSO was added to the calculated CRM₁₉₇ for reconstitution.

### c. Conjugation of Activated Polysaccharide to CRM₁₉₇ and Capping

Reconstituted activated polysaccharide was added to the reconstituted CRM₁₉₇ (in DMSO) in the conjugation reactor. The final polysaccharide concentration is 4 g/L. Conjugation was performed by adding 1.0 MEq of sodium cyanoborohydride to the reaction mixture. The reaction was incubated at 23±2° C. for 24 hrs. Termination of conjugation reaction was done by adding 2 MEq of sodium borohydride. The capping reaction was proceeded for 3 hrs.

### d. Purification of Conjugate

The conjugate solution was then diluted into 5-10× (by volume) 5 mM succinate/saline (pH 6.0) and a 50× diafiltration was performed against 5 mM succinate/saline (pH 6.0) using 100K MWCO Millipore ultrafiltration cassettes. The purified conjugate was filtered through a 0.45 μm/0.22 μm filter, and can be stored at 2-8° C.

Table 5 below comprises characterizing data for serotype 35B polysaccharide-CRM₁₉₇ conjugates obtained by the method of this invention.

TABLE 5
Serotype 35B Conjugates

| Conjugate# | 1 | 2 | 3 | 4 | 5 | 6 |
|---|---|---|---|---|---|---|
| DO | 8 | 8 | 8 | 8 | 11 | 16 |
| Act Poly Mw (kDa) | 32 | 32 | 32 | 32 | 46 | 60 |
| SPR (Input Ratio) | 0.8 | 0.8 | 0.8 | 0.8 | 0.8 | 0.8 |
| Conjugation yield | 38 | 50 | 50 | 57 | 64 | 55 |
| (%) | | | | | | |
| SPR (Output Ratio) | 0.62 | 0.70 | 0.76 | 0.70 | 0.97 | 0.74 |
| Free Saccharide (%) | 14 | 7 | 8 | 5 | 13 | 5 |
| Conjugate Mw (kDa) | 1746 | 1266 | 1705 | 2682 | 1610 | 3013 |
| Modified Lys (AAA) | N/A | N/A | N/A | N/A | 5.4 | 4.2 |

### Example 6: Structural Characterization of Hydrolyzed Serotype 23A Polysaccharide Via NMR

Polysaccharide conjugates are generated by conjugating the antigen (polysaccharide) with the carrier protein. The size of the final conjugates depends on the size of the polysaccharide used. Generally, the polysaccharide molecular weight as extracted and purified from the bacteria is not suitable to generate conjugate vaccine. Therefore, the polysaccharides are sized before they are made ready to conjugate with the carrier protein.

Surprisingly, it was observed that sizing 23A polysaccharide via acid hydrolysis (as recommended for example in WO2019050814 or in WO2019050818) followed by activation using NaIO4 resulted in poor conjugation. The impact of acid hydrolysis sizing on the structure of Pn-23A polysaccharide has been investigated.

Sample preparation: Native Pn-23A polysaccharide was incubated with 0.1N acetic acid at 80° C. for 2, 4 and 6 hrs. The acid hydrolyzed samples were dialyzed against water and the lyophilized. Lyophilized samples were weighed and dissolved in D20. The samples were bath sonicated for 20 minutes at 50° C. Final concentration of the sample was 14 mg/mL. Native sample was also sized using mechanical homogenizer with 10 pass at 15 k psi pressure. The sized samples were transferred into the NMR tube for NMR data collection and analysis.

NMR Data collection: All data was collected at 75° C. using Bruker 600 MHz spectrometer equipped with BBO cryoprobe. The NMR data was processed using NvX and analyzed using NMRViewJ (NvJ). The following experiments were carried out to do the NMR analysis:
1) 1D 1H (d1 5s; ns 32)
2) 2D 1H-13C HSQC (d1 1 s; ns 4; TD 256; SW 50@82 ppm)

### Results

The molecular weight of the sized Pn-23A was determined using SEC-MALLS. The sized Pn-23A molecular weights were respectively 214 kDa (2 hrs of acid hydrolysis), 137 kDa (4 hrs of acid hydrolysis) and 90 kDa (6 hrs of acid hydrolysis).

The structure of the Pn-23A is shown in FIG. 1. It has been found that acid hydrolysis can affect serotype 23A polysaccharide structure integrity.

FIG. 2 shows the stack plot of anomeric and methyl region of 1D 1 H spectra of Pn-23A polysaccharides sized via sonication (Sized Sonication) and acid hydrolysis for 2 hours (2 Hrs), 4 hours (4 Hrs) and 6 hours (6 Hrs). The anomeric and methyl signals are annotated. Highlighted bars (3 grey bars) clearly shows the increase of the unknown signals as the hydrolysis time is increasing

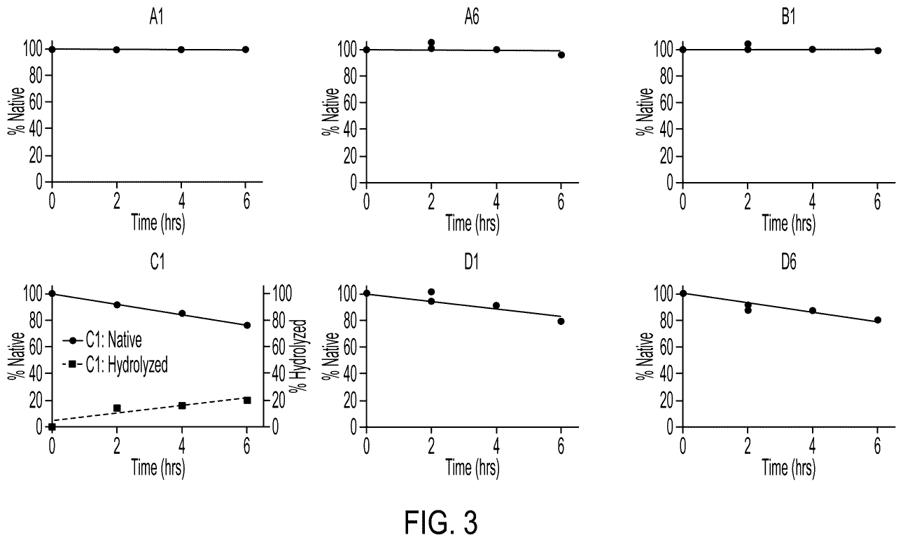

The plot of the signal intensities of selected resonances (representing from each sugar residues: A1, A6, B1, C1, D1 and D6) against the increasing hydrolysis time illustrates the structural changes upon hydrolysis (FIG. 3). There would be no change in the signal intensity if a certain resonance would not be affected by hydrolysis. It is evident from FIG. 3 that loss of the signal intensity is observed in residues C and D, thus suggesting that there are significant structural changes upon hydrolysis.

Complete structural analysis of the hydrolyzed sample was done using NMR spectroscopy. The new resonances that were observed (FIG. 2) were completely assigned. One of those resonances were anomeric signal from residue C of the hydrolyzed Pn-23A polysaccharide, which will show an increase in the signal intensity as hydrolysis time increases (FIG. 3, lower panel C1).

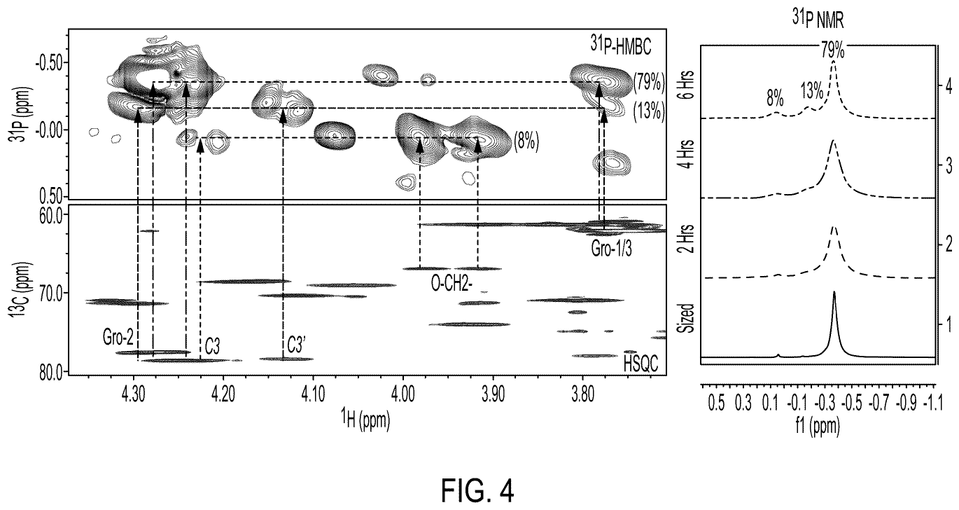

These changes were also observed in 1D ³¹P and 2D ¹H-³¹P HMBC spectra of the various sized samples were analyzed. FIG. 4 shows the 1D ³¹P NMR spectra of hydrolyzed Pn-23A polysaccharide (right panel) and 2D ¹H-³¹P HMBC spectrum (left panel) of 6 Hrs hydrolyzed Pn-23A polysaccharide. The 1D ³¹P NMR spectrum of Pn-23A polysaccharide hydrolyzed for 6 hrs clearly shows different populations of Pn-23A polysaccharide present after hydrolysis. The 2D ¹H-³¹P HMBC spectra was used to shows the Phosphorus to carbon correlations of these three different populations (respectively ˜79%, ˜13% and ˜8%).

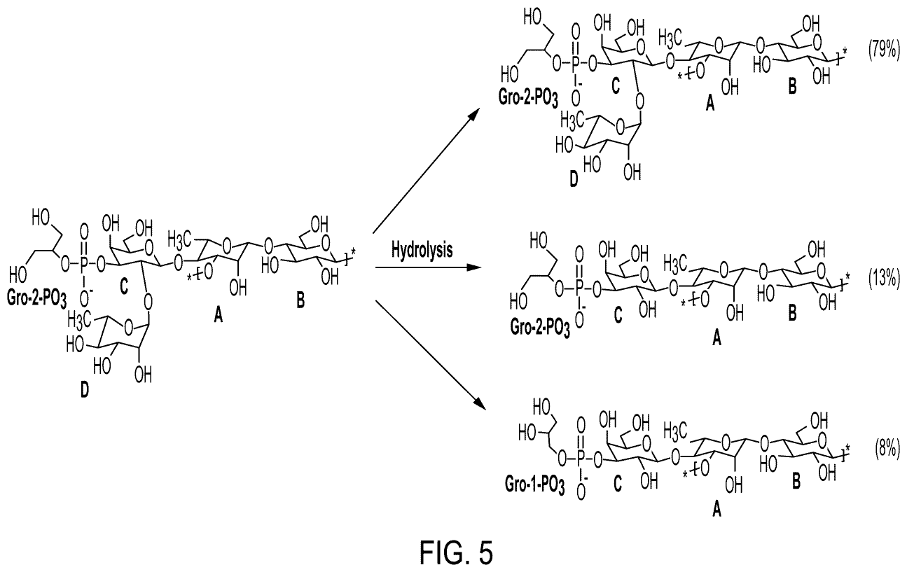

Complete structural analysis of the hydrolyzed Pn-23A polysaccharide suggest that about 79% of the Pn-23A polysaccharide is intact after 6 hrs of hydrolysis, whereas about 13% of Pn-23A polysaccharide loses the residue D (Rhamnose) sugar upon hydrolysis. Another about 8% of the population loses the Rhamnose sugar along with structural modification of the phospho-glycerol (see FIG. 5).

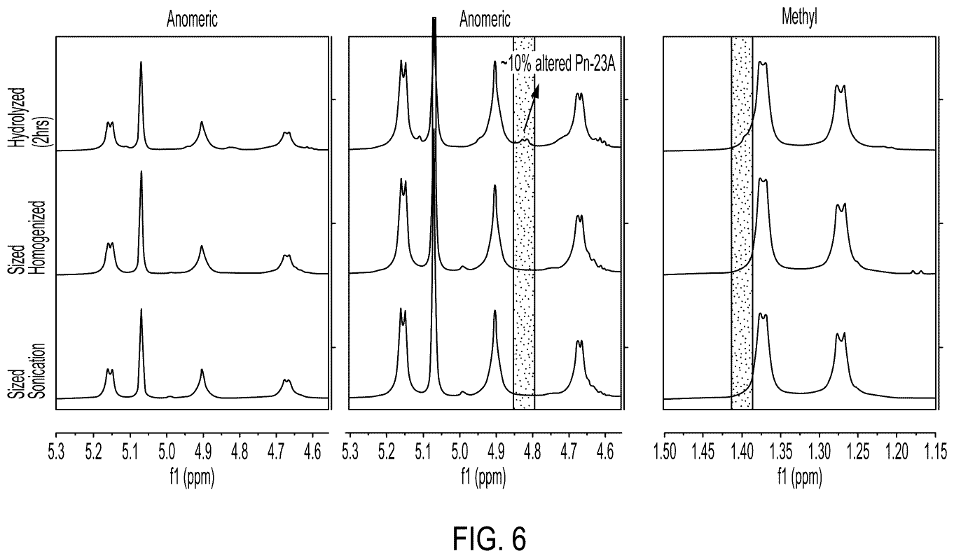

FIG. 6 shows the stack plot of anomeric and methyl regions of 1D ¹H spectra of sized via sonication (Sized Sonication), homogenization (Sized Homogenized) and hydrolysis (Hydrolized (2 hrs)) done under process condition using 0.1N acetic acid for 2 hrs at 80° C. Hydrolysis of Pn-23A polysaccharide under process condition generate about 10% of the hydrolyzed Pn-23A polysaccharide, where the Rhamnose sugar has been cleaved from the main chain, thus altering the structure of Pn-23A.

Activation of Pn-23A polysaccharide after sizing using acid hydrolysis leads to alteration of the polysaccharide structure and thus might have an impact on the immunogenicity of a conjugate obtained with this altered polysaccharide.

The mechanical sizing Pn-23A polysaccharide via sonication or homogenization results in no structural cleavage of the pendent Rhamnose sugar thus keeping the native polysaccharide structure intact.

### Example 7: Structural Characterization of Hydrolyzed Serotype 24F Polysaccharide Via NMR

Polysaccharide conjugates are generated by conjugating the antigen (polysaccharide) with the carrier protein. The size of the final conjugates depends on the size of the polysaccharide used. Generally, the polysaccharide molecular weight as extracted and purified from the bacteria is not suitable to generate conjugate vaccine. Therefore, the polysaccharides are sized before they are made ready to conjugate with the carrier protein.

Surprisingly, it was observed that sizing 24F polysaccharide via acid hydrolysis (as recommended for example in WO2019050815 or in WO2019050818) impacts the structure of the polysaccharide. The activation and conjugation of Pn-24F polysaccharide was not affected by the hydrolysis because primary activation site in Pn-24F was left intact upon hydrolysis. However, the structure of the polysaccharide is modified and thus might have an impact on the immunogenicity of a conjugate obtained with this altered polysaccharide.

Sample preparation: Native Pn-24F polysaccharide was incubated with 0.1N acetic acid at 80° C. for 2 hrs or with 0.2N Acetic acid, 90° C., 50 min. The acid hydrolyzed samples were dialyzed against water and the lyophilized. Hydrolyzed Pn-24F was weighed and dissolved in D₂O. The sample was bath sonicated for 20 minutes at 50° C. Final concentration of the sample was 25 mg/mL. The samples were transferred into the NMR tube for NMR data collection and analysis.

NMR Data collection: All data was collected at 75° C. using Bruker 600 MHz spectrometer equipped with TCI cryoprobe. The NMR data was processed using NvX and analyzed using NMRViewJ (NvJ). The following experiments were carried out to do the NMR analysis:
1) 1D ¹H (d1 5s; ns 32)
2) ¹H-¹³C HSQC (d1 1s; ns 4; TD1 256; SW 65 ppm@81 ppm)
3) 1D ³¹P (d1 1s; ns 32)
4) 2D ¹H-³¹P HMBC (d1 1s; ns 16; TD 64; SW 3@θppm)

The structure of the repeat unit of Pn-24F polysaccharide is shown at FIG. 7.

The molecular weight of the hydrolyzed Pn-24F was determined using SEC-MALLS. The native Pn-24F polysaccharide molecular weight was 790 kDa, whereas the two hydrolyzed Pn-24F polysaccharides studied have molecular weights of 72 KDa (hydrolysis in 0.2N Acetic acid, 90° C., 50 min) and 111 kDa (hydrolysis in 0.1N acetic acid at 80° C. for 2 hrs).

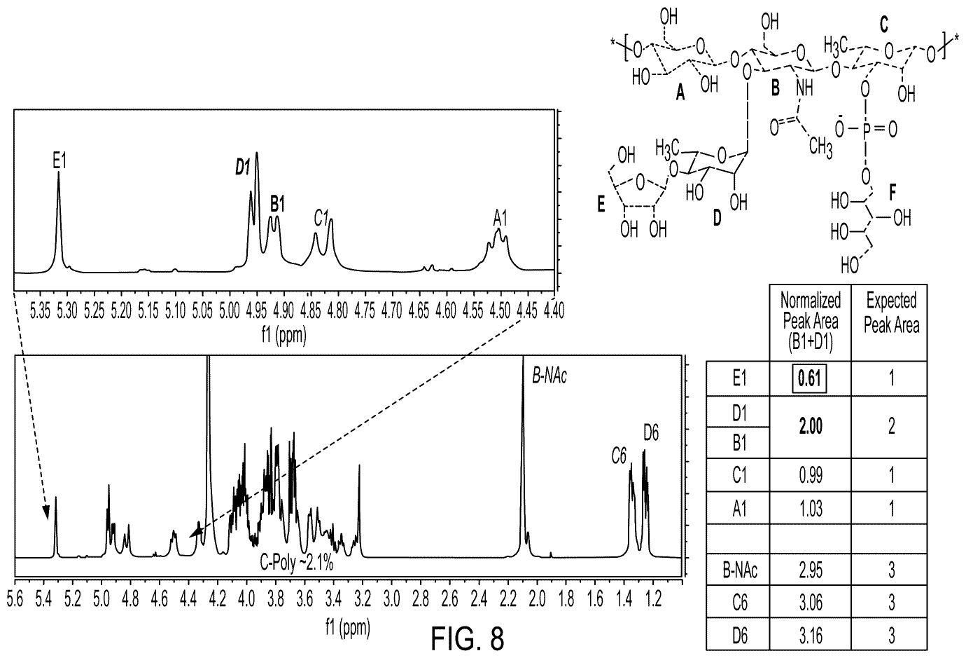

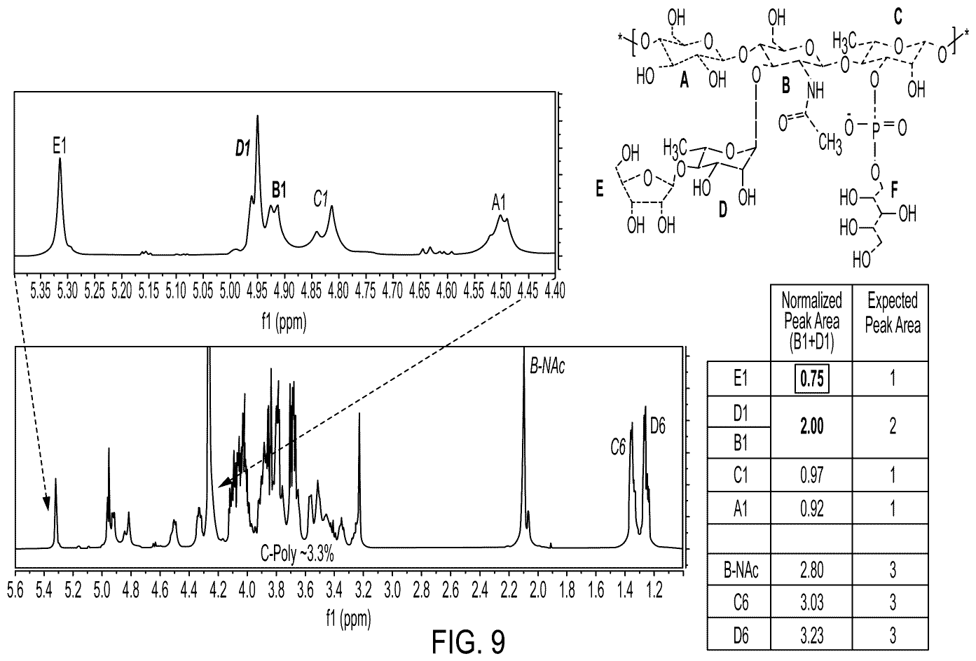

1D ¹H spectra of the hydrolyzed Pn-24F polysaccharides is provided at FIGS. 8 and 9. The insert tables in FIGS. 8 and 9 show the excellent agreement between the normalized area under the anomeric proton peaks and the expected theoretical area, except for the residue E1 suggesting a loss of Ribf sugar (highlighted in the insert Table).

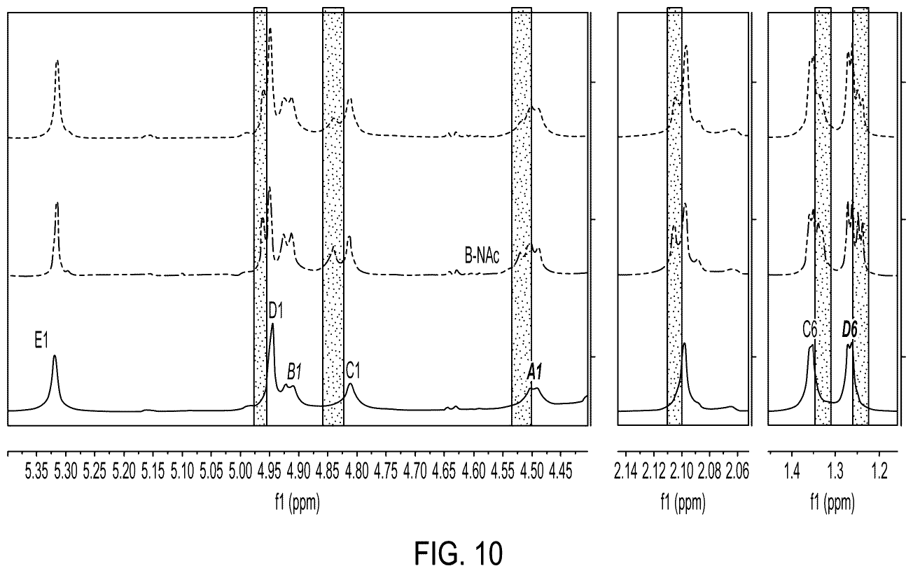

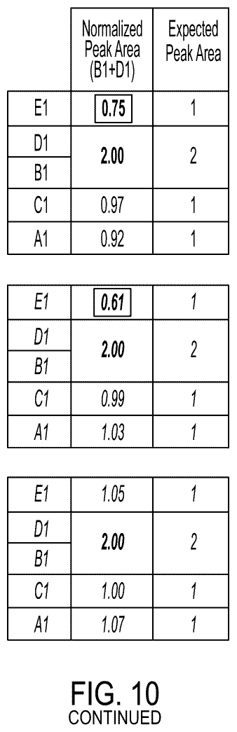

1D ¹H spectra of two lots of hydrolyzed Pn-24F polysaccharides (incubated with 0.1N acetic acid at 80° C. for 2 hrs (upper spectra) or with 0.2N Acetic acid, 90° C., 50 min (middle spectra)) is compared with the mechanically sized Pn-24 polysaccharide (sonication; lower spectra) (FIG. 10). The anomeric and some other signals are annotated. The grey bars denote the changes in the signal observed around anomeric, N-Acetyl and Rhamnose methyl signals. These differences in the signals are due to the structural changes seen in the polysaccharide upon hydrolysis. One of the significant changes observed is the decrease in the E1 anomeric signal intensity. The decrease in the E1 signal intensity (values 0.75 and 0.61 in the tables) suggest that upon hydrolysis the Ribf residue is being hydrolyzed.

Similarly, the overlaid 2D ¹H-¹³C HSQC spectra (not shown) of sized and hydrolyzed Pn-24F polysaccharides show changes in the anomeric, ring and methyl resonances. The new peaks upon hydrolysis are annotated on the spectra.

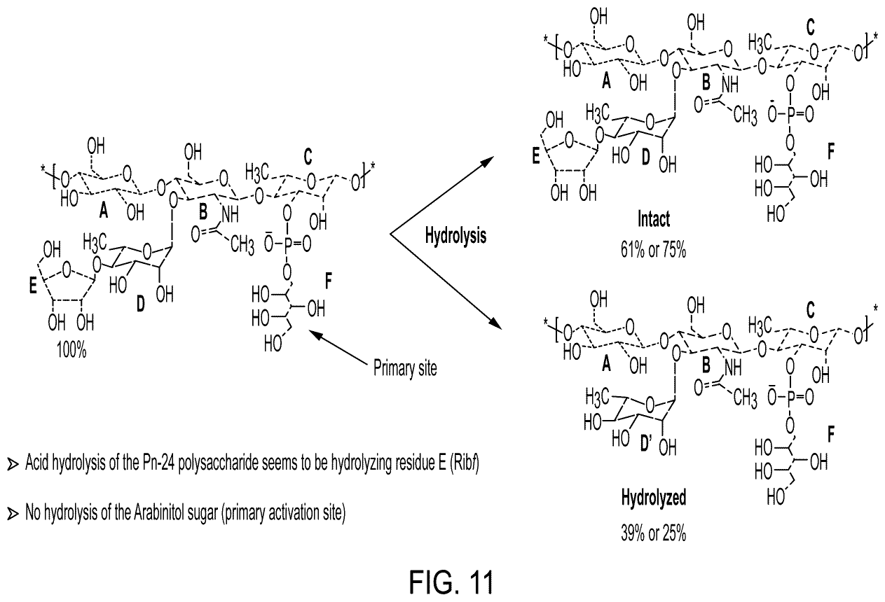

FIG. 11 shows the schematic of the structural changes seen upon hydrolysis of Pn-24F polysaccharide. From the NMR data it is quite evident that Ribf residue is being cleaved upon hydrolysis (in the two conditions tested, i.e. 0.1N acetic acid at 80° C. for 2 hrs or with 0.2N Acetic acid, 90° C., 50 min).

With 0.2N Acetic acid, 90° C., 50 min as hydrolysis conditions, 39% of the Ribf residue of the Pn-24 polysaccharide is hydrolyzed (only 61% of the repeating units remain intact), whereas 25% of polysaccharide is hydrolyzed using 0.1N acetic acid at 80° C. for 2 hrs (only 75% of the repeating units remain intact).

Acid hydrolysis of Pn-24F polysaccharide cleaves the Ribf sugar. About 25% and 39% of the Pn-24F polysaccharide has been found to have no Ribf sugar, whereas about 75% or 61% remain intact. There is no such a structural change when using mechanical sizing (sonication).

It was also observed that hydrolysis with 0.2N Acetic acid, 90° C., 50 min leads to a molecular weight reduced to about 72 kda, which is not conducive for conjugation. Milder hydrolysis conditions (0.1N acetic acid at 80° C. for 2 hrs) lead to a higher molecular weight but about a quarter of the Ribf sugar were still lost.

### Example 8: Formulation of a 25-Valent Pneumococcal Conjugate Vaccine

A 25 valent conjugates composition comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B (25vPnC) was formulated. All serotypes were individually conjugated to CRM₁₉₇ except serotype 3 which was conjugated to SCP with click chemistry.

S. pneumoniae glycoconjugates from serotypes 1, 4, 5, 6A, 6B, 7F, 9V, 14, 18C, 19A, 19F and 23F were produced as disclosed in WO 2006/110381, glycoconjugate from S. pneumoniae serotype 3 was produced as disclosed in WO2022/249106 (SCP with click chemistry) and glycoconjugates from S. pneumoniae from serotypes 8, 10A, 11A, 12F, 15B, 22F and 33F were produced as disclosed in WO2015/110941.

The required volumes of bulk concentrates were calculated based on the batch volume and the bulk saccharide concentrations. The preparation was filtered through a 0.2 μm Millipore PES membrane, followed by the addition of AIPO₄.
Form 1 consisted in 2.2 μg of each of glycoconjugates from S. pneumoniae serotypes 1, 3, 4, 5, 6A, 7F, 8, 9V, 10A, 11A, 12F, 14, 15A, 15B, 18C, 19A, 19F, 22F, 23A, 23B, 23F, 24F, 33F and 35B individually conjugated to CRM₁₉₇ (except serotype 3 which was conjugated to SCP), 4.4 μg of glycoconjugate from S. pneumoniae serotype 6B conjugated to CRM₁₉₇, 5 mM succinate buffer pH 5.8, 0.02% (w/v) polysorbate 80 (PS80), 150 mM NaCl and 0.25 mg/mL aluminum as AIPO4 for a dose of 0.5 mL. CRM₁₉₇, content was about 60 μg for a dose of 0.5 mL.
Form 2 consisted in 2.2 μg of each of glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B individually conjugated to CRM₁₉₇ (except serotype 3 which was conjugated to SCP), 4.4 μg of glycoconjugate from S. pneumoniae serotype 6B conjugated to CRM₁₉₇, 5 mM succinate buffer pH 5.8, 0.02% (w/v) PS80, 150 mM NaCl, 20 mM CaCl₂ and 0.25 mg/mL aluminum as AIPO4 for a dose of 0.5 mL. CRM₁₉₇, content was about 60 μg for a dose of 0.5 mL.
Form 3 consisted in 2.2 μg of each of glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B individually conjugated to CRM₁₉₇ (except serotype 3 which was conjugated to SCP), 4.4 μg of glycoconjugate from S. pneumoniae serotype 6B conjugated to CRM₁₉₇, 5 mM succinate buffer pH 5.8, 40 mM Sodium Phosphate, 0.02% (w/v) PS80, 245 mM NaCl and 0.25 mg/mL aluminum as AIPO₄ for a dose of 0.5 mL. CRM₁₉₇, content was about 60 μg for a dose of 0.5 mL.
Form 4 consisted in 2.2 μg of each of glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B individually conjugated to CRM₁₉₇ (except serotype 3 which was conjugated to SCP), 4.4 μg of glycoconjugate from S. pneumoniae serotype 6B conjugated to CRM₁₉₇, 30 mM Histidine pH 5.8, 0.02% (w/v) PS80, 245 mM NaCl and 0.25 mg/mL aluminum as AIPO4 for a dose of 0.5 mL. CRM₁₉₇, content was about 60 μg for a dose of 0.5 mL.

### Example 9: Immunogenicity of the 25-Valent Immunogenic Composition in Rats

The immunogenicity of 25-valent immunogenic compositions (see Example 8, S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B individually conjugated to CRM₁₉₇ (except serotype 3 which was conjugated to SCP)) was assessed in Rats using multiplexed direct Luminex immunoassays (dLIAs) to measure serotype-specific IgG concentrations in sera and serotype-specific OPAs.

All vaccine preparations were formulated to contain 0.44 μg for each pneumococcal conjugate including click-SCP serotype 3 (except 6B, at 0.88 μg/dose) with 62.5 μg AIPO₄ per 0.25 mL dose, in the matrix described at Example 8 (Form 1, Form 2, Form 3 and Form 4). Half these doses have also been tested (see Table 7 and 9)

Rats (8-10 weeks old; female Wistar han rats; 10 rats per group) were immunized subcutaneously (250 μL) at Weeks 0 and 3 and bled at Week 0 (Wk0) and 2 weeks following the second dose (Week 5 post vaccination (Wk5)). Weeks 0 and 5 sera were evaluated in dLIA and OPA.

To quantify the total polysaccharide binding antibody (IgG) specific to each pneumococcal polysaccharide (PnPS), rat sera were evaluated in three direct Luminex immunoassays (dLIAs; 13-plex dLIA, PREVNAR 13® serotypes, 7-plex dLIA, additional PREVNAR 20® serotypes, 5-plex dLIA for the 5 additional serotypes). The 13-plex assay measures anti-PnPS antibodies specific to the 13 serotypes included in the 13-valent pneumococcal conjugate (PnC) vaccine (1, 3, 4, 5, 6A, 6B, 7F, 9V, 14, 18C, 19A, 19F, and 23F) and the 7-plex assay measures anti-PnPS antibodies to the additional serotypes in PREVNAR 20® (8, 10A, 11A, 12F, 15B, 22F, 33F). The 5-plex assay measures anti-PnPS antibodies to additional serotypes (15A, 23A, 23B, 24F, and 35B). Each assay contains a combination of 13, or 7, or 5 spectrally distinct magnetic microspheres coupled to PnPS conjugates (PnPS-PLL conjugates: PnPS conjugated to poly-L-Lysine).

Briefly, reference standard, controls and test sera were first pre-adsorbed with two Pn absorbents; CWPS1 (cell wall polysaccharide from PnA containing C-polysaccharide) and CWPS2 (CWP from acapsular S. pneumoniae serotype 2) to block non-specific antibodies from binding to the PnPS coating antigen. Following reabsorption, the PnPS-coupled microspheres were incubated with appropriately diluted reference standard serum, controls or rat test sera. After incubation, each mixture was washed and an R-Phycoerythrin-conjugated goat anti-rat IgG secondary antibody was added. Fluorescent signals (expressed as median fluorescence intensities (MFIs)) were measured using a Bio-Plex reader and correlated to the amount of bound PnPS-specific IgG. Values for test sera are reported as (Units/mL, U/mL).

Table 6 below shows IgG titers of rats vaccinated with pneumococcal conjugate vaccine at a 0.44 μg/dose (except 6B, at 0.88 μg/dose).

TABLE 6

| | | Antigen | | | | |
|---|---|---|---|---|---|---|
| | | — | 25V | 25V | 25V | 25V |
| Serotype | Matrix | Form 1 | Form 1 | Form 2 | Form 3 | Form 4 |
| 1 | Wk0 GMC | 0.06 | 0.06 | 0.06 | 0.06 | 0.06 |
| | Wk5 GMC | 0.06 | 7.71 | 7.36 | 5.96 | 5.52 |
| | 95% CI | 0.06-0.06 | 3.96-15.00 | 2.33-23.27 | 1.92-18.52 | 1.90-16.05 |
| | GMC Ratio | 1 | 128 | 123 | 99 | 92 |
| 3 | Wk0 GMC | 0.17 | 0.13 | 0.13 | 0.13 | 0.13 |
| | Wk5 GMC | 0.17 | 736.66 | 720.98 | 195.25 | 626.50 |
| | 95% CI | 0.09-0.32 | 372.55-1456.63 | 310.29-1675.24 | 55.96-681.30 | 220.05-1783.74 |
| | GMC Ratio | 1 | 5667 | 5546 | 1502 | 4819 |
| 4 | Wk0 GMC | 0.06 | 0.06 | 0.06 | 0.06 | 0.06 |
| | Wk5 GMC | 0.06 | 74.27 | 47.72 | 24.75 | 49.02 |
| | 95% CI | 0.06-0.06 | 38.22-144.30 | 20.43-111.42 | 11.78-52.01 | 15.29-157.19 |
| | GMC Ratio | 1 | 1238 | 795 | 412 | 817 |
| 5 | Wk0 GMC | 0.06 | 0.06 | 0.06 | 0.06 | 0.06 |
| | Wk5 GMC | 0.06 | 9.81 | 17.82 | 3.64 | 3.89 |
| | 95% CI | 0.06-0.06 | 4.02-23.95 | 6.05-52.54 | 1.22-10.82 | 0.70-21.98 |
| | GMC Ratio | 1 | 163 | 297 | 61 | 65 |
| 6A | Wk0 GMC | 0.06 | 0.06 | 0.06 | 0.06 | 0.06 |
| | Wk5 GMC | 0.06 | 13.61 | 17.69 | 13.15 | 4.68 |
| | 95% CI | 0.06-0.06 | 4.91-37.73 | 7.50-41.68 | 5.72-30.21 | 0.73-30.49 |
| | GMC Ratio | 1 | 227 | 295 | 219 | 78 |
| 6B | Wk0 GMC | 0.06 | 0.06 | 0.06 | 0.06 | 0.06 |
| | Wk5 GMC | 0.06 | 67.21 | 44.57 | 63.20 | 8.75 |
| | 95% CI | 0.06-0.06 | 17.65-255.87 | 14.97-132.69 | 22.53-177.30 | 1.58-48.75 |
| | GMC Ratio | 1 | 1120 | 743 | 1053 | 146 |
| 7F | Wk0 GMC | 0.06 | 0.06 | 0.06 | 0.06 | 0.06 |
| | Wk5 GMC | 0.06 | 1.15 | 6.47 | 0.79 | 1.18 |
| | 95% CI | 0.06-0.06 | 0.37-3.56 | 1.63-25.72 | 0.15-4.19 | 0.43-3.22 |
| | GMC Ratio | 1 | 19 | 108 | 13 | 20 |
| 9V | Wk0 GMC | 0.06 | 0.06 | 0.06 | 0.06 | 0.06 |
| | Wk5 GMC | 0.06 | 115.31 | 87.92 | 82.45 | 37.29 |
| | 95% CI | 0.06-0.06 | 62.68-212.13 | 32.79-235.76 | 36.05-188.57 | 8.43-165.00 |
| | GMC Ratio | 1 | 1922 | 1465 | 1374 | 621 |
| 14 | Wk0 GMC | 0.06 | 0.06 | 0.06 | 0.06 | 0.06 |
| | Wk5 GMC | 0.06 | 0.13 | 0.16 | 0.09 | 0.14 |
| | 95% CI | 0.06-0.06 | 0.07-0.24 | 0.08-0.37 | 0.06-0.15 | 0.08-0.27 |
| | GMC Ratio | 1 | 2 | 3 | 2 | 2 |
| 18C | Wk0 GMC | 0.07 | 0.07 | 0.07 | 0.07 | 0.07 |
| | Wk5 GMC | 0.07 | 60.48 | 83.28 | 38.15 | 51.26 |
| | 95% CI | 0.07-0.07 | 24.10-151.78 | 39.94-173.63 | 19.62-74.18 | 25.60-102.68 |
| | GMC Ratio | 1 | 864 | 1190 | 545 | 732 |
| 19A | Wk0 GMC | 0.06 | 0.06 | 0.06 | 0.06 | 0.06 |
| | Wk5 GMC | 0.06 | 163.88 | 157.06 | 95.13 | 72.17 |
| | 95% CI | 0.06-0.06 | 104.61-256.73 | 100.36-245.79 | 59.48-152.13 | 30.78-169.24 |
| | GMC Ratio | 1 | 2731 | 2618 | 1585 | 1203 |
| 19F | Wk0 GMC | 0.07 | 0.07 | 0.07 | 0.07 | 0.07 |
| | Wk5 GMC | 0.07 | 96.09 | 161.23 | 116.91 | 95.13 |
| | 95% CI | 0.07-0.07 | 36.96-249.78 | 91.28-284.78 | 65.46-208.80 | 28.79-314.31 |
| | GMC Ratio | 1 | 1373 | 2303 | 1670 | 1359 |
| 23F | Wk0 GMC | 0.13 | 0.13 | 0.13 | 0.13 | 0.13 |
| | Wk5 GMC | 0.13 | 63.99 | 214.30 | 47.92 | 43.32 |
| | 95% CI | 0.13-0.13 | 15.52-263.79 | 46.24-993.19 | 18.30-125.47 | 20.25-92.66 |
| | GMC Ratio | 1 | 492 | 1648 | 369 | 333 |
| 8 | Wk0 GMC | 3.75 | 3.75 | 3.75 | 3.75 | 3.75 |
| | Wk5 GMC | 3.75 | 97.71 | 249.92 | 167.88 | 119.76 |
| | 95% CI | 3.75-3.75 | 38.52-247.88 | 139.94-446.34 | 99.56-283.07 | 59.89-239.49 |
| | GMC Ratio | 1 | 26 | 67 | 45 | 32 |
| 10A | Wk0 GMC | 4.28 | 3.76 | 3.76 | 3.76 | 3.76 |
| | Wk5 GMC | 4.26 | 14.53 | 73.00 | 11.50 | 34.87 |
| | 95% CI | 3.21-5.65 | 5.45-38.75 | 21.82-244.20 | 3.72-35.55 | 8.93-136.12 |
| | GMC Ratio | 1 | 4 | 19 | 3 | 9 |
| 11A | Wk0 GMC | 0.82 | 0.82 | 0.82 | 0.82 | 0.82 |
| | Wk5 GMC | 0.82 | 170.50 | 284.00 | 202.59 | 87.21 |
| | 95% CI | 0.82-0.82 | 94.02-309.19 | 130.38-618.61 | 115.52-355.27 | 39.29-193.61 |
| | GMC Ratio | 1 | 208 | 346 | 247 | 106 |
| 12F | Wk0 GMC | 1.00 | 1.00 | 1.00 | 1.00 | 1.00 |
| | Wk5 GMC | 1.00 | 13.72 | 12.58 | 4.50 | 17.08 |
| | 95% CI | 1.00-1.00 | 5.10-36.87 | 3.13-50.50 | 1.21-16.71 | 3.80-76.71 |
| | GMC Ratio | 1 | 14 | 13 | 4 | 17 |
| 15B | Wk0 GMC | 0.25 | 0.25 | 0.25 | 0.25 | 0.25 |
| | Wk5 GMC | 0.25 | 51.20 | 29.24 | 29.38 | 22.85 |
| | 95% CI | 0.25-0.25 | 30.34-86.39 | 13.99-61.09 | 13.04-66.19 | 8.96-58.28 |
| | GMC Ratio | 1 | 205 | 117 | 118 | 91 |
| 22F | Wk0 GMC | 0.92 | 0.92 | 0.92 | 0.92 | 0.92 |
| | Wk5 GMC | 0.92 | 518.64 | 560.03 | 426.95 | 316.80 |
| | 95% CI | 0.92-0.92 | 392.47-685.36 | 323.18-970.46 | 276.36-659.60 | 154.87-648.02 |
| | GMC Ratio | 1 | 564 | 609 | 464 | 344 |
| 33F | Wk0 GMC | 0.67 | 0.67 | 0.67 | 0.67 | 0.67 |
| | Wk5 GMC | 0.67 | 36.52 | 47.31 | 36.34 | 16.86 |
| | 95% CI | 0.67-0.67 | 17.48-76.29 | 18.06-123.94 | 13.12-100.63 | 7.25-39.21 |
| | GMC Ratio | 1 | 55 | 71 | 54 | 25 |
| 15A | Wk0 GMC | 0.03 | 0.03 | 0.05 | 0.04 | 0.03 |
| | Wk5 GMC | 0.06 | 8.94 | 8.34 | 1.73 | 2.73 |
| | 95% CI | 0.06-0.06 | 3.72-21.50 | 2.73-25.53 | 0.57-5.18 | 1.10-6.76 |
| | GMC Ratio | 2 | 329 | 184 | 41 | 107 |
| 23A | Wk0 GMC | 0.03 | 0.02 | 0.03 | 0.03 | 0.03 |
| | Wk5 GMC | 0.03 | 0.04 | 0.13 | 0.04 | 0.06 |
| | 95% CI | 0.03-0.03 | 0.02-0.07 | 0.02-0.75 | 0.02-0.09 | 0.02-0.19 |
| | GMC Ratio | 1 | 2 | 5 | 2 | 2 |
| 23B | Wk0 GMC | 0.01 | 0.01 | 0.01 | 0.01 | 0.01 |
| | Wk5 GMC | 0.01 | 8.44 | 19.27 | 7.02 | 1.81 |
| | 95% CI | 0.01-0.01 | 2.84-25.10 | 6.81-54.53 | 1.80-27.34 | 0.34-9.69 |
| | GMC Ratio | 1 | 993 | 2267 | 826 | 212 |
| 24F | WkO GMC | 0.03 | 0.03 | 0.03 | 0.03 | 0.03 |
| | Wk5 GMC | 0.03 | 0.12 | 0.36 | 0.05 | 0.05 |
| | 95% CI | 0.03-0.03 | 0.03-0.53 | 0.09-1.49 | 0.03-0.08 | 0.03-0.09 |
| | GMC Ratio | 1 | 5 | 14 | 2 | 2 |
| 35B | Wk0 GMC | 0.04 | 0.03 | 0.03 | 0.04 | 0.03 |
| | Wk5 GMC | 0.11 | 41.81 | 63.23 | 56.04 | 28.07 |
| | 95% CI | 0.03-0.39 | 19.39-90.17 | 41.97-95.25 | 34.97-89.83 | 9.26-85.06 |
| | GMC Ratio | 3 | 1491 | 2480 | 1597 | 1101 |

GMC: Geomean Concentration
95% CI: 95% Confidence Interval
GMC Ratio: Wk 5:Wk 0

Table 7 below shows IgG titers of rats vaccinated with pneumococcal conjugate vaccine at a 0.22 μg/dose (except 6B, at 0.44 μg/dose).

TABLE 7

| | | Antigen | | | | |
|---|---|---|---|---|---|---|
| | | — | 25V | 25V | 25V | 25V |
| Serotype | Matrix | Form 1 | Form 1 | Form 2 | Form 3 | Form 4 |
| 1 | Wk0 GMC | 0.06 | 0.06 | 0.06 | 0.06 | 0.06 |
| | Wk5 GMC | 0.06 | 10.77 | 13.52 | 5.93 | 12.21 |
| | 95% CI | 0.06-0.06 | 3.56-32.60 | 5.30-34.48 | 1.74-20.25 | 3.72-40.10 |
| | GMC Ratio | 1 | 179 | 225 | 99 | 203 |
| 3 | Wk0 GMC | 0.17 | 0.13 | 0.14 | 0.13 | 0.13 |
| | Wk5 GMC | 0.17 | 634.25 | 1314.56 | 476.17 | 323.14 |
| | 95% CI | 0.09-0.32 | 264.75-1519.47 | 576.77-2996.08 | 221.88-1021.92 | 87.92-1187.70 |
| | GMC Ratio | 1 | 4879 | 9358 | 3663 | 2486 |
| 4 | Wk0 GMC | 0.06 | 0.06 | 0.06 | 0.06 | 0.06 |
| | Wk5 GMC | 0.06 | 33.75 | 88.43 | 33.30 | 87.65 |
| | 95% CI | 0.06-0.06 | 11.16-102.08 | 32.91-237.64 | 14.29-77.59 | 24.90-308.51 |
| | GMC Ratio | 1 | 563 | 1474 | 555 | 1461 |
| 5 | Wk0 GMC | 0.06 | 0.06 | 0.06 | 0.06 | 0.06 |
| | Wk5 GMC | 0.06 | 9.16 | 26.76 | 6.73 | 11.61 |
| | 95% CI | 0.06-0.06 | 1.62-52.48 | 5.00-143.19 | 1.65-27.48 | 3.78-35.66 |
| | GMC Ratio | 1 | 153 | 446 | 112 | 193 |
| 6A | Wk0 GMC | 0.06 | 0.06 | 0.06 | 0.06 | 0.06 |
| | Wk5 GMC | 0.06 | 9.25 | 27.68 | 8.41 | 10.04 |
| | 95% CI | 0.06-0.06 | 2.43-35.22 | 10.46-73.25 | 3.29-21.47 | 2.30-44.26 |
| | GMC Ratio | 1 | 154 | 461 | 140 | 167 |
| 6B | Wk0 GMC | 0.06 | 0.06 | 0.06 | 0.06 | 0.06 |
| | Wk5 GMC | 0.06 | 53.07 | 28.21 | 22.55 | 45.72 |
| | 95% CI | 0.06-0.06 | 14.97-188.14 | 11.21-70.98 | 6.19-82.09 | 10.05-207.99 |
| | GMC Ratio | 1 | 884 | 470 | 376 | 762 |
| 7F | Wk0 GMC | 0.06 | 0.06 | 0.06 | 0.06 | 0.06 |
| | Wk5 GMC | 0.06 | 0.82 | 1.72 | 0.90 | 0.62 |
| | 95% CI | 0.06-0.06 | 0.21-3.10 | 0.33-8.91 | 0.31-2.61 | 0.19-2.04 |
| | GMC Ratio | 1 | 14 | 29 | 15 | 10 |
| 9V | Wk0 GMC | 0.06 | 0.06 | 0.06 | 0.06 | 0.06 |
| | Wk5 GMC | 0.06 | 113.14 | 79.07 | 62.79 | 79.26 |
| | 95% CI | 0.06-0.06 | 71.49-179.04 | 52.70-188.64 | 21.63-182.31 | 32.68-192.23 |
| | GMC Ratio | 1 | 1886 | 1318 | 1047 | 1321 |
| 14 | Wk0 GMC | 0.06 | 0.06 | 0.06 | 0.06 | 0.06 |
| | Wk5 GMC | 0.06 | 0.12 | 0.12 | 0.13 | 0.13 |
| | 95% CI | 0.06-0.06 | 0.08-0.20 | 0.09-0.18 | 0.09-0.18 | 0.10-0.18 |
| | GMC Ratio | 1 | 2 | 2 | 2 | 2 |
| 18C | Wk0 GMC | 0.07 | 0.07 | 0.07 | 0.07 | 0.07 |
| | Wk5 GMC | 0.07 | 94.90 | 65.96 | 31.12 | 21.46 |
| | 95% CI | 0.07-0.07 | 23.77-378.86 | 21.86-198.99 | 9.39-103.12 | 4.70-97.92 |
| | GMC Ratio | 1 | 1356 | 942 | 445 | 307 |
| 19A | Wk0 GMC | 0.06 | 0.06 | 0.06 | 0.06 | 0.06 |
| | Wk5 GMC | 0.06 | 192.00 | 268.25 | 100.83 | 88.00 |
| | 95% CI | 0.06-0.06 | 104.17-353.86 | 158.14-455.04 | 47.47-214.16 | 36.99-209.35 |
| | GMC Ratio | 1 | 3200 | 4471 | 1680 | 1467 |
| 19F | Wk0 GMC | 0.07 | 0.07 | 0.07 | 0.07 | 0.07 |
| | Wk5 GMC | 0.07 | 228.83 | 414.83 | 175.70 | 116.12 |
| | 95% CI | 0.07-0.07 | 95.19-550.12 | 218.52-787.49 | 117.75-262.17 | 47.93-281.34 |
| | GMC Ratio | 1 | 3269 | 5926 | 2510 | 1659 |
| 23F | Wk0 GMC | 0.13 | 0.13 | 0.13 | 0.13 | 0.13 |
| | Wk5 GMC | 0.13 | 125.24 | 142.59 | 27.45 | 51.80 |
| | 95% CI | 0.13-0.13 | 23.14-677.96 | 69.37-293.08 | 6.45-116.92 | 13.37-200.60 |
| | GMC Ratio | 1 | 963 | 1097 | 211 | 398 |
| 8 | Wk0 GMC | 3.75 | 3.75 | 3.75 | 3.75 | 3.75 |
| | Wk5 GMC | 3.75 | 220.26 | 360.03 | 77.03 | 209.84 |
| | 95% CI | 3.75-3.75 | 111.24-436.13 | 174.56-742.58 | 37.44-158.47 | 120.61-365.09 |
| | GMC Ratio | 1 | 59 | 96 | 21 | 56 |
| 10A | Wk0 GMC | 4.28 | 3.76 | 4.13 | 5.87 | 3.76 |
| | Wk5 GMC | 4.26 | 20.12 | 57.65 | 20.36 | 44.00 |
| | 95% CI | 3.21-5.65 | 6.43-63.00 | 11.92-278.92 | 6.51-63.63 | 21.31-90.85 |
| | GMC Ratio | 1 | 5 | 14 | 3 | 12 |
| 11A | Wk0 GMC | 0.82 | 0.82 | 0.82 | 0.82 | 0.82 |
| | Wk5 GMC | 0.82 | 118.74 | 283.52 | 74.22 | 156.01 |
| | 95% CI | 0.82-0.82 | 66.47-212.11 | 145.96-550.72 | 42.15-130.68 | 88.93-273.70 |
| | GMC Ratio | 1 | 145 | 346 | 91 | 190 |
| 12F | Wk0 GMC | 1.00 | 1.00 | 1.00 | 1.00 | 1.00 |
| | Wk5 GMC | 1.00 | 28.67 | 73.93 | 10.39 | 27.40 |
| | 95% CI | 1.00-1.00 | 6.01-136.65 | 19.77-276.47 | 2.74-39.35 | 7.35-102.15 |
| | GMC Ratio | 1 | 29 | 74 | 10 | 27 |
| 15B | Wk0 GMC | 0.25 | 0.25 | 0.25 | 0.25 | 0.25 |
| | Wk5 GMC | 0.25 | 26.21 | 83.16 | 22.25 | 32.32 |
| | 95% CI | 0.25-0.25 | 6.65-103.20 | 30.90-223.79 | 8.22-60.26 | 16.37-63.81 |
| | GMC Ratio | 1 | 105 | 333 | 89 | 129 |
| 22F | Wk0 GMC | 0.92 | 0.92 | 0.92 | 0.92 | 0.92 |
| | Wk5 GMC | 0.92 | 592.71 | 1000.79 | 331.24 | 537.25 |
| | 95% CI | 0.92-0.92 | 277.59-1265.55 | 514.28-1947.53 | 197.56-555.56 | 332.53-808.01 |
| | GMC Ratio | 1 | 644 | 1088 | 360 | 584 |
| 33F | Wk0 GMC | 0.67 | 0.67 | 0.67 | 0.67 | 0.67 |
| | Wk5 GMC | 0.67 | 21.54 | 38.49 | 15.34 | 26.61 |
| | 95% CI | 0.67-0.67 | 9.86-47.05 | 16.28-91.03 | 8.96-26.27 | 11.07-63.97 |
| | GMC Ratio | 1 | 32 | 57 | 23 | 40 |
| 15A | Wk0 GMC | 0.03 | 0.03 | 0.03 | 0.05 | 0.04 |
| | Wk5 GMC | 0.06 | 8.35 | 10.30 | 3.45 | 3.78 |
| | 95% CI | 0.06-0.06 | 2.53-27.52 | 1.73-61.15 | 0.84-14.25 | 1.23-11.57 |
| | GMC Ratio | 2 | 290 | 337 | 70 | 92 |
| 23A | Wk0 GMC | 0.03 | 0.02 | 0.03 | 0.03 | 0.03 |
| | Wk5 GMC | 0.03 | 0.17 | 0.12 | 0.05 | 0.08 |
| | 95% CI | 0.03-0.03 | 0.03-1.07 | 0.05-0.30 | 0.02-0.10 | 0.03-0.21 |
| | GMC Ratio | 1 | 8 | 5 | 2 | 3 |
| 23B | Wk0 GMC | 0.01 | 0.01 | 0.01 | 0.01 | 0.01 |
| | Wk5 GMC | 0.01 | 7.33 | 11.39 | 8.87 | 3.27 |
| | 95% CI | 0.01-0.01 | 2.65-20.22 | 1.12-117.62 | 1.67-47.13 | 0.96-10.75 |
| | GMC Ratio | 1 | 791 | 1340 | 1043 | 385 |
| 24F | Wk0 GMC | 0.03 | 0.03 | 0.03 | 0.03 | 0.03 |
| | Wk5 GMC | 0.03 | 0.49 | 0.64 | 0.14 | 0.32 |
| | 95% CI | 0.03-0.03 | 0.12-1.97 | 0.13-3.20 | 0.03-0.66 | 0.06-1.78 |
| | GMC Ratio | 1 | 19 | 25 | 6 | 12 |
| 35B | Wk0 GMC | 0.04 | 0.03 | 0.04 | 0.03 | 0.03 |
| | Wk5 GMC | 0.11 | 40.72 | 45.06 | 25.40 | 33.38 |
| | 95% CI | 0.03-0.39 | 16.58-100.00 | 24.28-83.63 | 9.69-66.55 | 12.44-89.53 |
| | GMC Ratio | 3 | 1597 | 1257 | 996 | 998 |

GMC: Geomean Concentration
95% CI: 95% Confidence Interval
GMC Ratio: Wk 5:Wk 0

OPA assays have been developed for S. pneumoniae serotypes present in the 25-valent vaccinea and several additional serotypes such as 15C, 29 and 35D. OPAs were performed for the analysis of the sera obtained from rats for select serotypes: 3, 6A, 6B, 11A, 15B, 19A, 19F, 15A, 23A, 23B, 24F, 35B, 15C, 29 and 35D. Heat-inactivated sera were serially diluted 2.5-fold in Hank's balanced saline solution supplemented with 0.1% gelatin. Target bacteria were added to assay plates and were incubated for 30 min at 25° C./37° C. on a shaker. Baby rabbit complement (3- to 4-week old, Pel-Freez, 12% final concentration) and differentiated HL-60 cells were then added to each well and the assay plates were incubated for 45 minutes at 37° C. with shaking. At the conclusion of the assay, aliquots of assay reaction mixture are plated onto microcolony filter plates, incubated at 37° C., 5% C02 overnight and the resulting colonies that were then stained with Coomassie Brilliant Blue stain. Colonies were imaged and enumerated on a Cellular Technology Limited (CTL) ImmunoSpot Analyzer®. Details of the assay conditions are serotype specific. The interpolated OPA antibody titer is the reciprocal of the dilution that yields a 50% reduction in the number of bacterial colonies when compared to the control wells that did not contain serum.

Table 8 below shows OPA GMT titers of rats vaccinated with pneumococcal conjugate vaccines at a 0.44 μg dose (except 6B, at 0.88 μg/dose).

TABLE 8

| | | Antigen | | | | |
|---|---|---|---|---|---|---|
| | | — | 25V | 25V | 25V | 25V |
| Serotype | Matrix | Form 1 | Form 1 | Form 2 | Form 3 | Form 4 |
| 3 | GMT | 5 | 702 | 771 | 57 | 206 |
| | 95% CI | 5-5 | 380-1294 | 234-2543 | 6-523 | 69-618 |
| | GMT Ratio | N/A | 140 | 154 | 11 | 41 |
| 6A | GMT | 7 | 2814 | 3178 | 996 | 544 |
| | 95% CI | 5-11 | 1334-5937 | 1745-5789 | 540-1836 | 158-1886 |
| | GMT Ratio | N/A | 389 | 439 | 138 | 75 |
| 6B | GMT | 5 | 1069 | 1654 | 875 | 242 |
| | 95% CI | 5-5 | 431-2652 | 969-2823 | 410-1870 | 74-794 |
| | GMT Ratio | N/A | 214 | 331 | 175 | 48 |
| 11A | GMT | 10689 | 21867 | 28097 | 22705 | 27708 |
| | 95% CI | 2568-44476 | 18195-26280 | 23656-33370 | 18334-28117 | 24214-31708 |
| | GMT Ratio | N/A | 2 | 3 | 2 | 3 |
| 15B | GMT | 6 | 19046 | 14718 | 8101 | 6171 |
| | 95% CI | 4-9 | 11668-31090 | 7951-27245 | 3979-16491 | 2060-18491 |
| | GMT Ratio | N/A | 3230 | 2496 | 1374 | 1047 |
| 19A | GMT | 216 | 989 | 1005 | 522 | 508 |
| | 95% CI | 83-564 | 638-1534 | 659-1532 | 420-649 | 341-756 |
| | GMT Ratio | N/A | 5 | 5 | 2 | 2 |
| 19F | GMT | 70 | 474 | 558 | 256 | 309 |
| | 95% CI | 36-138 | 240-935 | 388-802 | 150-438 | 172-555 |
| | GMT Ratio | N/A | 7 | 8 | 4 | 4 |
| 15A | GMT | 31 | 30884 | 15473 | 10116 | 9159 |
| | 95% CI | 20-48 | 24086-39601 | 6833-35038 | 4038-25343 | 2688-31204 |
| | GMT Ratio | N/A | 1009 | 506 | 331 | 299 |
| 23A | GMT | 5 | 324 | 238 | 448 | 96 |
| | 95% CI | 5-5 | 86-1229 | 54-1056 | 128-1570 | 20-456 |
| | GMT Ratio | N/A | 65 | 48 | 90 | 19 |
| 23B | GMT | 2107 | 2914 | 4306 | 2380 | 1988 |
| | 95% CI | 1729-2568 | 1907-4450 | 2958-6269 | 1732-3272 | 1543-2562 |
| | GMT Ratio | N/A | 1 | 2 | 1 | 1 |
| 24F | GMT | 23 | 365 | 836 | 116 | 135 |
| | 95% CI | 18-29 | 120-1105 | 216-3239 | 52-256 | 41-445 |
| | GMT Ratio | N/A | 16 | 36 | 5 | 6 |
| 35B | GMT | 88 | 16090 | 22842 | 20986 | 18590 |
| | 95% CI | 17-459 | 6466-40031 | 14872-35085 | 12941-34032 | 9676-35716 |
| | GMT Ratio | N/A | 183 | 259 | 238 | 211 |
| 15C | GMT | 6 | 15170 | 12002 | 8506 | 1584 |
| | 95% CI | 4-7 | 7348-31316 | 7064-20390 | 4438-16305 | 149-16838 |
| | GMT Ratio | N/A | 2755 | 2180 | 1545 | 288 |
| 29 | GMT | 6 | 1170 | 2720 | 1164 | 1093 |
| | 95% CI | 4-9 | 241-5682 | 1101-6721 | 563-2404 | 414-2882 |
| | GMT Ratio | N/A | 191 | 447 | 191 | 179 |
| 35D | GMT | 120 | 15951 | 30059 | 21421 | 21034 |
| | 95% CI | 29-503 | 6316-40286 | 24827-36394 | 15666-29291 | 13469-32847 |
| | GMT Ratio | N/A | 134 | 252 | 179 | 176 |

GMT: OPA Geomean Titers
95% CI: 95% Confidence Interval
GMT Ratio: Wk 5:Control
N/A: Not Applicable

Table 9 below shows OPA GMT titers of rats vaccinated with pneumococcal conjugate vaccines at a 0.22 μg/dose (except 6B, at 0.44 μg/dose).

TABLE 9

| | | Antigen | | | | |
|---|---|---|---|---|---|---|
| | | | 25V | 25V | 25V | 25V |
| Serotype | Matrix | Form 1 | Form 1 | Form 2 | Form 3 | Form 4 |
| 3 | GMT | 5 | 484 | 823 | 239 | 185 |
| | 95% CI | 5-5 | 204-1151 | 395-1711 | 120-475 | 47-728 |
| | GMT Ratio | N/A | 97 | 165 | 48 | 37 |
| 6A | GMT | 7 | 2033 | 2037 | 943 | 564 |
| | 95% CI | 5-11 | 1033-3999 | 1063-3903 | 469-1897 | 157-2027 |
| | GMT Ratio | N/A | 281 | 281 | 130 | 78 |
| 6B | GMT | 5 | 1234 | 1103 | 389 | 455 |
| | 95% CI | 5-5 | 468-3252 | 668-1821 | 157-965 | 112-1853 |
| | GMT Ratio | N/A | 247 | 221 | 78 | 91 |
| 11A | GMT | 10689 | 23541 | 24477 | 26428 | 21448 |
| | 95% CI | 2568-44476 | 19374-28605 | 17758-33739 | 20549-33989 | 16781-27415 |
| | GMT Ratio | N/A | 2 | 2 | 2 | 2 |
| 15B | GMT | 6 | 13520 | 18058 | 12740 | 8839 |
| | 95% CI | 4-9 | 6660-27447 | 10213-31931 | 7048-23028 | 3564-21924 |
| | GMT Ratio | N/A | 2293 | 3062 | 2161 | 1499 |
| 19A | GMT | 216 | 1394 | 1502 | 763 | 605 |
| | 95% CI | 83-564 | 723-2690 | 886-2544 | 470-1240 | 395-926 |
| | GMT Ratio | N/A | 6 | 7 | 4 | 3 |
| 19F | GMT | 70 | 714 | 800 | 436 | 389 |
| | 95% CI | 36-138 | 437-1164 | 471-1359 | 338-561 | 222-682 |
| | GMT Ratio | N/A | 10 | 11 | 6 | 6 |
| 15A | GMT | 31 | 20178 | 19730 | 18949 | 19019 |
| | 95% CI | 20-48 | 13351-30495 | 10840-35911 | 10816-33196 | 8666-41746 |
| | GMT Ratio | N/A | 659 | 645 | 619 | 621 |
| 23A | GMT | 5 | 493 | 1400 | 279 | 441 |
| | 95% CI | 5-5 | 96-2537 | 776-2527 | 67-1157 | 109-1776 |
| | GMT Ratio | N/A | 99 | 280 | 56 | 88 |
| 23B | GMT | 2107 | 3345 | 3806 | 2740 | 1636 |
| | 95% CI | 1729-2568 | 1916-5840 | 2664-5437 | 1724-4356 | 1198-2234 |
| | GMT Ratio | N/A | 2 | 2 | 1 | 1 |
| 24F | GMT | 23 | 852 | 761 | 276 | 352 |
| | 95% CI | 18-29 | 252-2883 | 262-2213 | 72-1059 | 114-1085 |
| | GMT Ratio | N/A | 37 | 33 | 12 | 15 |
| 35B | GMT | 88 | 24570 | 21388 | 21260 | 15289 |
| | 95% CI | 17-459 | 12786-47213 | 14980-30538 | 13114-34464 | 7939-29443 |
| | GMT Ratio | N/A | 279 | 243 | 241 | 174 |
| 15C | GMT | 6 | 10778 | 14907 | 10280 | 5289 |
| | 95% CI | 4-7 | 5010-23188 | 7688-28904 | 4323-24448 | 835-33518 |
| | GMT Ratio | N/A | 1958 | 2707 | 1867 | 961 |
| 29 | GMT | 6 | 1983 | 1996 | 3061 | 1208 |
| | 95% CI | 4-9 | 729-5392 | 965-4129 | 1866-5022 | 500-2918 |
| | GMT Ratio | N/A | 325 | 328 | 503 | 198 |
| 35D | GMT | 120 | 25472 | 23336 | 30055 | 17187 |
| | 95% CI | 29-503 | 14450-44900 | 17208-31647 | 23755-38026 | 9940-29716 |
| | GMT Ratio | N/A | 213 | 195 | 252 | 144 |

GMT: OPA Geomean Titers
95% CI: 95% Confidence Interval
GMT Ratio: Wk 5:Control
N/A: Not Applicable

There was an increase in serotype-specific IgG and OPA responses following 2 immunizations of rats with 25vPnC (Table 6). Serum IgG levels increased between 2 to 5546-fold above baseline based on the serotype and matrix. Pre immune sera (Week 0) had undetectable levels of PnPS-specific IgG for all the 25vPnC serotypes. Due to the limited sera available, OPA titers were tested only for a select number of serotypes (Table 8). Serum OPA titers increased between 2 to 2496-fold above titers measured in naïve rat sera, except for serotypes 11A and 23B, which had high background OPA titers in naïve animals. Overall, rats vaccinated with 25vPnC formulated with different matrices elicited antigen specific immune responses. 25vPnC formulated with different matrices when administered to rats, elicited a robust humoral response that was both specific for PnPS and associated with functional killing of the bacterium.

Rat sera was also analyzed for OPA titers for several non-vaccine serotypes. Pn35D, 29, and 15C OPA titers were high in all PCV25 vaccinated rats, indicating potential cross-reactivity.

### Example 10: Immunogenicity of the 25-Valent Immunogenic Composition in Mice

The immunogenicity of 25-valent immunogenic compositions (see Example 8, S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B individually conjugated to CRM₁₉₇ (except serotype 3 which was conjugated to SCP)) was assessed in mice using serotype-specific OPAs to measure immunogenicity.

All vaccine preparations were formulated to contain 0.11 μg for each pneumococcal conjugate including click-SCP serotype 3 (except 6B, at 0.22 μg/dose) with AIPO₄, in the matrix described at Example 8 (Form 1, Form 2, Form 3 and Form 4).

Groups of 25 mice were immunized via the subcutaneous route on week 0. The mice were boosted with the same dose of conjugate vaccine on week 3 and then bled at week 5. Serotype-specific OPAs were performed on week 5 sera samples.

OPAs were performed as explained above (see Example 9) for the analysis of the sera obtained from mice for select serotypes: 3, 6A, 6B, 9V, 11A, 15B, 19A, 19F.

Table 10 below shows OPA GMT titers of mice vaccinated with pneumococcal conjugate vaccines at a 0.11 μg/dose (except 6B, at 0.22 μg/dose).

TABLE 10

| | | Antigen | | | | |
|---|---|---|---|---|---|---|
| | | — | 25V | 25V | 25V | 25V |
| Serotype | Matrix | Form 1 | Form 1 | Form 2 | Form 3 | Form 4 |
| 3 | GMT | 12 | 2444 | 2548 | 1608 | 1965 |
| | 95% CI | 4-35 | 1522-3926 | 1693-3835 | 1135-2277 | 1256-3074 |
| 6A | GMT | 5 | 1473 | 1362 | 1496 | 1667 |
| | 95% CI | 5-5 | 517-4202 | 747-2485 | 1008-2219 | 1007-2759 |
| 6B | GMT | 5 | 270 | 102 | 226 | 122 |
| | 95% CI | 5-5 | 116-630 | 45-235 | 122-419 | 51-289 |
| 9V | GMT | 5 | 67 | 62 | 74 | 151 |
| | 95% CI | 5.5 | 35-129 | 29-128 | 41-133 | 81-282 |
| 11A | GMT | 262 | 3799 | 1983 | 1868 | 2029 |
| | 95% CI | 213-323 | 2528-5712 | 1327-2965 | 1310-2662 | 1313-3137 |
| 15B | GMT | 716 | 22781 | 4693 | 8050 | 9404 |
| | 95% CI | 447-1147 | 16901-30707 | 2934-7508 | 5395-12011 | 6305-14025 |
| 19A | GMT | 5 | 35 | 61 | 46 | 36 |
| | 95% CI | 5-5 | 18-71 | 29-132 | 23-93 | 16-77 |
| 19F | GMT | 5 | 89 | 93 | 162 | 151 |
| | 95% CI | 5.5 | 38-207 | 49-175 | 92-287 | 87-264 |

GMT: OPA Geomean
95% CI: 95% Confidence Interval

Table 11 below shows OPA GMT titers of mice vaccinated with pneumococcal conjugate vaccines at a 0.055 μg/dose (except 6B, at 0.11 μg/dose).

TABLE 11

| | | Antigen | | | | |
|---|---|---|---|---|---|---|
| | | — | 25V | 25V | 25V | 25V |
| Serotype | Matrix | Form 1 | Form 1 | Form 2 | Form 3 | Form 4 |
| 3 | GMT | 12 | 2945 | 2702 | 2184 | 1800 |
| | 95% CI | 4-35 | 2168-4001 | 1938-3766 | 1484-3215 | 1329-2437 |
| 6A | GMT | 5 | 742 | 845 | 1574 | 1082 |
| | 95% CI | 5-5 | 317-1738 | 424-1688 | 894-2774 | 659-1780 |
| 6B | GMT | 5 | 102 | 158 | 201 | 119 |
| | 95% CI | 5-5 | 42-244 | 77-326 | 92-440 | 51-276 |
| 9V | GMT | 5 | 77 | 57 | 97 | 81 |
| | 95% CI | 5.5 | 42-143 | 23-142 | 53-179 | 48-139 |
| 11A | GMT | 262 | 2323 | 1991 | 2056 | 2571 |
| | 95% CI | 213-323 | 1339-4028 | 1202-3298 | 1381-3061 | 1691-3909 |
| 15B | GMT | 716 | 12926 | 9813 | 7852 | 14229 |
| | 95% CI | 447-1147 | 9415-17747 | 6559-14680 | 4577-13473 | 9398-21545 |
| 19A | GMT | 5 | 63 | 39 | 39 | 48 |
| | 95% CI | 5-5 | 30-133 | 17-87 | 20-78 | 24-95 |
| 19F | GMT | 5 | 119 | 108 | 116 | 112 |
| | 95% CI | 5.5 | 57-249 | 48-245 | 69-195 | 60-209 |

GMT: OPA Geomean
95% CI: 95% Confidence Interval

### Example 11: Immunogenicity of the 25-Valent Immunogenic Composition in Infnat Rhesus Macaques (IRM)

Infant rhesus macaques (IRM) immunologically respond to conjugated but not unconjugated polysaccharides, and are a suitable model to predict serotype specific immune responses in human infants. Published studies also demonstrated that infant rhesus macaque OPA titers correlate with human infant OPA titers (Xie J et al., Pediatr Infect Dis J 2020; 39(1):70-7).

Age and sex matched IRM (3-6 months old) were randomly divided (Table 12).

TABLE 12

| | ST3 | Antigen | AlPO₄ | | Vaccina- |
|---|---|---|---|---|---|
| | Conjugate | Dose | Dose | Number | tion |
| Vaccine | Chemistry | (μg) | (μg) | IRM | Route |
| PCV25A | CRM-RAC | 2.2 (6B: 4.4 | 125 | 8 | IM |
| | | μg) | | | |
| PCV25B | SCP-Click | 2.2 (6B: 4.4 | 125 | 8 | IM |
| | | μg) | | | |

Infants were vaccinated intramuscular on both limbs with a dose of 0.25 mL/limb under sedation. IRM received 2.2 μg/dose of each conjugate (but for 6B at 4.4 μg/dose) of PCV25 which also contain 125 μg/dose of AIPO₄. The serotypes included in PCV25 vaccine are 1, 3, 4, 5, 6A, 6B, 7F, 8, 9V, 10A, 11A, 12F, 14, 15B, 18C, 19A, 19F, 22F, 23F, 33F, 15A, 23A, 23B, 24F, and 35B. Serotype 3 included were either a Click-SCP conjugate or a RAC/aqueous-CRM₁₉₇ conjugate (see WO2022/249106, RAC/aqueous for Reductive Amination in aqueous, Click for click chemistry). Pre-bleeds to assess baseline serotype-specific sera titers were collected 1 week (wk=−1) before primary vaccination (DO). Two repeat vaccinations were administered at week 8 and week 16 following primary vaccination. Whole blood for sera was collected at weeks 4 and 8 (post-dose 1; PD1); weeks 9, 12 and 16 (PD2) and weeks 17, 20, 32 and 52 (PD3).

Microcolony opsonophagocytic assays (mcOPA) were performed (see e.g. WO2022/249106). For the Pn3 mcOPA, reaction mixtures composed of target bacterial cells and heat-inactivated test serum are incubated for 30 minutes at 25° C. in an environmental shaker. Differentiated HL-60 tissue culture cells (effector cells) and baby rabbit complement are then added to the reaction mixture, and incubated for 45 minutes at 37° C. in an environmental shaker. Functional anti-S. pneumoniae antibody titers are determined by measuring bacterial survival in mcOPA reactions containing the test serum. The assay mixture is plated and grown overnight.

On day 2, the number of non-phagocytosed live bacteria is determined. The mcOPA antibody titer is the reciprocal of the serum dilution resulting in 50% reduction in the number of bacterial colonies when compared to the bacteria-effector cell-complement control wells that do not contain serum. Direct Luminex Immunoassays (dLIA) for Quantitation of Sera IgG were also conducted (see Example 9).

PCV25 with Click-SCP Conjugated serotype 3 (ST3) Induced Enhanced ST3-specific OPA/IgG Response Compared to PCV25 with RAC-CRM ST3

ST3 specific OPA titers were more than 10-fold higher (13.9 fold higher) in IRM vaccinated with PCV25/Click-SCP ST3 compared to IRM vaccinated with PCV25/RAC-CRM ST3 post dose 1 (Table 13, see GMT). ST3 specific IgG concentration were more than 20-fold higher (21 fold higher) in IRM vaccinated with PCV25/Click-SCP ST3 compared to IRM vaccinated with PCV25/RAC-CRM ST3 post dose 1 (Table 13, see GMC). and

TABLE 13

| | | GMC |
|---|---|---|
| Vaccine | GMT | (μg/ml) |
| PCV25A | 26 | 0.47 |
| PCV25B | 382 | 9.7 |

GMT: OPA Geomean titer PD1
GMC: Geomean Concentration (μg/ml) PD1

Following vaccination of IRM with PCV25/Click-SCP ST3 (PCV25B), serotype specific IgG response were observed for non ST3 vaccine serotypes post dose 1 which were boosted with additional doses as shown with post dose 3 data (Table 14).

TABLE 14

| | | | | | | | IgG |
|---|---|---|---|---|---|---|---|
| | | | | | | | Geo- |
| | Vaccines/Conjugate Dose | | | | | | mean |
| | PCV25B (Click/SCP-Pn3) | | | | | | Concen- |
| | 2.2 μg (6B-4.4 μg) | | | | | | tration |
| | PD1 week 4 | | | PD3 week 4 | | | Fold |
| | | Lower | Upper | | Lower | Upper | Increase |
| Sero- | | 95% | 95% | | 95% | 95% | (PD1: |
| type | GMC | CI | CI | GMC | CI | CI | PD3) |
| 1 | 0.130 | 0.043 | 0.420 | 0.900 | 0.270 | 3.100 | 6.92 |
| 3 | 9.700 | 4.700 | 20.000 | 6.400 | 3.400 | 12.000 | 0.66 |
| 4 | 0.240 | 0.130 | 0.460 | 1.600 | 0.500 | 5.300 | 6.67 |
| 5 | 0.024 | 0.007 | 0.083 | 0.100 | 0.020 | 0.540 | 4.17 |
| 6A | 0.010 | 0.004 | 0.029 | 0.440 | 0.071 | 2.800 | 44.00 |
| 6B | 0.014 | 0.007 | 0.027 | 0.310 | 0.060 | 1.700 | 22.14 |
| 7F | 1.700 | 0.490 | 5.700 | 1.600 | 0.530 | 4.800 | 0.94 |
| 9V | 0.016 | 0.007 | 0.039 | 0.680 | 0.200 | 2.300 | 42.50 |
| 14 | 0.009 | 0.004 | 0.024 | 0.300 | 0.068 | 1.300 | 32.26 |
| 18C | 1.000 | 0.310 | 3.500 | 4.500 | 2.100 | 9.600 | 4.50 |
| 19A | 0.043 | 0.018 | 0.100 | 0.420 | 0.180 | 1.000 | 9.77 |
| 19F | 0.120 | 0.023 | 0.590 | 1.000 | 0.400 | 2.700 | 8.33 |
| 23F | 0.006 | 0.004 | 0.008 | 0.130 | 0.032 | 0.520 | 23.21 |
| 8 | 0.630 | 0.330 | 1.200 | 0.530 | 0.270 | 1.000 | 0.84 |
| 10A | 1.400 | 0.760 | 2.600 | 2.700 | 1.400 | 5.000 | 1.93 |
| 11A | 4.000 | 2.000 | 7.800 | 2.200 | 1.000 | 4.800 | 0.55 |
| 12F | 0.012 | 0.004 | 0.040 | 0.310 | 0.082 | 1.100 | 25.83 |
| 15B | 1.300 | 0.330 | 4.800 | 3.400 | 1.200 | 9.400 | 2.62 |
| 22F | 1.200 | 0.320 | 4.800 | 3.300 | 1.100 | 9.900 | 2.75 |
| 33F | 0.140 | 0.058 | 0.340 | 0.570 | 0.230 | 1.400 | 4.07 |
| 15A | 0.041 | 0.011 | 0.150 | 0.160 | 0.037 | 0.710 | 3.90 |
| 23A | 0.020 | 0.007 | 0.059 | 0.033 | 0.007 | 0.160 | 1.65 |
| 23B | 0.033 | 0.008 | 0.130 | 0.100 | 0.021 | 0.470 | 3.03 |
| 24F | 0.020 | 0.007 | 0.059 | 0.056 | 0.030 | 0.100 | 2.80 |
| 35B | 0.460 | 0.210 | 0.990 | 1.300 | 0.790 | 2.100 | 2.83 |

Overall data suggest that compared to PCV25/RAC-CRM ST3, PCV25/Click-SCP ST3 substantially improved ST3 specific OPA/IgG response (>10 fold) while generating serotype specific response to other serotypes included in PCV25. The data suggest that PCV25 with Click-SCP ST3 can enhance protection against ST3 mediated IPD as compared to previous vaccine formulation, while broadening serotype coverage to emerging non-vaccine serotypes.

Although the foregoing invention has been described in some detail by way of illustration and example for purposes of clarity of understanding, certain changes and modifications may be practiced within the scope of the appended claims.

1. An immunogenic composition comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F and additionally comprises from 1 to 5 glycoconjugates from S. pneumoniae serotypes 15A, 23A, 23B, 24F and/or 35B.

2. The immunogenic composition of claim 1 which is a 21, 22, 23, 24 or 25-valent pneumococcal conjugate composition.

3. The immunogenic composition of claim 1 comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

4. The immunogenic composition of claim 3 which is a 25-valent pneumococcal conjugate composition.

5. The immunogenic composition of claim 1 wherein:
said S. pneumoniae serotype 15A glycoconjugate comprises a serotype 15A capsular polysaccharide having a weight average molecular weight (Mw) between 75 kDa and 250 kDa and/or;
said S. pneumoniae serotype 15A glycoconjugate has a weight average molecular weight (Mw) of between 1,000 kDa and 6,000 kDa and/or;
the serotype 15A polysaccharide to carrier protein ratio (w/w) in the S. pneumoniae serotype 15A glycoconjugate is between 0.5 and 1.5 and/or;
the degree of conjugation of said S. pneumoniae serotype 15A glycoconjugate is between 5 and 10 and/or;
said S. pneumoniae serotype 15A glycoconjugate comprises less than about 25% of free serotype 15A polysaccharide compared to the total amount of serotype 15A polysaccharide and/or;
between 50% and 90% of said serotype 15A glycoconjugate has a Kd below or equal to 0.3 in a CL-4B column.

6. The immunogenic composition of claim 1 wherein the carrier protein of said S. pneumoniae serotype 15A glycoconjugate is CRM₁₉₇.

7. The immunogenic composition of claim 1 wherein said S. pneumoniae serotype 15A glycoconjugate is prepared using reductive amination chemistry.

8. The immunogenic composition of claim 1 wherein:
said S. pneumoniae serotype 23A glycoconjugate comprises a serotype 23A capsular polysaccharide having a weight average molecular weight (Mw) between 110 kDa and 250 kDa and/or;
said S. pneumoniae serotype 23A glycoconjugate has a weight average molecular weight (Mw) of between 1,000 kDa and 7,500 kDa and/or;
the serotype 23A polysaccharide to carrier protein ratio (w/w) in the S. pneumoniae serotype 23A glycoconjugate is between 0.5 and 1.7 and/or;
the degree of conjugation of said S. pneumoniae serotype 23A glycoconjugate is between 5 and 15 and/or;
said S. pneumoniae serotype 23A glycoconjugate comprises less than about 25% of free serotype 23A polysaccharide compared to the total amount of serotype 23A polysaccharide and/or;
between 50% and 90% of said serotype 23A glycoconjugate has a Kd below or equal to 0.3 in a CL-4B column and/or;
said S. pneumoniae serotype 23A glycoconjugate comprises a S. pneumoniae serotype 23A capsular polysaccharide wherein said S. pneumoniae serotype 23A capsular polysaccharide has a branched Rhamnose content greater than 95% when compared to native S. pneumoniae serotype 23A capsular polysaccharide wherein the branched Rhamnose content is considered to be about 100%.

9. The immunogenic composition of claim 1 wherein the carrier protein of said S. pneumoniae serotype 23A glycoconjugate is CRM₁₉₇.

10. The immunogenic composition of claim 1 wherein said S. pneumoniae serotype 23A glycoconjugate is prepared using reductive amination chemistry.

11. The immunogenic composition of claim 1 wherein:
said S. pneumoniae serotype 23B glycoconjugate comprises a serotype 23B capsular polysaccharide having a weight average molecular weight (Mw) between 75 kDa and 250 kDa.
said S. pneumoniae serotype 23B glycoconjugate has a weight average molecular weight (Mw) of between 500 kDa and 4,000 kDa and/or;
the serotype 23B polysaccharide to carrier protein ratio (w/w) in the S. pneumoniae serotype 23B glycoconjugate is between 0.5 and 1.5 and/or;
the degree of conjugation of said S. pneumoniae serotype 23B glycoconjugate is between 5 and 12 and/or;
said S. pneumoniae serotype 23B glycoconjugate comprises less than about 25% of free serotype 23B polysaccharide compared to the total amount of serotype 23B polysaccharide and/or;
between 40% and 60% of said serotype 23B glycoconjugate has a Kd below or equal to 0.3 in a CL-4B column.

12. The immunogenic composition of claim 1 wherein the carrier protein of said S. pneumoniae serotype 23B glycoconjugate is CRM₁₉₇.

13. The immunogenic composition of claim 1 wherein said S. pneumoniae serotype 23B glycoconjugate is prepared using reductive amination chemistry.

14. The immunogenic composition of claim 1 wherein:
said S. pneumoniae serotype 24F glycoconjugate comprises a serotype 24F capsular polysaccharide having a weight average molecular weight (Mw) between 120 kDa and 250 kDa and/or;
said S. pneumoniae serotype 24F glycoconjugate has a weight average molecular weight (Mw) of between 1,500 kDa and 7,500 kDa and/or;
the serotype 24F polysaccharide to carrier protein ratio (w/w) in the S. pneumoniae serotype 24F glycoconjugate is between 0.7 and 1.5 and/or;
the degree of conjugation of said S. pneumoniae serotype 24F glycoconjugate is between 5 and 12 and/or;
said S. pneumoniae serotype 24F glycoconjugate comprises less than about 25% of free serotype 24F polysaccharide compared to the total amount of serotype 24F polysaccharide and/or;
between 50% and 90% of said serotype 24F glycoconjugate has a Kd below or equal to 0.3 in a CL-4B column and/or;
said S. pneumoniae serotype 24F glycoconjugate comprises a S. pneumoniae serotype 24F capsular polysaccharide wherein said S. pneumoniae serotype 24F capsular polysaccharide has a Ribose content greater than 95% when compared to native S. pneumoniae serotype 24F capsular polysaccharide wherein the Ribose content is considered to be about 100%.

15. The immunogenic composition of claim 1 wherein the carrier protein of said S. pneumoniae serotype 24F glycoconjugate is CRM₁₉₇.

16. The immunogenic composition of claim 1 wherein said S. pneumoniae serotype 24F glycoconjugate is prepared using reductive amination chemistry.

17. The immunogenic composition of claim 1 wherein:
said S. pneumoniae serotype 35B glycoconjugate comprises a serotype 35B capsular polysaccharide having a weight average molecular weight (Mw) between 25 kDa and 50 kDa and/or;
said S. pneumoniae serotype 35B glycoconjugate has a weight average molecular weight (Mw) of between 500 kDa and 5,000 kDa and/or;
the serotype 35B polysaccharide to carrier protein ratio (w/w) in the S. pneumoniae serotype 35B glycoconjugate is between 0.4 and 2.0 and/or;
the degree of conjugation of said S. pneumoniae serotype 35B glycoconjugate is between 5 and 10 and/or;
S. pneumoniae serotype 35B glycoconjugate comprises less than about 20% of free serotype 35B polysaccharide compared to the total amount of serotype 35B polysaccharide and/or;
between 50% and 80% of said serotype 35B glycoconjugate has a Kd below or equal to 0.3 in a CL-4B column.

18. The immunogenic composition of claim 1 wherein the carrier protein of said S. pneumoniae serotype 35B glycoconjugate is CRM₁₉₇.

19. The immunogenic composition of claim 1 wherein said S. pneumoniae serotype 35B glycoconjugate is prepared using reductive amination chemistry.

20. The immunogenic composition of claim 1 wherein all the glycoconjugates are individually conjugated to the carrier protein.

21. The immunogenic composition of claim 1 wherein the glycoconjugate from S. pneumoniae serotype 3 is conjugated to CRM₁₉₇.

22. The immunogenic composition of claim 1 wherein the glycoconjugate from S. pneumoniae serotype 3 is conjugated to SCP.

23. The immunogenic composition of claim 1 wherein the glycoconjugates from S. pneumoniae serotype 8, 10A, 11A, 12F, 15B, 22F, 33F are conjugated to CRM₁₉₇.

24. The immunogenic composition of claim 1 wherein the glycoconjugates from S. pneumoniae serotypes 1, 4, 5, 6A, 6B, 7F, 9V, 14, 18C, 19A, 19F and 23F are conjugated to CRM₁₉₇.

25. (canceled)

26. The immunogenic composition of claim 1 wherein the glycoconjugate from S. pneumoniae serotype 3 is conjugated to SCP and the other glycoconjugates are all individually conjugated to CRM₁₉₇.

27. The immunogenic composition of claim 1 wherein said glycoconjugate from S. pneumoniae serotype 3 comprises a serotype 3 saccharide covalently conjugated to a carrier protein (CP) through a spacer and have the general formula (VII): 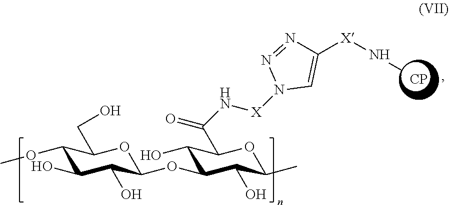
wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4,
and wherein X is selected from the group consisting of CH₂O(CH₂)_{n″}CH₂C═O, CH₂O(CH₂CH₂O)_{m′}(CH₂)_{n″}CH₂C═O, where n″ is selected from 0 to 10 and m′ is selected from 0 to 4.


SEQUENCE LISTING (2 sequences)

SEQ ID NO: 1 - protein, 950 aa, synthetic construct
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 2 - protein, 949 aa, synthetic construct
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ